(19) United States
(12) Patent Application Publication
(10) Pub. No.: US 2024/0366608 A1
(43) Pub. Date: Nov. 7, 2024

(54) PHARMACEUTICAL COMBINATIONS OF SOS1 INHIBITORS FOR TREATING AND/OR PREVENTING CANCER

(71) Applicant: Lupin Limited, Mumbai (IN)

(72) Inventors: Mandar Ramesh Bhonde, Pune (IN); Sukanya Patra, Hyderabad (IN); Venkata P. Palle, Pune (IN); Rajender Kumar Kamboj, Pune (IN)

(73) Assignee: Lupin Limited, Mumbai (IN)

(21) Appl. No.: 18/262,155

(22) PCT Filed: Jan. 19, 2022
(86) PCT No.: PCT/IB2022/050415

§ 371 Date: Jul. 19, 2023

(30) Foreign Application Priority Data
Jan. 19, 2021 (IN) 202121002487

(51) Int. Cl. A61K 31/519; A61K 31/415; A61K 31/4439; A61K 31/496; A61K 31/506; A61K 31/517; A61K 31/5377; A61K 31/7068; A61P 35/00

(52) U.S. Cl. CPC A61K 31/519; A61K 31/415; A61K 31/4439; A61K 31/496; A61K 31/506; A61K 31/517; A61K 31/5377; A61K 31/7068; A61P 35/00

(57) ABSTRACT

This disclosure relates to pharmaceutical combinations for treating and/or preventing cancer and methods and uses thereof. More particularly, provided is a pharmaceutical combination comprising a SOS1 Inhibitor and an additional active ingredient selected from a KRAS inhibitor such as a KRAS G12C inhibitor and a KRASG12D inhibitor, KRAS G13C inhibitor, and panKRAS inhibitor; an EGFR inhibitor; an ERK1/2 inhibitor; a BRAF inhibitor; a pan-RAF inhibitor; a MEK inhibitor; a AKT inhibitor; a SHP2 inhibitor; protein arginine methyltransferases (PRMTs) inhibitor such as a PRMT5 inhibitor and Type 1 PRMT inhibitor; a PI3K inhibitor; a cyclin-dependent kinase (CDK) inhibitor such as CDK4/6 inhibitor; a FGFR inhibitor; a c-Met inhibitor; a RTK inhibitor; a non-receptor tyrosine kinase inhibitor; a histone methyltransferases (HMTs) inhibitor; a DNA methyltransferases (DNMTs) inhibitor; a Focal Adhesion Kinase (FAK) inhibitor; a Bcr-Abl tyrosine kinase inhibitor; a mTOR inhibitor; a PD1 inhibitor; a PD-L1 inhibitor; CTLA4 inhibitor; and chemotherapeutic agents such as gemcitabine, doxorubicin, cisplatin, carboplatin, paclitaxel, docetaxel, topotecan, irinotecan and temozolomide.

### CROSS-REFERENCE TO RELATED APPLICATIONS

This PCT application claims priority in and to Indian Provisional Patent Application No. 202121002487 filed Jan. 19, 2021, the contents of which are hereby incorporated by reference herein in their entirety.

### FIELD OF INVENTION

The present invention relates to a pharmaceutical combination comprising a SOS1 inhibitor and an additional active ingredient selected from a KRAS inhibitor such as a KRAS G12C inhibitor and a KRASG12D inhibitor, KRAS G13C inhibitor, and panKRAS inhibitor; an EGFR inhibitor; an ERK1/2 inhibitor; a BRAF inhibitor; a pan-RAF inhibitor; a MEK inhibitor; a AKT inhibitor; a SHP2 inhibitor; protein arginine methyltransferases (PRMTs) inhibitor such as a PRMT5 inhibitor and Type 1 PRMT inhibitor; a PI3K inhibitor; a cyclin-dependent kinase (CDK) inhibitor such as CDK4/6 inhibitor; a FGFR inhibitor; a c-Met inhibitor; a RTK inhibitor; a non-receptor tyrosine kinase inhibitor; a histone methyltransferases (HMTs) inhibitor; a DNA methyltransferases (DNMTs) inhibitor; a Focal Adhesion Kinase (FAK) inhibitor; a Bcr-Abl tyrosine kinase inhibitor; a mTOR inhibitor; a PD1 inhibitor; a PD-L1 inhibitor; CTLA4 inhibitor; and chemotherapeutic agents such as gemcitabine, doxorubicin, cisplatin, carboplatin, paclitaxel, docetaxel, topotecan, irinotecan and temozolomide; wherein, the SOS1 inhibitor is selected from compound of formula (I) or compound of formula (II),

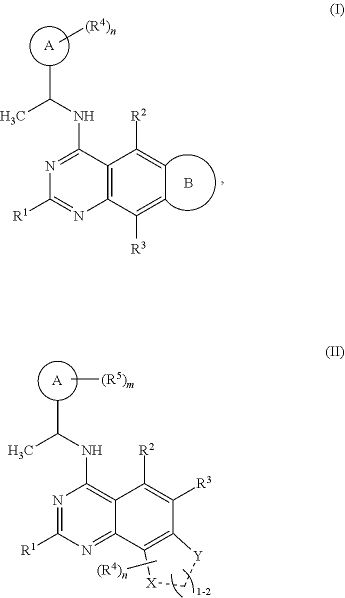

their tautomeric form, their stereoisomers, their pharmaceutically acceptable salt, their polymorph, or solvate thereof, for use in the treatment and/or prevention of cancer.

The present invention also relates to the treatment and/or prevention of cancer using a pharmaceutical combination as described hereinabove.

### BACKGROUND OF THE INVENTION

Multiple signaling pathways control the initiation, progression, spread, metastasis, immune evasion of cancer. Key signaling pathways include RTK/RAS pathway, PI3K pathway, Wnt pathway, Myc pathway and the cell cycle pathway (Francisco Sanchez-Vega et al., Cell, 2018, 173(2):321-337.e10). RAS-family proteins (KRAS, HRAS and NRAs and their respective mutants) are small GTPases that exist in cells in either GTP-bound (active) or GDP-bound (inactive) states (Siqi Li et al., Nat. Rev. Cancer, 2018, 18(12):767-777). The activity of RAS proteins is modulated by proteins known as GTPase Activating Proteins (GAPs) or Guanine Nucleotide Exchange Factors (GEFs). The GAP proteins belonging to the RAS family include members such as NF1, TSC2, IQGAP1, etc. which activate the GTPase function of the RAS proteins and thus terminate the signaling by catalyzing the hydrolysis of GTP to GDP. In contrast, the RAS family GEFs include proteins such as SOS1, SOS2, RASGRP, RASGRF2, etc. which activate the RAS proteins by exchanging GTP for GDP (Biochim Biophys Acta Rev Cancer. 2020, 1874(2):188445; Johannes L. Bos et al., Cell, 2007, 129(5):865-77). SOS proteins has been implicated in the regulation of RAS in multiple cancers, with more impetus on the role of targeting SOS1 for cancer therapy.

Ras-GTP binds to effector proteins such as Raf and PI3K which in turn leads to activation of the RAF-MEK-ERK (MAPK) and PI3K-mTOR-AKT (PI3K) signaling pathways (Suzanne Schubbert et al., Nat. Rev. Cancer, 2007, 7(4):295-308). Triggering of one or more of these cellular signaling pathways leads to the initiation and maintenance of the oncogenic phenotype involving enhanced cell proliferation, increased cell survival, altered metabolism, angiogenesis, migratory potential and immune evasion eventually leading to establishment and metastasis of cancers (Yousef Ahmed Fouad et al., Am. J. Cancer Res., 2017 7(5):1016-1036; Douglas Hanahan et al., Cell, 2011, 144(5):646-74). RAS proteins undergo point mutations at several amino acid residues—the key hot spots being positions G12, G13 and Q61. These mutations render the RAS proteins constitutively active since the proteins are predominantly in the active GTP-bound form (Ian A. Prior et al., Cancer Res., 2012, 72(10): 2457-2467; Adrienne D. Cox, et al., Nat. Rev. Drug. Discov., 2014, 13(11):828-51). Interaction of RAS proteins with GEFs such as Son of Sevenless 1 (SOS1) plays a crucial role in relaying the signals to downstream effectors. The SOS1 protein harbors several domains such as the Db1 homology domain (DH), a Pleckstrin homology domain (PH), RAS exchanger motif (REM), CDC25 homology domain and a C-terminal proline rich domain (PxxP) (Pradeep Bandaru et al., Cold Spring Harb Perspect Med., 2019, 9(2). pii:a031 534). SOS1 has been shown to have a catalytic site as well as an allosteric site. The catalytic site is preferentially bound by RAS-GDP whereas RAS-GTP binds with the allosteric site with better affinity than RAS-GDP (S. Mariana Margarit et al., Cell, 2003, 112(5):685-95; Hao-Hsuan Jeng et al., Nat. Commun., 2012; 3:1168). Furthermore, binding of oncogenic KRAS to SOS1 promotes the activation of wild type HRAS and NRAS (Hao-Hsuan Jeng et al., Nat. Commun., 2012; 3:1168). The catalytic (guanine nucleotide exchange) function of SOS1 is critical for KRAS oncogenic activity in cancer cells (You X et al., Blood. 2018, 132(24):2575-2579; Erin Sheffels et al., Sci Signal. 2018, 11(546). pii: eaar8371). SOS1 plays a key role in signal transmission following cellular activation by Receptor Tyrosine Kinases (RTKs) (Frank McCormick et al., Nature, 1993, 363(6424):45-51; Stephane Pierre et al., Biochem Pharmacol. 2011 82(9):1049-56). Additionally, SOS1 is required for function of receptors on lymphocytes (B cell and T cell receptor) (Mateusz Poltorak et al., Eur J Immunol. 2014, 44(5):1535-40; Stephen R. Brooks et al., J Immunol. 2000, 164(6):3123-31) and hematopoietic cells (Mario N. Lioubin et al., Mol Cell Biol., 1994, 14(9):5682-91).

The role of SOS1 in the RAS-mediated signaling pathways make it an attractive target for cancer therapy. Pharmacological intervention with SOS1 inhibitors has been shown to attenuate or eliminate the downstream effector events of the RAS-mediated pathways (Roman C. Hillig et al., Proc. Natl. Acad. Sci. USA. 2019, 116(7):2551-2560; Chris R. Evelyn et al., J Biol Chem., 2015, 290(20):12879-98).

Furthermore, alterations in SOS1 have been implicated in cancer. SOS1 mutations are found in embryonal rhabdomyosarcomas, sertoli cell testis tumors, granular cell tumors of the skin (Denayer et al. Genes Chromosomes Cancer, 2010, 49(3):242-52) and lung adenocarcinoma (Cancer Genome Atlas Research Network., Nature. 2014,511 (7511):543-50). Meanwhile over-expression of SOS1 has been described in bladder cancer (Watanabe at al. IUBMB Life., 2000, 49(4):317-20) and prostate cancer (Timofeeva et al. Int. J. Oncol., 2009, 35(4):751-60). In addition to cancer, hereditary SOS1 mutations are implicated in the pathogenesis of RASopathies like e.g. Noonan syndrome (NS), cardio-facio-cutaneous syndrome (CFC), hereditary gingival fibromatosis type I Noonan Syndrome with Multiple Lentigines (NSML) (LEOPARD syndrome), Capillary Malformation-Arteriovenous Malformation Syndrome (CM-AVM), Costello Syndrome (CS), Legius Syndrome (NF1-like Syndrome) (Pierre et al., Biochem. Pharmacol., 2011, 82(9):1049-56).

Pharmaceutical combinations of SOS1 inhibitors are disclosed in WO2018115380, WO2020254451, WO2021259972, Marco H H. et al., Cancer Discov. 2021, 11(1):142-157

### SUMMARY OF THE INVENTION

The invention described and claimed herein has many attributes and aspects, including but not limited to, those set forth or described or referenced in this summary. It is not intended to be all-inclusive and the invention described and claimed herein are not limited to or by features or embodiments identified in this summary, which is included for purposes of illustration only and not restriction.

In consideration above problems, in accordance with the one aspect disclosed herein, the present invention relates to a pharmaceutical combination comprising a SOS1 inhibitor and at least one additional active ingredient selected from a KRAS inhibitor such as a KRAS G12C inhibitor and a KRASG12D inhibitor, KRAS G13C inhibitor, and panKRAS inhibitor; an EGFR inhibitor; an ERK1/2 inhibitor; a BRAF inhibitor; a pan-RAF inhibitor; a MEK inhibitor; a AKT inhibitor; a SHP2 inhibitor; protein arginine methyltransferases (PRMTs) inhibitor such as a PRMT5 inhibitor and Type 1 PRMT inhibitor; a PI3K inhibitor; a cyclin-dependent kinase (CDK) inhibitor such as CDK4/6 inhibitor; a FGFR inhibitor; a c-Met inhibitor; a RTK inhibitor; a non-receptor tyrosine kinase inhibitor; a histone methyltransferases (HMTs) inhibitor; a DNA methyltransferases (DNMTs) inhibitor; a Focal Adhesion Kinase (FAK) inhibitor; a Bcr-Abl tyrosine kinase inhibitor; a mTOR inhibitor; a PD1 inhibitor; a PD-L1 inhibitor; CTLA4 inhibitor; and chemotherapeutic agents such as gemcitabine, doxorubicin, cisplatin, carboplatin, paclitaxel, docetaxel, topotecan, irinotecan and temozolomide; wherein the SOS1 inhibitor is selected from compound of formula (I) or formula (II),

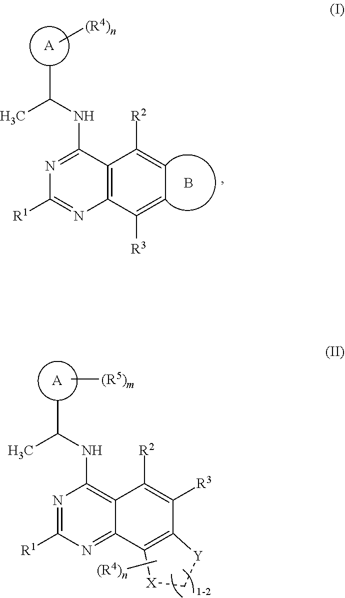

their tautomeric form, their stereoisomer, their pharmaceutically acceptable salt, their polymorph, or solvate thereof, for use in the treatment and/or prevention of cancer.

R¹, R², R³, R⁴, R⁵, Ring A, Ring B, m, n, X, Y are described herein below respectively for each compound.

In accordance with another aspect disclosed herein, the SOS1 inhibitor compound is administered simultaneously, concurrently, sequentially, successively, alternately, or separately with the at least one additional active ingredient.

In accordance with yet another aspect disclosed herein, a method of treating and/or preventing cancer, wherein the method comprises administering to the subject in need the pharmaceutical combination of any one of the pharmaceutical combinations disclosed herein.

In accordance with other aspect disclosed herein, the cancer is selected from glioblastoma multiforme, prostate cancer, pancreatic cancer, mantle cell lymphoma, non-Hodgkin's lymphomas and diffuse large B-cell lymphoma, acute myeloid leukemia, chronic myelogenous leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, head and neck cancer, cervical cancer, cancers harboring overexpression, amplification of wild type KRAS, NRAS or HRAS, cancers having amplification, overexpression or mutation of KRAS, NRAS, or HRAS, cancers harboring KRAS mutations such as G12C, G12D, G12V, G12S, G12A, G12R, G12F, G12W, G13C, G13D, G13R, G13V, G13S, G13A, Q61H, Q61R, Q61P, Q61E, Q61K, Q61L, A59S, A59T, R68M, R68S, Q99L, M72I, H95D, H95Q, H95R, Y96D, Y96S, Y96C, cancers harboring NRAS mutations such G12A, G12V, G12D, G12C, G12S, G12R, G13V, G13D, G13R, G13S, G13C, G13A, Q61K, Q61L, Q61H, Q61P, Q61R, A146T, A146V, cancers harboring HRAS mutations such as G12C, G12V, G12S, G12A, G12R, G12F, G12D, G13C, G13D, G13R, G13V, G13S, G13A, Q61K, Q61L, Q61H, Q61P, Q61R.

### BRIEF DESCRIPTION OF FIGURES

The drawing form part of the present specification and are included to further demonstrate certain aspects of embodiments described herein. These embodiments may be better understood by reference to one or more of the following drawings in combination with detailed description.

FIG. 1 shows the in vitro inhibition effect of a representative combination of the invention, Compound 4b with KRAS G12C inhibitor AMG510, in MIA PaCa-2 cells.

FIG. 2 shows the in vitro inhibition effect of a representative combination of the invention, Compound 4b with EGFR inhibitor Afatinib, in MIA PaCa-2 cells.

FIG. 3 shows the in vitro inhibition effect of a representative combination of the invention, Compound 4b with ERK1/2 inhibitor LY3214996, in MIA PaCa-2 cells.

FIG. 4 shows the in vitro inhibition effect of a representative combination of the invention, Compound 4b with ERK1/2 inhibitor BVD-523, in MIA PaCa-2 cells.

FIG. 5 shows the in vitro inhibition effect of a representative combination of the invention, Compound 4b with RAF inhibitor Encorafenib, in MIA PaCa-2 cells.

FIG. 6 shows the in vitro inhibition effect of a representative combination of the invention, Compound 4b with PRMT5 inhibitor Compound 24 of WO 2019116302, in MIA PaCa-2 cells.

FIG. 7 shows the in vitro inhibition effect of a representative combination of the invention, Compound 1 with EGFR inhibitor Afatinib, in MIA PaCa-2 cells.

FIG. 8 shows the in vitro inhibition effect of a representative combination of the invention, Compound 1 with KRAS G12C inhibitor AMG510, in MIA PaCa-2 cells.

FIG. 9 shows the in vitro inhibition effect of a representative combination of the invention, Compound 1 with ERK1/2 inhibitor LY3214996, in MIA PaCa-2 cells.

FIG. 10 shows the in vitro inhibition effect of a representative combination of the invention, Compound 1 with ERK1/2 inhibitor BVD-523, in MIA PaCa-2 cells.

FIG. 11 shows the in vitro inhibition effect of a representative combination of the invention, Compound 1 with RAF inhibitor Encorafenib, in MIA PaCa-2 cells.

FIG. 12 shows the in vitro inhibition effect of a representative combination of the invention, Compound 1 with PRMT5 inhibitor Compound 24 of WO 2019116302, in MIA PaCa-2 cells.

FIG. 13 shows the in vitro inhibition effect of a representative combination of the invention, Compound 6a with EGFR inhibitor Afatinib, in MIA PaCa-2 cells.

FIG. 14 shows the in vitro inhibition effect of a representative combination of the invention, Compound 6a with KRAS G12C inhibitor AMG510, in MIA PaCa-2 cells.

FIG. 15 shows the in vitro inhibition effect of a representative combination of the invention, Compound 6a with ERK1/2 inhibitor LY3214996, in MIA PaCa-2 cells.

FIG. 16 shows the in vitro inhibition effect of a representative combination of the invention, Compound 6a with ERK1/2 inhibitor BVD-523, in MIA PaCa-2 cells.

FIG. 17 shows the in vitro inhibition effect of a representative combination of the invention, Compound 5 with ERK1/2 inhibitor BVD-523, in MIA PaCa-2 cells.

FIG. 18 shows the in vitro inhibition effect of a representative combination of the invention, Compound 5 with ERK1/2 inhibitor LY3214996, in MIA PaCa-2 cells.

FIG. 19 shows the in vitro inhibition effect of a representative combination of the invention, Compound 5 with EGFR inhibitor Afatinib, in MIA PaCa-2 cells.

FIG. 20 shows the in vitro inhibition effect of a representative combination of the invention, Compound 5 with KRAS G12C inhibitor AMG510, in MIA PaCa-2 cells.

FIG. 21 shows the in vitro inhibition effect of a representative combination of the invention, Compound 5 with Compound 24 of WO 2019116302, in MIA PaCa-2 cells.

FIG. 22 shows the in vitro inhibition effect of a representative combination of the invention, Compound 2 with KRAS G12C inhibitor AMG510, in MIA PaCa-2 cells.

FIG. 23 shows the in vitro inhibition effect of a representative combination of the invention, Compound 3a with KRAS G12C inhibitor AMG510, in MIA PaCa-2 cells.

FIG. 24 shows the in vitro inhibition effect of a representative combination of the invention, Compound 3a with ERK1/2 inhibitor LY3214996, in MIA PaCa-2 cells.

FIG. 25 shows the in vitro inhibition effect of a representative combination of the invention, Compound 3a with RAF inhibitor Encorafenib, in MIA PaCa-2 cells.

FIG. 26 shows the in vitro inhibition effect of a representative combination of the invention, Compound 4b with pan-RAF inhibitor LXH254, in MIA PaCa-2 cells.

FIG. 27 shows the in vitro inhibition effect of a representative combination of the invention, Compound 4b with SHP2 inhibitor TNO155, in MIA PaCa-2 cells.

FIG. 28 shows the in vitro inhibition effect of a representative combination of the invention, Compound 7 with KRAS G12C inhibitor AMG510, in MIA PaCa-2 cells.

FIG. 29 shows the in vitro inhibition effect of a representative combination of the invention, Compound 7 with EGFR inhibitor Afatinib, in MIA PaCa-2 cells.

FIG. 30 shows the in vitro inhibition effect of a representative combination of the invention, Compound 7 with ERK1/2 inhibitor LY3214996, in MIA PaCa-2 cells.

FIG. 31 shows the in vitro inhibition effect of a representative combination of the invention, Compound 7 with ERK1/2 inhibitor BVD-523, in MIA PaCa-2 cells.

FIG. 32 shows the in vitro inhibition effect of a representative combination of the invention, Compound 7 with RAF inhibitor Encorafenib, in MIA PaCa-2 cells.

FIG. 33 shows the in vitro inhibition effect of a representative combination of the invention, Compound 7 with pan-RAF inhibitor LXH254, in MIA PaCa-2 cells.

FIG. 34 shows the in vitro inhibition effect of a representative combination of the invention, Compound 7 with SHP2 inhibitor TNO155, in MIA PaCa-2 cells.

FIG. 35 shows the in vitro inhibition effect of a representative combination of the invention, Compound 7 with KRAS G12C inhibitor MRTX849, in MIA PaCa-2 cells.

FIG. 36 shows the in vitro inhibition effect of a representative combination of the invention, Compound 7 with PI3K inhibitor BYL-719 in MIA PaCa-2 cells.

FIG. 37 shows the in vitro inhibition effect of a representative combination of the invention, Compound 7 with Type I PRMT inhibitor GSK3368715 in MIA PaCa-2 cells.

FIG. 38 shows the in vitro inhibition effect of a representative combination of the invention, Compound 7 with FGFR inhibitor Nintedanib in MIA PaCa-2 cells.

FIG. 39 shows the in vitro inhibition effect of a representative combination of the invention, Compound 7 with CDK4/6 inhibitor Abemaciclib in MIA PaCa-2 cells.

FIG. 40 shows the in vitro inhibition effect of a representative combination of the invention, Compound 7 with MRTX1133 in SW-1990 cells.

FIG. 41 shows the in vitro inhibition effect of a representative combination of the invention, Compound 7 with Gemcitabine in MIA PaCa-2 cells.

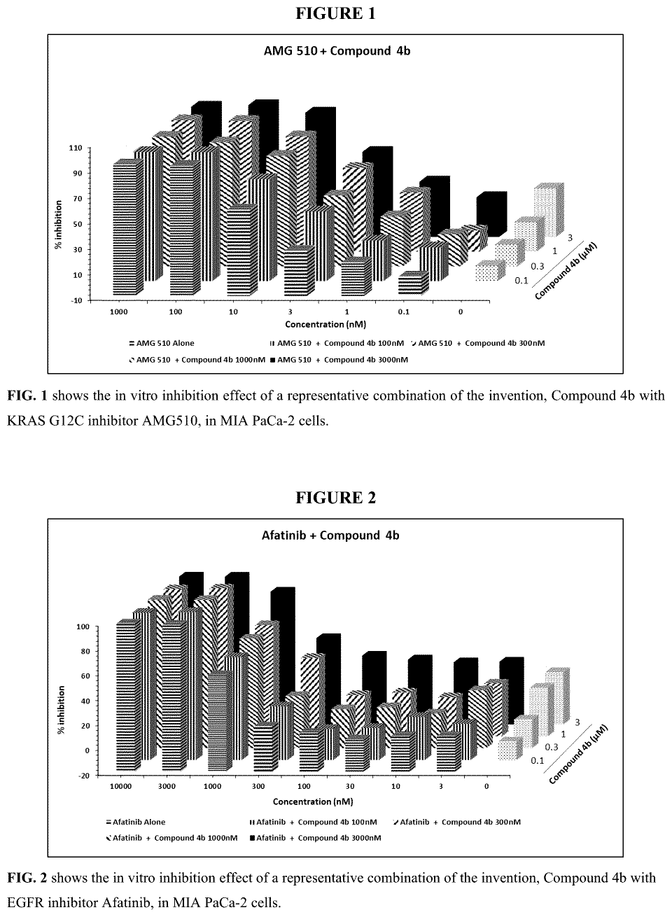

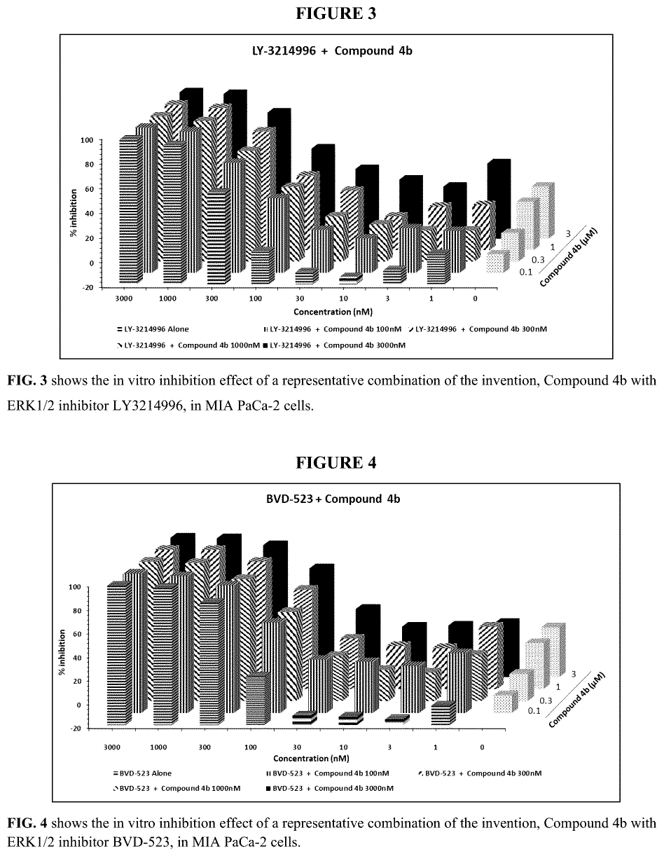

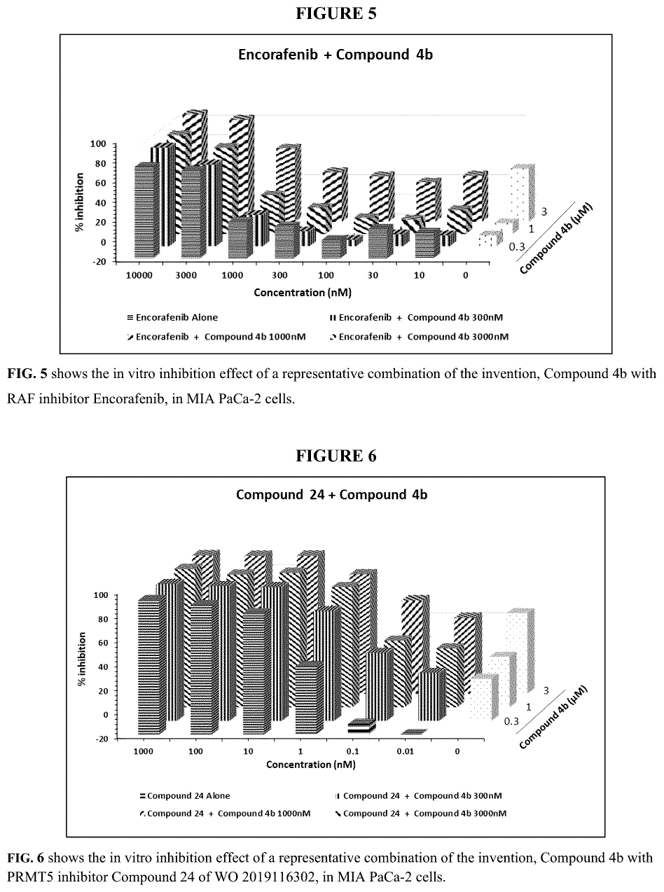

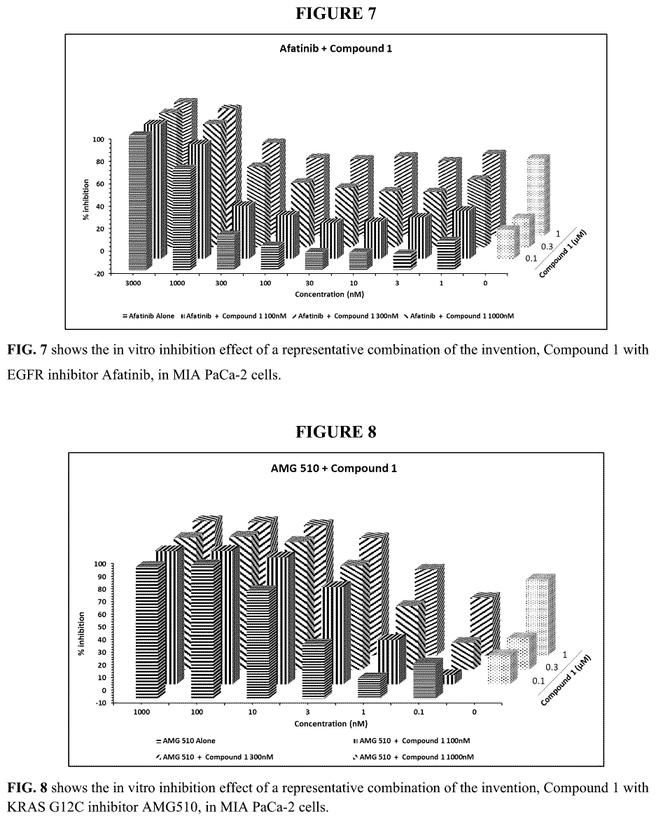

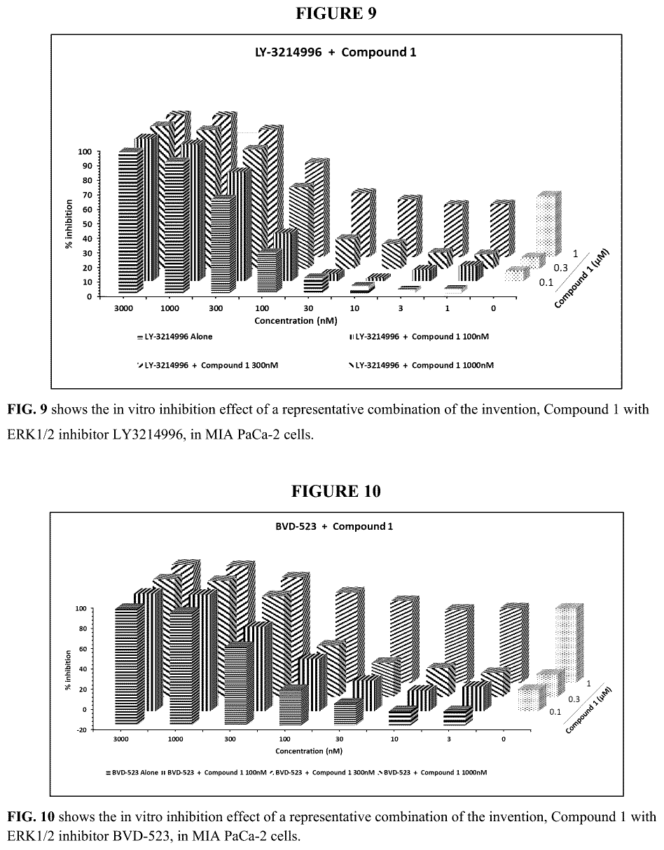

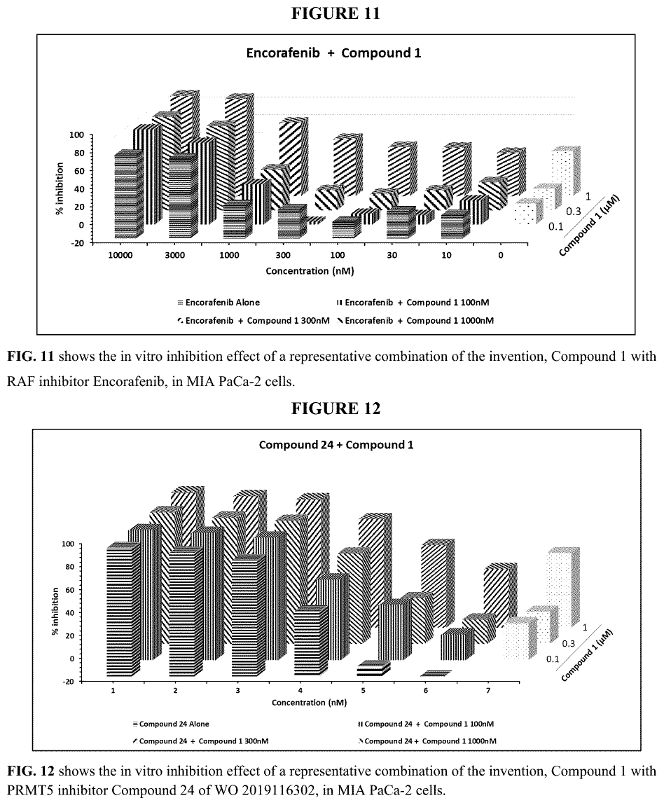

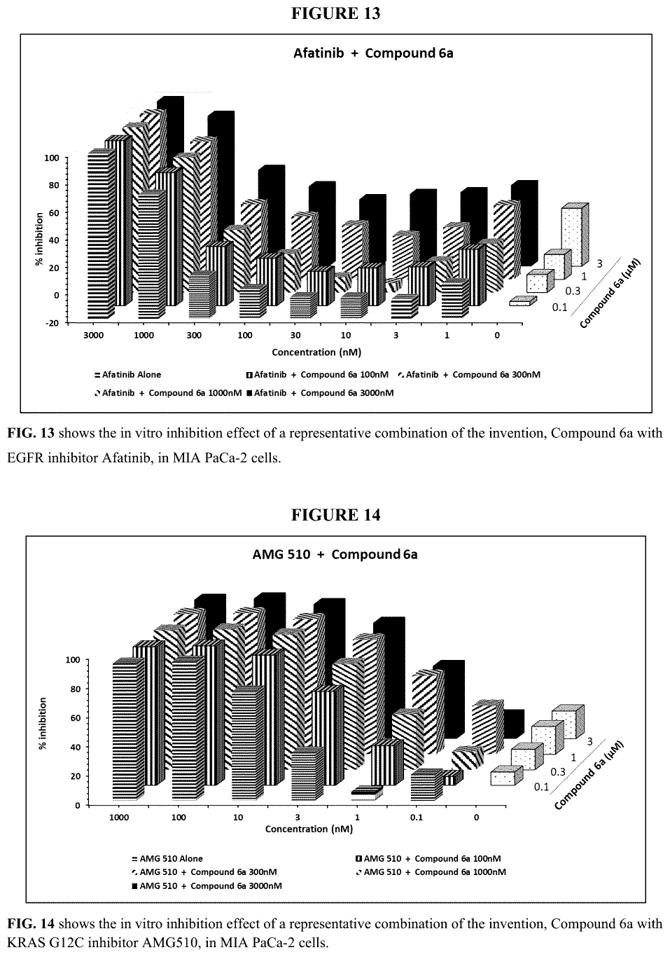

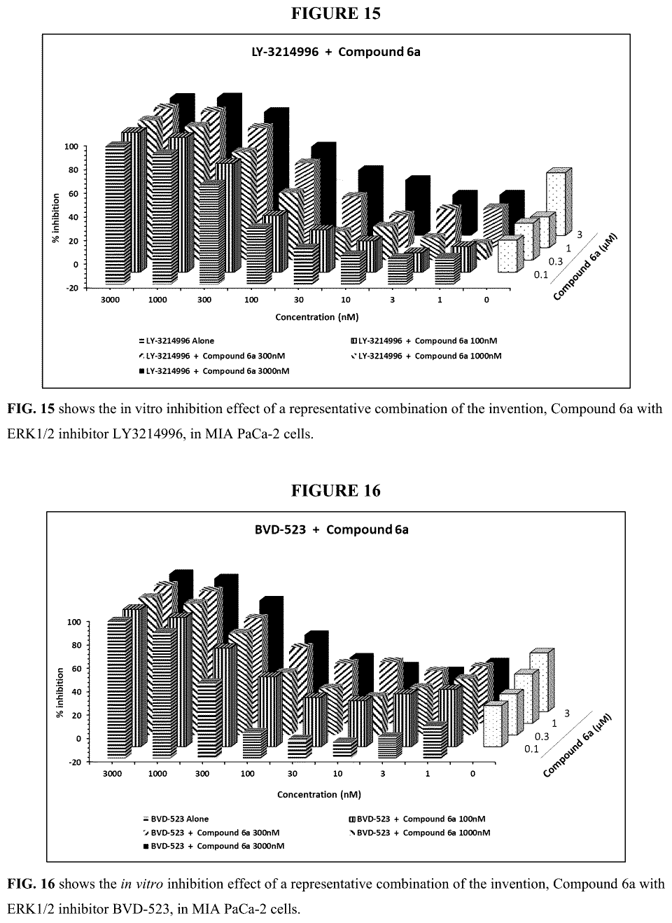

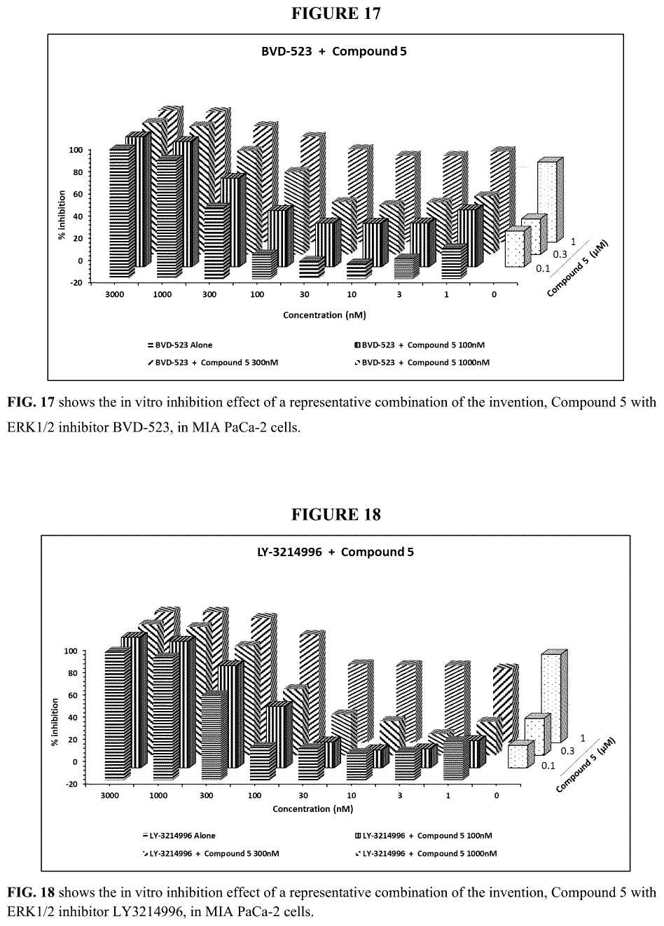

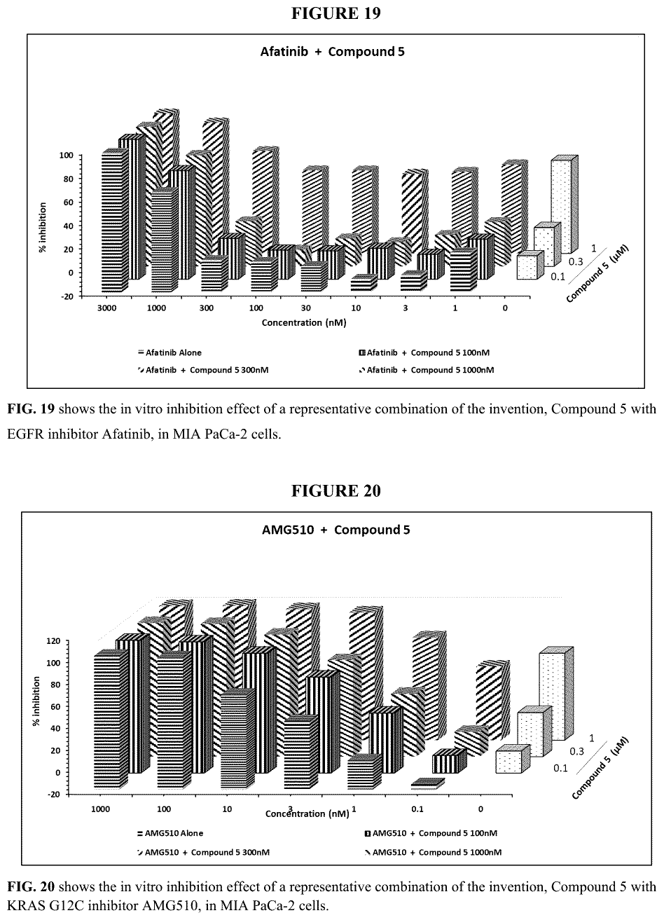

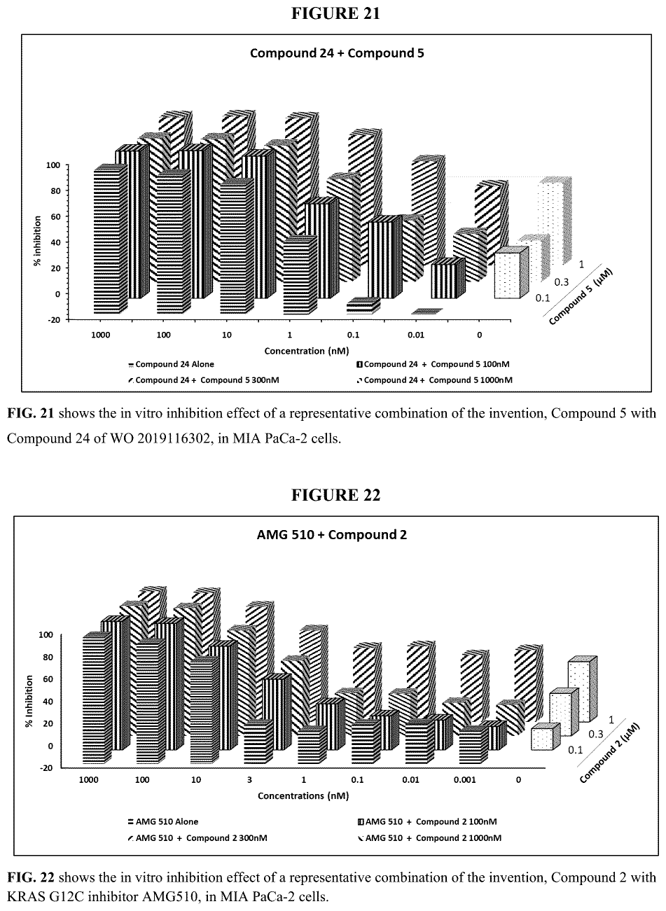

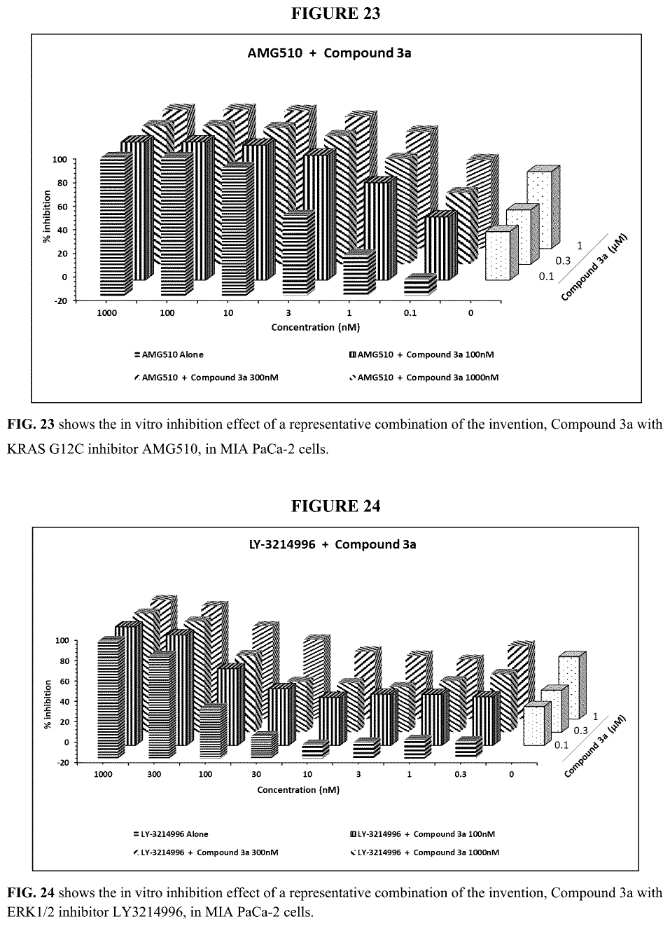

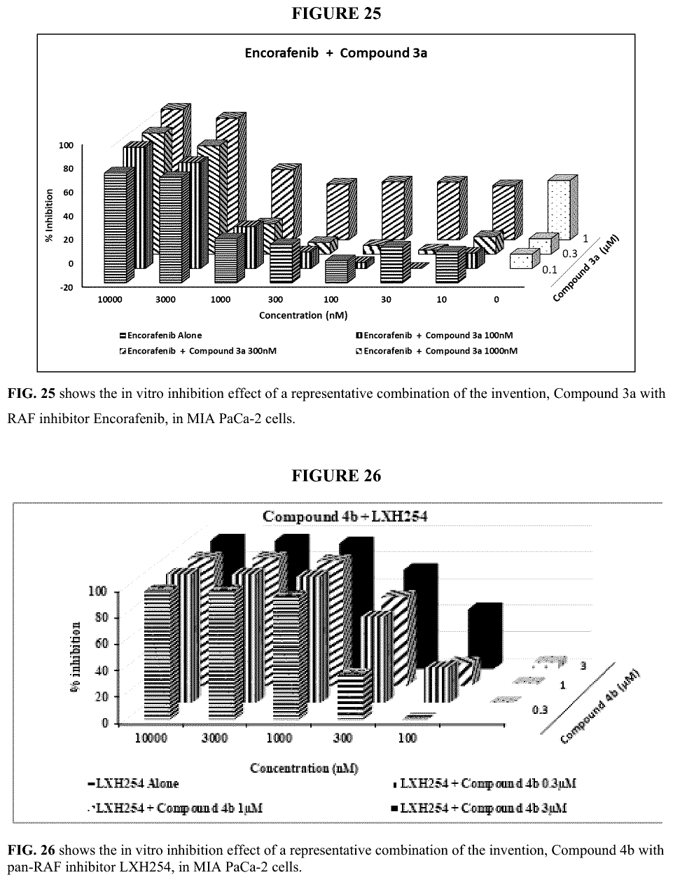

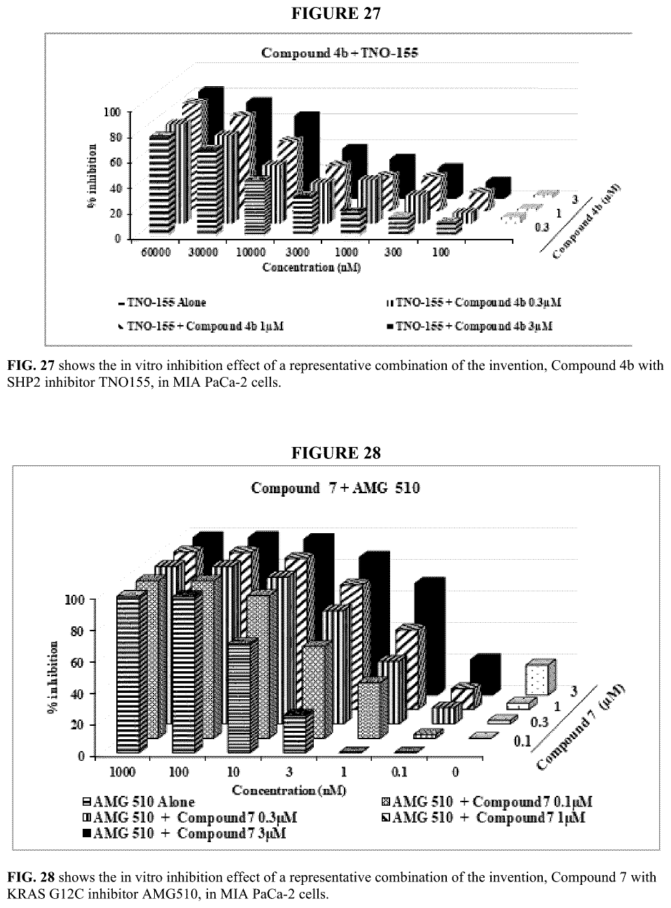

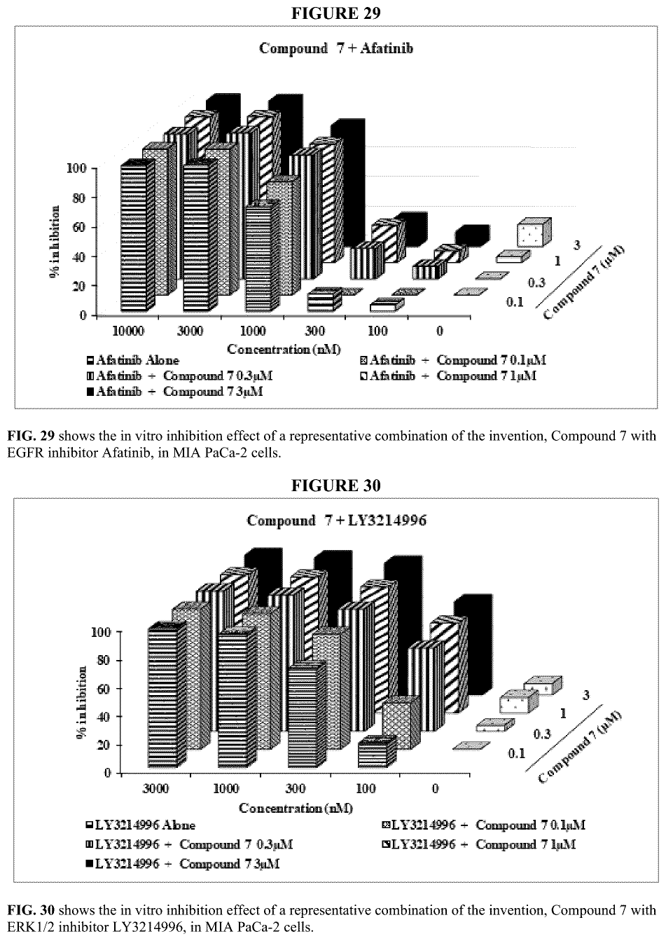

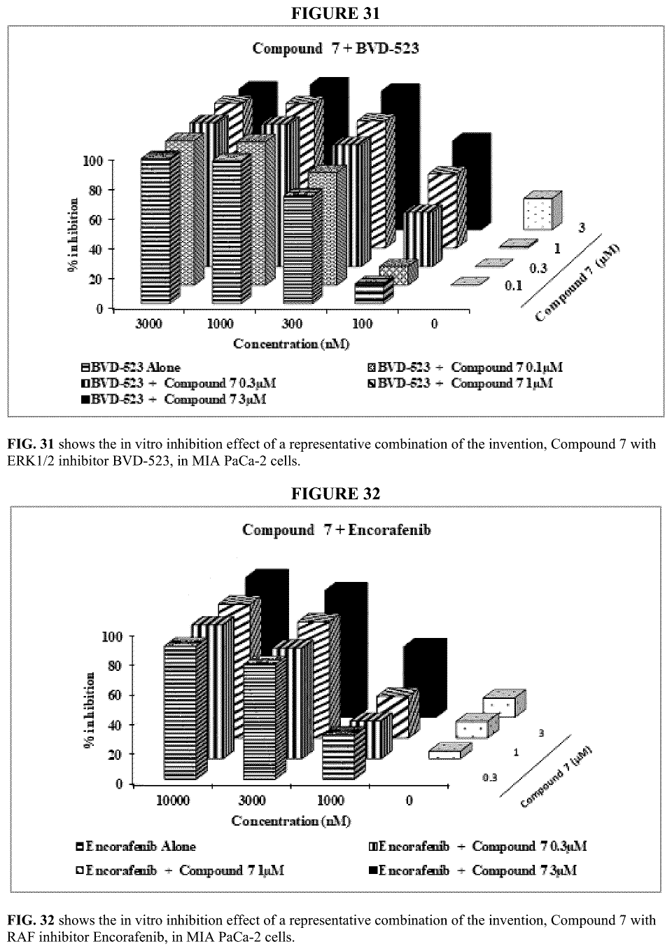

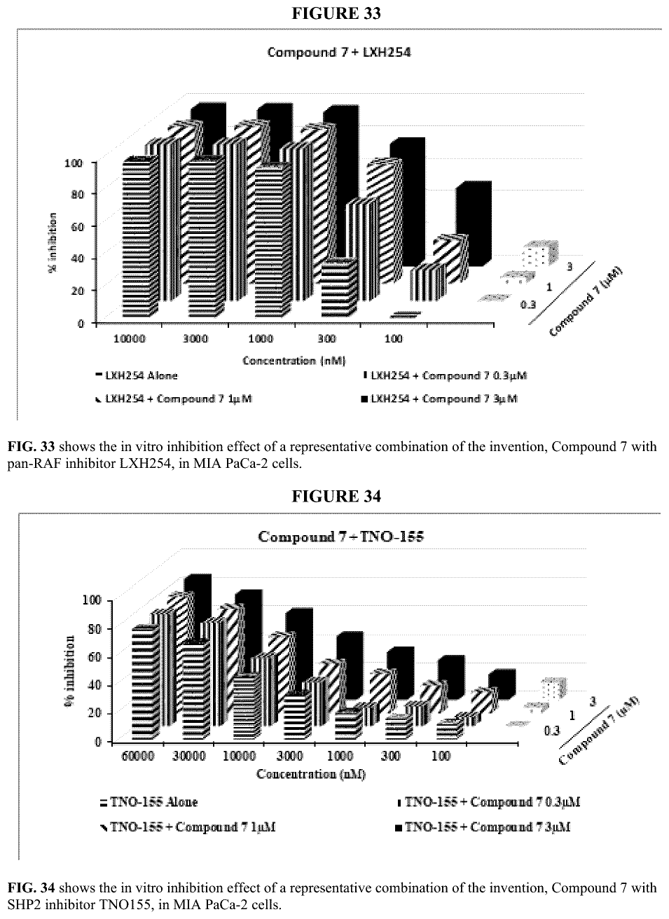

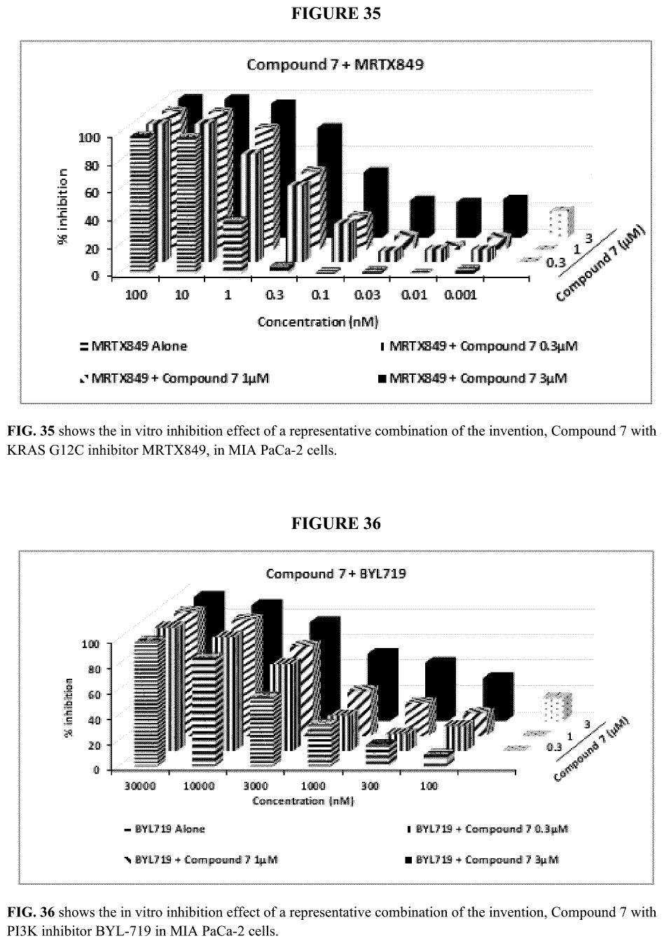

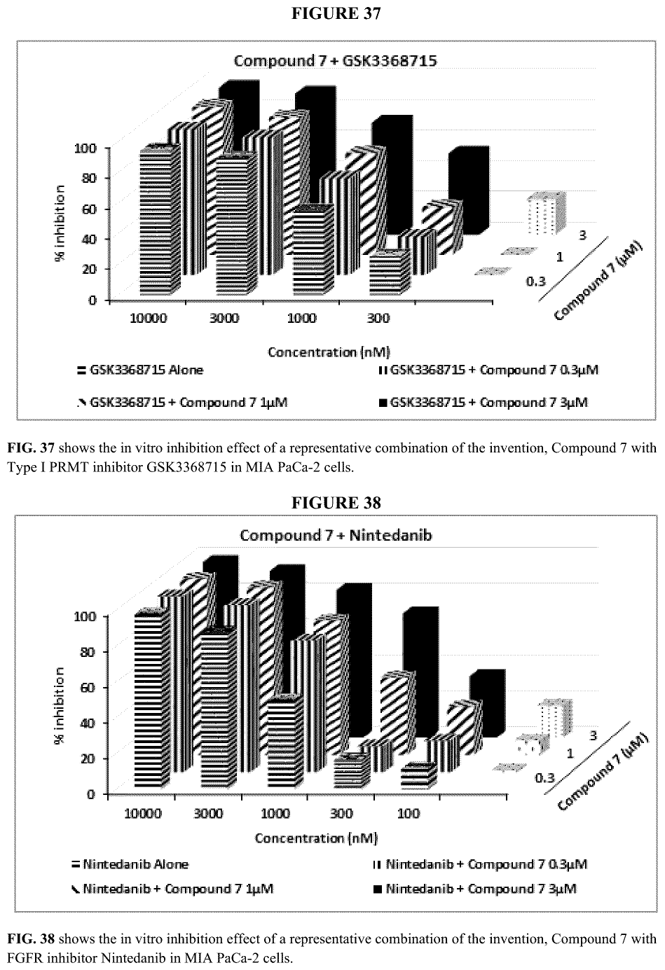

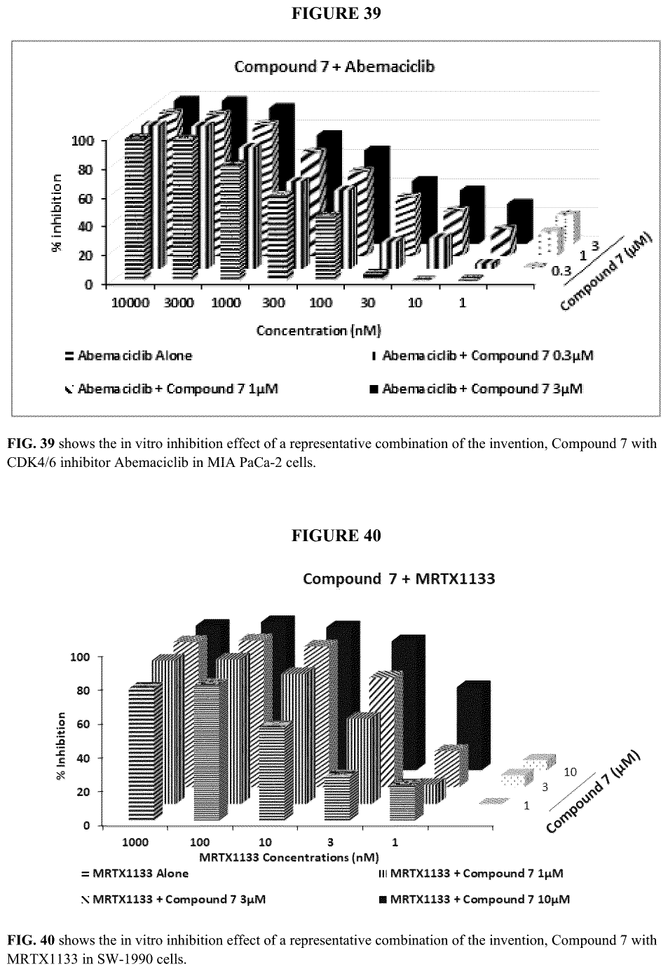

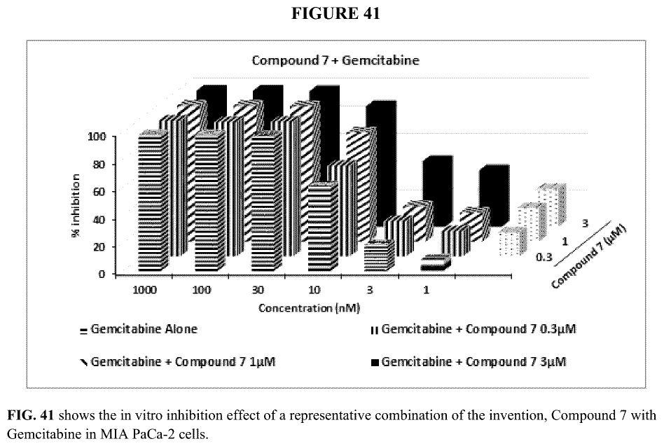

### DETAILED DESCRIPTION OF INVENTION

RAS mutated cancers continue to be dependent on upstream regulators like SOS1 for uninterrupted downstream oncogenic signaling (Bivona T. G., Science. 2019, 363(6433):1280-1281). Thus, concomitant inhibition of SOS1 and RAS may lead to sustained inhibition of the cancer growth signaling pathway resulting in more effective anticancer activity. The KRAS inhibitors that can be used along with SOS1 inhibitors include KRAS-G12C inhibitors (AMG 510, MRTX849 or any other agent that inhibits KRAS-G12C activity) or Pan KRAS inhibitors (inhibiting G12D, G12V, G12S, etc) like BI-2852 (Kessler, Dirk et al., PNAS., 2019, 116(32):15823-15829.). SOS1 is required for 3D spheroid growth of EGFR mutated NSCLC cells. Combined EGFR- and SOS1-inhibition markedly inhibited Raf/MEK/ERK and PI3K/AKT signaling and demonstrated strong synergy in mitigating RAS effector signaling (Theard, P. L. et al., eLife, 2020, 9:e58204). SOS1 is positioned proximal to RAS and RAF as downstream effector of RAS in the RAS/RAF/MEK/ERK pathway. Current approved RAF inhibitors, as single agent, demonstrate only modest efficacy in the clinic, and have rapid emergence of resistance (Packer, L. M. et al., Pigment Cell Melanoma Res. 2009, 22, 785-798; Saei, Azad et al., Cancers, 2019, 11(8), 1176). Thus, combination with a proximal regulator of the pathway, like SOS1, is expected to have more effective and sustained anticancer activity. ERK is a kinase positioned downstream in the RAS/RAF/MEK/ERK pathway. Activated ERK triggers the negative-feedback loop formed by inactivation of the Ras activating exchange factor complex Grb2-SOS by SOS1 phosphorylation and inactivation (Sung-Young Shin et al., Journal of Cell Science, 2009, 122(3), 425-435). Phosphatidylinositol 3-kinase (PI3K) is one of the main effector pathways of RAS, regulating cell growth, cell cycle entry, cell survival, cytoskeleton reorganization, and metabolism, and cancer. (Castellano, E. et al., Genes & Cancer, 2011, 2(3):261-74). PI3K mutations that hinder its interaction with RAS are highly resistant to RAS induced mutagenesis. Thus, combination of proximal regulator of RAS pathway, SOS1 with PI3K inhibitors is expected to have enhanced antitumor activity. AKT is an essential downstream effector of the PI3K pathway, having intersection with RAS/RAF pathway during oncogenic signaling. Combination of SOS1 and AKT inhibitors should interfere with both RAS/RAF and PI3K/AKT pathway and thus result in more complete and sustained tumor growth inhibition. c-MET activation stimulates the activity of the RAS guanine nucleotide exchanger son of sevenless (SOS) via binding with SHC and GRB2. This leads to leading to the activation of RAS/RAF/MEK/ERK pathway responsible for regulating a large number of genes, including those involved in cell proliferation, cell motility and cell cycle progression (Organ, S. L. et al., Ther Adv Med Oncol. 2011, 3(1 Suppl):S7-S19). Thus, combined inhibition of c-MET and SOS1 is expected to have enhanced antitumor effect as compared to individual treatment. The c-Met inhibitors that can be used along with SOS1 inhibitors include Tivantinib, Cabozantinib, Crizotinib, Capmatinib or antibodies targeting c-Met. SOS1 has also been implicated in hematological malignancies such as CML (Leukemia (2018) 32, 820-827). Combined treatment of CML cells with Brc-Abl kinase inhibitors along with SOS1 inhibitor provides a special opportunity to target both sensitive and resistant versions of CML. Recent reports indicate the emergence of acquired resistance to KRAS-targeted therapies and targeting SOS1 offers a possibility to overcome this resistance (NPJ Precis Oncol. 2021, 5(1):98; Sci Signal. 2019, 12(583):eaaw9450; J Thorac Oncol. 2021, 16(8):1321-1332).

SOS1 inhibitors can be used in combination with other therapies such as radiation, chemotherapy and/or treatment with a other targeted agents in multiple cancers and their subtypes as mentioned above. The agents that can be used for combination therapy are a KRAS inhibitor such as a KRAS G12C inhibitor and a KRASG12D inhibitor, KRAS G13C inhibitor, and panKRAS inhibitor; an EGFR inhibitor; an ERK1/2 inhibitor; a BRAF inhibitor; a pan-RAF inhibitor; a MEK inhibitor; a AKT inhibitor; a SHP2 inhibitor; protein arginine methyltransferases (PRMTs) inhibitor such as a PRMT5 inhibitor and Type I PRMT inhibitor; a PI3K inhibitor; a cyclin-dependent kinase (CDK) inhibitor such as CDK4/6 inhibitor; a FGFR inhibitor; a c-Met inhibitor; a RTK inhibitor; a non-receptor tyrosine kinase inhibitor; a histone methyltransferases (HMTs) inhibitor; a DNA methyltransferases (DNMTs) inhibitor; a Focal Adhesion Kinase (FAK) inhibitor; a Bcr-Abl tyrosine kinase inhibitor; a mTOR inhibitor; a PD1 inhibitor; a PD-L1 inhibitor; CTLA4 inhibitor; and chemotherapeutic agents such as gemcitabine, doxorubicin, cisplatin, carboplatin, paclitaxel, docetaxel, topotecan, irinotecan and temozolomide.

The KRAS inhibitors that can be used along with SOS1 inhibitors include KRAS-G12C inhibitors such as AMG 510, MRTX849, JDQ443, LY-3537982, JNJ-74699157, JAB-21822, GDC-6036, MK-1084, ZG-19018, D-1553, YL-15293, ICP-915, BI-1823911, BEBT-607, ERAS-3490, BPI-421286, JMX-1899 or KRAS-G12D inhibitors such as MRTX1133 or agents inhibiting multiple oncogenic RAS mutants such as BI-2852 (PNAS 2019; 116:32, 15823-15829), or KRAS G13C inhibitor (as disclosed in the US patent Application 20210130326A1 and US patent Application 20210130369A1), panRAS inhibitors (as disclosed in the US patent Application 20210130326A1 and US patent Application 20210130369A1).

The EGFR inhibitors that can be used along with SOS1 inhibitors include Afatinib, Osimertinib, Erlotinib or Gefitinib or any other agent that inhibits activity of the enzymes EGFR or its oncogenic variants.

The ERK inhibitors that can be used along with SOS1 inhibitors include BVD-523 (Ulixertinib), LY3214996, ASTX029, MK-8353 or ravoxertinib or any other agent that inhibits activity of the ERKli2 kinases.

The BRAF inhibitors that can be used along with SOS1 inhibitors include Dabrafenib, Regorafenib, Encorafenib or pan-RAF inhibitors such as LXH254 or any other agent that inhibits activity of the RAF isoforms (ARAF, BRAF and CRAF).

The AKT inhibitors that can be used along with SOS1 inhibitors include GSK690693, AZD5363, Ipatasertib or any other agent that inhibits the activity of one or more AKT isoforms (1, 2 and 3).

The SHP2 inhibitors that can be used along with SOS1 inhibitors include TNO155, JAB-3068, RMC-4630 or RLY-1971 or any other agent that inhibits activity of the SHP2 phosphatase.

The PRMT inhibitors that can be used along with SOS1 inhibitors include JNJ-64619178, PF-06939999, GSK-3326595, PRT543, PRT811, MS023, GSK3368715, Type I PRMT inhibitors or Compound 24 of WO 2019116302 or any other agent that inhibits the activity of PRMT methyltransferases.

SOS1 inhibitors also have the potential to target cancers with class III BRAF mutation (Clin Cancer Res 2019, 25(23), 6896). This includes cancers such as NSCLC, CRc and melanoma (Nature 2017, 548, 234-238).

The PI3K inhibitors that can be used along with SOS1 inhibitors include Alpelisib (BYL719), Copanlisib, Duvelisib, BEZ-235, Gedatolisib, Buparlisib or agents that inhibits the activity of one or more PI3K isoforms (α, β, δ and γ) or PI3K-mTOR dual inhibitors.

The CDK4/6 inhibitors that can be used along with SOS1 inhibitor is Abemaciclib or any other agent that inhibits activity of the CDK.

The FGFR inhibitors that can be used along with SOS1 inhibitors include Nintedanib, Dovitinib, AZD4547, BGJ398, JNJ 42756493 or any other agent that inhibits the activity of FGFR isoforms (1, 2, 3 and 4).

The c-Met inhibitors that can be used along with SOS1 inhibitors include Tivantinib, Cabozantinib, Crizotinib, Capmatinib or antibodies targeting c-Met.

SOS1 inhibitors can be combined with Bcr-Abl inhibitors that target CML. Examples of such agents include imatinib, dasatinib, nilotinib, ponatinib, etc.

SOS1 inhibitors also have the potential to be combined with immune-oncological (10) agents such as PD1 inhibitor (Pembrolizumab, Nivolumab), PD-L1 inhibitor (Atezolizumab, Avelumab), CTLA4 inhibitor (Ipilimumab), etc.

The chemotherapeutic agents that can be used along with SOS1 inhibitors include gemcitabine, topotecan, irinotecan, paclitaxel, cisplatin, carboplatin, doxorubicin or any other agent that is classified as chemotherapeutic.

SOS1 is involved in progression of Chronic Myelogenous leukemia (Leukemia 2018, volume 32, 820-827; Science. 2015; 350(6264): 1096-1101) and KRAS-G12D-mediated leukemogenesis (Blood. 2018; 132(24):2575-2579).

Present invention relates to a pharmaceutical combination for treating and/or preventing cancer comprising a SOS1 inhibitor of formula (I) or formula (II), its stereoisomer, or its pharmaceutical acceptable salt, and at least one additional active ingredient selected from a a KRAS inhibitor such as a KRAS G12C inhibitor and a KRASG12D inhibitor, KRAS G13C inhibitor, and panKRAS inhibitor; an EGFR inhibitor; an ERK1/2 inhibitor; a BRAF inhibitor; a pan-RAF inhibitor; a MEK inhibitor; a AKT inhibitor; a SHP2 inhibitor; protein arginine methyltransferases (PRMTs) inhibitor such as a PRMT5 inhibitor and Type 1 PRMT inhibitor; a PI3K inhibitor; a cyclin-dependent kinase (CDK) inhibitor such as CDK4/6 inhibitor; a FGFR inhibitor; a c-Met inhibitor; a RTK inhibitor; a non-receptor tyrosine kinase inhibitor; a histone methyltransferases (HMTs) inhibitor; a DNA methyltransferases (DNMTs) inhibitor; a Focal Adhesion Kinase (FAK) inhibitor; a Bcr-Abl tyrosine kinase inhibitor; a mTOR inhibitor; a PD1 inhibitor; a PD-L1 inhibitor; CTLA4 inhibitor; and chemotherapeutic agents such as gemcitabine, doxorubicin, cisplatin, carboplatin, paclitaxel, docetaxel, topotecan, irinotecan and temozolomide; wherein the SOS1 inhibitor of formula (I) is,

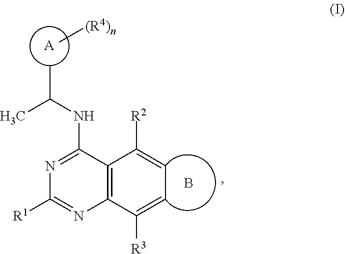

its tautomeric form, its stereoisomers, its pharmaceutically acceptable salt, their polymorph, and solvate thereof,
Wherein,
Ring A is selected from aryl, heteroaryl, and heterocyclyl;
Ring B is selected from substituted or unsubstituted 5 or 6 membered carbocyclic ring and substituted or unsubstituted 5 or 6 membered heterocyclic ring containing 1 to 3 heteroatoms independently selected from S, O, and N;
When ring B is carbocyclic ring, it is substituted with 1 to 8 substituents independently selected from R^{c} and R^{d};
when ring B is heterocyclic ring, it is substituted with 1 to 7 substituents; when it is substituted on a ring nitrogen atom, it is substituted with substituents selected from R^{a} and R^{b}; and when it is substituted on a ring carbon atom, it is substituted with substituents selected from R^{c} and R^{d};
R^{a} and R^{b} are independently selected from hydrogen, —C(═O)R^{g}, —C(═O)NR^{h}(Rⁱ), substituted or unsubstituted alkyl, substituted or unsubstituted cycloalkyl, substituted or unsubstituted aryl, substituted or unsubstituted heteroaryl, and substituted or unsubstituted heterocyclyl;
R^{c} and R^{d} are independently selected from hydrogen, halogen, oxo, —C(═O)R^{g}, —NR^{h}(Rⁱ)C(═O)NR^{h}(Rⁱ), —OR^{j}, substituted or unsubstituted alkyl, substituted or unsubstituted cycloalkyl, substituted or unsubstituted aryl, substituted or unsubstituted heteroaryl, and substituted or unsubstituted heterocyclyl; optionally R^{c} and R^{d} groups together with the carbon atom which they are attached forming a substituted or unsubstituted carbocyclic ring and substituted or unsubstituted heterocycle;
R¹ is selected from hydrogen, substituted or unsubstituted alkyl and substituted or unsubstituted cycloalkyl
R² and R³ are independently selected from hydrogen, halogen, cyano, substituted or unsubstituted alkyl, and substituted or unsubstituted cycloalkyl;
R⁴ is selected from halogen, cyano, —NR^{e}R^{f}, —OR^{j}, —C(═O)R^{g}, —C(═O)NR^{h}(Rⁱ), substituted or unsubstituted alkyl, substituted or unsubstituted cycloalkyl, cycloalkyl substituted with substituted or unsubstituted alkyl, substituted or unsubstituted aryl, substituted or unsubstituted heteroaryl, substituted or unsubstituted heterocyclyl, and heterocyclyl substituted with substituted alkyl;
R^{e} and R^{f} are independently selected from hydrogen, —C(═O)R^{g}, —C(═O)NR^{h}(Rⁱ), substituted or unsubstituted alkyl, alkyl substituted with substituted or unsubstituted heterocyclyl, substituted or unsubstituted cycloalkyl, substituted or unsubstituted aryl, substituted or unsubstituted heteroaryl, and substituted or unsubstituted heterocyclyl;
R^{g} is selected from substituted or unsubstituted alkyl, substituted or unsubstituted cycloalkyl, substituted or unsubstituted aryl, substituted or unsubstituted heteroaryl, and substituted or unsubstituted heterocyclyl;
R^{h} and Rⁱ are independently selected from hydrogen, substituted or unsubstituted alkyl, substituted or unsubstituted cycloalkyl, and substituted or unsubstituted heterocyclyl;
optionally R^{h} and Rⁱ groups together with the nitrogen atom to which they are attached forming a substituted or unsubstituted heterocycle;
R^{j} is selected from hydrogen, substituted or unsubstituted alkyl, alkyl substituted with substituted or unsubstituted cycloalkyl, and substituted or unsubstituted cycloalkyl;
‘n’ is an integer selected from 0, 1, 2, and 3;
when an alkyl group is substituted, it is substituted with 1 to 5 substituents independently selected from oxo (═O), halogen, cyano, cycloalkyl, aryl, heteroaryl, heterocyclyl, —OR⁵, —C(═O)OH, —C(═O)O(alkyl), —NR⁶R^{6a}, —NR⁶C(═O)R⁷, and —C(═O)NR⁶R^{6a};
when an cycloalkyl group is substituted, it is substituted with 1 to 4 substituents independently selected from oxo (═O), halogen, alkyl, hydroxyalkyl, cyano, aryl, heteroaryl, heterocyclyl, —OR⁵, —C(═O)OH, —C(═O)O(alkyl), —NR⁶R^{6a}, —NR⁶C(═O)R⁷, and —C(═O)NR⁶R^{6a};
when the aryl group is substituted, it is substituted with 1 to 4 substituents independently selected from halogen, nitro, cyano, alkyl, perhaloalkyl, cycloalkyl, heterocyclyl, heteroaryl, —OR⁵, —NR⁶R^{6a}, —NR⁶C(═O)R⁷, —C(═O)R⁷, —C(═O)NR⁶R^{6a}, —SO2-alkyl, —C(═O)OH, —C(═O)O-alkyl, and haloalkyl;
when the heteroaryl group is substituted, it is substituted with 1 to 4 substituents independently selected from halogen, nitro, cyano, alkyl, haloalkyl, perhaloalkyl, cycloalkyl, heterocyclyl, aryl, heteroaryl, —OR⁵, —NR⁶R^{6a}, —NR⁵C(═O)R⁷, —C(═O)R⁷, —C(═O)NR⁶R^{6a}, —SO2-alkyl, —C(═O)OH, and —C(═O)O-alkyl;
when the heterocycle group is substituted, it is substituted either on a ring carbon atom or on a ring hetero atom, and when it is substituted on a ring carbon atom, it is substituted with 1 to 4 substituents independently selected from oxo (═O), halogen, cyano, alkyl, alkoxyalkyl, hydroxyalkyl, cycloalkyl, perhaloalkyl, —OR⁵, —C(═O)NR⁶R^{6a}, —C(═O)OH, —C(═O)O-alkyl, —N(H)C(═O)(alkyl), —N(H)R⁶, and —N(alkyl)2; and when the heterocycle group is substituted on a ring nitrogen, it is substituted with substituents independently selected from alkyl, cycloalkyl, aryl, heteroaryl, —SO2(alkyl), —C(═O)R⁷, and —C(═O)O(alkyl); when the heterocycle group is substituted on a ring sulfur, it is substituted with 1 or 2 oxo (═O) group(s);
R⁵ is selected from hydrogen, alkyl, perhaloalkyl, and cycloalkyl;
R⁶ and R^{6a} are each independently selected from hydrogen, alkyl, and cycloalkyl;
or R⁶ and R^{6a} together with nitrogen to which they are attached form a heterocyclyl ring; and
R⁷ is selected from alkyl and cycloalkyl;
and wherein the SOS1 inhibitor of formula (II) is,

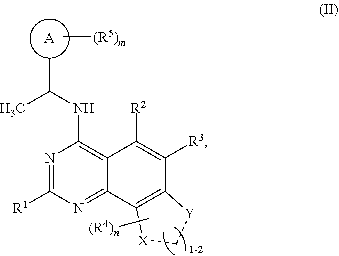

its tautomeric form, its stereoisomer, its pharmaceutical acceptable salt, its polymorph, or solvate thereof,
wherein
Ring A is selected from aryl, heteroaryl, and heterocyclyl;
‘ [symbol] ’ is either a single bond or double bond;
X and Y are independently selected from C, O, and NRc, provided that both X and Y cannot be O at the same time;
R¹ is selected from hydrogen and substituted or unsubstituted alkyl;
R² is selected from hydrogen, halogen, alkyl, and cycloalkyl;
R³ is selected from —OR⁶, —NR^{a}R^{b}, substituted or unsubstituted alkyl, substituted or unsubstituted cycloalkyl, alkyl substituted with substituted or unsubstituted heterocyclyl, substituted or unsubstituted heteroaryl, and substituted or unsubstituted heterocyclyl;
R⁴ is selected from oxo and substituted or unsubstituted alkyl;
R⁵ is selected from halogen, cyano, —NR^{c}R^{d}, substituted or unsubstituted alkyl, —C(═O) substituted or unsubstituted alkyl, substituted or unsubstituted cycloalkyl, substituted or unsubstituted aryl, and substituted or unsubstituted heteroaryl; optionally two R⁵ groups attached to the adjacent carbon atoms forming substituted or unsubstituted heterocycle:
R⁶ is selected from substituted or unsubstituted alkyl, substituted or unsubstituted heterocyclyl, 5 and alkyl substituted with substituted heterocyclyl;
R^{a} and R^{b} are independently selected from hydrogen, substituted or unsubstituted alkyl, and substituted or unsubstituted heterocyclyl;
R^{c} and R^{d} are independently selected from hydrogen and alkyl:
m is an integer selected from 0, 1, 2, and 3;
n is an integer selected from 0, 1, 2, 3, and 4;
when an alkyl group is substituted, it is substituted with 1 to 5 substituents independently selected from oxo (═O), halogen, cyano, cycloalkyl, aryl, heteroaryl, heterocyclyl, —OR7, —C(═O)OH, —C(═O)O(alkyl), —N^{R8}R^{8a}, —NR⁸C(═O)R⁹, and —C(═O)NR⁸R^{8a};
when an cycloalkyl group is substituted, it is substituted with 1 to 4 substituents independently selected from oxo (═O), halogen, alkyl, hydroxyalkyl, cyano, aryl, heteroaryl, heterocyclyl, —OR⁷, —C(═O)OH, —C(═O)O(alkyl), —NR⁸R^{8a}, —NR⁸C(═O)R⁹, and —C(═O)NR⁸R^{8a} aryl, heteroaryl, —OR⁷, —NR⁸R^{8a}., —NR⁷C(═O)R⁹, —C(═O)R⁹, —C(═O)NR⁸R^{8a}, —SO₂-alkyl, —C(═O)OH, and —C(═O)O-alkyl;
when the aryl group is substituted, it is substituted with 1 to 4 substituents independently selected from halogen, nitro, cyano, alkyl, haloalkyl, perhaloalkyl, cycloalkyl, heterocyclyl, heteroaryl, —OR⁷, —NR⁸R^{8a}, —NR⁸C(═O)R⁹, —C(═O)R⁹, —C(═O)NR⁸R^{8a}, —SO₂-alkyl, —C(═O)OH, and —C(═O)O-alkyl;
when the heteroaryl group is substituted, it is substituted with 1 to 4 substituents independently selected from halogen, nitro, cyano, alkyl, haloalkyl, perhaloalkyl, cycloalkyl, heterocyclyl, aryl, heteroaryl, —OR⁷, —NR⁸R^{8a}, —NR7C(═O)R⁹, —C(═O)R⁹, —C(═O)NR⁸R^{8a}, —SO₂-alkyl, —C(═O)OH, and —C(═O)O-alkyl;
when the heterocycle group is substituted, it is substituted either on a ring carbon atom or on a ring hetero atom, and when it is substituted on a ring carbon atom, it is substituted with 1 to 4 substituents independently selected from oxo (═O), halogen, cyano, alkyl, haloalkyl, alkoxyalkyl, hydroxyalkyl, cycloalkyl, perhaloalkyl, —OR⁷, —C(═O)NR⁸R^{8a}, —C(═O)OH, —C(═O)O-alkyl, —N(H)C(═O)(alkyl), —N(H)R⁸, and —N(alkyl)₂; and when the heterocycle group is substituted on a ring nitrogen, it is substituted with substituents independently selected from alkyl, haloalkyl, cycloalkyl, aryl, heteroaryl, —SO₂(alkyl), —C(═O)R⁹, and —C(═O)O(alkyl); when the heterocycle group is substituted on a ring sulfur, it is substituted with 1 or 2 oxo (═O) group(s);
R⁷ is selected from hydrogen, alkyl, perhaloalkyl, and cycloalkyl;
R⁸ and R^{8a} are each independently selected from hydrogen, alkyl, and cycloalkyl; and
R⁹ is selected from alkyl and cycloalkyl.

In accordance with another aspect the invention compound 1 of the Pharmaceutical combination of the present invention is selected from the group consisting of:
(R)-4-((1-(3-(1,1-Difluoro-2-hydroxy-2-methylpropyl)-2-fluorophenyl)ethyl)amino)-2,6,8,8-tetramethyl-6,8-dihydro-7H-pyrrolo[2,3-g]quinazolin-7-one (Compound 1);
(R/S)-4-((1-(3-(1,1-difluoro-2-hydroxy-2-methylpropyl)phenyl)ethyl)amino)-2,6,8,8-tetramethyl-6,8-dihydro-7H-pyrrolo[2,3-g]quinazolin-7-one (Compound 2);
4-(((R)-1-(3-((R&S)-1,1-Difluoro-2,3-dihydroxy-2-methylpropyl)-2-fluorophenyl) ethyl)amino)-2,6,8,8-tetramethyl-6,8-dihydro-7H-pyrrolo[2,3-g]quinazolin-7-one (Compound 3);
4-(((R)-1-(3-((R'S)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3a);
4-(((R)-1-(3-((S/R)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3b);
(R&S)-4-(((R)-1-(3-(1,1-difluoro-2-hydroxy-2-methylpropyl)-2-fluorophenyl) ethyl) amino)-8-methoxy-2,6,8-trimethyl-6,8-dihydro-7H-pyrrolo[2,3-g]quinazolin-7-one (Compound 4);
(S/R)-4-(((R)-1-(3-(1,1-difluoro-2-hydroxy-2-methylpropyl)-2-fluorophenyl)ethyl)amino)-8-methoxy-2,6,8-trimethyl-6H-pyrrolo[2,3-g]quinazolin-7(8H)-one (Compound 4a);
(R/S)-4-(((R)-1-(3-(1,1-difluoro-2-hydroxy-2-methylpropyl)-2-fluorophenyl)ethyl) amino)-8-methoxy-2,6,8-trimethyl-6H-pyrrolo[2,3-g]quinazolin-7(8H)-one (Compound 4b);
(R&S)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6):
(S/R)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6a):
(R/S)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6b); and
(S)-4-(((R)-1-(3-amino-5-(trifluoromethyl) phenyl) ethyl)amino)-8-methoxy-2,6,8-trimethyl-6,8-dihydro-7H-pyrrolo[2,3-g]quinazolin-7-one (Compound 7);
   or a pharmaceutically acceptable salt, a hydrate, or a stereoisomer thereof.

In accordance with yet another aspect the invention compound II of the pharmaceutical combination of the present invention is
(R)-5-(4-((1-(3-amino-5-(trifluoromethyl) phenyl) ethyl) amino)-2-methyl-8,9-dihydro-7H-cyclopenta[h]quinazolin-6-yl)-1-methylpyridin-2(1H)-one (Compound 5);
   or a pharmaceutically acceptable salt, a hydrate, or a stereoisomer thereof.

In some embodiments of the invention, the pharmaceutical combination comprising SOS 1 inhibitor selected from formula (I) or formula (II) and an additional active ingredient selected from KRAS, inhibitor, KRASG12C inhibitor, KRAS-G12D inhibitors, KRAS G13C inhibitor, and pan KRAS inhibitor. In some embodiments, KRASG12C inhibitor is selected from Sotorasib (AMG510) 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one, (Hong D S. et al. New England Journal of Medicine 2020, 383(13):1207-17); MRTX849 (1-(4-(7-(8-chloronaphthalen-1-yl)-2-((1-methylpyrrolidin-2-yl)methoxy)-5,6,7,8-tetrahydropyrido[3,4-d]pyrimidin-4-yl)-2-methylpiperazin-1-yl)-2-fluoroprop-2-en-1-one), (Hallin J., et al. Cancer discovery. 2020 10(1):54-71); JDQ443 (Brachmann S M, et. al. Mol Cancer Ther. 2021, 20 (12):P124); LY-3537982 (Peng, Sheng-Bin, et al. Cncer Res. 2021, 81(13):1259-1259); JNJ-74699157, (Nagasaka M., et. al. Cancer treatment reviews 2020, 84:101974); JAB-21822 (Li Y. et al. Current Opinion in Oncology 2022, 34(1):66-76); GDC-6036 (Chen H., et al. Journal of medicinal chemistry, 2020 63(23):14404-24); D-1553 (Zhe Shi. et al. Cancer Res 2021 81(13):932), YL-15293 (Herdeis L., et al. Current opinion in structural biology. 2021 71:136-47), BI-1823911 (Nagasaka M. et al. Cancer treatment reviews. 2021 101:102309) BEBT-607:

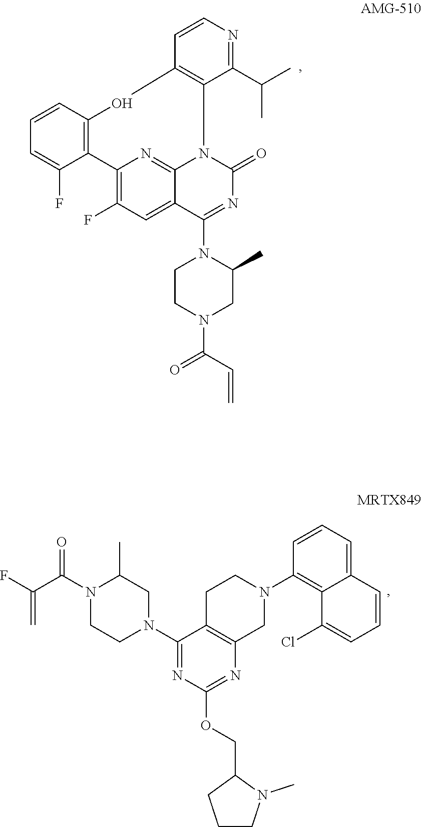 [SMILES: C=CC(=O)N1CCN(c2nc(=O)n3c4nc(c(F)cc24)-c2c(F)cccc2Oc2ccnc(C(C)C)c2-3)[C@@H](C)C1 ; C=C(F)C(=O)N1CCN(c2nc(OCC3CCCN3C)nc3c2CCN(c2cccc4cccc(Cl)c24)C3)CC1C]

In some embodiments, KRASG12D inhibitor is selected from MRTX1133 (4-(4-((1R,5S)-3,8-diazabicyclo[3.2.1]octan-3-yl)-8-fluoro-2-(((2S)-2-fluorotetrahydro-1H-pyrrolizin-7a(5H)-yl)methoxy)pyrido[4,3-d]pyrimidin-7-yl)-5-ethynyl-6-fluoronaphthalen-2-ol) (Wang X. et al. Journal of medicinal chemistry, 2021, 71:136-147) and BI-2852 ((3S)-5-hydroxy-3-(2-((((1-((1-methyl-1H-pyrrol-3-yl)methyl)-1H-inden-5-yl)methyl)amino)methyl)-1H-inden-3-yl)isoindolin-1-one) (Tran T H et al., Proceedings of the National Academy of Sciences. 2020, 17(7):3363-4):

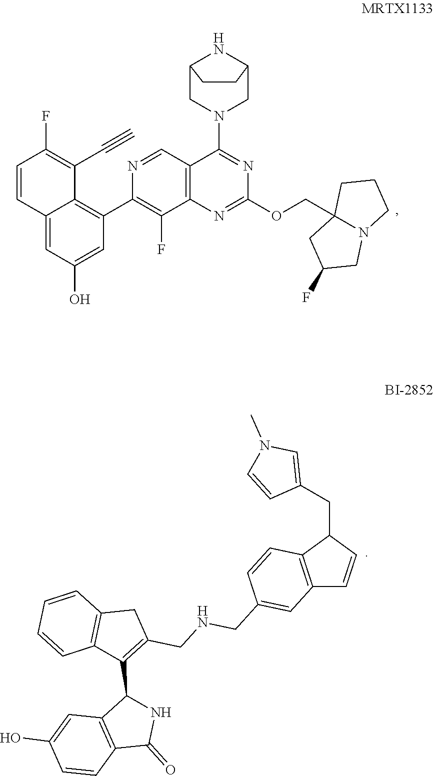 [SMILES: C#Cc1c(F)ccc2cc(O)cc(-c3ncc4c(N5CC6CCC(C5)N6)nc(OCC56CCCN5C[C@@H](F)C6)nc4c3F)c12 ; Cn1ccc(CC2C=Cc3cc(CNCC4=C([C@H]5NC(=O)c6ccc(O)cc65)c5ccccc5C4)ccc32)c1]

In some embodiments of the invention, the pharmaceutical combination comprises SOS 1 inhibitor selected from formula (I) or formula (II) and additional active ingredient is an EGFR inhibitor; wherein EGFR inhibitor is selected from Afatinib ((S,E)-N-(4-((3-chloro-4-fluorophenyl)amino)-7-((tetrahydrofuran-3-yl)oxy)quinazolin-6-yl)-4-(dimethylamino)but-2-enamide) (Dungo R T. et al., Drugs. 2013, 73(13):1503-15), Osimertinib (N-(2-((2-(dimethylamino)ethyl)(methyl)amino)-4-methoxy-5-((4-(1-methyl-1H-indol-3-yl)pyrimidin-2-yl)amino)phenyl)acrylamide) (Greig S L. Et al., Drugs. 2016, 76(2):263-73), Erlotinib (N-(3-ethynylphenyl)-6,7-bis(2-methoxyethoxy)quinazolin-4-amine) (Dowell, J. et al., Nature Reviews Drug Discovery, 2005 4(1)); and Gefitinib (N-(3-chloro-4-fluorophenyl)-7-methoxy-6-(3-morpholinopropoxy)quinazolin-4-amine) (Sanford M., et al. Drugs 2009, 69(16):2303-28):

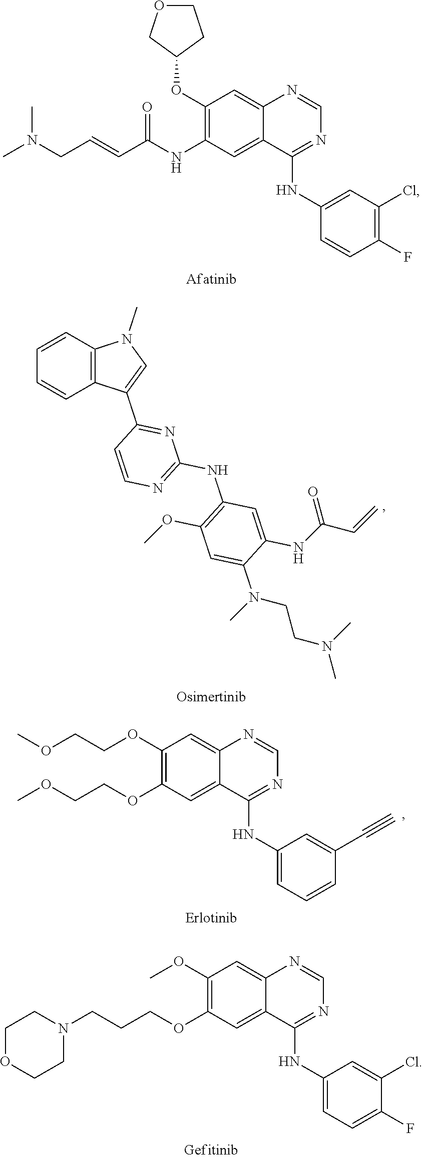

In some embodiments the invention, the pharmaceutical combination comprises SOS 1 inhibitor selected from formula (I) or formula (I1) and additional active ingredient is an ERK1/2 inhibitor, wherein, the ERK1/2 inhibitor is selected from LY-3214996 (6,6-Dimethyl-2-[2-[(2-methylpyrazol-3-yl)amino]pyrimidin-4-yl]-5-(2-morpholin-4-ylethyl)thieno[2,3-c]pyrrol-4-one), (Yan Q., et al. Journal of Biomedical Nanotechnology 2021, 17(7):1380-91), BVD-523 (Ulixertinib) ((S)-4-(5-chloro-2-(isopropylamino)pyridin-4-yl)-N-(1-(3-chlorophenyl)-2-hydroxyethyl)-1H-pyrrole-2-carboxamide) (Sullivan R J., et al. Cancer discovery. 2018, 8(2):184-95), ASTX-029 (Moon H., et al. Cancers. 2021, 13(12):3026), MK-8353 ((3S)-3-methylsulfanyl-1-[2-[4-[4-(1-methyl-1,2,4-triazol-3-yl)phenyl]-3,6-dihydro-2H-pyridin-1-yl]-2-oxoethyl]-N-[3-(6-propan-2-yloxypyridin-3-yl)-1H-indazol-5-yl]pyrrolidine-3-carboxamide) (Moschos S J., et al. JCI insight 2018, 3(4):e92352) and ravoxertinib ((S)-1-(1-(4-chloro-3-fluorophenyl)-2-hydroxyethyl)-4-(2-((1-methyl-1H-pyrazol-5-yl)amino)pyrimidin-4-yl)pyridin-2(1H)-one) (Park S J., et al. Annals of Oncology. 2020, 31:S1281):

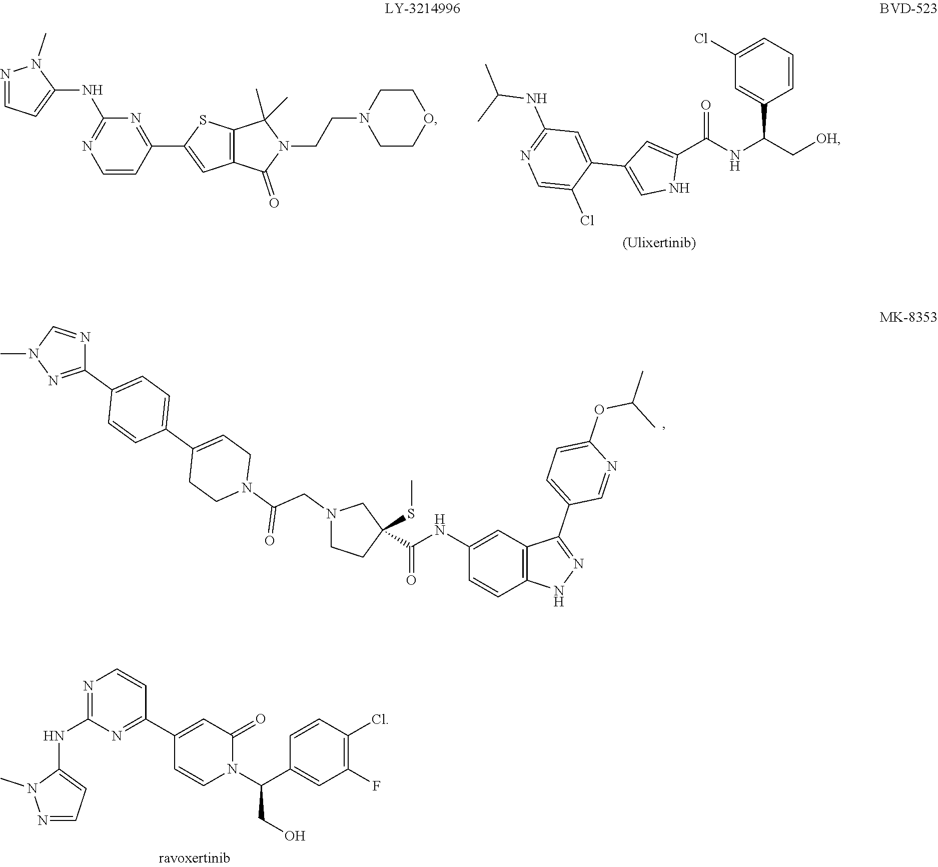

In some embodiments of the invention, the pharmaceutical combination comprising SOS 1 inhibitor selected from formula (I) or formula (II) and an additional active ingredient is a pan-RAF, wherein the pan-RAF inhibitor is selected from Dabrafenib (N-(3-(5-(2-aminopyrimidin-4-yl)-2-(tert-butyl)thiazol-4-yl)-2-fluorophenyl)-2,6-difluorobenzenesulfonamide) (Menzies A M., et al. Drug design, development and therapy 2012, 6:391); Regorafenib (4-(4-(3-(4-chloro-3-(trifluoromethyl)phenyl)ureido)-3-fluorophenoxy)-N-methylpicolinamide) (Grothey A., et al. The Lancet 2013, 381(9863):303-12); Encorafenib (methyl (S)-(1-((4-(3-(5-chloro-2-fluoro-3-(methylsulfonamido)phenyl)-1-isopropyl-1H-pyrazol-4-yl)pyrimidin-2-yl)amino)propan-2-yl)carbamate) (Dummer R., et al. The Lancet Oncology 2018, 19(5):603-15.); and LXH254 N-(3-(2-(2-hydroxyethoxy)-6-morpholinopyridin-4-yl)-4-methylphenyl)-2-(trifluoromethyl)isonicotinamide (Monaco K A., et al. Clinical cancer research 2021, 27(7):2061-73):

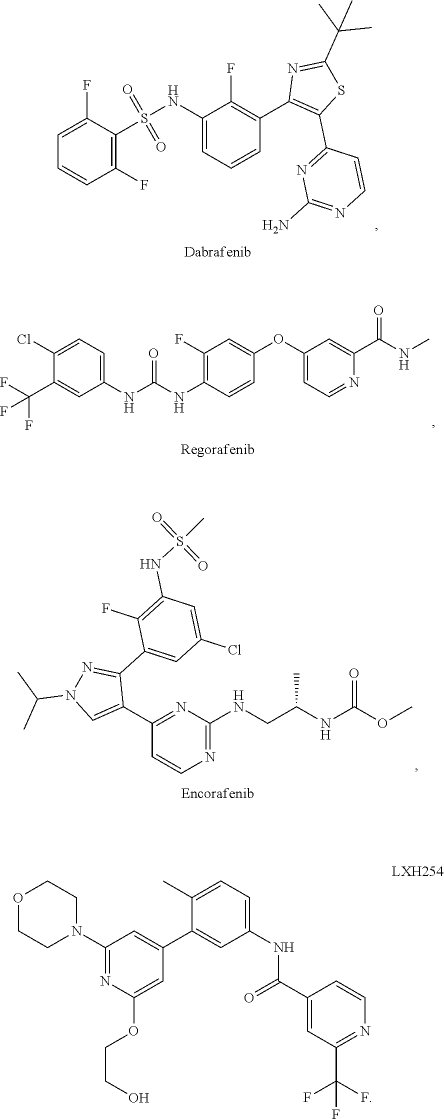

In some embodiments of the invention, the pharmaceutical combination comprising SOS 1 inhibitor selected from formula (I) or formula (II) and additional active ingredient is AKT inhibitor, wherein, the AKT inhibitor is selected from GSK690693 ((S)-4-(2-(4-amino-1,2,5-oxadiazol-3-yl)-1-ethyl-7-(piperidin-3-ylmethoxy)-1H-imidazo[4,5-c]pyridin-4-yl)-2-methylbut-3-yn-2-ol), Levy D S., et al. The Journal of the American Society of Hematology. 2009, 113(8):1723-9); AZD5363 (S)-4-amino-N-(1-(4-chlorophenyl)-3-hydroxypropyl)-1-(7H-pyrrolo[2,3-d]pyrimidin-4-yl)piperidine-4-carboxamide (Davies B R., et al. Molecular cancer therapeutics 2012, 11(4):873-87) and Ipatasertib ((S)-2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropylamino)propan-1-one) (Kim S B., et al., The Lancet Oncology. 2017, 18(10):1360-72):

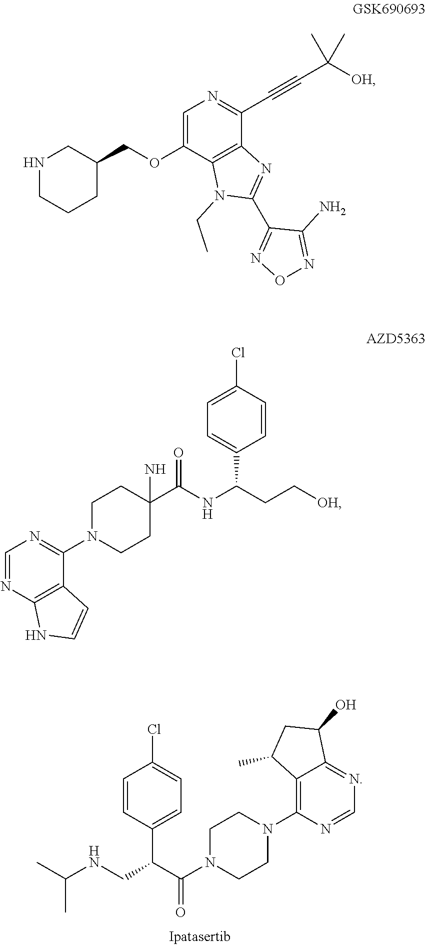 [SMILES: CCn1c(-c2nonc2N)nc2c(C#CC(C)(C)O)ncc(OC[C@H]3CCCNC3)c21 ; NC1(C(=O)N[C@@H](CCO)c2ccc(Cl)cc2)CCN(c2ncnc3[nH]ccc23)CC1 ; CC(C)NC[C@@H](C(=O)N1CCN(c2ncnc3c2[C@H](C)C[C@H]3O)CC1)c1ccc(Cl)cc1]

In some embodiments of the invention, the pharmaceutical combination comprises SOS1 inhibitor selected from formula (I) and formula (II) and an additional active ingredient is a SHP2 inhibitor, wherein, the SHP2 inhibitor is selected from TNO155 ((3S,4S)-8-(6-amino-5-((2-amino-3-chloropyridin-4-yl)thio)pyrazin-2-yl)-3-methyl-2-oxa-8-azaspiro[4.5]decan-4-amine) (LaMarche M J., et al. Journal of Medicinal Chemistry 2020, 63(22):13578-94); JAB-3068, (Liu Q., et al. Pharmacological research. 2020, 152:104595). RMC-4630 (Ou, S. I., et al. Journal of Thoracic Oncology, 15(2), 15-16) and RLY-1971 (Tang, Kai, et al. European Journal of Medicinal Chemistry 2020, 204:112657):

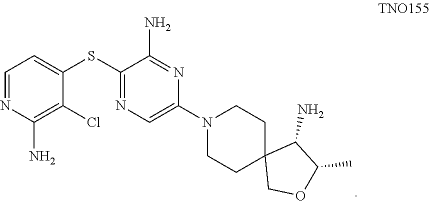 [SMILES: C[C@@H]1OCC2(CCN(c3cnc(Sc4ccnc(N)c4Cl)c(N)n3)CC2)[C@@H]1N]

In some embodiments of the invention, the pharmaceutical combination comprising SOS 1 inhibitor selected from formula (I) or formula (II) and an additional active ingredient is PRMT inhibitor, wherein the PRMT inhibitor is selected from JNJ-64619178 ((1S,2R,3S,5R)-3-(2-(2-amino-3-bromoquinolin-7-yl)ethyl)-5-(4-amino-7H-pyrrolo[2,3-d]pyrimidin-7-yl)cyclopentane-1,2-diol) (Tongfei Wu. et al Cancer Res. 2018, 78(13):4859); PF-06939999 (Jensen-Pergakes K, et al. Molecular cancer therapeutics. 2022, 21(1):3-15); GSK-3326595((R)-6-((1-acetylpiperidin-4-yl)amino)-N-(3-(3,4-dihydroisoquinolin-2(1H)-yl)-2-hydroxypropyl)pyrimidine-4-carboxamide) (Zhu K., et al Bioorganic & medicinal chemistry letters. 2018, 28(23-24):3693-9); PRT543, (Bhagwat N, et al. In Cancer Research 2020, 80(16) 19106-44040); PRT811, (Falchook, Gerald S., et al. 2021 20(12):P044-P044); MS023 (N¹-((4-(4-isopropoxyphenyl)-1H-pyrrol-3-yl)methyl)-N1-methylethane-1,2-diamine), (Eram M S., et al. ACS chemical biology. 2016 11(3):772-81); GSK3368715 (N¹-((3-(4,4-bis(ethoxymethyl)cyclohexyl)-1H-pyrazol-4-yl)methyl)-N1,N2-dimethylethane-1,2-diamine), (Fedoriw A., et al. Cancer Cell 2019 36(1):100-14) and Compound 24 of WO2019116302 ((1S,2R,5R)-3-(2-(2-amino-3-chloro-5-fluoroquinolin-7-yl)ethyl)-5-(4-amino-7H-pyrrolo[2,3-d]pyrimidin-7-yl)cyclopent-3-ene-1,2-diol):

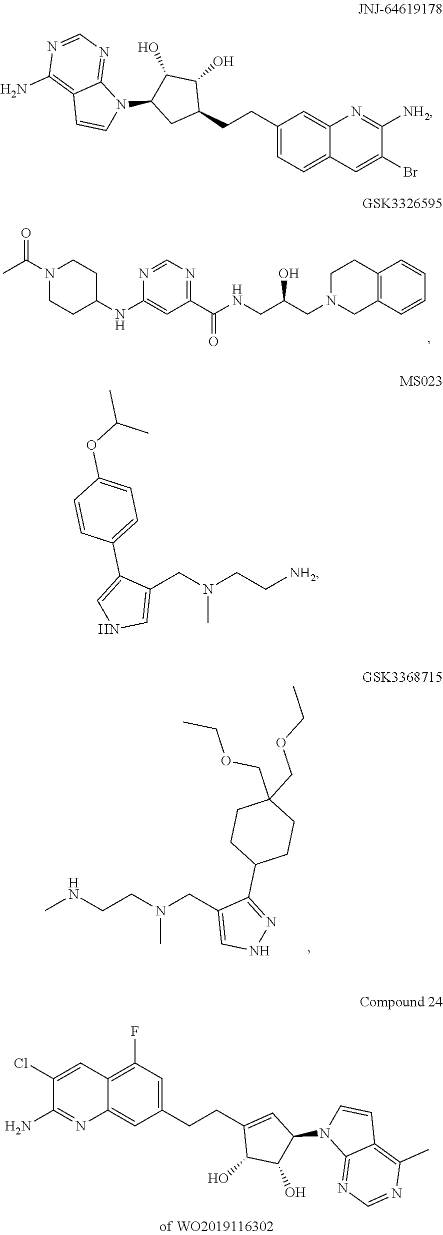 [SMILES: Nc1nc2cc(CC[C@H]3C[C@@H](n4ccc5c(N)ncnc54)[C@H](O)[C@@H]3O)ccc2cc1Br ; CC(=O)N1CCC(Nc2cc(C(=O)NC[C@@H](O)CN3CCc4ccccc4C3)ncn2)CC1 ; CC(C)Oc1ccc(-c2c[nH]cc2CN(C)CCN)cc1 ; CCOCC1(COCC)CCC(c2n[nH]cc2CN(C)CCNC)CC1 ; Cc1ncnc2c1ccn2[C@@H]1C=C(CCc2cc(F)c3cc(Cl)c(N)nc3c2)[C@@H](O)[C@H]1O]

In some embodiments of the invention, the pharmaceutical combination comprising SOS 1 inhibitor selected from formula (I) or formula (II) and an additional active ingredient is PI3K inhibitor, wherein, the PI3K inhibitor is selected from Alpelisib ((S)-N1-(4-methyl-5-(2-(1,1,1-trifluoro-2-methylpropan-2-yl)pyridin-4-yl)thiazol-2-yl)pyrrolidine-1,2-dicarboxamide), (Andre F, et al. New England Journal of Medicine 201, 9 380(20):192940) Copanlisib (2-amino-N-(7-methoxy-8-(3-morpholinopropoxy)-2,3-dihydroimidazo[1,2-c]quinazolin-5-yl)pyrimidine-5-carboxamide), (Dreyling M., et al. Journal of Clinical Oncology 2017, 35(35):3898-905) Duvelisib ((S)-3-(1-((7H-purin-6-yl)amino)ethyl)-8-chloro-2-phenylisoquinolin-1(2H)-one), (Flinn 1W., et al., The Journal of the American Society of Hematology 2018, 131(8):877-87); BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile), (Chen J., et al. Clinical and Experimental Pharmacology and Physiology. 2015, 42(12):1317-26); Gedatolisib (1-(4-(4-(dimethylamino)piperidine-1-carbonyl)phenyl)-3-(4-(4,6-dimorpholino-1,3,5-triazin-2-yl)phenyl)urea) (Del Campo J M., et al. Gynecologic oncology 2016, 142(1):62-9) and Buparlisib (5-(2,6-dimorpholinopyrimidin-4-yl)-4-(trifluoromethyl)pyridin-2-amine) (Baselga J., et al. The Lancet Oncology. 2017, 18(7):904-16):

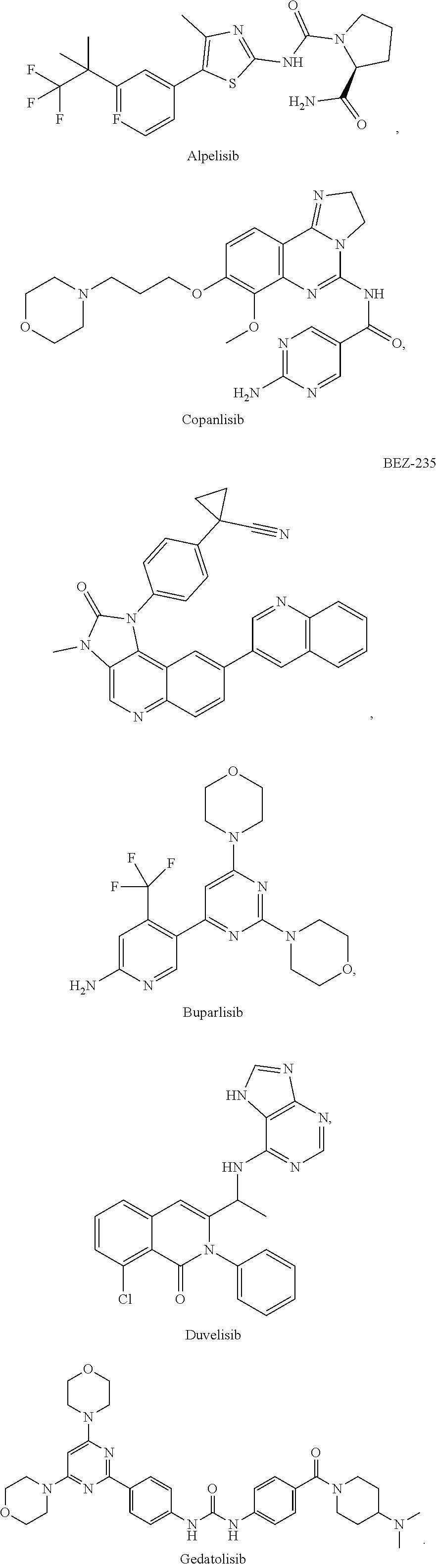

In some embodiments of the invention, the pharmaceutical combination comprising SOS 1 inhibitor selected from formula (I) or formula (II) and an additional active ingredient is CDK4/6 inhibitor, wherein, the CDK4/6 inhibitor is Abemaciclib (N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine) (Patnaik A., et al. Cancer discovery. 2016 6(7):740-53):

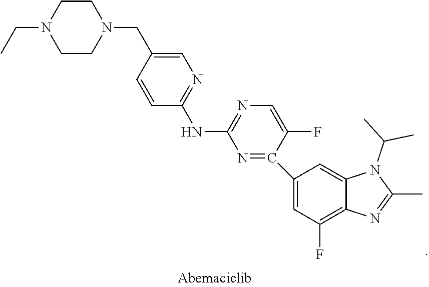 [SMILES: CCN1CCN(Cc2ccc(Nc3ncc(F)c(-c4cc(F)c5nc(C)n(C(C)C)c5c4)n3)nc2)CC1]

In some embodiments of the invention, the pharmaceutical combination comprising SOS 1 inhibitor selected from formula (I) or formula (II) and an additional active ingredient is the FGFR inhibitor, wherein, the FGFR inhibitor is selected from Nintedanib (methyl (Z)-3-(((4-(N-methyl-2-(4-methylpiperazin-1-yl)acetamido)phenyl)amino)(phenyl)methylene)-2-oxoindoline-6-carboxylate), (Richeldi L., et al. New England Journal of Medicine 2014, 370(22):2071-82) Dovitinib (4-amino-5-fluoro-3-(6-(4-methylpiperazin-1-yl)-1H-benzo[d]imidazol-2-yl)-4a,8a-dihydroquinolin-2(1H)-one), (Andre F., et al. Clinical cancer research 2013, 19(13):3693-702); JNJ 42756493 (N¹-(3,5-dimethoxyphenyl)-N2-isopropyl-N1-(3-(1-methyl-1H-pyrazol-4-yl)quinoxalin-6-yl)ethane-1,2-diamine), (Loriot, Yohann, et al. New England Journal of Medicine 2019, 381(4) 338-348); AZD4547 (N-(5-(3,5-dimethoxyphenethyl)-1H-pyrazol-3-yl)-4-((3R,5S)-3,5-dimethylpiperazin-1-yl)benzamide), (Gavine P R., et al. Cancer research. 2012, 72(8):2045-56) and BGJ398 (3-(2,6-dichloro-3,5-dimethoxyphenyl)-1-(6-((4-(4-ethylpiperazin-1-yl)phenyl)amino)pyrimidin-4-yl)-1-methylurea), (Guagnano V. et al. Journal of medicinal chemistry 2011, 54(20):7066-83):

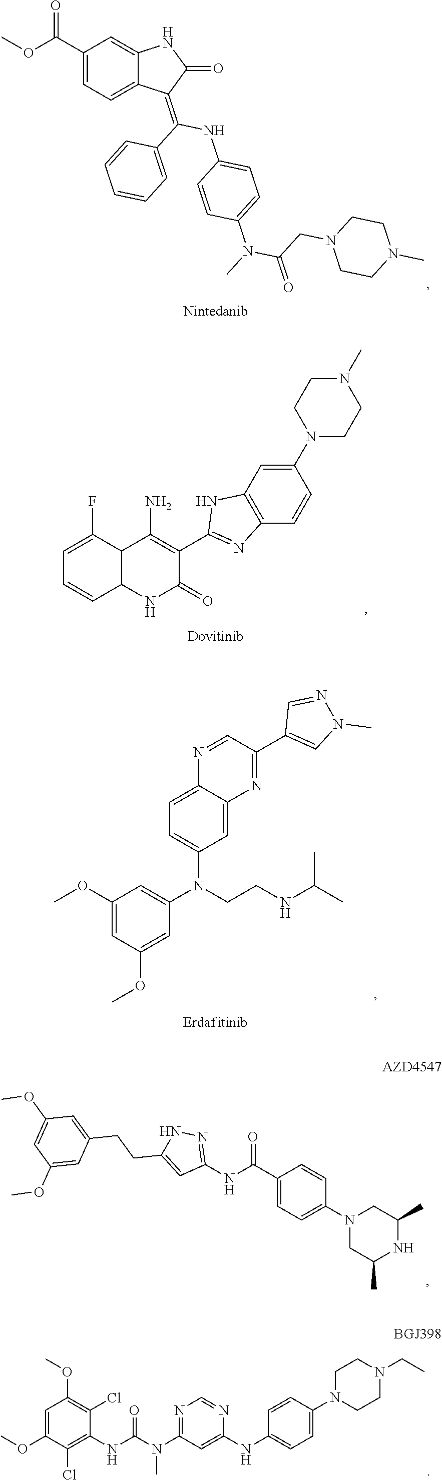

In some embodiments of the invention, the pharmaceutical combination comprising SOS 1 inhibitor selected from formula (I) or formula (II) and an additional active ingredient is c-Met inhibitor, wherein, the c-Met inhibitor is selected from Tivantinib ((3R,4R)-3-(5,6-dihydro-4H-pyrrolo[3,2,1-ij]quinolin-1-yl)-4-(1H-indol-3-yl)pyrrolidine-2,5-dione) (Santoro A., et al. The lancet oncology 2013, 14(1):55-63); Cabozantinib (N-(4-((6,7-dimethoxyquinolin-4-yl)oxy)phenyl)-N-(4-fluorophenyl)cyclopropane-1,1-dicarboxamide), (Abou-Alfa G K., et al. New England Journal of Medicine 2018, 379(1):54-63); Crizotinib ((R)-3-(1-(2,6-dichloro-3-fluorophenyl)ethoxy)-5-(1-(piperidin-4-yl)-1H-pyrazol-4-yl)pyridin-2-amine) (Shaw A T., et al. New England Journal of Medicine 2013, 368(25):2385-94) and Capmatinib (2-fluoro-N-methyl-4-(7-(quinolin-6-ylmethyl)imidazo[1,2-b][1,2,4]triazin-2-yl)benzamide) (Wolf J., et al. New England Journal of Medicine 2020, 383(10):944-57):

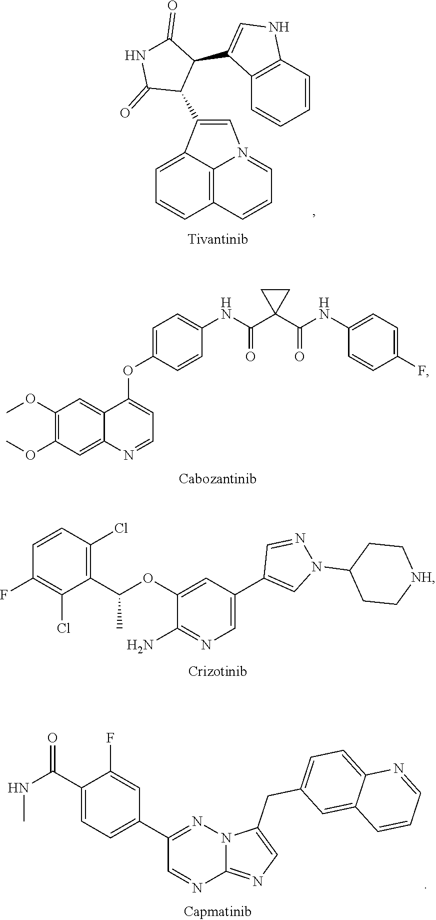

In some embodiments of the invention, the pharmaceutical combination comprising SOS1 inhibitor selected from formula (I) or formula (II) and additional active ingredient is Bcr-Abl kinase inhibitor, wherein, the Bcr-Abl kinase inhibitor is selected from imatinib (N-(4-methyl-3-((4-(pyridin-3-yl)pyrimidin-2-yl)amino)phenyl)-4-((4-methylpiperazin-1-yl)methyl)benzamide); (Peng B., et al. Clinical pharmacokinetics 2005, 44(9):879-94) Dasatinib (N-(2-chloro-6-methylphenyl)-2-((6-(4-(2-hydroxyethyl)piperazin-1-yl)-2-methylpyrimidin-4-yl)amino)thiazole-5-carboxamide) (Kantarjian H., et al. Nature reviews Drug discovery 2006, 5(9):717-9.); nilotinib (4-methyl-N-(3-(4-methyl-1H-imidazol-1-yl)-5-(trifluoromethyl)phenyl)-3-((4-(pyridin-3-yl)pyrimidin-2-yl)amino)benzamide) (Weisberg E., et al. British journal of cancer 2006 94(12):1765-9) and ponatinib (3-(imidazo[1,2-b]pyridazin-3-ylethynyl)-4-methyl-N-(4-((4-methylpiperazin-1-yl)methyl)-3-(trifluoromethyl)phenyl)benzamide) (Cortes J E., et al. New England Journal of Medicine 2012 367(22):2075-88):

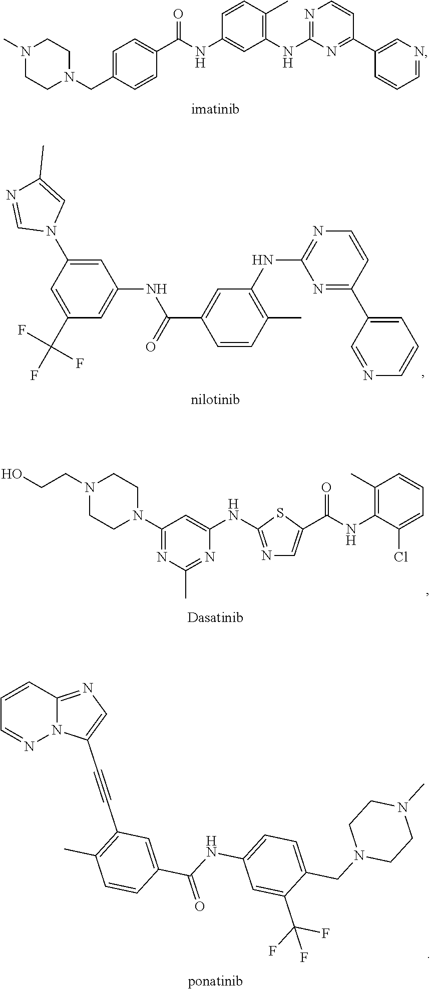 [SMILES: Cc1ccc(NC(=O)c2ccc(CN3CCN(C)CC3)cc2)cc1Nc1nccc(-c2cccnc2)n1 ; Cc1cn(-c2cc(NC(=O)c3ccc(C)c(Nc4nccc(-c5cccnc5)n4)c3)cc(C(F)(F)F)c2)cn1 ; Cc1nc(Nc2ncc(C(=O)Nc3c(C)cccc3Cl)s2)cc(N2CCN(CCO)CC2)n1 ; Cc1ccc(C(=O)Nc2ccc(CN3CCN(C)CC3)c(C(F)(F)F)c2)cc1C#Cc1cnc2cccnn12]

In some embodiments of the invention, the pharmaceutical combination comprising SOS 1 inhibitor selected from formula (I) or formula (II) and additional active ingredient is PD-1 inhibitor, wherein, the PD1 inhibitor is selected from Pembrolizumab (Garon E B., et al. New England Journal of Medicine 2015, 372(21):2018-28) and Nivolumab (Wolchok J D., et al. N Engl J Med. 2013, 369:122-33). In some embodiments of the invention, the pharmaceutical combination comprising SOS 1 inhibitor selected from formula (I) or formula (II) and additional active ingredient is PD-L1 inhibitor, wherein, the PD-Li inhibitor is selected from Atezolizumab (Schmid P., et al. New England Journal of Medicine 2018, 379(22):2108-21) and Avelumab (Motzer R J., et al. New England Journal of Medicine 2019, 380(12):1103-15).

In some embodiments of the invention, the pharmaceutical combination comprising SOS 1 inhibitor selected from formula (I) or formula (II) and additional active ingredient is CTLA-4 inhibitor, wherein, the CTLA-4 inhibitor is Ipilimumab ((Hodi F S., et al., New England Journal of Medicine. 2010, 363(8):711-23).

In some embodiments of the invention, the pharmaceutical combination comprising SOS 1 inhibitor selected from formula (I) or formula (II) and an additional active ingredient is gemcitabine(4-amino-1-((2R,4R,5R)-3,3-difluoro-4-hydroxy-5-(hydroxymethyl)tetrahydrofuran-2-yl)pyrimidin-2(1H)-one), (Plunkett W., Anti-cancer drugs. 1995, 6:7-13); Topotecan ((S)-10-((dimethylamino)methyl)-4-ethyl-4,9-dihydroxy-1,12-dihydro-14H-pyrano[3′,4′:6,7]indolizino[1,2-b]quinoline-3,14(4H)-dione), (Herben V M., et al. Clinical pharmacokinetics 1996, 31(2):85-102); Irinotecan ((S)-4,11-diethyl-4-hydroxy-3,14-dioxo-3,4,12,14-tetrahydro-1H-pyrano[3′,4′:6,7]indolizino[1,2-b]quinolin-9-yl [1,4′-bipiperidine]-1′-carboxylate), (Vanhoefer U., et al. Journal of clinical oncology 2001, 19(5):1501-18); Paclitaxel ((2aR,4S,4aS,6R,9S,11 S,12S,12aR,12bS)-9-(((2R,3S)-3-benzamido-2-hydroxy-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate), (Rowinsky E K. et al. New England journal of medicine 1995, 332(15):1004-14.); Cisplatin (diaminoplatinum(IV) chloride), carboplatin, (LOEHRER PJ., et al. Annals of internal medicine 1984, 100(5):704-13) doxorubicin ((8S,10S)-10-(((2R,4S,5R,6S)-4-amino-5-hydroxy-6-methyltetrahydro-2H-pyran-2-yl)oxy)-6,8,11-trihydroxy-8-(2-hydroxyacetyl)-1-methoxy-7,8,9,10-tetrahydrotetracene-5,12-dione), (Weiss R B., et al. In Seminars in oncology 1992, 19(6):670-686) and Temozolomide (3-methyl-4-oxo-3,4-dihydroimidazo[5,1-d][1,2,3,5]tetrazine-8-carboxamide) (Friedman H S., et al. Clinical cancer research. 2000, 6(7):2585-97):

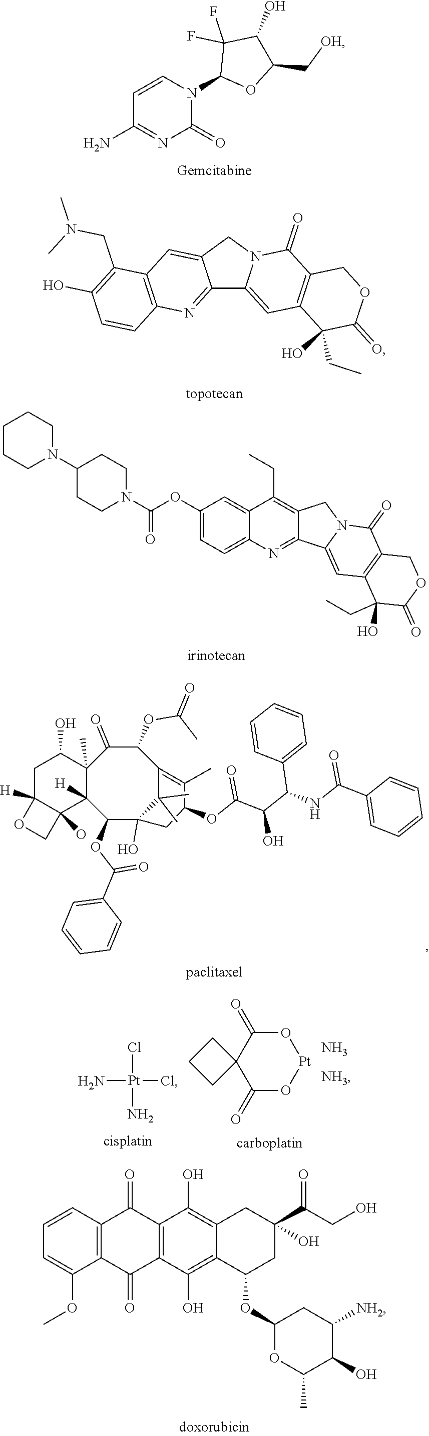

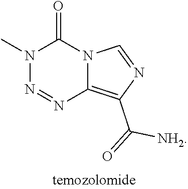

According to a feature of the present invention, the SOS1 inhibitor formula (I) and formula (II),

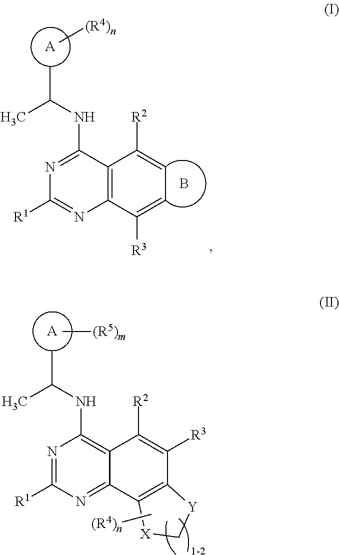

wherein all the symbols are as defined earlier, can be prepared by methods illustrated in the schemes and examples provided herein below. However, the disclosure should not be construed to limit the scope of the invention arriving at compound of formula (I) as disclosed hereinabove. Further, in the following schemes, where specific bases, acids, reagents, solvents, coupling agents, etc., are mentioned, it is understood that other bases, acids, reagents, solvents, coupling agents etc., known in the art may also be used and are therefore included within the scope of the present invention. Variations in reaction conditions, for example, temperature and/or duration of the reaction, which may be used as known in the art, are also within the scope of the present invention. All the isomers of the compound of formula in described in these schemes, unless otherwise specified, are also encompassed within the scope of this invention.

### General Synthetic Procedures for SOS1 Inhibitor of Formula I,

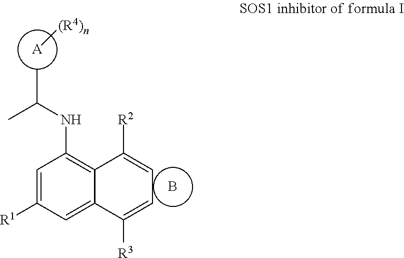

The corresponding α-methyl amine derivatives represented as formula (A5) could be prepared by following the sequential transformations as depicted in Scheme-A herein below—

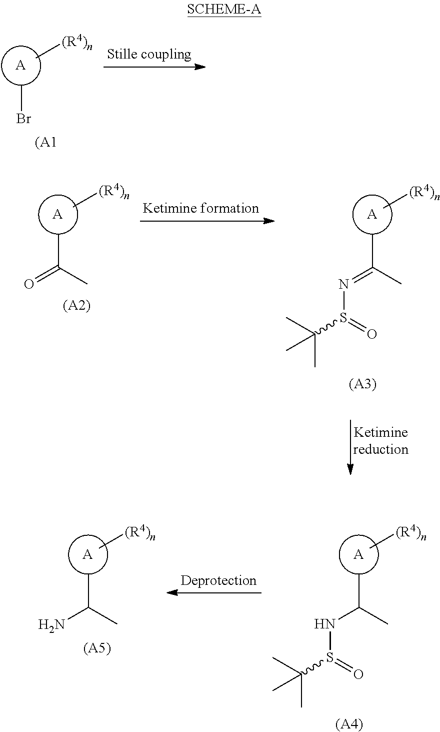

The compound of formula (A1) undergoes a metal catalyzed cross coupling with alkoxy vinyl stannane, e.g. tributyl(1-ethoxyvinyl)tin in presence of palladium catalysts such as Pd(Ph₃P)₂Cl₂, Pd₂(dba)₃ and like; optionally using bases such as triethylamine, N,N-Diisopropylethylamine and like, in hydrocarbon solvents like toluene or ether solvents like 1,4-dioxane to furnish the alkoxy vinyl intermediate which in turn provide compound of formula (A2) in acidic condition by employing aqueous mineral acids such as hydrochloric acid in ether solvent such as THF, 1,4-dioxane and like. The similar transformation can be carried out by reaction of compound of formula (A1) with n-alkylvinyl ether using catalysts such as palladium (II) acetate and like, ligands such as 1,3-Bis(diphenylphosphino)propane and like, in presence of organic bases such as DIPEA, TEA and like in alcoholic solvents such as ethylene glycol and at elevated temperatures, in solvents such as 1,4-dioxane, THF and mixtures thereof to give alkoxy vinyl intermediate which in turn provide compound of formula (A2) in acidic condition by employing aqueous mineral acids such as hydrochloric acid in ether solvent such as THF, 1,4-dioxane and like

The compound of formula (A2) was then reacted with corresponding chirally pure t-butanesulfinamide in presence of Lewis acid such as Titanium alkoxides e.g. titanium tetraethoxide, titanium isopropoxide and the like, in ether solvents such as 1,4-dioxane, THF and like, to obtain the compound of formula (A3).

The compound of formula (A3) reacted with reducing agent such as metal hydrides e.g. sodium borohydride, L-selectride and like, in solvents such as THF, 1,4-dioxane, methanol and the like, optionally in presence of water to provide sulfinamide of formula (A4). Major diastereoisomer in the compound of formula (A4) after reduction was separated or taken ahead as such.

The compound of formula (A4) under acidic condition undergoes cleavage of reduced ketimine derivative to generate amine of formula (A5) as a free base or salt. The acids employed for the transformation may involve mineral acids such as hydrochloric acid, organic acids like trifluoroacetic acid and thereof.

The compounds of formula (I) was prepared by following the sequential transformations as depicted and described in Scheme-B herein below—

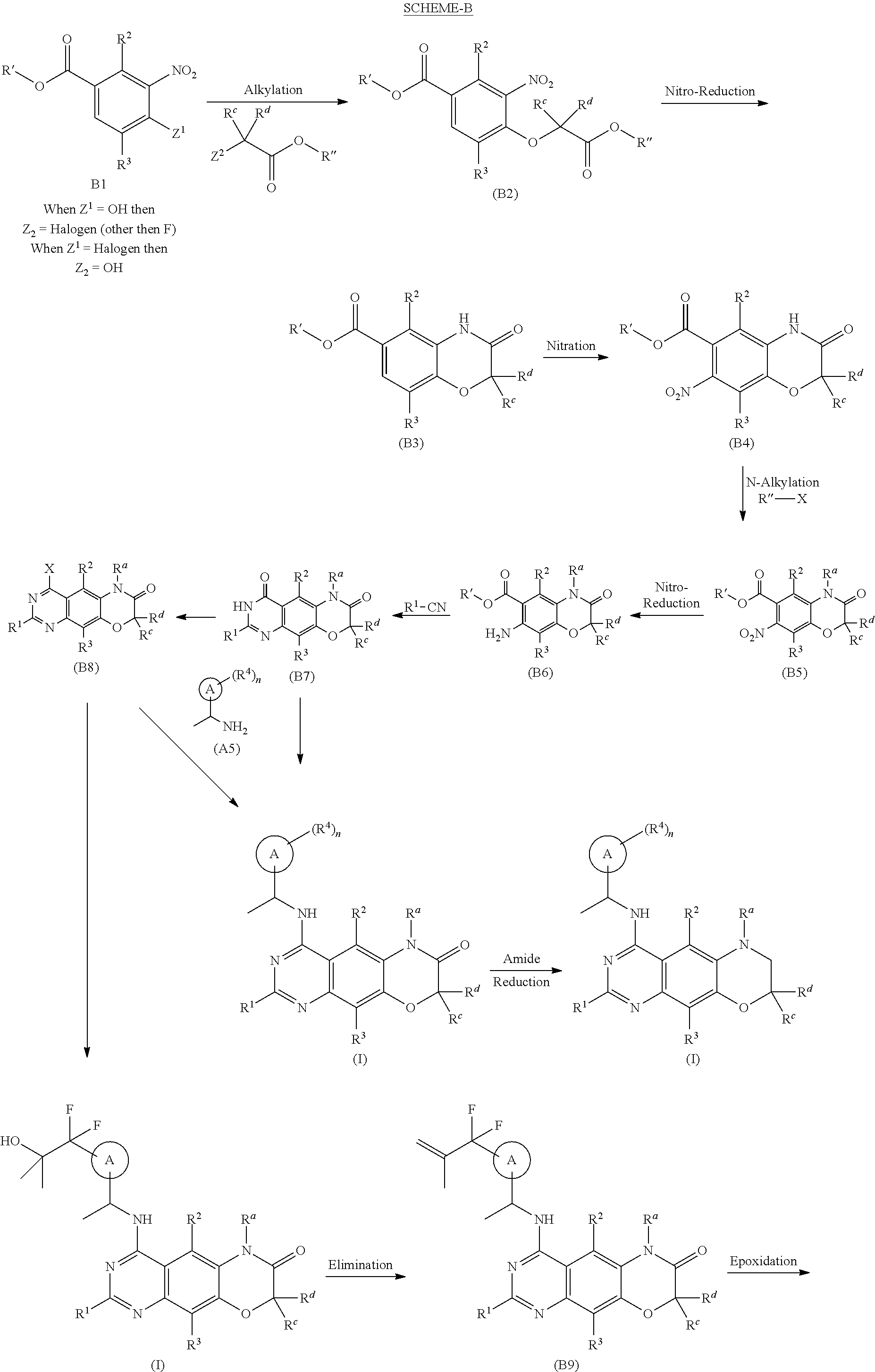

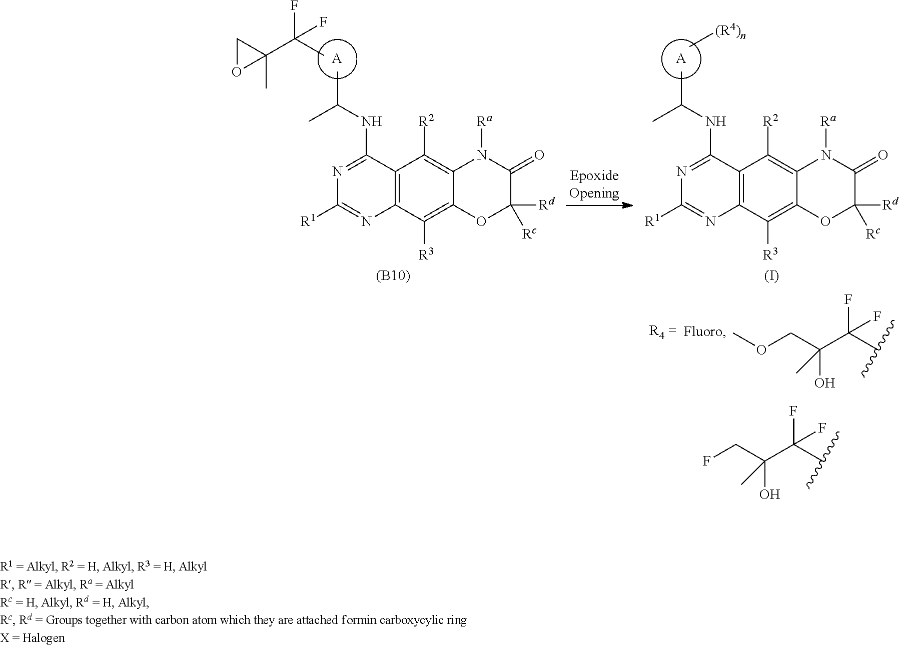

Compound of formula (B2) can be synthesized from compound of formula (B1) by following the reaction protocol as mentioned in EP2243779 (R^{a}═R^{b}═CH₃) and WO2015164480 (R^{a} and R^{b} together forms a ring). Compound of formula (B2) was converted to corresponding cyclic amide of formula (B3) through selective reduction of nitro group by using different reducing agents. Although not limited, such reducing agents include hydrogenation with palladium on carbon, metal reductions like iron, tin or tin chloride and the like. Such reduction of the compound of formula (B2) can be carried out in one or more solvents, e.g., ethers such as THF, 1,4-dioxane, and the like; alcohol such as methanol, ethanol and the like; under acidic conditions involving ammonium chloride, acetic acid, hydrochloric acid and mixtures thereof. Nitration of compound of formula (B3) with nitrating reagents such as, although not limited to fuming nitric acid, potassium nitrate, and the like in acids such as, although not limited to tin (IV) chloride, sulphuric acid, trifluroacetic acid, acetic acid and the like, anhydrides like acetic anhydride, trifluroacetic anhydride and the like, or mixture(s) thereof to provide compound of formula (B4). Compound of formula (B4) can be further alkylated by using corresponding alkyl halide in presence of bases such as Na₂CO₃, K₂CO₃, Cs₂CO₃ etc. in polar aprotic solvents like DMF, DMSO etc. at temperature 20° C.-60° C. leading to compound of formula (B5). An alternative synthetic route towards the compound of formula (B5) is the transformation of intermediate of compound of formula (B4) via Mitsunobu reaction with corresponding alcohol, using different reagents such as but not limited to DEAD, DIAD etc. Such reactions can be carried out in aprotic solvents like, e.g., ethers such as THF, Dioxane and the like; hydrocarbons, e.g., toluene or mixtures thereof, at temperature 25° C.-90° C. Compound of formula (B5) was converted to corresponding aniline derivative compound of formula (B6) through selective reduction of nitro group by using different reducing agents. Although not limited, such reducing agents include hydrogenation with palladium on carbon, metal reductions like iron, tin or tin chloride and the like. Such reduction of the compound of formula (B5) can be carried out in one or more solvents, e.g., ethers such as THF, 1,4-dioxane, and the like; alcohol such as methanol, ethanol and the like, under acidic conditions involving ammonium chloride, acetic acid, hydrochloric acid and the like mixtures thereof. Compound of formula (B6) upon treatment with corresponding alkylnitriles using acids such as but not limited to Methane sulfonic acid, HCl etc. at 25° C.-120° C. to afford compound of formula (B7), which could be further coupled with different chiral benzyl amine (A5) derivatives using different coupling reagents such as but not limited to BOP, PyBop etc. and organic bases such as DBU, DIPEA etc. in a polar aprotic solvent like DMF, DMSO etc. at 0°-120° C. to afford a compound of formula (I).

Alternatively, compound of formula (I) can be prepared from compound of formula (B7) by reacting with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (B8).

Compound of formula (B8) undergoes a nucleophilic substitution reaction with different chiral benzylic amines (A5) leading to the final compound of formula (I) using organic basic reagents such as but not limited to DIPEA, TEA etc. optionally neat or in a polar aprotic solvents like dioxane, THF etc. at 0° C.-130° C. Carbonyl functional group in Compound of formula (I) on further reduction using different reducing reagents such as but not limited to borane DMS, borane THF, LiAlH₄ in polar aprotic solvents like THF, dioxane etc. at temperature 70-90° C. leading to final compound of formula (I).

Compound of formula (I) allowed to react with fluorinating reagent such as DAST, martin sulfurane in solvents such as DCM, chloroform, THF, ether, 1,4-dioxane to provide compound of formula (B9).

Compound of formula (B9) undergoes epoxidation reaction to provide compound of formula (B10). This reaction is effected by hydrogen peroxide in presence of acidic medium using organic acids such as formic acid and like.

Compound of formula (B10) on epoxide opening by nucleophilic reagent provide compound of formula (I). Such transformations can be effected by reaction of epoxide compound with various nucleophilic reagents such as sodium alkoxides, primary or secondary amines in alcohol solvents like ethanol, methanol, and like and at room temperature or elevated temperature.

The compounds of formula (I) was prepared by following the sequential transformations as depicted and described in Scheme-C herein below—

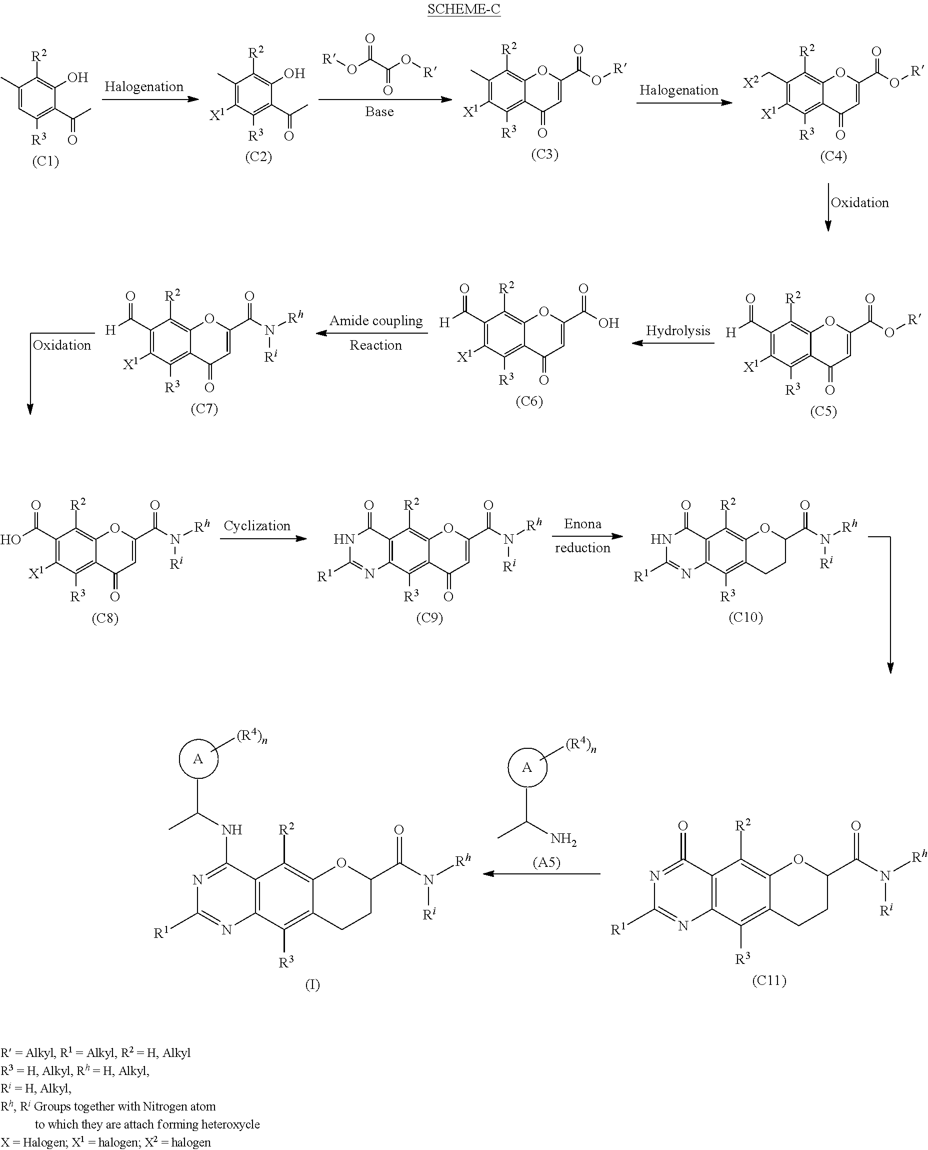

Compound of formula (C2) is prepared by following a procedure reported in Chemistry—A European Journal, 2015, vol. 21, #4, p. 1482-1487. The compound of formula (C2) is converted to corresponding 4-oxo chromene carboxylic ester derivative of compound of formula (C3) using corresponding alpha diketo ester and basic reagents such as but not limited to NaOMe, NaOEt, K^{t}OBu etc. in a polar aprotic solvents like DMF, DMA etc. at 0° C.-75° C. Halogenation of compound of formula (C3) using N-halosuccinamide reagent such as but not limited to NBS, NIS and NCS gives corresponding dihalo compound of formula (C4) via e.g. benzylic halogenation in a aprotic halogenated solvents like CCl₄, DCM etc. at 0°-80° C. The compound of formula (C5) aldehyde derivative can be synthesized by oxidation of compound of formula (C4). Compound of formula (C5) undergoes an acidic hydrolysis leading to compound of formula (C6), that can be further functionalized to corresponding amide of compound of formula (C7) using coupling reagent such as but not limited to PyBop in a polar aprotic solvents like DMF, DMSO etc. at temperature ranging from 0° C.-30° C. for about 1-16h. Compound of formula (C8) can be achieved by oxidation of compound of formula (C7) with suitable oxidizing reagent such as but not limited to sulphamic acid and sodium chlorite. Compound of formula (C8) when condensed with corresponding amidine by coupling reaction affords a quinazoline enone derivative of compound of formula (C9). Reduction of enone compound of formula (C9) using reagents such as but not limited to H₂—Pd/C leading to corresponding compound of formula (C10). The compound of formula (C10) can be transformed to the corresponding compound of formula (C11) via halogenation using reagents such as phosphorus oxyhalide, thionyl chloride and like, in aprotic solvents like chlorobenzene, toluene and mixtures thereof. Compound of formula (C11) undergoes a coupling with different chiral benzylic amines (A5) leading to the final compound of formula (I). This reaction can be effected by organic base such as DIPEA, TEA, DBU or the like, or using coupling reagents such as DCC, EDC, BOP, pyBOP, HBTU or the like; optionally neat or in etheral solvents such as THF, 1,4 dioxane and like or polar aprotic solvents like DMF, DMA, DMSO and thereof at temperature ranging from 20-130° C. The compounds of formula (I) was prepared by following the sequential transformations as depicted and described in Scheme-D herein below.

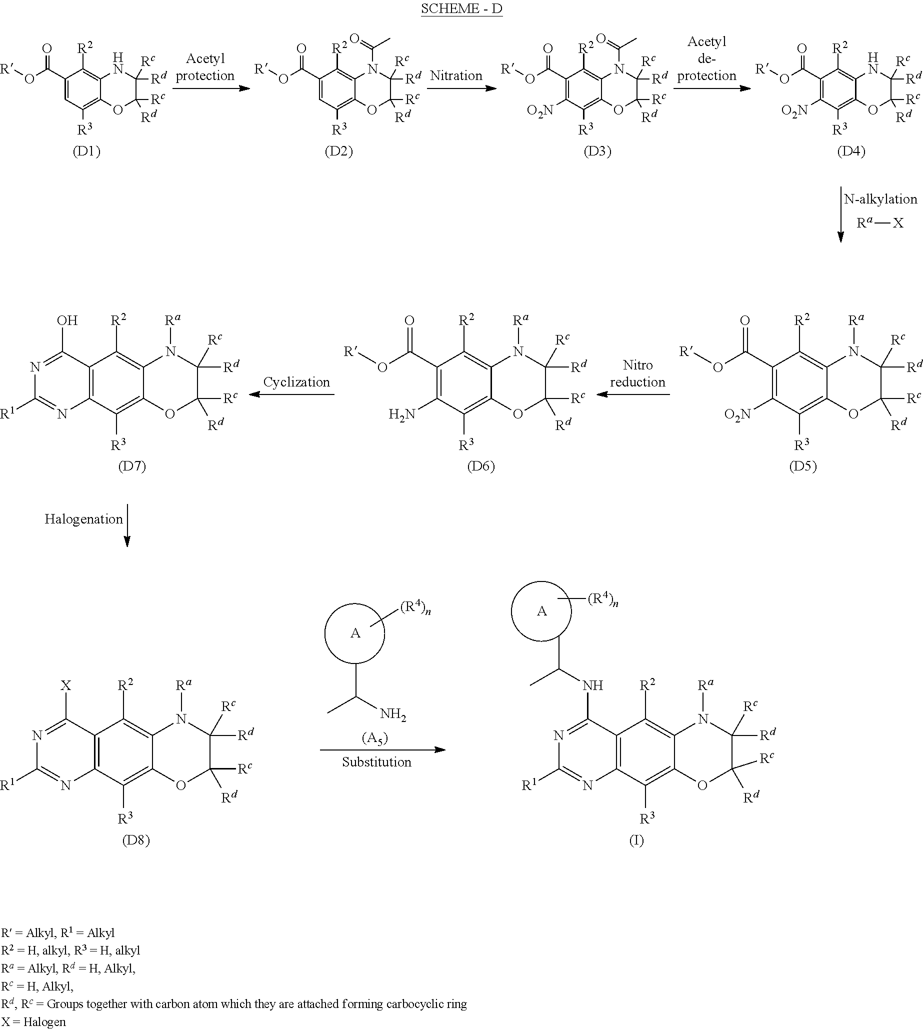

The compound of formula (D1) is converted to corresponding acetyl derivative of compound of formula (D2) via N-acylation reaction using acetyl chloride & using organic basic reagents such as but not limited to pyridine, DIPEA, TEA etc in halogenated solvents such as, although not limited chloroform, dichloromethane, and the like mixtures thereof. Nitration of compound of formula (D2) with nitrating reagents such as, although not limited to fuming nitric acid, potassium nitrate, and the like in acids such as, although not limited to tin (IV) chloride, sulphuric acid, trifluroacetic acid, acetic acid and the like, anhydrides like acetic anhydride, trifluroacetic anhydride and the like, or mixture(s) thereof to provide compound of formula (D3).

Acetyl deprotection of compound of formula (D3) using inorganic bases such as Na₂CO₃, K₂CO₃, Cs₂CO₃, etc in polar protic solvents like methanol, ethanol etc at appropriate temperature afforded compound of formula (D4).

Compound of formula (D4) can be further alkylated by using alkyl halides and bases such as NaH, Na₂CO₃, K₂CO₃, Cs₂CO₃ etc. in polar aprotic solvents like THF, DMF, and DMSO etc. at temperature 20° C.-60° C. leading to compound of formula (D5). Compound of formula (D5) can be converted to corresponding aniline derivative, compound of formula (D6) through selective reduction of nitro group by using different reducing agents. Although not limited, such reducing agents include hydrogenation with palladium on carbon, metal reductions like iron, tin or tin chloride and the like. Such reduction of the compound of formula (D6) can be carried out in one or more solvents, such as methanol, ethanol and the like; under acidic conditions involving ammonium chloride, acetic acid, hydrochloric acid and the like mixtures thereof.

Compound of formula (D6) allowed to react with alkylnitrile in presence of the acidic reagents such as methane sulfonic acid, sulfuric acid, hydrochloric acid or the like to obtain compound of formula (D7).

Compound of formula (D7) was reacted with POCl₃ or POBr₃ optionally in solvents such as toluene, xylene or the like or the mixtures thereof, optionally using organic base such as triethylamine, diisopropylethylamine or the like to provide compound of formula (D8).

Compound of formula (D8) was reacted with compound of formula (A5) in the presence DIPEA, TEA, DBU or the like, or using coupling reagents such as DCC, EDC, BOP, pyBOP, HBTU or the like; optionally neat or in etheral solvents such as THF, 1,4 dioxane and like or polar aprotic solvents like DMF, DMA, DMSO and thereof at temperature ranging from 20-130° C. to provide compound of formula (I).

The compounds of formula (I) was prepared by following the sequential transformations as depicted and described in Scheme-E herein below.

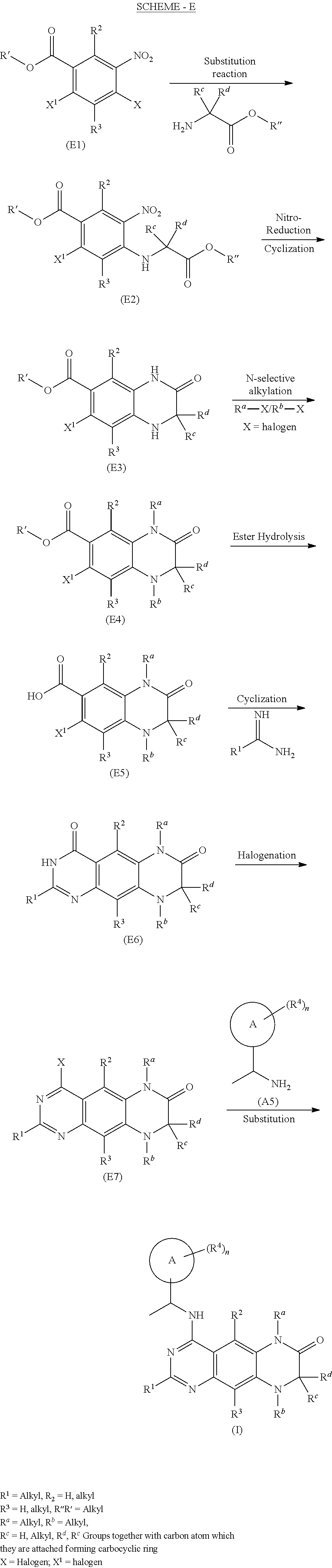

Compound of formula (E1) can be synthesized following a reaction protocol described in WO200879759. Compound of formula (E2) can be synthesized by appropriate displacement of aromatic halogen with corresponding alkyl amine using appropriate bases such as TEA, NaH, Na₂CO₃, K₂CO₃, Cs₂CO₃ etc. in polar aprotic solvents like THF, DMF, DMSO etc. at temperature 20° C.-120° C.

Compound of formula (E2) can be converted to corresponding cyclic amide of formula (E3) through selective reduction of nitro group by using different reducing agents. Although not limited, such reducing agents include hydrogenation with palladium on carbon, metal reductions like iron, tin or tin chloride and the like. Such reduction of the compound of formula (E2) can be carried out in one or more solvents, alcohol such as methanol, ethanol and the like; under acidic conditions involving ammonium chloride, acetic acid, hydrochloric acid and the like mixtures thereof. Compound of formula (E3) can be further alkylated by using bases such as NaH, Na₂CO₃, K₂CO₃, Cs₂CO₃ etc. in polar aprotic solvents like THF, DMF, and DMSO etc. at temperature 20° C.-60° C. leading to compound of formula (E4). Compound of formula (E5) can be synthesized by ester hydrolysis of compound of formula (E4) using bases such as NaOH, LIOH and KOH etc. Compound of formula (E5) which on coupling with different amidines such as acetamidine, formamidine etc. in polar aprotic solvents like DMF, DMSO etc. at temperature 80° C.-100° C. leading to compound of formula (E6). Compound of formula (E6) can be converted to the corresponding compound of formula (E7) by halogenation using reagents such as POCl₃, POBr₃, SOCl₂ etc.

Compound of formula (E7) undergoes a nucleophilic substitution reaction with different chiral benzyl amine (A5) leading to compound of formula (I) using aprotic solvents like dioxane, THF and like, at temperature 0° C.-130° C. and bases such as but limited to DIPEA, TEA and thereof.

The compounds of formula (I) was prepared by following the sequential transformations as depicted and described in Scheme-F herein below.

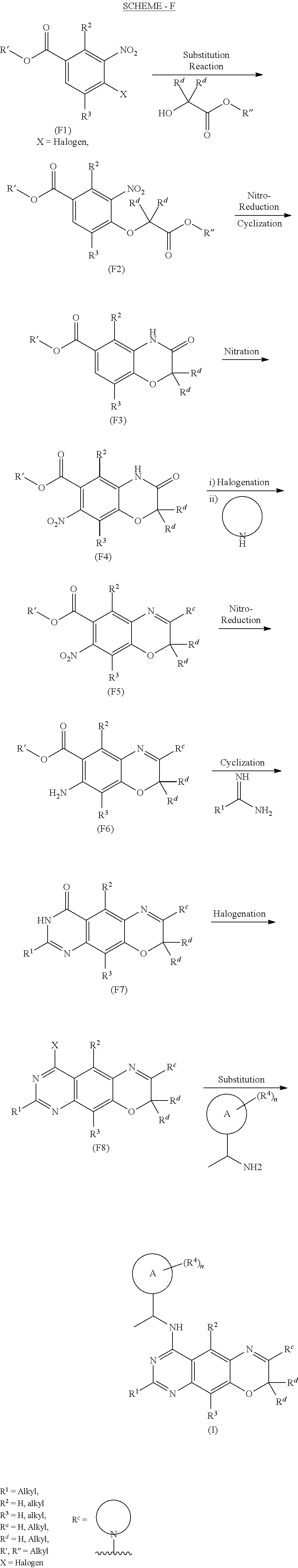

Compound of formula (F2) can be synthesized by following the reaction protocol as mentioned in EP2243779 (R^{c}═R^{d}═CH₃) and WO2015164480 (R^{c} and R^{d} together forms a ring). Compound of formula (F2) was converted to corresponding cyclic amide of formula (F3) through selective reduction of nitro group by using different reducing agents. Although not limited, such reducing agents include hydrogenation with palladium on carbon, metal reductions like iron, tin or tin chloride and the like. Such reduction of the compound of formula (F2) can be carried out in one or more solvents, such as methanol, ethanol and the like; under acidic conditions involving ammonium chloride, acetic acid, hydrochloric acid and the like mixtures thereof. Nitration of compound of formula (F3) with nitrating reagents such as, although not limited to fuming nitric acid, potassium nitrate, and the like in acids such as, although not limited to tin (IV) chloride, sulphuric acid, trifluroacetic acid, acetic acid and the like, anhydrides like acetic anhydride, trifluroacetic anhydride and the like, or mixture(s) thereof to provide compound of formula (F4).

Compound of formula (F4) can be treated with SOCl, POCl₃, POBr₃ and thereof using DMF to give an intermediate (Halogenation reaction intermediate), which undergoes a nucleophilic substitution reaction with appropriate amines leading to the compound of formula (F5), using organic basic reagents such as but not limited to DIPEA, TEA etc. in a polar aprotic solvent like dioxane, THF etc. at appropriate temperature.

Compound of formula (F5) can be converted to corresponding aniline derivative, compound of formula (F6) through selective reduction of nitro group by using different reducing agents. Although not limited, such reducing agents include hydrogenation with palladium on carbon, metal reductions like iron, tin or tin chloride and the like. Such reduction of the compound of formula (F5) can be carried out in one or more solvents, such as methanol, ethanol and the like; under acidic conditions involving ammonium chloride, acetic acid, hydrochloric acid and mixtures thereof. Compound of formula (F6) upon treatment with corresponding nitrile solvents such as but not limited to acetonitrile using acids such as but not limited to methane sulfonic acid, HCl etc. at 25° C.-120° C. to afford compound of formula (F7), which can be transformed to intermediate (F8), via e.g. triflate or halogenation etc. of the corresponding compound of formula (F7). Compound of formula (F8) undergoes a nucleophilic substitution reaction with different chiral benzyl amine (A5), using aprotic solvents like dioxane, THF etc., at temperature 0° C.-130° C. and bases such as but limited to DIPEA, TEA etc. leading to final compound of formula (I).

The compounds of formula (I) was prepared by following the sequential transformations as depicted and described in Scheme-G herein below.

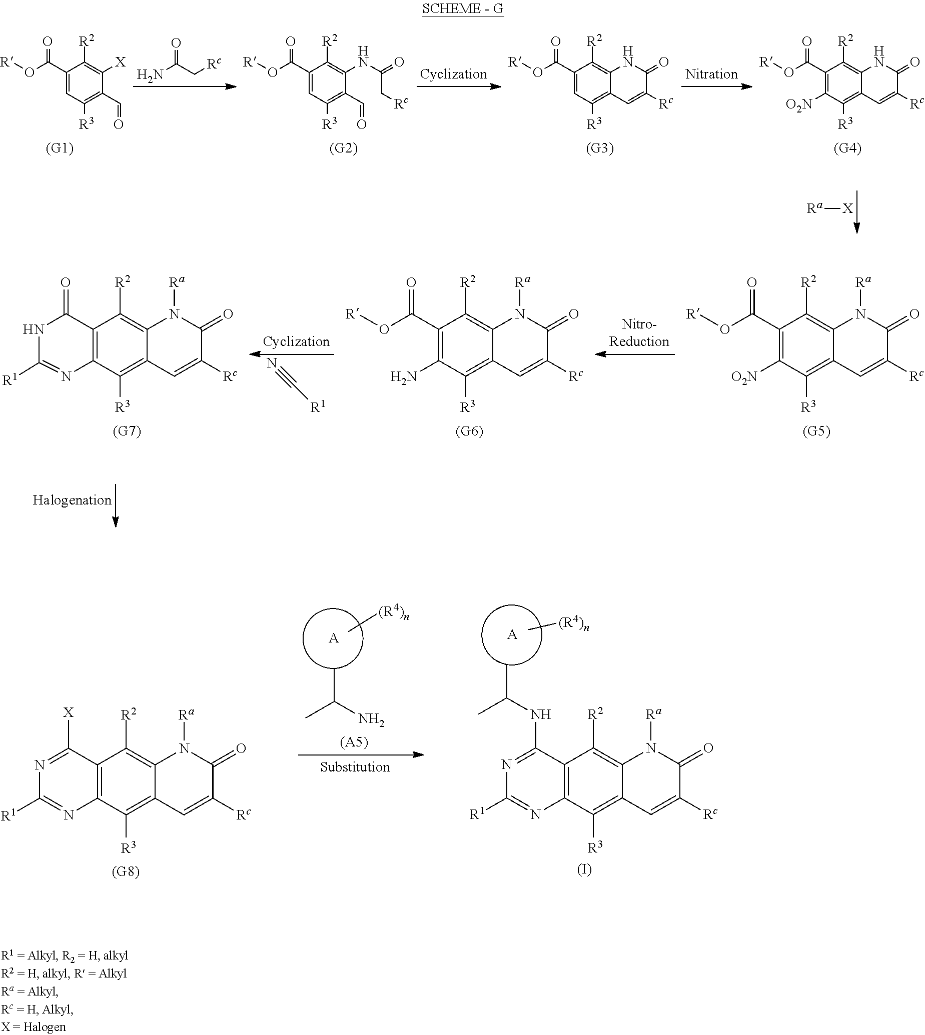

Compound of formula (G1) was allowed to react with corresponding carbamate in the presence of catalyst such as (tris(dibenzylideneacetone)dipalladium( ), palladium(II) acetate, Bis(dibenzylideneacetone)2 Pd(0), rac 2,2′-Bis(diphenylphosphino)-1,1′-binaphthyl, 2,5 bis(tri-t-butylphosphine) palladium (0) and the like; in presence of ligands such as RuPhos, Xanthphos, Davephos, BINAP, or the like; using a suitable base such as sodium carbonate, cesium carbonate, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (G2)

Cyclization of compound of formula (G2) provided compound of formula (G3), in the presence of suitable base, preferably inorganic bases such as alkali metal carbonates, e.g., Na₂CO₃, K₂CO₃, Cs₂CO₃, NaO^{t}Bu, Potassium phosphate, or mixture thereof. Such reactions can be carried out in solvents like, e.g., ethers such as THF, Dioxane and the like; hydrocarbons, e.g., toluene; amides such as DMF, DMA or mixtures thereof.

Nitration of compound of formula (G3) with nitrating reagents such as, although not limited to fuming nitric acid, potassium nitrate, and the like in acids such as, although not limited to tin (IV) chloride, sulphuric acid, trifluroacetic acid, acetic acid and the like, anhydrides like acetic anhydride, trifluroacetic anhydride and the like, or mixture(s) thereof to provide compound of formula (G4).

The compound of formula (G4) was alkylated to give compound of formula (G5). This conversion was effected in presence alkali hydrides like sodium hydride and like; or bases such as potassium carbonate and like; and alkylating reagents alkyl halides e.g. Methyl iodide and like; in presence of solvents such as THF, DMF or mixture(s) thereof.

Compound of the formula (G6) was obtained from compound of formula (G5) using by metal reductions using iron, tin or tin chloride or the like in solvents selected from THF, 1,4-dioxane methanol, ethanol or the like or mixtures thereof under acidic condition using ammonium chloride, acetic acid, hydrochloric acid or the like or mixture(s) thereof. This transformation can also be carried out by catalytic hydrogenation using Pd/C and thereof in solvents ethyl acetate, Methanol or mixture(s) thereof.

Compound of formula (G6) reacted with alkylnitriles in presence of the reagent such as methane sulfonic acid, sulfuric acid, hydrochloric acid or the like to obtain compound of formula (G7).

Compound of formula (G7) was reacted with POCl₃ or POBr₃ optionally in solvents such as toluene, xylene or the like or the mixtures thereof, optionally using organic base such as triethylamine, diisopropylethylamine or the like to provide compound of formula (G8).

Compound of formula (G8) was reacted with compound of formula (A5) in the presence of triethyl amine, N,N-ethyldiisopropyl amine, pyridine, DBU or the like in solvents such as THF, 1,4-Dioxane, toluene, DCM. DMSO or mixture(s) thereof to provide compound of formula (I).

The compounds of formula (I) was prepared by following the sequential transformations as depicted and described in Scheme-H herein below.

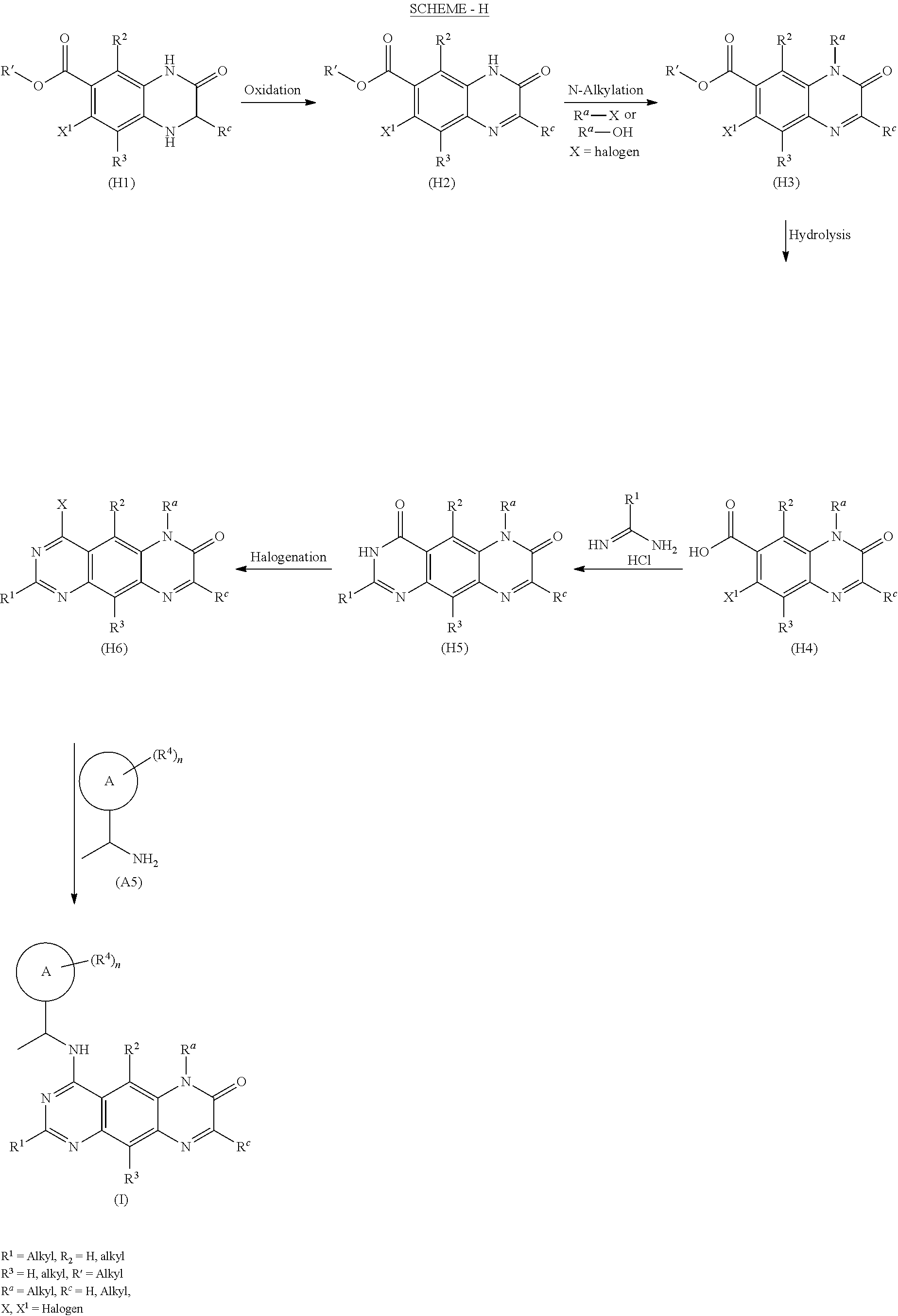

Compound of formula (H1) can be synthesized by reaction protocol as mentioned in (WO243823). Compound of formula (H2) can be synthesized from compound of formula (H1) by using oxidizing agents like MnO₂, H₂O₂, AgNO₃, DDQ and thereof. Compound of formula (H2) undergoes alkylation reaction using alkyl halides in presence of bases such as K₂CO₃, Na₂CO₃, Cs₂CO₃ and like; in polar aprotic solvents like DMF, DMSO and thereof; at temperature 20° C.-60° C. afforded compound of formula (H3).

An alternative synthetic route towards the compound of formula (H3) is the transformation of intermediate of compound of formula (H2) via Mitsunobu reaction with corresponding alcohol, using different reagents such as but not limited to DEAD, DIAD etc. Such reactions can be carried out in aprotic solvents like, e.g., ethers such as THF, Dioxane and the like; hydrocarbons, e.g., toluene or mixtures thereof, at temperature 25° C.-90° C.

Compound of formula (H4) can be synthesized by ester hydrolysis of formula (H3) using bases such as NaOH, LiOH, KOH and like; in polar protic solvents such as methanol, ethanol and like.

Compound of formula (H4) on reaction with acetamidine, formamidine and like; in polar aprotic solvents like DMF, DMSO and thereof at temperature elevated temperatures afforded compound of formula (H5).

Compound of formula (H7) was reacted with POCl₃ or POBr₃ optionally in solvents such as toluene, xylene or the like or the mixtures thereof, optionally using organic base such as triethylamine, diisopropylethylamine or the like to provide compound of formula (H6).

Compound of formula (H6) was reacted with compound of formula (A5) in the presence of triethyl amine, N,N-ethyldiisopropyl amine, pyridine, DBU or the like in solvents such as THF, 1,4-Dioxane, toluene, DCM, DMSO or mixture(s) thereof to provide compound of formula (I).

The compounds of formula (I) was prepared by following the sequential transformations as depicted and described in Scheme-I herein below.

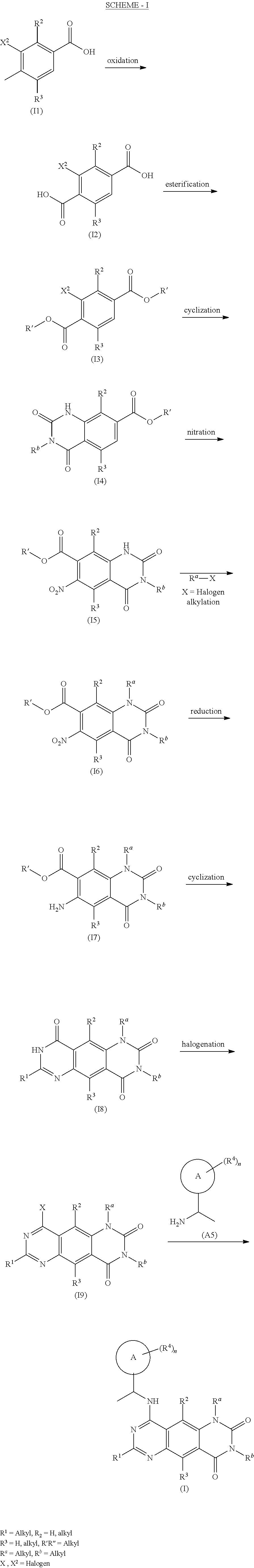

The compound of the formula (I2) obtained by treating compound of the formula (II) with oxidizing agent potassium permanganate, potassium dichromate, sodium dichromate in presence of acids like sulphuric acid, acetic acid and like, in 1:1 mixture of t-butanol and Water as Solvent.

The compound of formula (I2) was subjected to esterification in alcoholic solvents like methanol ethanol and thereof in presence of chlorinating agents such as thionyl chloride, oxalyl chloride and thereof, or in presence of acidic reagents such as sulfuric and methane sulfonic acid thereof to provide the compound of formula (I3).

The compound of formula (I3) was subjected to C—N coupling reaction e.g. Buchwald reaction with 1-methylurea provided compound of formula (I4). This reaction can mediated by a suitable catalyst such as, e.g., Pd(PPh₃)₂Cl₂, Pd₂dba₃, Pd(PPh₃)₄, Pd(OAc)₂ or mixtures thereof; a suitable ligand such as Xantphos, BINAP, Ru-Phos, XPhos, or mixtures thereof; in the presence of suitable base, preferably inorganic bases such as alkali metal carbonates, e.g., K₂CO₃, Na₂CO₃, Cs₂CO₃, NaO^{t}Bu, Potassium phosphate, or mixture thereof. Such reactions can be carried out in solvents like, e.g., ethers such as THF, Dioxane and the like; hydrocarbons, e.g., toluene; amides such as DMF, DMA or mixtures thereof.

Nitration of compound of formula (I4) with nitrating reagents such as, although not limited to fuming nitric acid, potassium nitrate, and the like in acids such as, although not limited to tin (IV) chloride, sulphuric acid, trifluroacetic acid, acetic acid and the like, anhydrides like acetic anhydride, trifluroacetic anhydride and the like, or mixture(s) thereof to provide compound of formula (I5).

The compound of formula (I5) was alkylated to give compound of formula (I6). This conversion was effected in presence alkali hydrides like sodium hydride and like; or bases such as potassium carbonate and like; and alkylating reagents alkyl halides e.g. Methyl iodide and like; in presence of solvents such as THF, DMF or mixture(s) thereof.

Compound of the formula (I7) was obtained from compound of formula (I6) using by metal reductions using iron, tin or tin chloride or the like in solvents selected from THF, 1,4-dioxane methanol, ethanol or the like or mixtures thereof under acidic condition using ammonium chloride, acetic acid, hydrochloric acid or the like or mixture(s) thereof. This transformation can also be carried out by catalytic hydrogenation using Pd/C and thereof in solvents ethyl acetate, Methanol or mixture(s) thereof.

Compound of formula (I7) reacted with acetonitrile in presence of the reagent such as methane sulfonic acid, sulfuric acid, hydrochloric acid or the like to obtain compound of formula (I8).

Compound of formula (I8) was reacted with POCl₃ or POBr₃ optionally in solvents such as toluene, xylene or the like or the mixtures thereof, optionally using organic base such as triethylamine, diisopropylethylamine or the like to provide compound of formula (I9).

Compound of formula (I9) was reacted with compound of formula (A5) in the presence of triethyl amine, N,N-ethyldiisopropyl amine, pyridine, DBU or the like in solvents such as THF, 1,4-Dioxane, toluene, DCM, DMSO or mixture(s) thereof to provide compound of formula (I).

The compounds of formula (I) was prepared by following the sequential transformations as depicted and described in Scheme-J herein below.

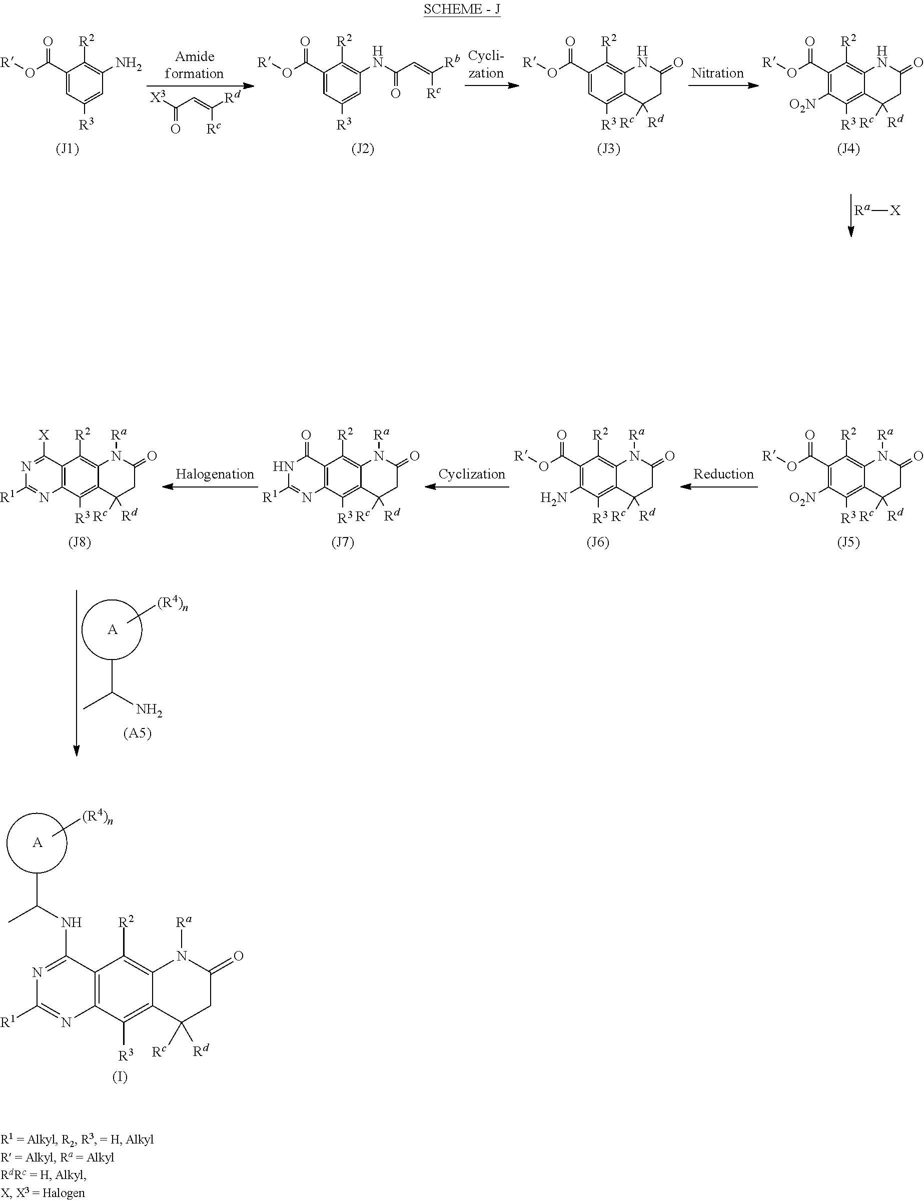

Compound of formula (J2) can be synthesized from compound of formula (J1) by following the reaction protocol as mentioned in ACS Medicinal Chemistry Letters, 2018, vol. 9, #8, p. 827-831 (R^{b}═R^{c}═CH). Upon thermal cyclization at elevated temperature(s) the compound of the formula (J2) can undergo ring cyclization to produce compound of formula (J3). Such reaction can be carried out by using Lewis acids such as, although not limited to AIC1₃, BF₃, etc., either neat or by using solvents such as DCM, DCE, chlrobenzene, toluene, xylene, etc. and the like or mixture(s) thereof. Nitration of compound of formula (J3) with nitrating reagents such as, although not limited to fuming nitric acid, potassium nitrate, and the like in acids such as, although not limited to tin (IV) chloride, sulphuric acid, trifluroacetic acid, acetic acid and the like, anhydrides like acetic anhydride, trifluroacetic anhydride and the like, or mixture(s) thereof to provide compound of formula (J4). Compound of formula (J4) can be further alkylated by using bases such as NaH, K₂CO₃, Na₂CO₃, Cs₂CO₃ etc. in polar aprotic solvents like THF, DMF, DMSO etc. at appropriate temperature leading to compound of formula (J5). Compound of formula (J5) was converted to corresponding aniline derivative compound of formula (J6) through selective reduction of nitro group by using different reducing agents. Such reducing agents include hydrogenation with palladium on carbon, metal reductions like iron, tin or tin chloride and the like. Such reduction can be carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof. Compound of formula (J6) upon treatment with corresponding alkylnitriles using acids such as but not limited to Methane sulfonic acid, HCl etc. at appropriate temperature to afford compound of formula (J7). The halogenation of compound of formula (J7) to produce the compound of formula (J8). Such reaction can be carried out by using neat halogenating reagents, such as but not limited to POCl₃, POBr₃, SOCl₂ and the like at appropriate temperature. This reaction can also be caried out by using combination of halogenating reagents and organic bases such as POC₃, POBr₃, SOCl₂ and the like; and organic bases like DIPEA, TEA, N,N-Dimethylaniline and the like; using solvents such as DCE, DCM, chlorobenzene, toluene and the like or mixture(s) thereof at appropriate temperature. The compound of formula (I) can be obtained by using nucleophilic substitution of benzyl amines (A5) with the compound of the formula (J8). Such reaction can be carried out at appropriate temperature in presence of bases like DIPEA, TEA and the like; in solvents such as THF, 1,4-Dioxane, DCE, ACN, DMSO, etc., and the like or mixture(s) thereof.

The compounds of formula (I) was prepared by following the sequential transformations as depicted and described in Scheme-K herein below.

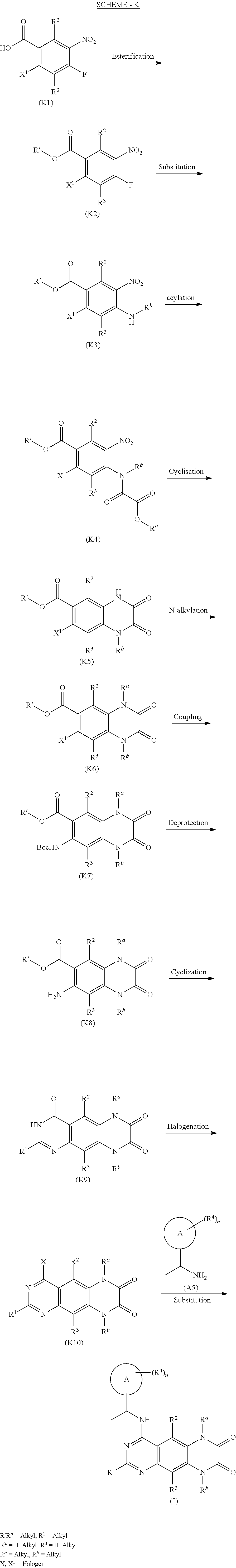

The compound of formula (K1) was subjected to esterification in alcoholic solvents like methanol ethanol and thereof in presence of chlorinating agents such as thionyl chloride, oxalyl chloride and thereof, or in presence of acidic reagents such as sulfuric and methane sulfonic acid thereof to provide the compound of formula (K2).

Compound of formula (K3) can be synthesized by appropriate displacement of aromatic halogen with corresponding alkyl amine in alcoholic solvents like methanol ethanol and thereof.

Compound of formula (K3) was reacted with oxalyl chloride in the presence of bases like triethyl amine, N,N-ethyldiisopropyl amine, pyridine, DBU or the like in solvents such as THF, 1,4-Dioxane, toluene, DCM, or mixture(s) thereof to provide compound of formula (K4).

Compound of formula (K4) was subjected to cyclisation using dithionate salts in the presence of mixture of solvents such as THF, 1,4-Dioxane, in alcoholic solvents like methanol ethanol and water, mixture(s) thereof to provide compound of formula (K5). The compound of formula (K5) was alkylated to give compound of formula (K6). This conversion was effected in presence alkali hydrides like sodium hydride and like; or bases such as potassium carbonate and like; and alkylating reagents alkyl halides e.g. Methyl iodide and like; in presence of solvents such as THF, DMF or mixture(s) thereof.

The compound of formula (K6) was subjected to C—N coupling reaction e.g. Buchwald reaction with tert-butyl carbamate provided compound of formula (K7). This reaction can mediated by a suitable catalyst such as, e.g., Pd(PPh₃)₂Cl₂, Pd₂dba₃, Pd(PPh₃)₄, Pd(OAc)₂ or mixtures thereof; a suitable ligand such as Xantphos, BINAP, Ru-Phos, XPhos, or mixtures thereof; in the presence of suitable base, preferably inorganic bases such as alkali metal carbonates, e.g., K₂CO₃, Na₂CO₃, Cs₂CO₃, NaOtBu, Potassium phosphate, or mixture thereof. Such reactions can be carried out in solvents like, e.g., ethers such as THF, Dioxane and the like; hydrocarbons, e.g., toluene; amides such as DMF, DMA or mixtures thereof.

Compound of formula (K7) undergoes deprotection using acids like organic acids such as trifluoroacetic acid, Methane sulfonic acid and like, mineral acids like hydrochloric acid, acetic acid (Aqueous or in etheral solvents), sulfuric acid and the like; using solvents like dichloromethane, dichloroethane, THF, 1,4-dioxane and like thereof to provide compound of formula (K8).

Compound of formula (K8) reacted with alkyl nitriles in presence of the reagent such as methane sulfonic acid, sulfuric acid, hydrochloric acid, or the like to obtain compound of formula (K9).

Compound of formula (K9) was reacted with POCl₃ or POBr₃ optionally in solvents such as toluene, xylene or the like or the mixtures thereof, optionally using organic base such as triethylamine, diisopropylethylamine or the like to provide compound of formula (K10).

Compound of formula (K10) was reacted with compound of formula (A5) in the presence of triethyl amine, N,N-ethyldiisopropyl amine, pyridine, DBU or the like in solvents such as THF, 1,4-Dioxane, toluene, DCM. DMSO or mixture(s) thereof to provide compound of formula (I).

The compounds of formula (I) was prepared by following the sequential transformations as depicted and described in Scheme-L herein below.

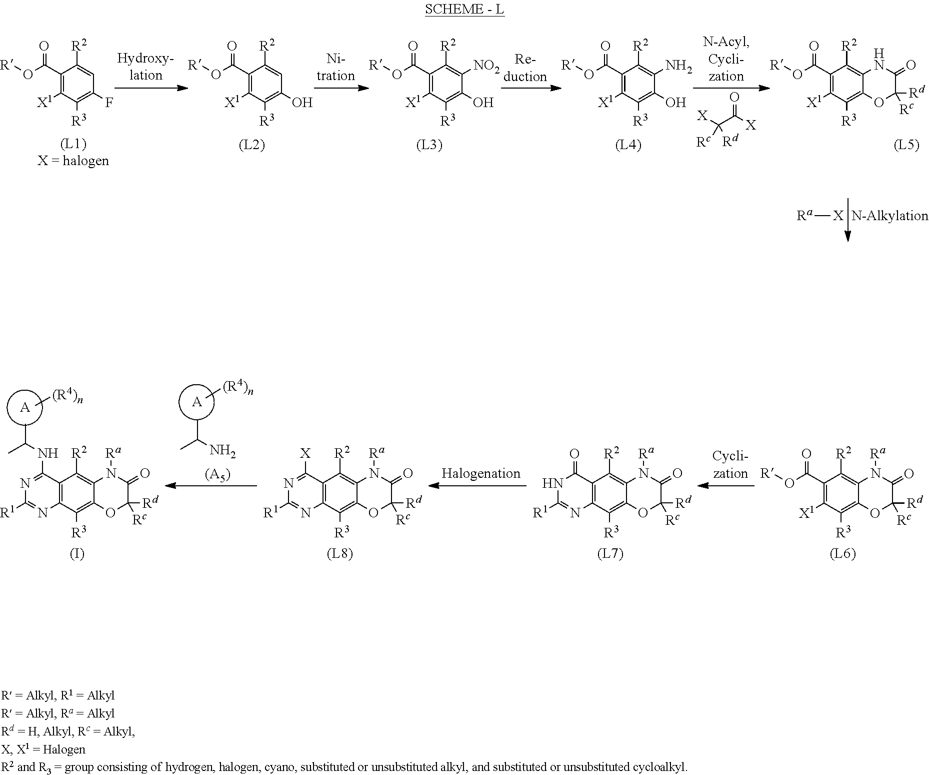

Compound of formula (L1) allowed to react with N-hydroxyacetamide in presence of the bases such as K₂C03, Na₂CO₃, Cs₂CO₃ etc. in polar aprotic solvents like DMF, DMSO etc. at temperature 20° C.-80° C. leading to compound of formula (L2). Nitration of compound of formula (L2) with nitrating reagents such as, although not limited to fuming nitric acid, potassium nitrate, and the like in acids such as, although not limited to tin (IV) chloride, sulphuric acid, trifluroacetic acid, acetic acid and the like, anhydrides like acetic anhydride, trifluroacetic anhydride and the like, or mixture(s) thereof to provide compound of formula (L3). Compound of formula (L3) was converted to corresponding aniline derivative compound of formula (L4) through selective reduction of nitro group by using different reducing agents. Although not limited, such reducing agents include hydrogenation with palladium on carbon, metal reductions like iron, tin or tin chloride and the like. Such reduction of the compound of formula (L3) can be carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof. Compound of formula (L4) allowed to react with corresponding acyl halide in presence of the organic basic reagents such as but not limited to DIPEA, TEA etc. in polar aprotic solvents like DMF, DMSO etc. at temperature 20° C.-80° C. leading to compound of formula (L5). Compound of formula (L5) can be further alkylated by using bases such as K₂CO₃, Na₂CO₃, Cs₂CO₃ etc. in polar aprotic solvents like DMF, DMSO etc. at temperature 20° C.-60° C. leading to compound of formula (L6). Compound of formula (L6) which on coupling with different amidines such as acetamidine, formamidine etc. in polar aprotic solvents like DMF, DMSO etc. at temperature 80° C.-100° C. leading to compound of formula (L7).

Compound of formula (L8) can be prepared from compound of formula (L7) by reacting with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (L8).

Compound of formula (L8) undergoes a nucleophilic substitution reaction with different chiral benzylic amines (A5) leading to the final compound of formula (I) using organic basic reagents such as but not limited to DIPEA, TEA etc. in a polar aprotic solvents like dioxane, THF etc. at 0° C.-130° C.

The compounds of formula (I) was prepared by following the sequential transformations as depicted and described in Scheme-M herein below.

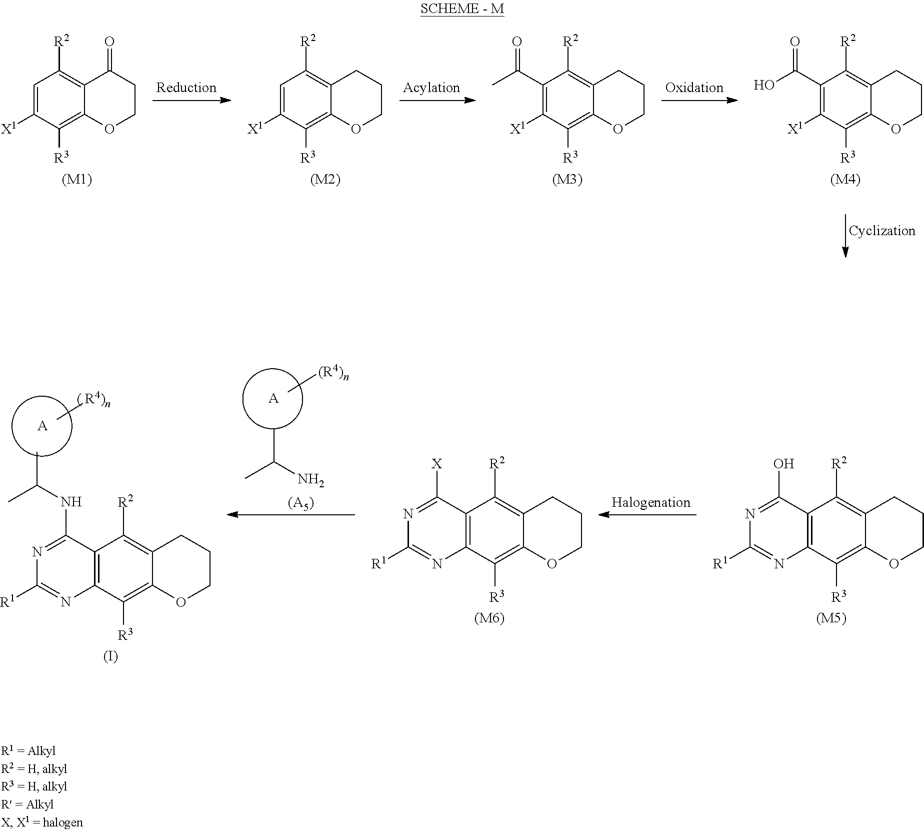

Carbonyl functional group in Compound of formula (M1) on further reduction using different reducing reagents such as but not limited to triethyl silane, borane DMS, borane THF, LiAlH₄ in polar aprotic solvents like THF, dioxane etc or like in acids such as, although not limited to trifluroacetic acid, sulphuric acid, acetic acid and the like, or mixture(s) thereof to provide compound of formula (M2).

Compound of formula (M2) converted to compound of formula (M3) using Friedel craft acylation. This transformation was carried out by reaction of Compound of formula (M2) with corresponding acyl halide in presence of Lewis acids such as aluminum trichloride, zinc chloride, boron trifluoride etherate and like, in halogenated solvents like dichloromethane, dichloroethane and like.

Compound of formula (M3) was allowed to react with mixture of bromine & aqueous metal hydroxides like NaOH, KOH or the like or mixtures thereof to provide compound of formula (M4).

Compound of formula (M4) which on coupling with different amidines such as acetamidine, formamidine etc. in polar aprotic solvents like DMF, DMSO etc. at temperature 80° C.-100° C. leading to compound of formula (M5).

Compound of formula (M6) can be prepared from compound of formula (M5) by reacting with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (M6).

Compound of formula (M6) undergoes a nucleophilic substitution reaction with different chiral benzylic amines (A5) leading to the final compound of formula (I) using organic basic reagents such as but not limited to DIPEA, TEA etc. in a polar aprotic solvents like dioxane, THE etc. at 0° C.-130° C.

The compounds of formula (I) was prepared by following the sequential transformations as depicted and described in Scheme-N herein below.

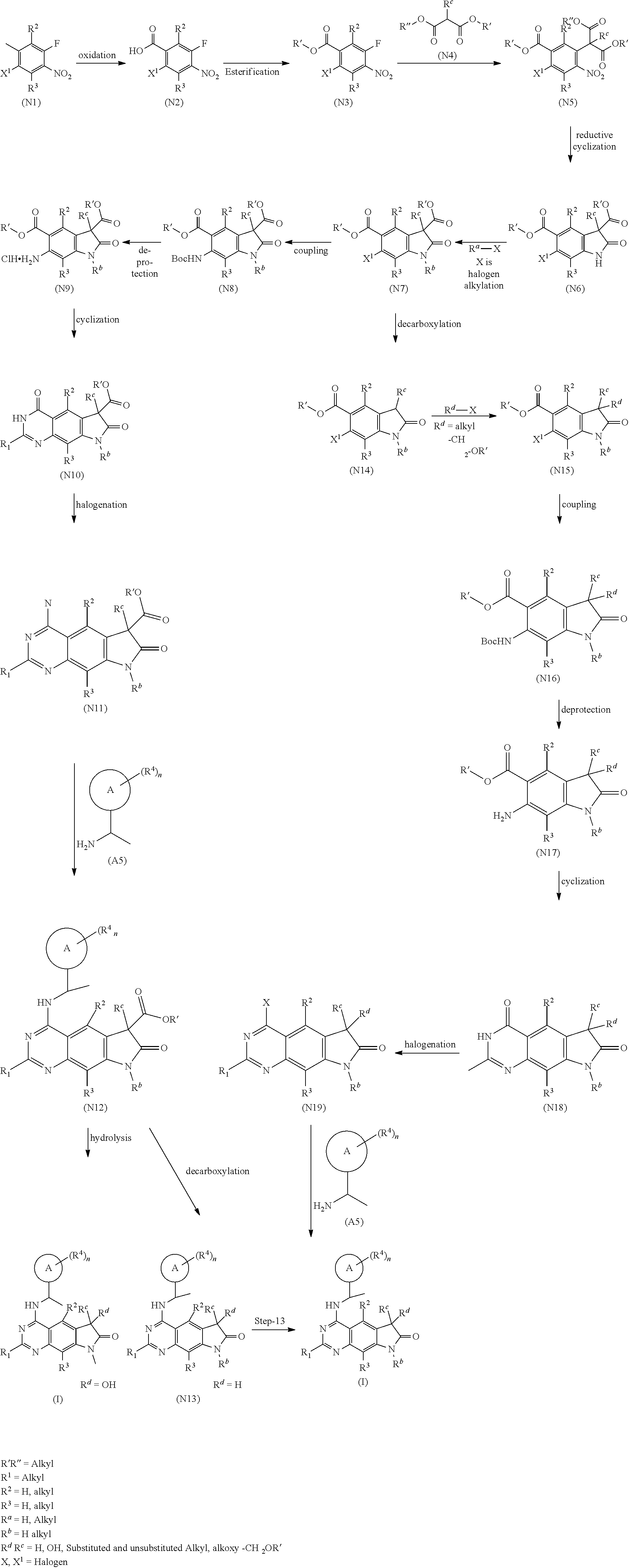

Compound of the formula (N2) was obtained by oxidation of compound of the formula (N1). This transformation can be effected by oxidizing reagents such as potassium permanganate, potassium dichromate, sodium dichromate and like; in presence of acids like H₂SO₄, acetic acid and like.

Compound of the formula (N3) was obtained from compound of the formula (N2) by esterification reaction. This transformation can be effected by reaction of alcohols such as methanol, ethanol and like; in presence of mineral acids like sulfuric acid, organic acids like methane sulfonic acid and like, or in presence of chloride reagents like thionyl chloride, oxalyl chloride and thereof. This transformation can also be effected by Mitsonobu reaction between acid (N3) and corresponding alcohols in presence of Triaryl phosphines and azo carboxylates such as DEAD, DIAD and like.

The reaction between compound of formula (N3) and substituted dialkyl dicarboxylates (compound of the formula (N4)) in presence of base provided compound of the formula (N5). This type of transformations can be carried out either at room temperature or at elevated temperatures using alkali bases such as NaOH, KOH and like; carbonates such as potassium carbonate, cesium carbonate and like; or organic bases like Triethylamine, diisopropylethyl amine and thereof; in amidic solvents like DMF, DMA and like; etheral solvents like 1, 4-dioxane, THF and thereof.

Compound of formula (N5) undergo reductive cyclization to provide compound of formula (N6). The reduction of nitro group was carried out using different reagents; although not limited, such reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and mixtures thereof.

Compound of formula (N6) undergoes N-alkylation using alkyl halides and bases such as K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (N7).

Compound of formula (N7) allowed to react with tert-butyl carbamate in the presence of catalyst such as (tris(dibenzylideneacetone) dipalladium(0), palladium (II) acetate, Bis(dibenzylideneacetone)₂ Pd(0), racemic 2,2′-Bis(diphenylphosphino)-1,1′-binaphthyl, 2,5 bis(tri-t-butylphosphine) palladium (0) and the like; in presence of ligands such as RuPhos, Xanthphos, Davephos, BINAP, or the like; using a suitable base such as sodium carbonate, cesium carbonate, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (N8).

Compound of formula (N8) undergoes deprotection using acids like organic acids such as trifluoroacetic acid, Methane sulfonic acid and like, mineral acids like hydrochloric acid, acetic acid (aqueous or in etheral solvents), sulfuric acid and the like; using solvents like dichloromethane, dichloroethane, THF, 1,4-dioxane and like thereof to provide compound of formula (N9).

Compound of formula (N9) allowed to react with alkylnitrile in presence of the acidic reagents such as methane sulfonic acid, sulfuric acid, hydrochloric acid or the like to obtain compound of formula (N10). The same transformation can be carried out using trialkyl orthoacetate in presence of ammonium acetate, in corresponding polar protic solvents like ethanol, methanol and thereof.

Alternatively, compound of formula (N8) on reaction with alkylnitrile in presence of the acidic reagents such as methane sulfonic acid, sulfuric acid, hydrochloric acid or the like can directly give compound of formula (N10)

Compound of formula (N10) can also be obtained directly from compound of formula (N8) by reaction alkylnitrile in presence of the acidic reagents such as methane sulfonic acid, sulfuric acid, hydrochloric acid and thereof.

Compound of formula (N10) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (N11).

Compound of formula (N11) allowed to react with compound of formula (A5) in presence of suitable coupling reagent to provide compound of formula (N12). The reaction can be carried out in presence of organic base such as diisopropylethylamine, triethylamine, DBU or the like, or using coupling reagents such as DCC, EDC, BOP, pyBOP, HBTU or the like; in etheral solvents such as THF, 1,4 dioxane and like or polar aprotic solvents like DMF, DMA, DMSO and thereof.

Compound of formula (N12) converted to compound of formula (I) in presence of alkali hydroxides such as NaOH, LiOH and thereof, in solvents like methanol, ethanol and thereof or using tetrabutyl ammonium halide in etheral solvents like THF, 1,4-dioxane and thereof.

Compound of formula (N12) undergoes decarboxylation reaction to furnish compound of the formula (N13). This transformation can be effected by acidic reagents such as mineral acids like sulfuric acid, organic acids like trifluoroacetic acid and thereof; similar transformation can be achieved using sodium chloride, lithium chloride and thereof, in solvents such as dimethyl sulfoxide and like; at elevated temperatures.

Compound of formula (N13) converted to compound of formula (I) using ceric ammonium nitrate, thallium nitrate and thereof in present of alcoholic solvents like methanol, ethanol and thereof.

Further, Compound of formula (N7) undergoes decarboxylation reaction to furnish compound of the formula (N14). This transformation can be achieved using sodium chloride, lithium chloride and thereof, in solvents such as dimethyl sulfoxide and like, at elevated temperatures. Similar transformation can be effected by acidic reagents such as mineral acids like sulfuric acid, organic acids like trifluoroacetic acid and thereof.

Compound of formula (N14) undergoes C-alkylation reaction with alkyl halides in presence of bases such as NaH, sodium/potassium alkoxides, K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1, 4-dioxane and like, at room temperature or elevated temperatures provide compound of formula (N15)

Compound of formula (N15) can be converted to compound of formula (I) in five steps by employing analogous protocol mentioned above in scheme—N for the conversion of compound of formula (N7) to compound of formula (N12).

The compounds of formula (I) was prepared by following the sequential transformations as depicted and described in Scheme-0 herein below.

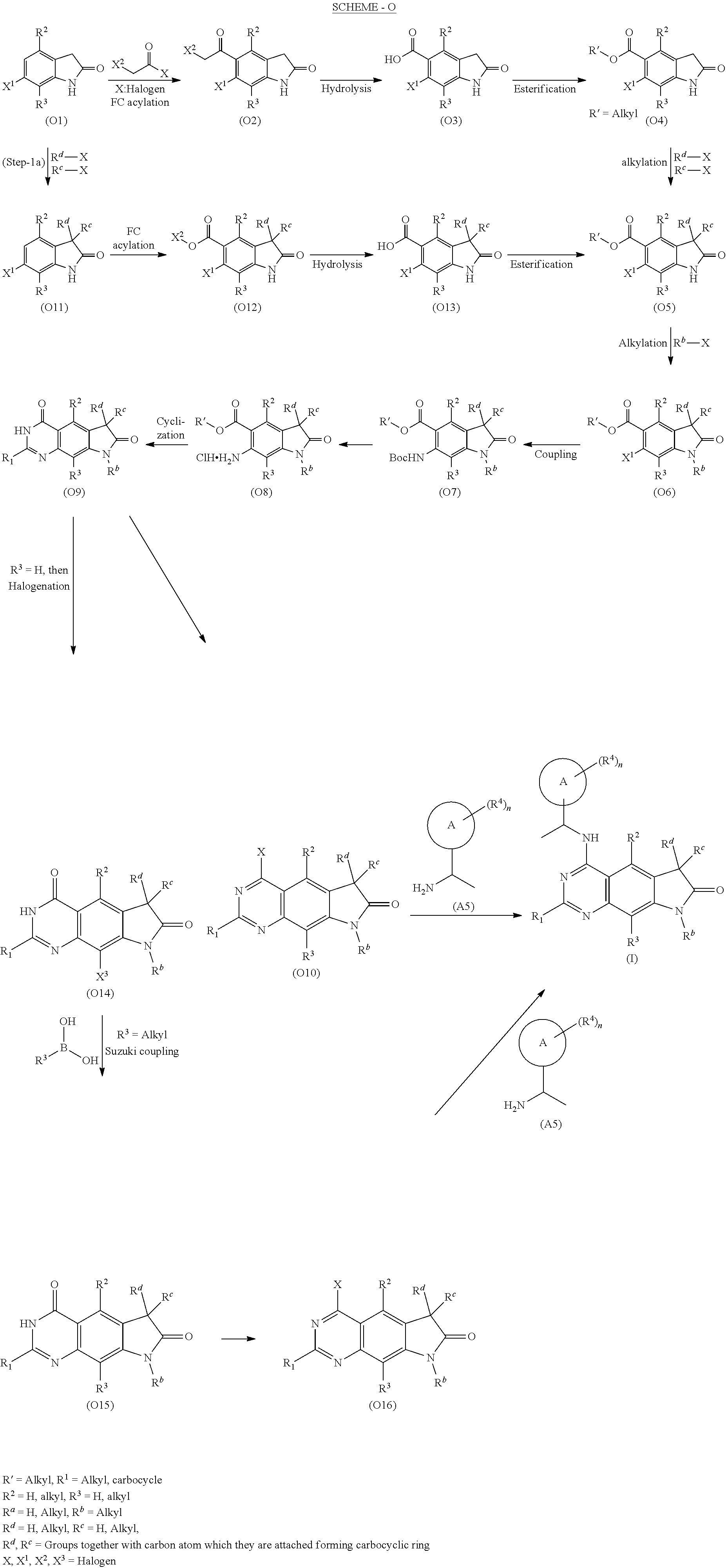

Compound of formula (O1) converted to compound of formula (O2) using Friedel craft acylation. This transformation was carried out by reaction of Compound of formula (O1) with corresponding acyl halide in presence of Lewis acids such as aluminum trichloride, zinc chloride, boron trifluoride etherate and like, in halogenated solvents like dichloromethane, dichloroethane and like.

Compound of formula (O2) was allowed to react with pyridine, optionally in solvents such as THF, toluene, xylene or the like or the mixtures thereof, followed by treatment of aqueous metal hydroxides like NaOH, KOH or the like or mixtures thereof to provide compound of formula (O3).

Compound of formula (O3) acid derivative undergoes esterification reaction to corresponding compound of formula (O4) using solvents such as methanol, ethanol, propanol, tert-butanol using acidic conditions like hydrochloric acid, sulfuric acid, thionyl chloride or the like or mixture(s) thereof.

Compound of formula (O4) was undergoes coupling with alkyl/substituted alkyl halide/dihalides to the corresponding formula (O5) using bases like Lithium diisopropylamide, butyl lithium, lithium bis(trimethylsilyl)amide, sodium bis(trimethylsilyl)amide, sodium tert-butoxide, potassium tertbutoxide, sodium ethoxide, sodium methoxide, cesium carbonate, potassium carbonate or the like possibly in the presence of additives such as N,N,N′,N′-Tetramethylethane-1,2-diamine in solvents selected from THF, 1,4-dioxane, DMF and like.

Alternatively, the compound of formula (O1) undergoes alkylation/acylation reaction to give compound of formula (O11) the reaction was carried out using alkyl halides/acyl halide and bases like Lithium diisopropylamide, butyl lithium, lithium bis(trimethylsilyl)amide, sodium bis(trimethylsilyl)amide, sodium tert-butoxide, potassium tertbutoxide, sodium ethoxide, sodium methoxide, cesium carbonate, potassium carbonate or the like possibly in the presence of additives such as N,N,N′,N′-Tetramethylethane-1,2-diamine in solvents selected from THF, 1,4-dioxane, DMF and like

Compound of formula (O11) was converted to compound of formula (O13) by employing similar protocol mentioned above for conversion of compound of formula (O1) to compound of formula (O3).

Compound of formula (O13) undergoes esterification reaction to corresponding compound of formula (O5) using solvents such as methanol, ethanol, propanol, tert-butanol using acidic conditions like hydrochloric acid, sulfuric acid, thionyl chloride or the like or mixture(s) thereof.

Compound of formula (O5) can be further reacted with alkyl halide, acyl chlorides using bases such as K₂CO₃, Na₂CO₃, Cs₂CO₃ etc. in polar aprotic solvents like DMF, DMSO etc. at elevated temperatures leading to compound of formula (O6)

Compound of formula (O6) allowed to react with tert-butyl carbamate in the presence of catalyst such as (tris(dibenzylideneacetone) dipalladium(0), palladium (II) acetate, Bis(dibenzylideneacetone)₂ Pd(0), racemic 2,2′-Bis(diphenylphosphino)-1,1′-binaphthyl, 2,5 bis(tri-t-butylphosphine) palladium (0) and the like; in presence of ligands such as RuPhos, Xanthphos, Davephos, BINAP, or the like; using a suitable base such as sodium carbonate, cesium carbonate, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (O7).

Compound of formula (O7) undergoes deprotection using acids like organic acids such as trifluoroacetic acid, Methane sulfonic acid and like, mineral acids like hydrochloric acid, acetic acid (aqueous or in etheral solvents), sulfuric acid and the like; using solvents like dichloromethane, dichloroethane, THF, 1,4-dioxane and like, to provide compound of formula (O8).

Compound of formula (O8) allowed to react with alkylnitrile in presence of the acidic reagents such as methane sulfonic acid, sulfuric acid, hydrochloric acid and the like to obtain compound of formula (O9). The same transformation can be carried out using trialkyl orthoacetate in presence of ammonium acetate, in corresponding polar protic solvents like ethanol, methanol and thereof.

Further, compound of formula (O7) on reaction with alkylnitrile in presence of the acidic reagents such as methane sulfonic acid, sulfuric acid, hydrochloric acid or the like can directly give compound of formula (O9)

Compound of formula (O9) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (O10).

Compound of formula (O10) allowed to react with compound of formula (A5) in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; in etheral solvents such as THF, 1,4 dioxane and like or polar aprotic solvents like DMF, DMA, DMSO and thereof.

Further, compound of formula (O10) converted to compound of formula (O14) using halogenating reagents such as NBS, NCS, bromine and like, in polar solvents such as DMF, AcOH, DCM and like.

Compound of formula (O15) was prepared from compound of formula (O14) using C—C coupling reactions such as Suzuki coupling reaction using corresponding boronic acid in presence of Pd catalyst such as tris(dibenzylideneacetone) dipalladium(0), palladium(II)acetate, Bis(dibenzylideneacetone)₂Pd(0), rac 2,2′-Bis(diphenylphosphino)-1,1′-binaphthyl, 2,5 bis(tri-t-butylphosphine) palladium (0), Pd(PPh₃)₄ and like in base such as K₂CO₃, Na₂CO₃, Cs₂CO₃, Potassium phosphate and like, in solvents such as toluene, 1,4-dioxane, DMA, DMF and like

The compound of formula (O14) can be converted to compound of formula (I) using similar protocol used earlier for conversion of compound of formula (O9) to compound of formula (I) in two steps.

The compounds of formula (I) was prepared by following the sequential transformations as depicted and described in Scheme-P herein below.

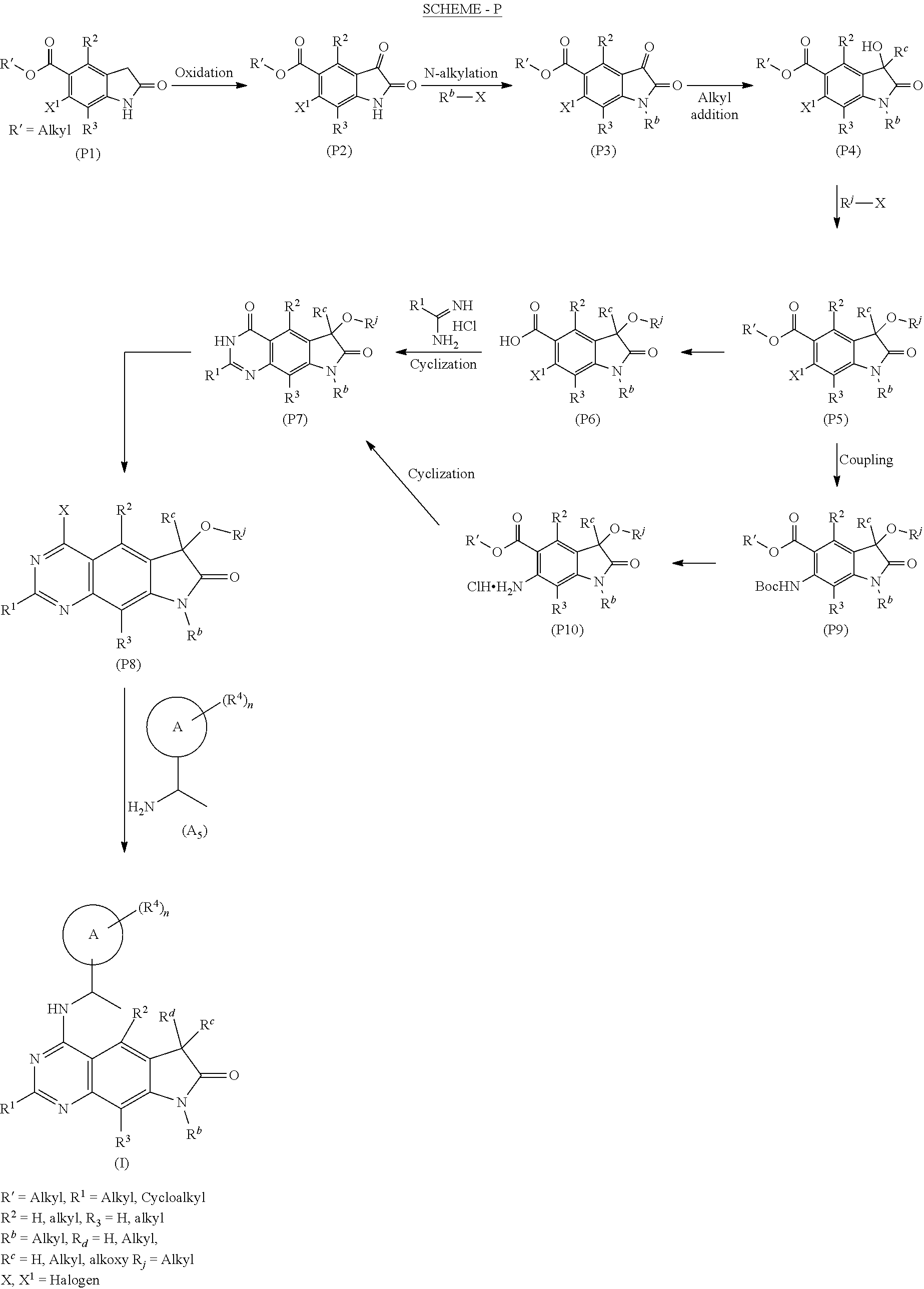

Compound of the formula (P2) was obtained by oxidation of compound of the formula (P1). This transformation can be effected by oxidizing reagents such as potassium permanganate, potassium dichromate, sodium dichromate and like; in presence of acids like H₂SO₄, acetic acid and like.

Compound of formula (P2) undergoes N-alkylation using alkyl halides in presence of bases such as NaH, Potassium/sodium alkoxides, K₂CO₃, Na₂CO₃, Cs₂CO₃ organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (P3).

Compound of formula (P3) undergoes reaction with organometallic reagents such as grignard reagent, dialkyl zinc, alkyl lithiums, and thereof; silane reagents such as trifluromethyl trimethyl silane and thereof; in etheral solvents such as THF, MTBE and like to provide compounds of formula (P4)

Compound of formula (P4)undergoes O-alkylation using alkyl halides in presence of bases such as sodium hydride, potassium/sodium alkoxide K₂CO₃, Na₂CO₃, Cs₂CO₃, NaH and thereof; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (P5).

Compound of formula (P5) converted to compound of formula (P6) in presence of alkali hydroxides such as NaOH, LiOH and thereof, in solvents like methanol, ethanol and thereof or using solvents like THF, 1,4-dioxane and thereof.

Compound of formula (P6) on reaction with acetamidine, formamidine and like; in polar aprotic solvents like DMF, DMSO and metals like copper, thereof at temperature elevated temperatures afforded compound of formula (P7).

Alternatively, Compound of formula (P5) allowed to react with tert-butyl carbamate in the presence of catalyst such as (tris(dibenzylideneacetone) dipalladium(0), palladium (II) acetate, Bis(dibenzylideneacetone)₂ Pd(0), racemic 2,2′-Bis(diphenylphosphino)-1,1′-binaphthyl, 2,5 bis(tri-t-butylphosphine) palladium (0) and the like; in presence of ligands such as RuPhos, Xanthphos, Davephos, BINAP, or the like; using a suitable base such as sodium carbonate, cesium carbonate, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (P9).

Compound of formula (P9) undergoes deprotection using acids like organic acids such as trifluoroacetic acid, Methane sulfonic acid and like, mineral acids like hydrochloric acid, acetic acid (aqueous or in etheral solvents), sulfuric acid and the like; using solvents like dichloromethane, dichloroethane, THF, 1,4-dioxane and like thereof to provide compound of formula (P10).

Compound of formula (P10) allowed to react with alkylnitrile in presence of the acidic reagents such as methane sulfonic acid, sulfuric acid, hydrochloric acid or the like to obtain compound of formula (P7). The same transformation can be carried out using trialkyl orthoacetate in presence of ammonium acetate, in corresponding polar protic solvents like ethanol, methanol and thereof.

Alternatively, compound of formula (P9) on reaction with alkylnitrile in presence of the acidic reagents such as methane sulfonic acid, sulfuric acid, hydrochloric acid or the like can directly give compound of formula (P7)

Compound of formula (P7) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (P8).

Compound of formula (P8) allowed to react with compound of formula (A5) in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; in etheral solvents such as THE 1,4 dioxane and like or polar aprotic solvents like DMF, DMA, DMSO and thereof.

The compounds of formula (I) was prepared by following the sequential transformations as depicted and described in Scheme-Q herein below

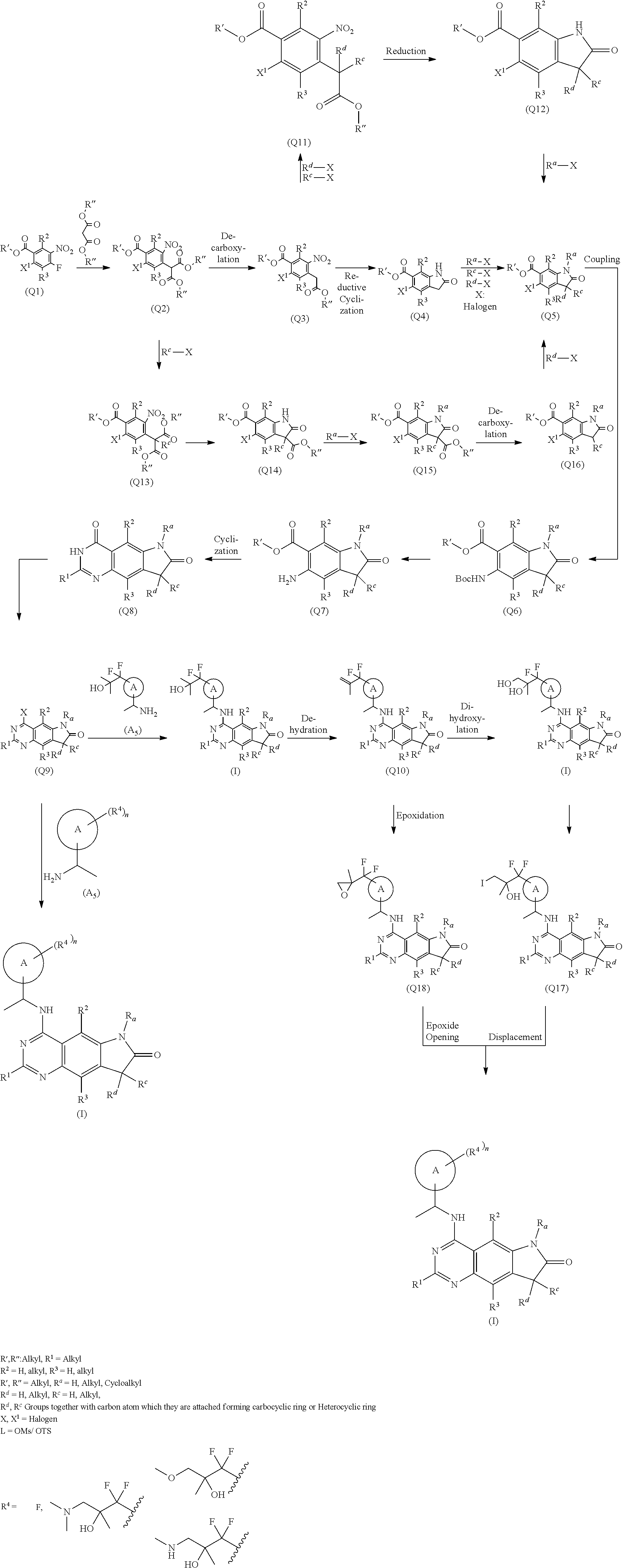

The reaction between compound of formula (Q1) and substituted dialkyl dicarboxylates in presence of base provided compound of the formula (Q2). This type of transformations can be carried out at appropriate temperature using alkali bases such as NaOH, KOH and like; carbonates such as potassium carbonate, cesium carbonate and like; or organic bases like Triethylamine, diisopropyl ethyl amine and the like; in amidic solvents like DMF, DMA and like; etheral solvents like 1, 4-dioxane, THF and mixtures thereof.

Compound of formula (Q2) undergoes decarboxylation reaction to furnish compound of formula (Q3). This transformation was carried out in polar solvents like DMSO, DMF, and like, using sodium chloride, lithium chloride and like. Similar transformation can be done using acids such as sulfuric acid, trifluoroacetic acid and like, at appropriate temperature.

Reductive cyclization of compound of the formula (Q3) provide compound of formula (Q4). The reduction of nitro group was carried out using different reagents; although not limited, such reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof.

Compound of formula (Q4) undergoes alkylation reaction by reacting with corresponding alkyl halide in presence of bases such as sodium hydride, potassium tert butoxide, K₂CO₃, Na₂CO₃, Cs₂CO₃; organic bases like diisopropyl ethyl amine, DBU, DABCO and the like; in polar aprotic solvents like DMF, DMSO, acetone and like, etheral solvents such as THF, 1,4-dioxane and like, at appropriate temperature provided compound of formula (Q5).

Alternatively, Compound of formula (Q3) undergoes C-alkylation reaction by reacting with corresponding alkyl halide in presence of bases such as sodium hydride, potassium tert butoxide and like; in polar aprotic solvents like DMF, DMSO, acetone and like, etheral solvents such as THF, 1,4-dioxane and like, to provide compound of formula (Q11). Compound of formula (Q11) undergoes reductive cyclization similar to conversion of compound of formula (Q3) to compound of formula (Q4) to provide compound of formula (Q12). Compound of formula (Q12) undergoes N-alkylation reaction with alkyl halides in presence of bases such as NaH, Potassium/sodium alkoxides, K₂CO₃, Na₂CO₃, Cs₂CO₃ organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (Q5)

Alternatively, Compound of formula (Q2)undergoes C-alkylation using corresponding alkyl halide in presence of bases such as sodium hydride, potassium tert butoxide, K₂CO₃, Na₂CO₃, Cs₂CO₃ and like; in polar aprotic solvents like DMF, DMSO, acetone and like, etheral solvents such as THF, 1,4-dioxane and like to provide compound of formula (Q13).

The compound of formula (Q13) was converted to compound of formula (Q15) in two steps viz. reductive cyclization and N-alkylation by following similar reactions employed for conversion of compound of formula (Q3) to compound of formula (Q5). Compound of formula (Q15) undergoes decarboxylation reaction to furnish compound of formula (Q16). This transformation was carried out in polar solvents like DMSO, DMF, and like, using sodium chloride, lithium chloride and like. Similar transformation can be done using acids such as sulfuric acid, trifluoroacetic acid and like, at elevated temperatures.

Compound of formula (Q16) undergoes C-alkylation using corresponding alkyl halide in presence of bases such as sodium hydride, potassium tert butoxide, K₂C03, Na₂CO₃, Cs₂CO₃ and like; in polar aprotic solvents like DMF, DMSO, acetone and like, etheral solvents such as THF, 1,4-dioxane and like to provide compound of formula (Q5).

Compound of formula (Q5) allowed to react with tert-butyl carbamate in the presence of catalyst such as (tris(dibenzylideneacetone)dipalladium(0), palladium(II) acetate, Bis(dibenzylideneacetone)₂ Pd(0), rac 2,2′-Bis(diphenylphosphino)-1,1′-binaphthyl, 2,5 bis(tri-t-butylphosphine) palladium (0) and the like; in presence of ligands such as RuPhos, Xanthphos, Davephos, BINAP, or the like; using a suitable base such as sodium carbonate, cesium carbonate, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (Q6).

Compound of formula (Q6) undergoes deprotection using acids like organic acids such as trifluoroacetic acid, Methane sulfonic acid and like, mineral acids like hydrochloric acid, acetic acid (Aqueous or in etheral solvents), sulfuric acid and the like; using solvents like dichloromethane, dichloroethane, THF, 1,4-dioxane and like thereof to provide compound of formula (Q7).

Compound of formula (Q7) allowed to react with alkylnitrile in presence of the acidic reagents such as methane sulfonic acid, sulfuric acid, hydrochloric acid or the like to obtain compound of formula (Q8). The same transformation can be carried out using trialkyl orthoacetate in presence of ammonium acetate, in corresponding polar protic solvents like ethanol, methanol and thereof.

Alternatively, compound of formula (Q6) on reaction with alkylnitrile in presence of the acidic reagents such as methane sulfonic acid, sulfuric acid, hydrochloric acid or the like can directly give compound of formula (Q8)

Compound of formula (Q8) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (Q9).

Compound of formula (Q9) allowed to react with compound of formula (A5) in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; in etheral solvents such as THE 1,4 dioxane and like or polar aprotic solvents like DMF, DMA, DMSO and thereof.

Compound of formula (Q9) allowed to react with 1-(3-(1-aminoethyl)-2-fluorophenyl)-1,1-difluoro-2-methylpropan-2-ol hydrochloride in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; in etheral solvents such as THF, 1,4 dioxane and like or polar aprotic solvents like DMF, DMA, DMSO and thereof.

Compound of formula (I) allowed to react with fluorinating reagent such as DAST, martin sulfurane in solvents such as DCM, chloroform, THF, ether, 1,4-dioxane to provide compound of formula (Q10).

Compound of formula (Q10) allowed to react with osmium tetra oxide, potassium osmate dihydrate (Sharpless asymmetric dihydroxylation method) using potassium chlorate, hydrogen peroxide, potassium ferricyanide, N-methylmorpholine N-oxide, chiral quinine or the like, in solvents like acetone, tert butanol water system to provide compound of formula (I).

Compound of formula (I) undergoes mesylation, tosylation and thereof, reactions in presence of organic bases such as TEA, DIPEA, Pyridine and like, in solvents such as THF, DCM and mixtures thereof, to provide compound of formula (Q17) Compound of formula (Q17) undergoes displacement reaction with primary or secondary amines in presence of alcohol solvents such as ethanol, IPA and mixtures thereof to provide compound of formula (I).

Compound of formula (Q10) undergoes epoxidation reaction to provide compound of formula (Q18). This reaction is effected by hydrogen peroxide in presence of acidic medium using organic acids such as formic acid and like.

Compound of formula (Q18) on epoxide opening by nucleophilic reagent provide compound of formula (I). Such transformations can be effected by reaction of epoxide compound with various nucleophilic reagents such as sodium alkoxides, primary or secondary amines in alcohol solvents like ethanol, methanol, and like and at room temperature or elevated temperature.

The compounds of formula (I) was prepared by following the sequential transformations as depicted and described in Scheme-R herein below

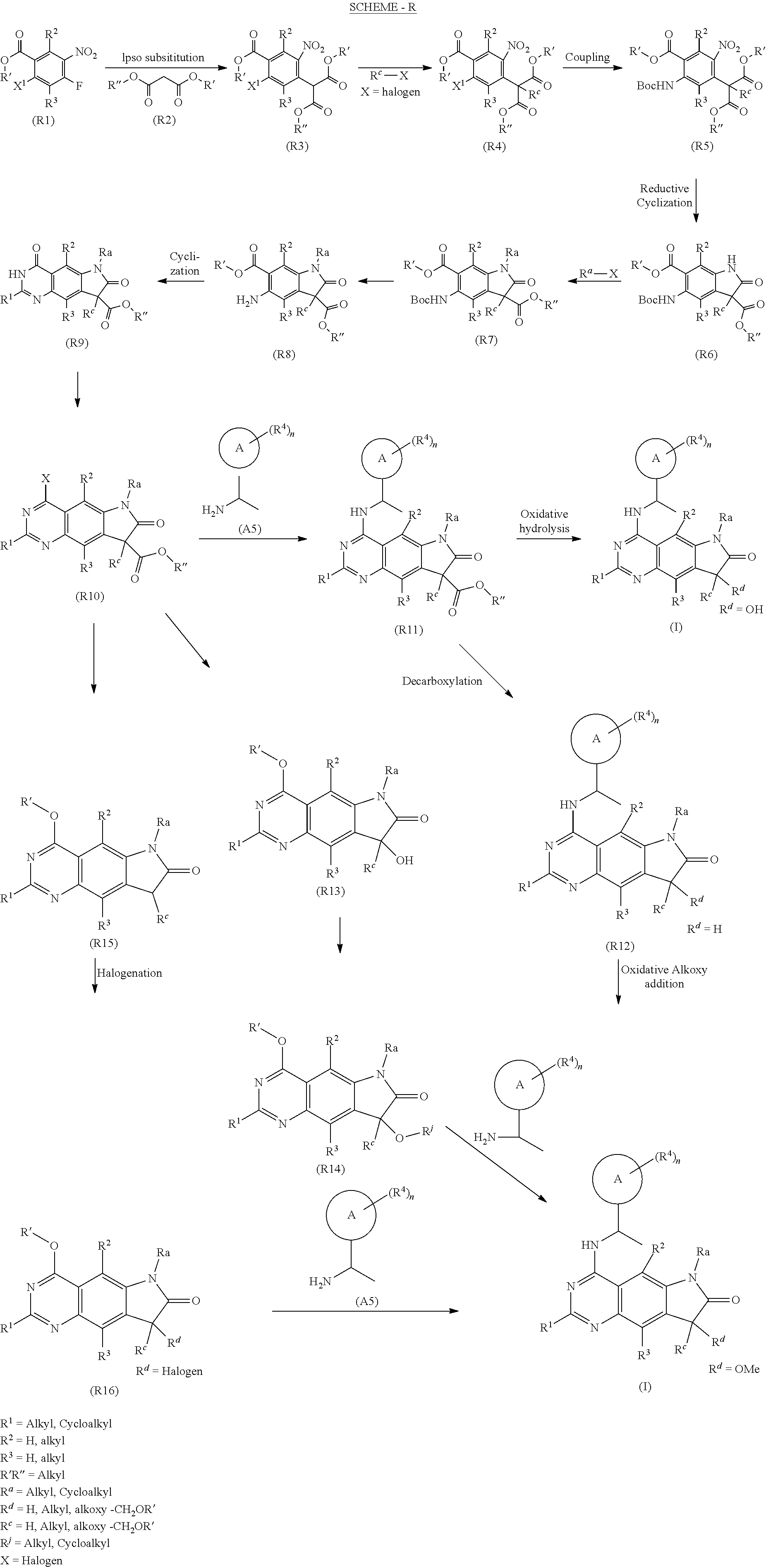

The reaction between compound of formula (R1) and substituted dialkyl dicarboxylates (compound of the formula (R2) in presence of base provided compound of the formula (R3). This type of transformations can be carried out either at room temperature or at elevated temperatures using alkali bases such as NaOH, KOH and like; carbonates such as potassium carbonate, cesium carbonate and like; or organic bases like triethylamine, diisopropylethyl amine and thereof; in amidic solvents like DMF, DMA and like; etheral solvents like dioxane, THF and thereof.

Compound of formula (R3) undergoes alkylation using alkyl halides in presence of bases such as sodium hydride, potassium/sodium alkoxide bases such as K₂CO₃, Na₂CO₃, Cs₂CO₃; organic bases like diisopropylethyl amine, DBU, DABCO and so on; in polar aprotic solvents like DMF, DMSO and like, at room temperature or elevated temperatures provide compound of formula (R⁴).

Compound of formula (R⁴) allowed to react with tert-butyl carbamate in the presence of catalyst such as (tris(dibenzylideneacetone) dipalladium(0), palladium (II) acetate, Bis(dibenzylideneacetone)₂ Pd(0), rac 2,2′-Bis(diphenylphosphino)-1,1′-binaphthyl,2,5 bis(tri-t-butylphosphine) palladium (0) and the like; in presence of ligands such as RuPhos, Xanthphos, Davephos, BINAP, or the like; using a suitable base such as sodium carbonate, cesium carbonate, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (R5).

Compound of formula (R5) undergo reductive cyclization to provide compound of formula (R6). This nitro reduction can be achieved by reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof.

Compound of formula (R6) undergoes N-alkylation using alkyl halides in presence of bases such as sodium hydride, potassium/sodium alkoxide bases such as K₂CO₃, Na₂CO₃, Cs₂CO₃; organic bases like diisopropylethyl amine, DBU, DABCO and so on; in polar aprotic solvents like DMF, DMSO and like, at room temperature or elevated temperatures provide compound of formula (R7).

Compound of formula (R7) can be converted to the compound of formula (R11) by employing 4 step protocol mentioned in conversion of compound of formula (N8) to compound of formula (N12)

Compound of formula (R11) undergoes decarboxylation reaction to furnish compound of the formula (R12). This transformation can be effected by acidic reagents such as mineral acids like sulfuric acid, organic acids like trifluoroacetic acid and thereof; similar transformation can be achieved using sodium chloride, lithium chloride and thereof, in solvents such as dimethyl sulfoxide and like; at elevated temperatures.

Compound of formula (R12) converted to compound of formula (I) using ceric ammonium nitrate, thallium nitrate and thereof in present of alcoholic solvents like methanol, ethanol and thereof.

Further, Compound of formula (R11) on reaction with alkalis such as NaOH, LiOH and like, in alcoholic solvents like methanol ethanol and thereof, provide compound of formula (I) where (R^{d}═—OH)

Compound of formula (R10) undergoes nucleophilic substitution along with air oxidation in presence of bases like LiOH and like, in alcoholic solvent such as methanol in presence of air, provide compound of formula (R13).

Compound of formula (R13) on O-alkylation using corresponding alkyl halide in presence of bases such as sodium hydride, potassium tert butoxide, K₂CO₃, Na₂CO₃, Cs₂CO₃ and like; in polar aprotic solvents like DMF, DMSO, acetone and like, etheral solvents such as THF, 1,4-dioxane and like to provide compound of formula (R14). This reaction Yielded decarboxylation product viz. compound of formula (R15).

Compound of formula (R14) undergoes coupling reaction with compound of formula (A5) to furnish compound of formula (I). The reaction can be carried out in presence of organic base such as diisopropylethylamine, triethylamine, DBU or the like, or using coupling reagents such as DCC, EDC, BOP, pyBOP, HBTU or the like; either neat reaction in base or in etheral solvents such as THE 1,4 dioxane and like or polar aprotic solvents like DMF, DMA, DMSO and thereof.

Compound of formula (R¹⁵) undergoes fluorination reaction by fluorinating reagents such as DAST, select flour and thereof. or C-alkylation reaction with various alkyl halides in presence of bases such as sodium hydride, potassium tert butoxide, K₂CO₃, Na₂CO₃, Cs₂CO₃ and like; in polar aprotic solvents like DMF, DMSO, acetone and like, etheral solvents such as THF, 1,4-dioxane and like to give compound of formula (R16).

Compound of formula (R16) can be converted to compound of formula (I) by analogous protocol mentioned above for the conversion of (R14) to compound of formula (I).

The compounds of formula (I) was prepared by following the sequential transformations as depicted and described in Scheme S:

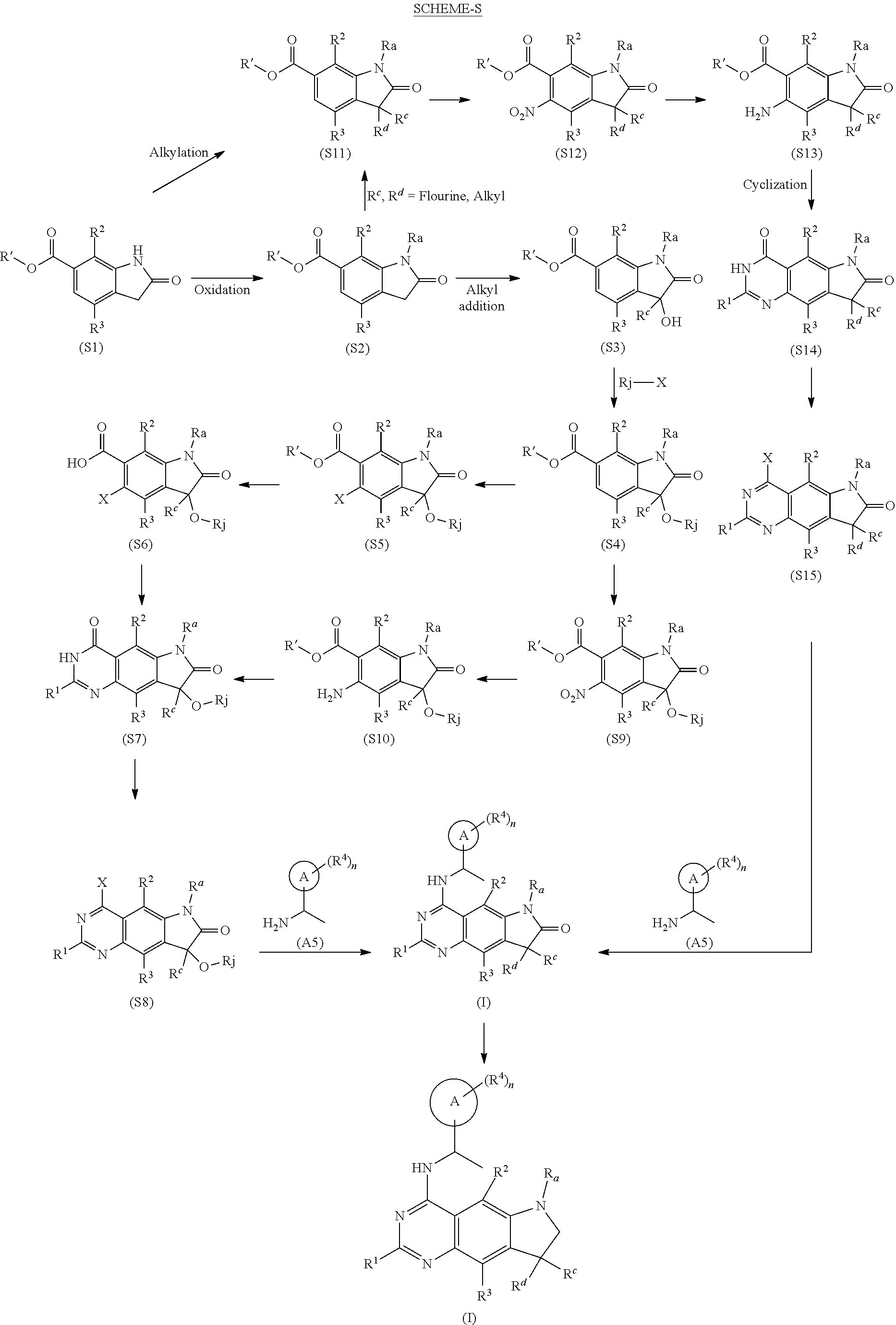

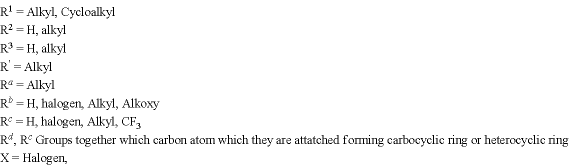

Compound of formula (S2) was prepared from compound of formula (Si) by oxidation reaction followed by N-alkylation reaction. This oxidation was effected by reagents like tertiary butyl hydroperoxide, selenium dioxide, manganese dioxide and like; in presence of catalytic CuI, Cu(I) reagents and thereof. Further the N-alkylation was carried out by using alkyl halides in presence of bases such as sodium hydride, potassium/sodium alkoxide bases such as K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (S2)

Compound of formula (S2) undergoes reaction with organometallic reagents such as Grignard reagent, dialkyl zinc, alkyl lithiums, and thereof; silane reagents such as trifluoromethyl trimethyl silane and thereof; in etheral solvents such as THF, MTBE and like to provide compounds of formula (S3)

Compound of formula (S3) undergoes O-alkylation to provide compound of formula (S4). This transformation can be effected by using alkyl halides in presence of bases such as sodium hydride, potassium/sodium alkoxide K₂CO₃, Na₂CO₃, Cs₂CO₃, sodium hydride; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures.

Compound of formula (S5) can be prepared from compound of formula (S4) by employing halogenation reaction. Such reactions can be carried out in presence of halogenating reagents such as N-halo succinamide, hydrohalic acid and likes; in solvents like DMF, Acetic acid and thereof; optionally in presence additives such as trifluoroacetic acid and like, in catalytic or molar proportions; and at room temperature or at elevated temperatures.

Compound of formula (S5) undergoes hydrolysis of ester group to provide compound of formula (S6). This transformation can be effected in presence of alkali hydroxides such as NaOH, LiOH and thereof, in solvents like methanol, ethanol and thereof or using solvents like THF, 1,4-dioxane and thereof

Compound of formula (S6) on reaction with acetamidine, formamidine and like; in polar aprotic solvents like DMF, DMSO and metals copper and like; optionally in presence of additives like proline and thereof, at room temperature or elevated temperatures afforded compound of formula (S7).

Alternatively compound of formula (S7) can be prepared in three steps. Compound of formula (S4) undergoes nitration reaction to provide compound of formula (S9). This reaction was carried out in presence of nitrating reagents such as potassium nitrate, sodium nitrate nitric acid and like; in acidic solvents such as sulfuric acid and thereof.

Compound of formula (S9) undergoes reduction reaction to provide compound of formula (S10). These transformations can be carried out using reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof

Compound of formula (S10) allowed to react with alkylnitrile in presence of the acidic reagents such as methane sulfonic acid, sulfuric acid, hydrochloric acid or the like to obtain compound of formula (S7). The same transformation can be carried out using trialkyl orthoacetate in presence of ammonium acetate, in corresponding polar protic solvents like ethanol, methanol and thereof.

Compound of formula (S7) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (S8).

Compound of formula (S8) allowed to react with compound of formula (A5) in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; either neat or in etheral solvents such as THF, 1,4 dioxane and like or polar aprotic solvents like DMF, DMA, DMSO and thereof

Further, compound of formula (S2) undergoes defluorination reaction with reagents such as DAST, selectfluor and like, in chlorinated solvent like dichloromethane and like; provided compound of formula (S11) (R^{c}, R^{d}═F)

Also, compound of formula (S1) undergoes c-alkylation and N-alkylation simultaneously in presence of alkyl halides in presence of bases such as sodium hydride, potassium/sodium alkoxide, K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (S11) (R^{c}, R^{d}=alkyl)

Compound of formula (S11) can be converted to compound of formula (I) by employing analogous five step protocol as mentioned above for conversion of compound of formula (S4) to compound of formula (I).

Further, Compound of formula (S11) can be converted to compound of formula (S14) by employing analogous three step protocol as mentioned above for conversion of compound of formula (S4) to compound of formula (S7) via compound of formula (S5) followed by compound of formula (S6).

Compound of formula (S14) can be converted to compound of formula (I) by employing analogous two step protocol as mentioned above for conversion of compound of formula (S7) to compound of formula (I).

Compound of formula (I) further on reaction with various organometallic reagents like LiAlH₄,BH₃-DMS and like provide compound of formula (I) These reactions are carried out in one or more solvents, e.g., ethers such as THF, 1,4-dioxane, and the like The compounds of formula (I) was prepared by following the sequential transformations as depicted and described in Scheme T:

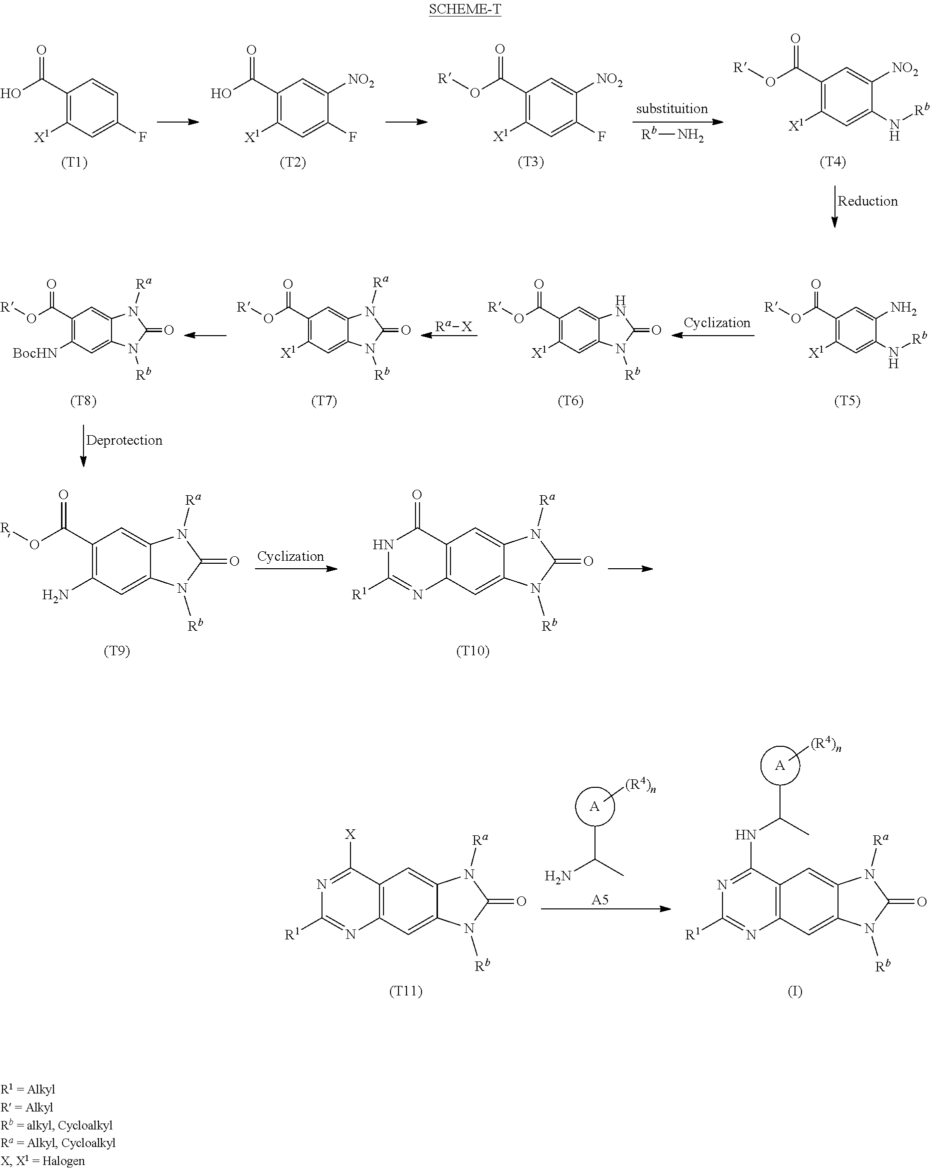

Nitration of compound of formula (T1) with nitrating reagents such as, although not limited to fuming nitric acid, potassium nitrate, and the like in acids such as, although not limited to tin (TV) chloride, sulphuric acid, trifluoracetic acid, acetic acid and the like, anhydrides like acetic anhydride, trifluoracetic anhydride and the like, or mixture(s) thereof to provide compound of formula (T2).

Compound of formula (T2) undergoes esterification reaction to corresponding compound of formula (T3) using solvents such as methanol, ethanol, propanol, tert-butanol using acidic conditions like hydrochloric acid, sulfuric acid, thionyl chloride or the like or mixture(s) thereof.

Compound of formula (T3) derivative undergoes N-alkylation reaction to corresponding compound of formula (T4) using alkylamine and solvents such as methanol, ethanol, propanol, tert-butanol.

Compound of the formula (T4) on reduction of nitro group to corresponding anilinic compound of formula (T5). The reduction of nitro group was carried out using different reagents; although not limited, such reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof.

Cyclization of compound of the formula (T5) using CDI in polar aprotic solvents like DMF, DMSO, halogenated solvents like DCM, chloroform, ethereal solvents like THF, 1,4-dioxane, at room temperature or elevated temperatures provided compound of formula (T6).

Compound of formula (T6) undergoes N-alkylation using alkyl halides in presence of bases such as sodium hydride, potassium/sodium alkoxide K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (T7).

Compound of formula (T7) allowed to react with tert-butyl carbamate in the presence of catalyst such as (tris(dibenzylideneacetone)dipalladium(0), palladium(II) acetate, Bis(dibenzylideneacetone)₂ Pd(0), rac 2,2′-Bis(diphenylphosphino)-1,1′-binaphthyl, 2,5 bis(tri-t-butylphosphine) palladium (0) and the like; in presence of ligands such as RuPhos, Xanthphos, Davephos, BINAP, or the like; using a suitable base such as sodium carbonate, cesium carbonate, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (T8).

Compound of formula (T8) undergoes deprotection using acids like organic acids such as trifluoroacetic acid, Methane sulfonic acid and like, mineral acids like hydrochloric acid, acetic acid (Aqueous or in etheral solvents), sulfuric acid and the like, using solvents like dichloromethane, dichloroethane, THF, 1,4-dioxane and like thereof to provide compound of formula (T9).

Compound of formula (T9) allowed to react with alkyl nitrile in presence of the acidic reagents such as methane sulfonic acid, sulfuric acid, hydrochloric acid or the like to obtain compound of formula (T10). The same transformation can be carried out using trialkyl orthoacetate in presence of ammonium acetate, in corresponding polar protic solvents like ethanol, methanol and thereof.

Compound of formula (T10) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (T11).

Compound of formula (T11) allowed to react with compound of formula (A5) in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; in etheral solvents such as THF, 1,4 dioxane and like or polar aprotic solvents like DMF, DMA, DMSO and thereof.

The compounds of formula (I) was prepared by following the sequential transformations as depicted and described in Scheme U:

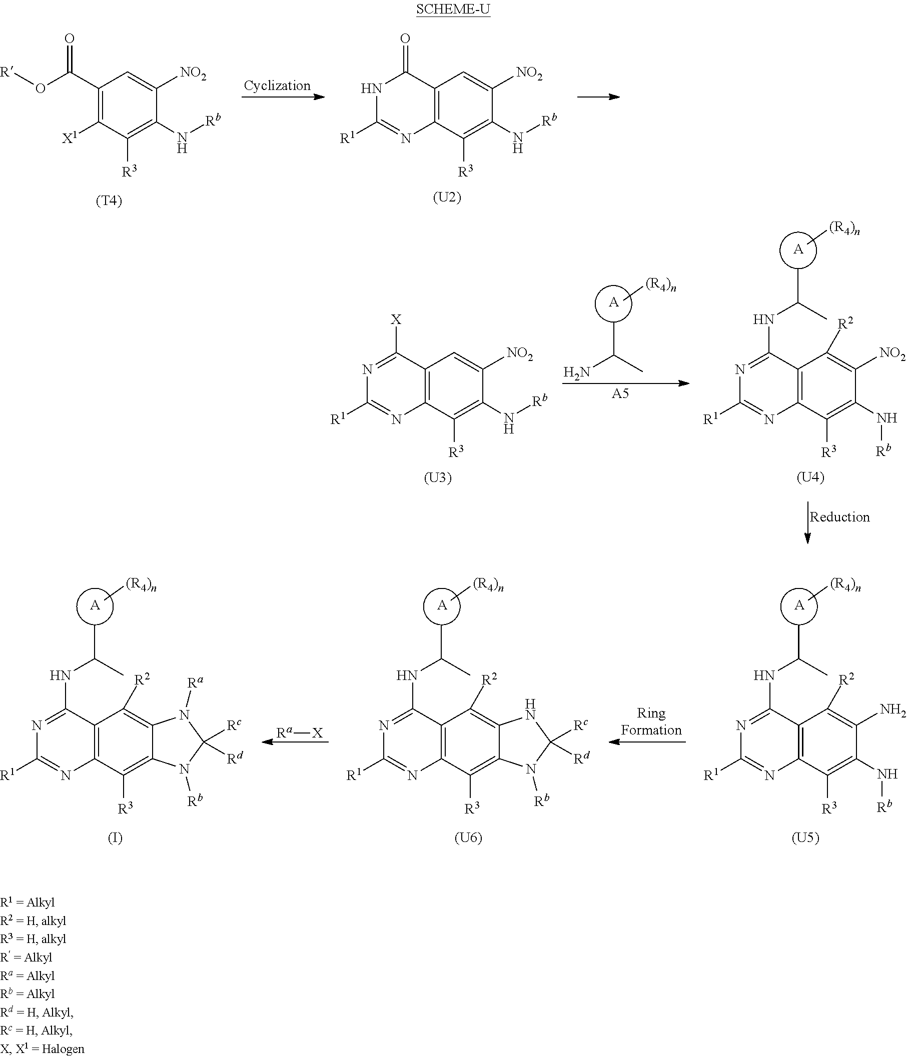

Compound of formula (T4) on reaction with acetamidine, formamidine and like; in polar aprotic solvents like DMF, DMSO and thereof at temperature elevated temperatures afforded compound of formula (U2).

Compound of formula (U2) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (U3).

Compound of formula (U3) allowed to react with compound of formula (A5) in presence of suitable coupling reagent to provide compound of formula (U4). The reaction can be carried out in presence of organic base such as diisopropylethylamine, triethylamine, DBU or the like, or using coupling reagents such as DCC, EDC, BOP, pyBOP, HBTU or the like; in etheral solvents such as THE 1,4 dioxane and like or polar aprotic solvents like DMF, DMA, DMSO and thereof.

Reduction of compound of the formula (U4) provide compound of formula (U5). The reduction of nitro group was carried out using different reagents; although not limited, such reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof.

Cyclization of compound of the formula (U5) using corresponding ketone in acid catalyst like pTsOH, Benzene sulphonic acid, sulfuric acid and acetic acid at room temperature or elevated temperatures provided compound of formula (U6).

Compound of formula (U6) undergoes N-alkylation using alkyl halides in presence of bases such as sodium hydride, potassium/sodium alkoxide K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (I).

The compounds of formula (I) was prepared by following the sequential transformations as depicted and described in Scheme V:

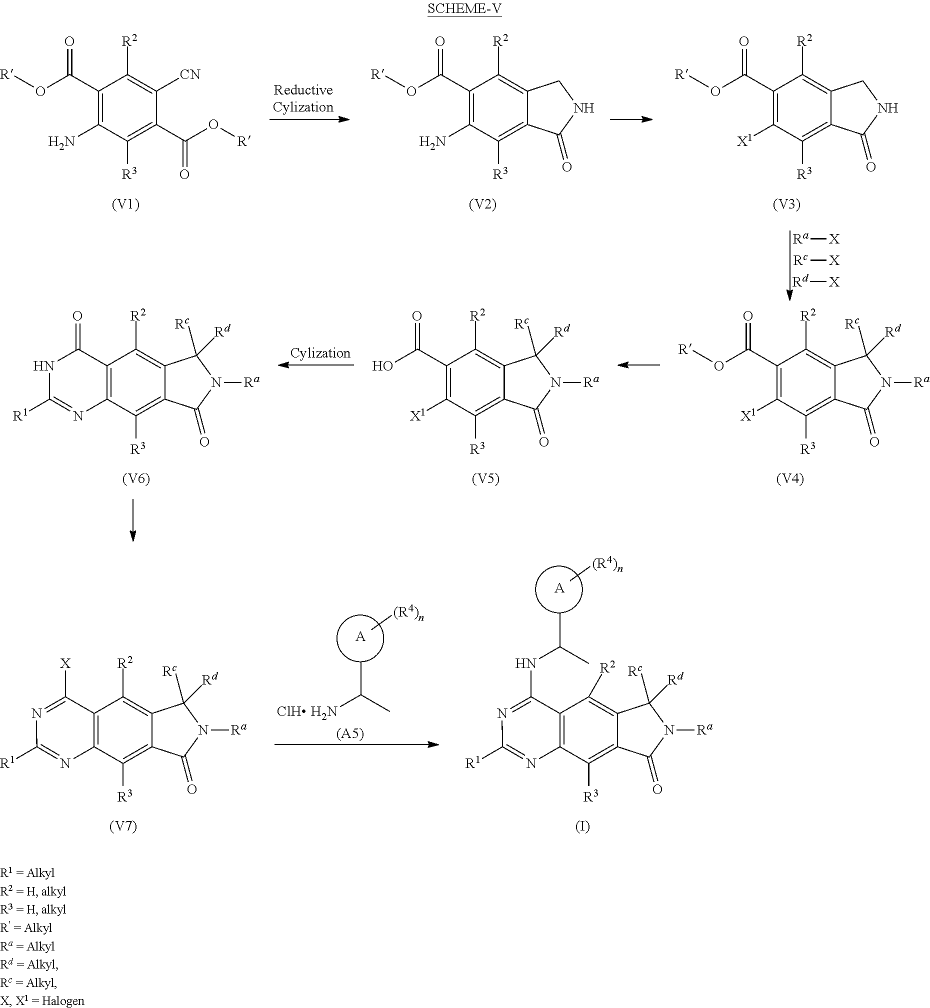

Compound of formula (V2) can be prepared from compound of formula (V1) via reductive cyclization reaction. This transformations can be carried out using reducing reagents such as contact hydrogenation in presence of Raney nickel, Pd/C, Pt/C and like; in etheral solvents such as 1,4-dioxane and like; optionally at room temperature or at elevated temperatures.

Compound of formula (V2) undergoes diazotization reaction using tert-butyl nitrite, isoamyl nitrite, sodium nitrite and like, followed by reaction with copper halides and like; can provide compound of formula (V3)

Compound of formula (V3) undergoes C-alkylation and N-alkylation simultaneously in presence of alkyl halides in presence of bases such as sodium hydride, potassium/sodium alkoxide K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (V4)

Compound of formula (V4) can be converted to compound of formula (I) by employing analogous 3 step protocol mentioned in scheme-P for conversion of compound of formula (P6) to compound of formula (I).

The compounds of formula (I) was prepared by following the sequential transformations as depicted and described in Scheme W:

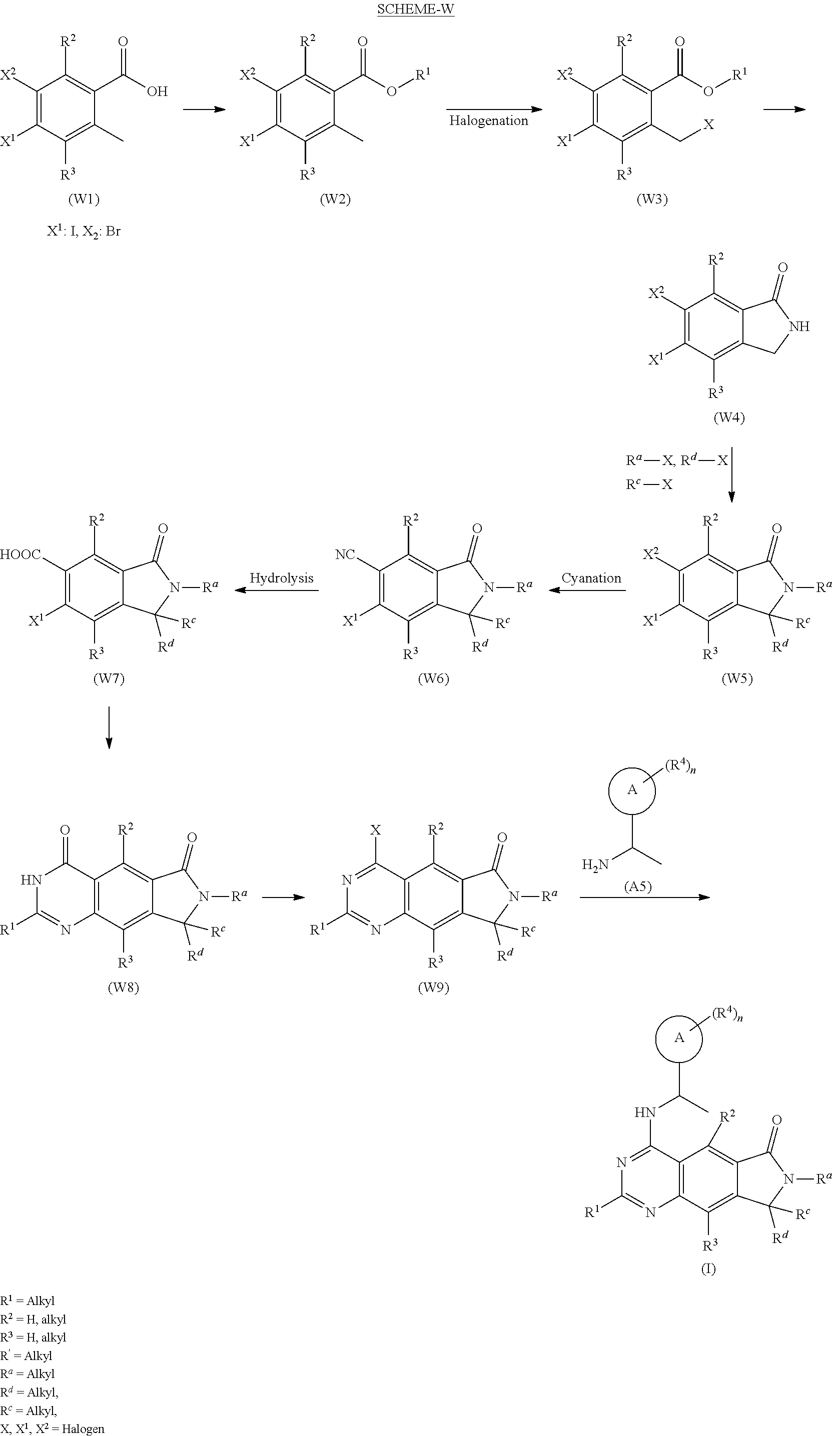

Compound of formula (W1) undergoes esterification reaction to provide compound of formula (W2). This transformation can be effected by reaction of alcohols such as methanol, ethanol and like; in presence of mineral acids like sulfuric acid, organic acids like methane sulfonic acid and like, or in presence of chloride reagents like thionyl chloride, oxalyl chloride and thereof. This transformation can also be effected by Mitsonobu reaction between acid (W1) and corresponding alcohols in presence of Triaryl phosphines and azocarboxylates such as DEAD, DIAD and like.

Compound of formula (W2) undergoes benzylic halogenation reaction using halogenating reagents like N-halo succinimide and thereof; in presence of initiators such as benzoyl peroxide, AIBN and like, in solvents such as carbon tetrachloride and thereof; at elevated temperature provide compound of formula (W3).

Compound of formula (W3) on reaction with ammonium hydroxide at room temperature or at elevated temperatures in alcoholic solvents like methanol, ethanol and like; undergoes cyclization reaction to provide compound of formula (W4).

Compound of formula (W4) undergoes C-alkylation and N-alkylation simultaneously in presence of alkyl halides in presence of bases such as sodium hydride, potassium/sodium alkoxide K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (W5).

Compound of formula (W5) on reaction with metal cyanides such as Copper(I) cyanide and like in polar aprotic solvent such as DMF and like at elevated temperatures afford compound of formula (W6).

Compound of formula (W6) undergoes hydrolysis reaction to furnish compound of formula (W7). This transformation can be carried out in presence of alkali hydroxides such as NaOH, LiOH and thereof, in solvents like methanol, ethanol and thereof or using solvents like DMF, THF, 1,4-dioxane.

Compound of formula (W7) can be converted to compound of formula (I) by employing analogous 3 step protocol mentioned in scheme P for conversion of compound of formula (P6) to compound of formula (I).

The compounds of formula (I) was prepared by following the sequential transformations as depicted and described in Scheme-Y herein below

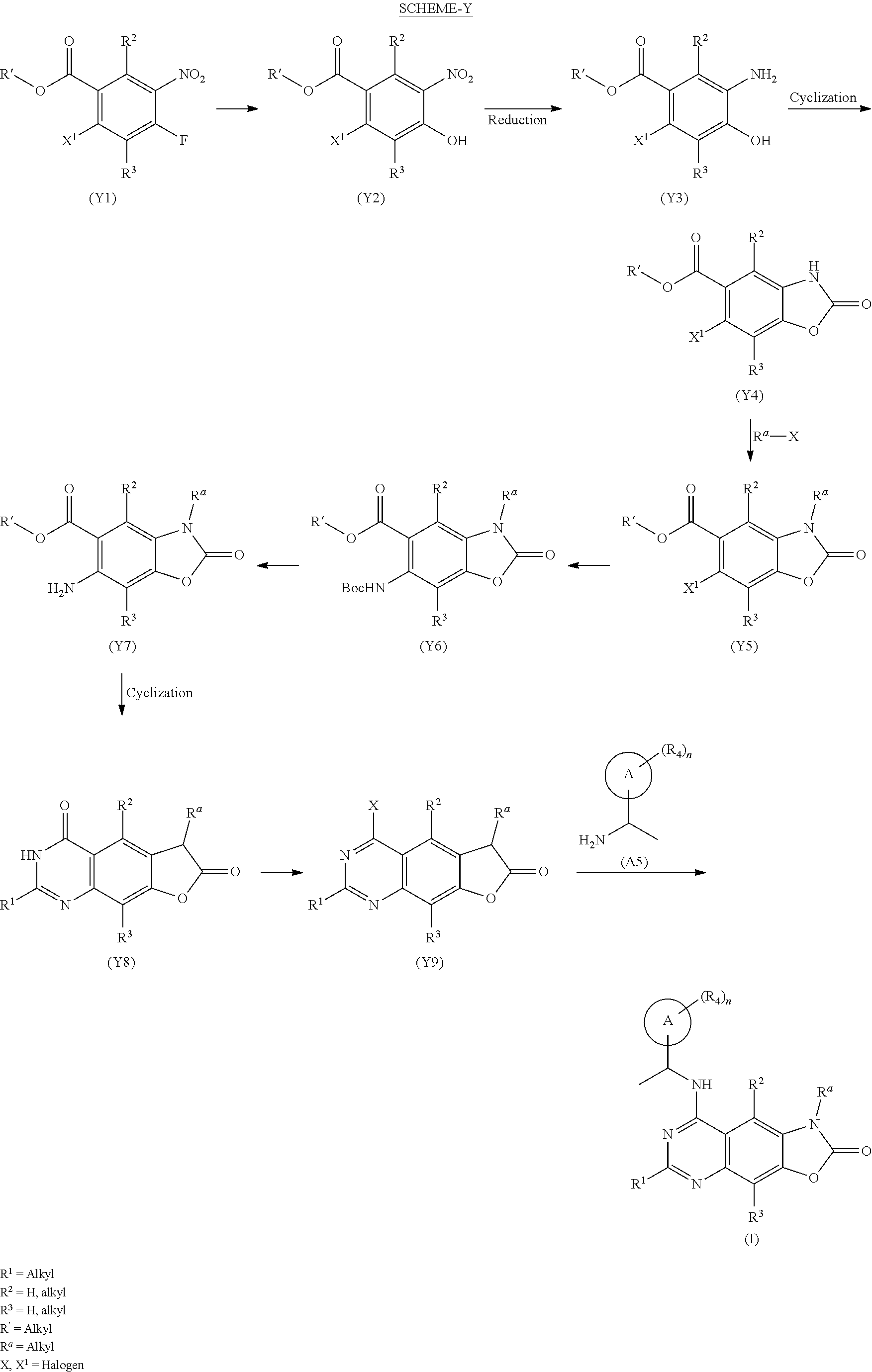

Compound of formula (Y1) undergoes reaction with bases such as K₂CO₃, Na₂CO₃, Cs₂CO₃ and like; in solvents such as DMF, DMSO and thereof, at elevated temperatures to afford compound of formula (Y2)

Compound of formula (Y2) undergoes reduction reaction to furnish compound of formula (Y3). Such reductions of nitro group were carried out using different reagents; although not limited, such reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like, under acidic conditions involving ammonium chloride, acetic acid, hydrochloric acid and the like mixtures thereof. Cyclization of compound of the formula (Y3) using CDI in polar aprotic solvents like DMF, DMSO, halogenated solvents like DCM, chloroform, ethereal solvents like THF, 1,4-dioxane, at room temperature or elevated temperatures provided compound of formula (Y4).

Compound of formula (Y4) undergoes N-alkylation using alkyl halides in presence of bases such as NaH, Potassium/sodium alkoxides, K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (Y5). Compound of formula (Y5) allowed to react with tert-butyl carbamate in the presence of catalyst such as (tris(dibenzylideneacetone) dipalladium(0), palladium (II) acetate, Bis(dibenzylideneacetone)₂ Pd(0), racemic 2,2′-Bis(diphenylphosphino)-1,1′-binaphthyl, 2,5 bis(tri-t-butylphosphine) palladium (0) and the like; in presence of ligands such as RuPhos, Xanthphos, Davephos, BINAP, or the like; using a suitable base such as K₂CO₃, Na₂CO₃, Cs₂CO₃, sodium tert-butoxide, potassium tert-butoxide, DIPEA, Potassium triphosphate and thereof; in a suitable solvent selected from THF, 1,4-dioxane, dimethoxyethane, DMF, DMA, toluene and the like to provide compound of formula (Y6).

Compound of formula (Y6) undergoes deprotection in acidic conditions using organic acids such as trifluoroacetic acid, Methane sulfonic acid and like, mineral acids like hydrochloric acid, acetic acid (aqueous or in etheral solvents), sulfuric acid and the like; using solvents like dichloromethane, dichloroethane, THF, 1,4-dioxane and like thereof to provide compound of formula (Y7).

Compound of formula (Y7) allowed to react with alkylnitrile in presence of the acidic reagents such as methane sulfonic acid, sulfuric acid, hydrochloric acid or the like to obtain compound of formula (Y8). The same transformation can be carried out using trialkyl orthoacetate in presence of ammonium acetate, in corresponding polar protic solvents like ethanol, methanol and thereof.

Compound of formula (Y8) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like at room temperature or elevated temperatures to provide compound of formula (Y9).

Compound of formula (Y9) allowed to react with compound of formula (A5) in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; in etheral solvents such as THF, 1,4 dioxane and like or polar aprotic solvents like DMF, DMA, DMSO and like, at elevated temperatures.

The compounds of formula (I) was prepared by following the sequential transformations as depicted and described in Scheme-Z herein

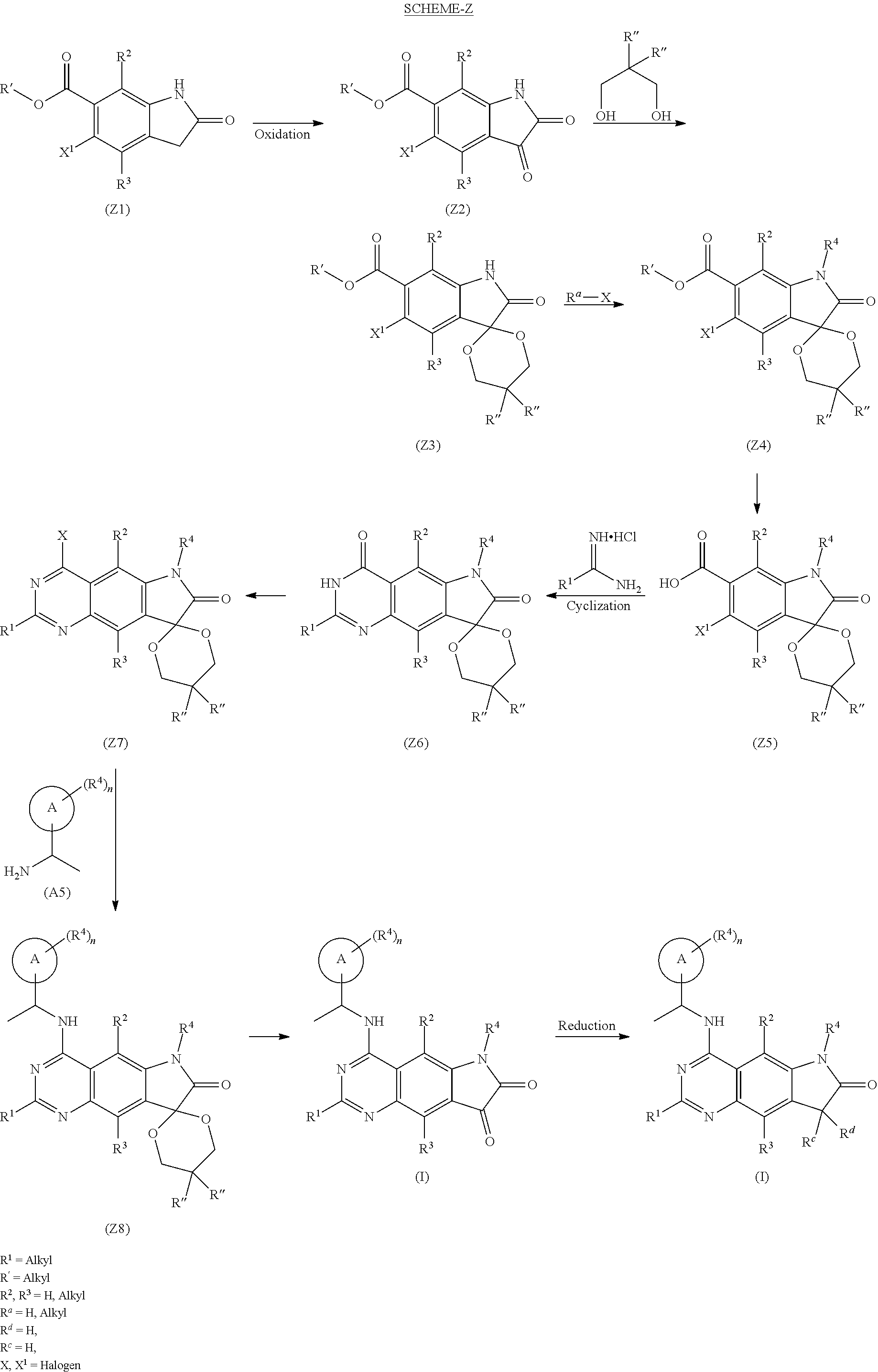

Compound of formula (Z1)undergoes oxidation reaction using oxidizing reagents such as tertiary butyl hydroperoxide, selenium dioxide, manganese dioxide and like; in presence of catalytic CuI, Cu(I) reagents and thereof; to provide compound of formula (Z2).

Compound of formula (Z3) can be obtained from compound of formula (Z2) by employing carbonyl protection reaction using diols such as 2,2-dimethylpropane-1,3-diol and like; in presence of mild acidic reagents such as PTSA and thereof; using hydrocarbon solvents like cyclohexane and like.

Compound of formula (Z3) on N-alkylation using alkyl halides and bases such as K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (Z4)

Compound of formula (Z4) undergoes hydrolysis of ester group in presence of alkali hydroxides such as NaOH, LiOH and thereof, using solvents like methanol, ethanol and thereof or using solvents like THF, 1,4-dioxane and thereof; to afford compound of formula (Z5).

Compound of formula (Z5) can be converted to compound of formula (Z8) by employing analogous 3 step protocol mentioned in scheme P for conversion of compound of formula (P6) to compound of formula (I).

Compound of formula (Z8) on ketal deprotection in acidic medium provide compound of formula (I). This transformation was done by employing mineral acids such as HCl, H₂SO₄ and like; by employing solvents such as 1,4-dioxane. THF, acetic acid and like. Further, Compound of formula (I) can be converted to compound of formula (I) by Wolff kishner reduction using hydroxyl amine hydrochloride reduction in alkaline medium. Such transformation can also be carried out by using Clemmensen reduction reaction in acidic medium.

The compounds of formula (I) was prepared by following the sequential transformations as depicted and described in Scheme-AA herein below

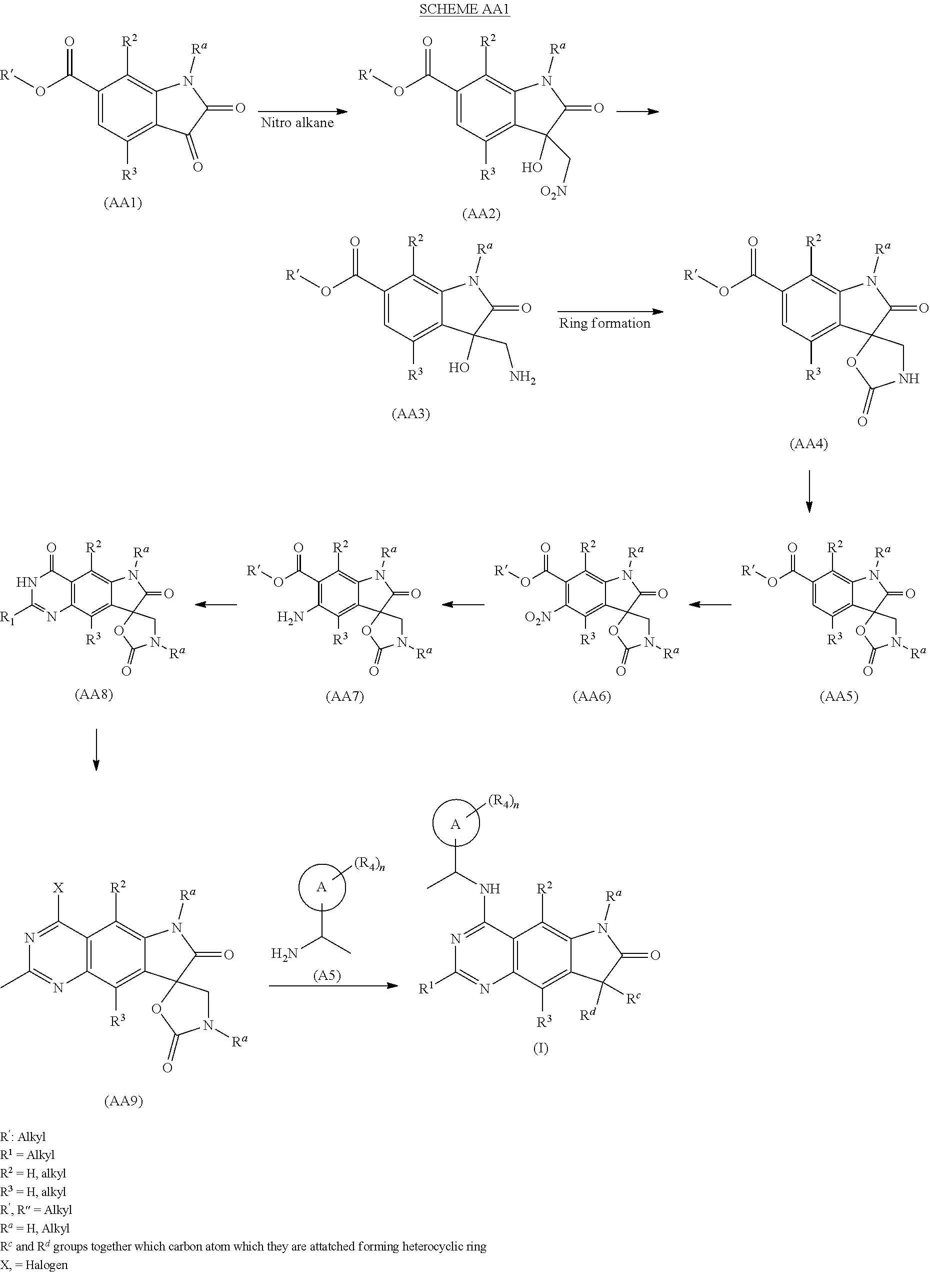

Compound of formula (AA1) on henry's reaction with nitroalkane in basic medium provided compound of formula (AA2). Such transformations can be carried out in presence of organic bases such as DIPEA, DABCO, and DBU and like, using nitroalkanes as solvent.

Compound of formula (AA2) on nitro reduction provided compound of formula (AA3). The reduction of nitro group was carried out using different reagents; although not limited, such reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and mixtures thereof.

Compound of formula (AA3) undergoes carbamate formation reaction mediated by reagents such as using CDI in polar aprotic solvents like DMF. DMSO, halogenated solvents like DCM, chloroform, ethereal solvents like THE, 1,4-dioxane, at room temperature or elevated temperatures provided compound of formula (AA4)

Compound of formula (AA4) undergoes N-alkylation using alkyl halides and bases such as K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1,4-dioxane and like, at room temperature or elevated temperatures provide compound of formula (AA5)

Compound of formula (AA5) can be transformed to compound of formula (I) in five steps analogous to protocol mentioned in scheme-S for conversion of compound of formula (S4) to compound of formula (I).

The compounds of formula (I) was prepared by following the sequential transformations as depicted and described in Scheme-AB herein below

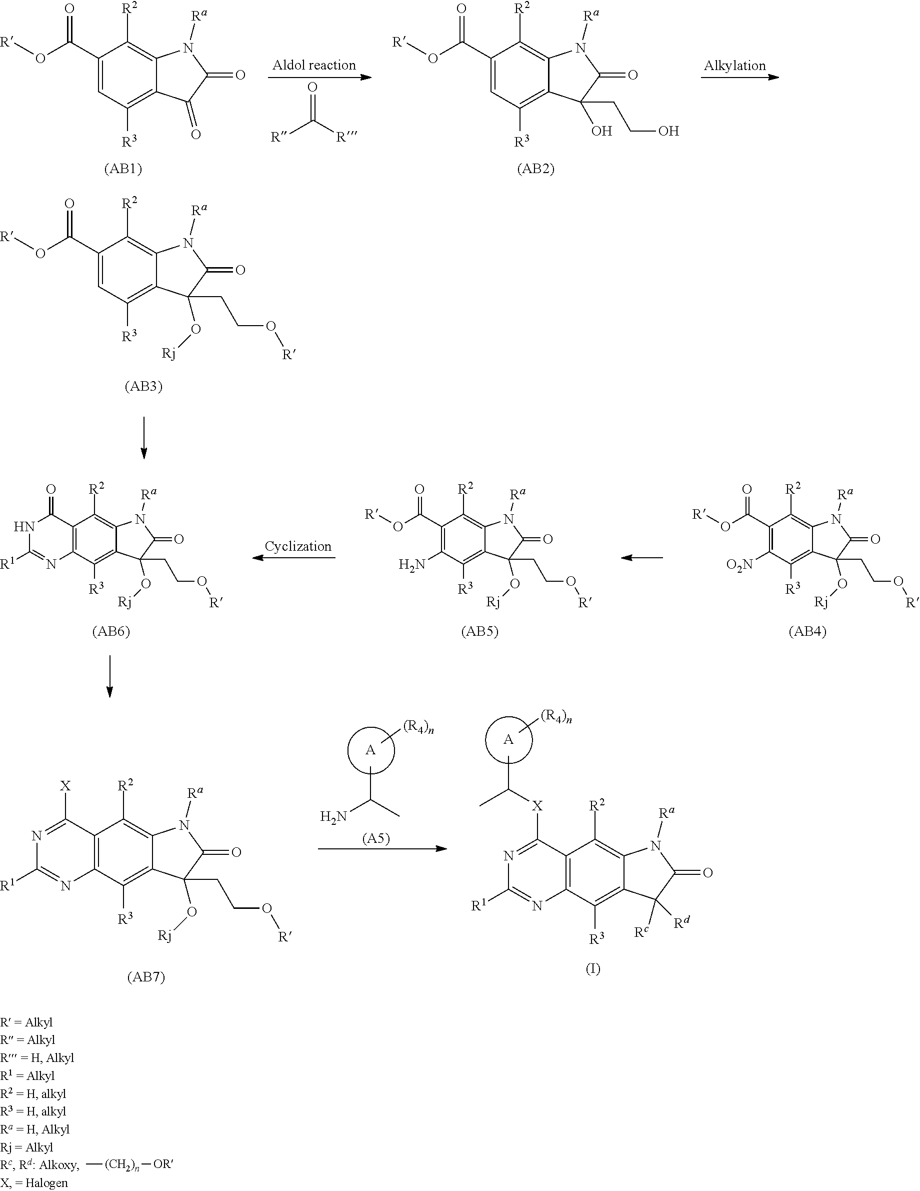

Compound of formula (AB1) undergoes Aldol type reaction with aldehydes and ketones viz. acetaldehyde in presence of secondary amines such as diethyl amine, pyrrolidine and like, provide aldol intermediate which further on carbonyl reduction using NaBH₄ and like, in alcohol solvents such as methanol, ethanol and mixtures thereof provide diol compound of formula (AB2)

Compound of formula (AB2) undergoes O-alkylation reaction with alkyl halides in presence of bases such as NaH, sodium/potassium alkoxides, K₂CO₃, Na₂CO₃, Cs₂CO₃; organic bases like diisopropylethyl amine, DBU, DABCO and so on; in polar aprotic solvents like DMF, DMSO, acetone and like, etheral solvents such as THF, 1, 4-dioxane and like, at room temperature or elevated temperatures provide compound of formula (AB3)

Compound of formula (AB3) undergoes nitration reaction to provide compound of formula (AB4). This reaction was carried out in presence of nitrating reagents such as potassium nitrate, sodium nitrate nitric acid and like; in acidic solvents such as sulfuric acid and thereof.

Compound of formula (AB4) undergoes reduction reaction to provide compound of formula (AB5). These transformations can be carried out using reducing agents include hydrogenation with palladium on carbon, metal reductions like iron, tin or tin chloride and the like. These reactions are carried out in one or more solvents, e.g., ethers such as THF, 1,4-dioxane, and the like; alcohol such as methanol, ethanol and the like; under acidic conditions involving ammonium chloride, acetic acid, hydrochloric acid and the like mixtures thereof

Compound of formula (AB5) allowed to react with alkyl nitrile in presence of the acidic reagents such as methanesulfonic acid, sulfuric acid, hydrochloric acid, or the like to obtain compound of formula (AB6). The same transformation can be carried out using trialkyl orthoacetate in presence of ammonium acetate, in corresponding polar protic solvents like ethanol, methanol and thereof.

Compound of formula (AB6) allowed to react with phosphoryl halides such as POCl₃ or POBr₃ optionally in solvents such as toluene, xylene, chlorobenzene or the like or the mixtures thereof, optionally using organic base such as triethylamine, diisopropylethylamine or the like to provide compound of formula (AB7).

Compound of formula (AB7) allowed to react with compound of formula (A5) in presence of suitable coupling reagent to provide compound of formula (I). The reaction can be carried out in presence of organic base such as diisopropylethylamine, triethylamine, DBU or the like, or using coupling reagents such as DCC, EDC, BOP, pyBOP, HBTU or the like; in etheral solvents such as THF, 1,4 dioxane and like or polar aprotic solvents like DMF, DMA, DMSO and thereof

The compounds of formula (I) was prepared by following the sequential transformations as depicted and described in Scheme-AC herein below

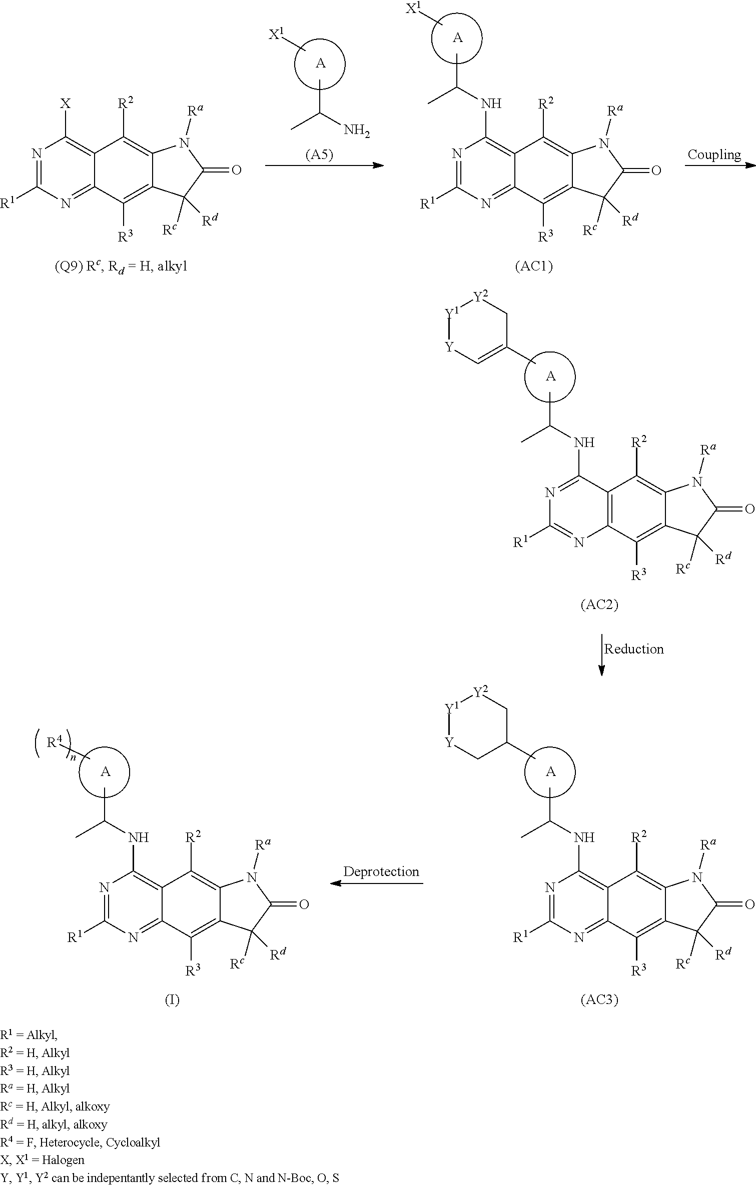

Compound of formula (Q9) undergoes coupling reaction with compound of formula (A5) to furnish compound of formula (I). The reaction can be carried out in presence of organic base such as diisopropylethylamine, triethylamine, DBU or the like, or using coupling reagents such as DCC, EDC, BOP, pyBOP, HBTU or the like; either neat reaction in base or in etheral solvents such as THE 1,4 dioxane and like or polar aprotic solvents like DMF, DMA, DMSO and thereof

The compound of formula (AC1) was subjected to C—C coupling reaction e.g. suzuki coupling reaction with corresponding boronic acid or boronic ester to provide compound of formula (AC2). This reaction can mediated by a suitable catalyst such as, e.g., Pd(PPh₃)₂Cl₂, Pd₂dba₃, Pd(PPh₃)₄, PdCl₂(dppf). DCM adduct or mixtures thereof, in the presence of suitable base, preferably inorganic bases such as K₂CO₃, Na₂CO₃, Cs₂CO₃, NaO^{t}Bu, Potassium phosphate, or mixture thereof. Such reactions can be carried out in solvents like, e.g., ethers such as THF, 1,4-Dioxane and the like; hydrocarbons, e.g., toluene; amides such as DMF, DMA or mixtures thereof

Compound of formula (AC2) undergoes hydrogenation reaction in presence of catalyst such as Pd(OH)₂ on carbon, palladium on carbon, and the like; in one or more solvents e.g alcohol such as methanol, ethanol and the like; under acidic conditions involving ammonium chloride, acetic acid, hydrochloric acid and mixtures thereof, optionally in presence of water to provide compound of formula (AC3)

Compound of formula (AC3) undergoes deprotection reaction mediated by acids such as organic acids e,g trifluoroacetic acid, Methane sulfonic acid and the like, mineral acids e.g hydrochloric acid, acetic acid (Aqueous or in etheral solvents), sulfuric acid and the like; using solvents like dichloromethane, dichloroethane, THF, 1,4-dioxane and mixtures thereof to provide compound of formula (I)

### General Synthetic Procedures for SOS1 Inhibitor of Formula H,

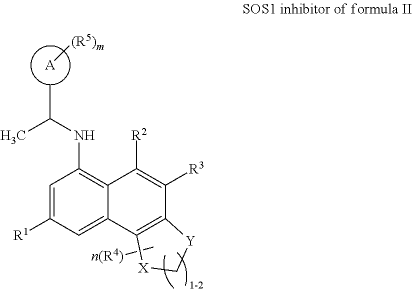

### Scheme-A Illustrates the Synthesis of Compound of Formula (A5)

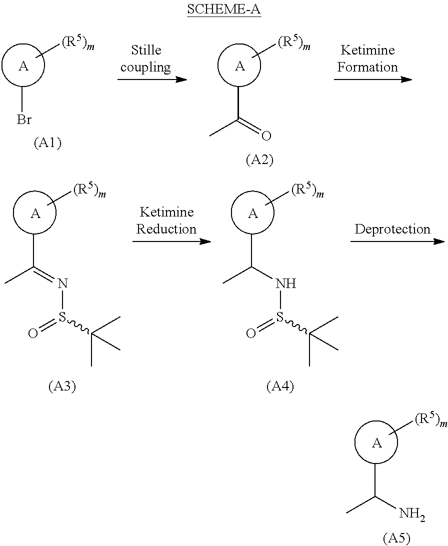

The compound of formula (A1) undergoes a metal catalyzed cross coupling with alkoxy vinyl stannane, e.g. tributyl(1-ethoxyvinyl)tin in presence of palladium catalysts such as Pd(Ph₃P)₂Cl₂, Pd₂(dba)₃, and the like; optionally using bases such as triethylamine, N,N-Diisopropylethylamine, and the like, in hydrocarbon solvents like toluene or ether solvents like 1,4-dioxane to furnish the alkoxy vinyl intermediate which in turn provide compound of formula (A2) in acidic condition by employing aqueous mineral acids such as hydrochloric acid in ether solvent such as THF, 1,4-dioxane and the like.

The compound of formula (A2) was then reacted with corresponding chirally pure tert-butanesulfinamide in presence of Lewis acid such as titanium alkoxides e.g. titanium tetraethoxide, titanium isopropoxide, and the like, in ether solvents such as 1,4-dioxane, THF, and the like, to obtain the compound of formula (A3).

The compound of formula (A3) reacted with reducing agent such as metal hydrides e.g. sodium borohydride, L-selectride, and the like in solvents such as THF, 1,4-dioxane, methanol, and the like, optionally in presence of water to provide compound of formula (A4). Major diastereoisomer in the compound of formula (A4) after reduction was separated or taken ahead as such.

The compound of formula (A4) under acidic condition undergoes cleavage of sulfinyl derivative to generate amine of formula (A5) as a free base or salt. The acids employed for the transformation may involve mineral acids such as hydrochloric acid or organic acids such as trifluoroacetic acid.

### Scheme-B Illustrates the Synthesis of SOS1 Inhibitor of Formula II and (II-A)

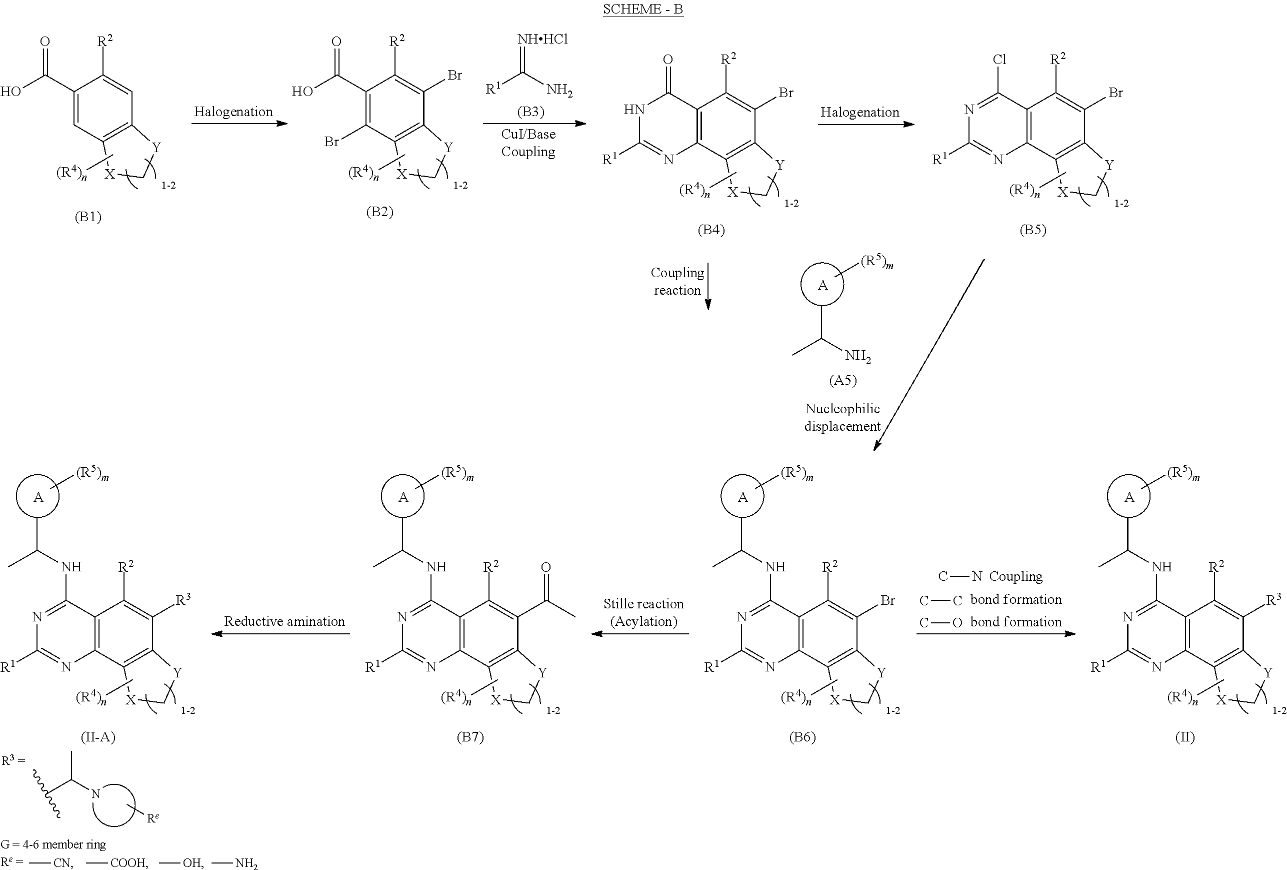

Compound of formula (B1) was either commercially purchased or prepared by following a procedure reported in Russian Journal of Organic Chemistry, 2002, vol. 38, #12, p. 1764-1768. Halogenation of carboxylic acid (B1) using N-halosuccinimide reagent such as but not limited to NBS, NIS, and NCS gives corresponding dihalo compound of formula (B2), which on coupling with different amidines of formula (B3) gives compound of formula (B4) (where R¹=alkyl).

The compound of formula (B4) could be either directly converted to compound of formula (B6) using different benzylic amines (A5) and coupling reagents such as but not limited to BOP, etc in polar solvents such as but not limited to ACN, DMF, and DMSO, or compound of formula (B4) could be further halogenated by using reagents such as but not limited to chlorinating agents like POCl₃, POBr₃, Oxalyl chloride, or SOCl₂ and bases such as but not limited to DIPEA, TEA, and N,N-dimethyl aniline in solvents such as but not limited to chloroform, dichloroethane, and chlorobenzene to give compound of formula (B5).

Compound of formula (B5) undergoes a nucleophilic substitution reaction with different benzylic amines (A5) leading to compound of formula (B6). The compound of formula (B6) could be further acylated using Stille reaction condition to compound of formula (B7) which could be further converted into compound of formula (II-A) through reductive amination using appropriate substituted amine. The compound of formula (B6) could be further functionalized e.g. transition metal catalyzed C—C coupling, C—N bond formation or C—O bond formation reactions like Suzuki or Buchwald reaction utilizing corresponding counterpart, i.e. substituted amine or substituted boronate to get compound of formula (II).

### Scheme-C Illustrates the Synthesis of Compound of Formula (B4)

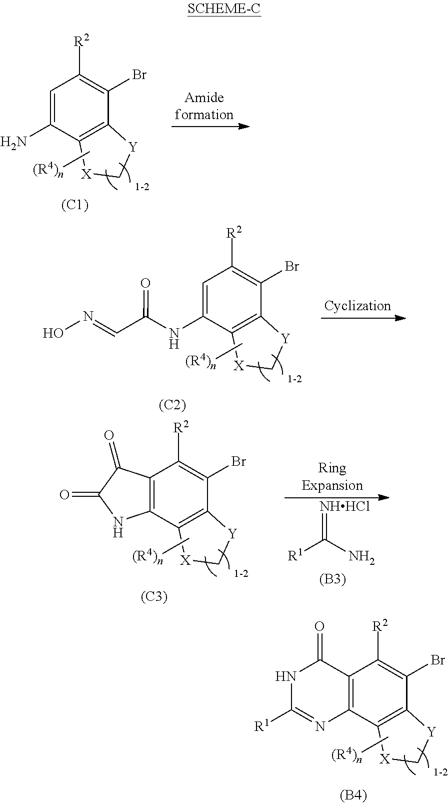

Compound of formula (C1) was obtained commercially or can be obtained through following a procedure reported in WO2017139778 and Helvetica Chimica Acta, 1981, vol. 64, #2, p. 572-578.

The compound of formula (C1) was treated with Chloral hydrate and hydroxylamine to afford the compound of formula (C2) at appropriate temperature.

The compound of formula (C2) which upon treating with inorganic acids like H₂SO₄ gets cyclized at appropriate temperature leading to isatin derivative as compound of formula (C3) which on coupling with different amidines (B3) by using bases such as K₃PO₄, K₂CO₃, Na₂CO₃, Cs₂CO₃ etc in polar aprotic solvents like DMF, DMSO etc at appropriate temperature leading to compound of formula (B4) (where R¹=alkyl).

### Scheme-D Illustrates the Synthesis of Compound of Formula (II-B)

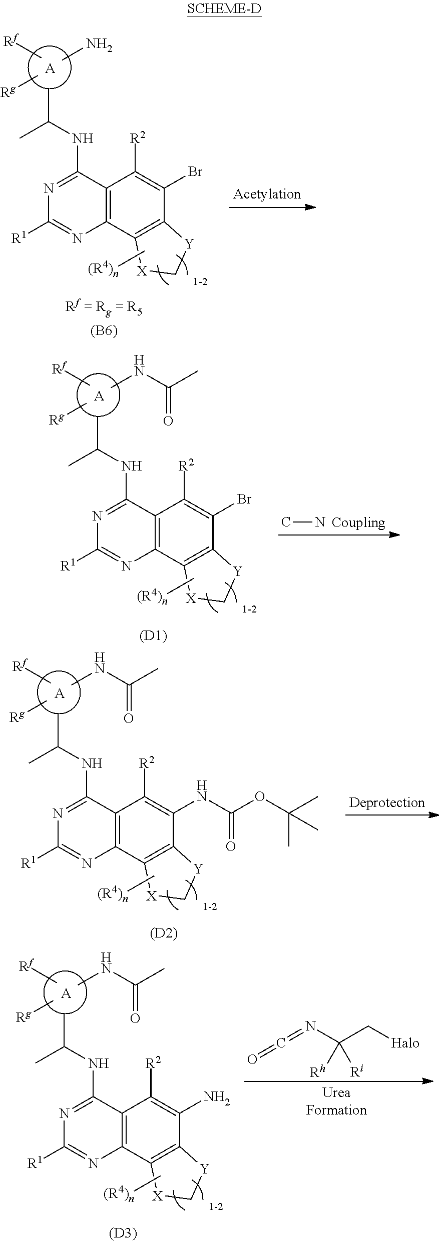

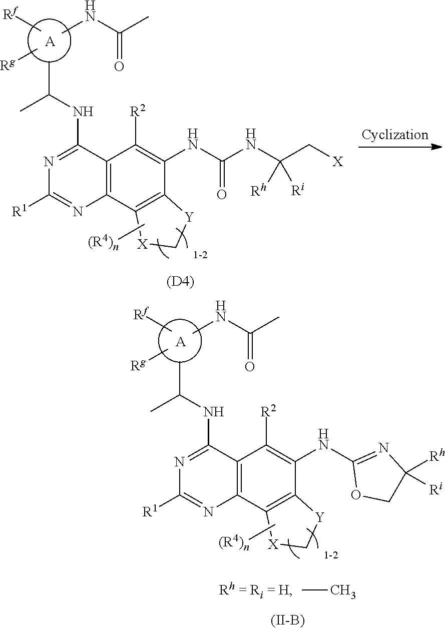

The compound of formula (D1) can be synthesized via acetylation of corresponding aniline compound of formula (B6) as mentioned in above Scheme-B.

The compound of formula (D1) was converted to corresponding carbamate compound of formula (D2) using transition metal catalyzed cross coupling such as via Buchwald Hartwig coupling, which further upon deprotection lead to intermediate compound of formula (D3).

The compound of formula (D3) could be further functionalized to urea compound of formula (D4) by treating with corresponding isocyanates (where R^{h}═Rⁱ═H, CH₃).

The compound of formula (D4) could be further cyclized leading to final compound of formula (f-B) using bases such as KO^{t}Bu, NaH etc in a polar aprotic solvent like DMF, DMSO etc at appropriate temperature.

### Scheme-E Illustrates the Synthesis of Compound of Formula (II-C)

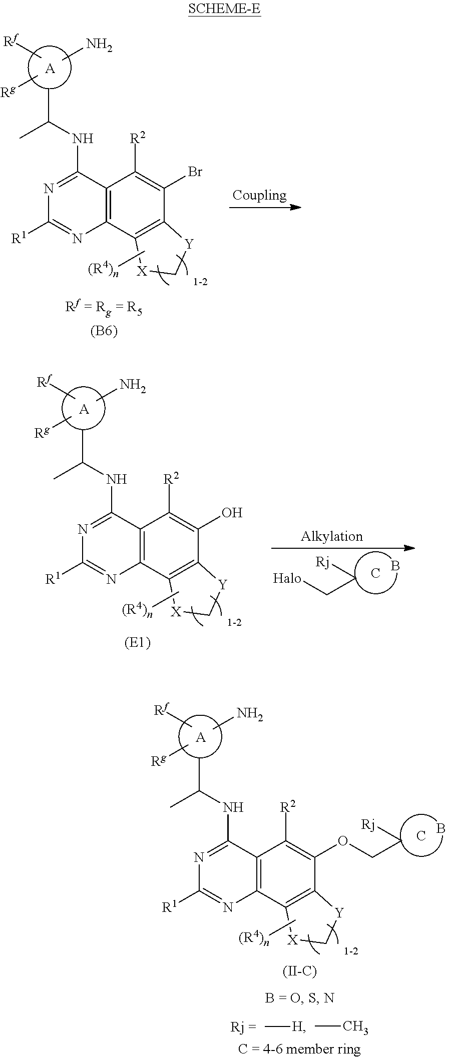

The compound of formula (B6) as prepared following Scheme-B, could be converted to corresponding hydroxy derivative of compound of formula (E1) via e.g. transition metal catalyzed cross coupling.

Compound of formula (E1) could be further alkylated by using bases such as K₂CO₃, Na₂CO₃, to the compound of formula (II-C).

Scheme-F Illustrates Formation of Compound of Formula (II-D) Starting from Commercially Available Compound of Formula (F1)

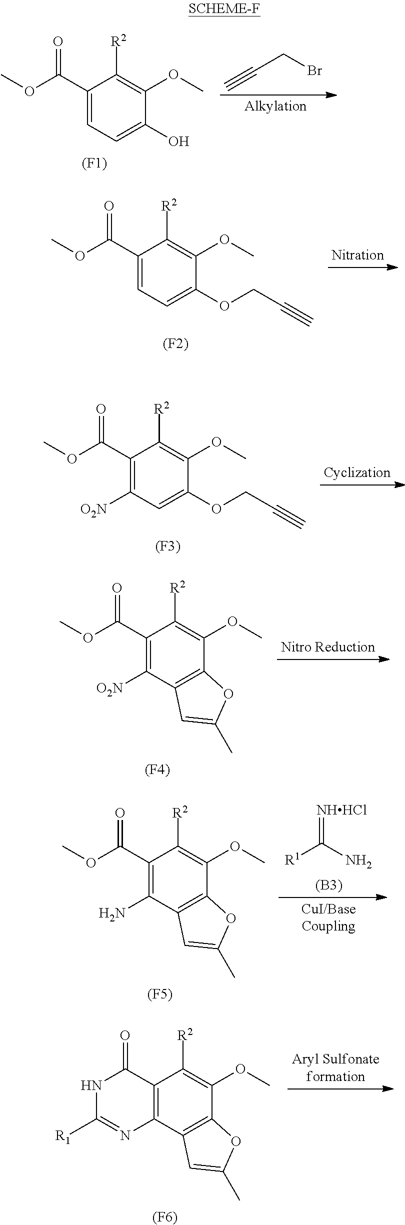

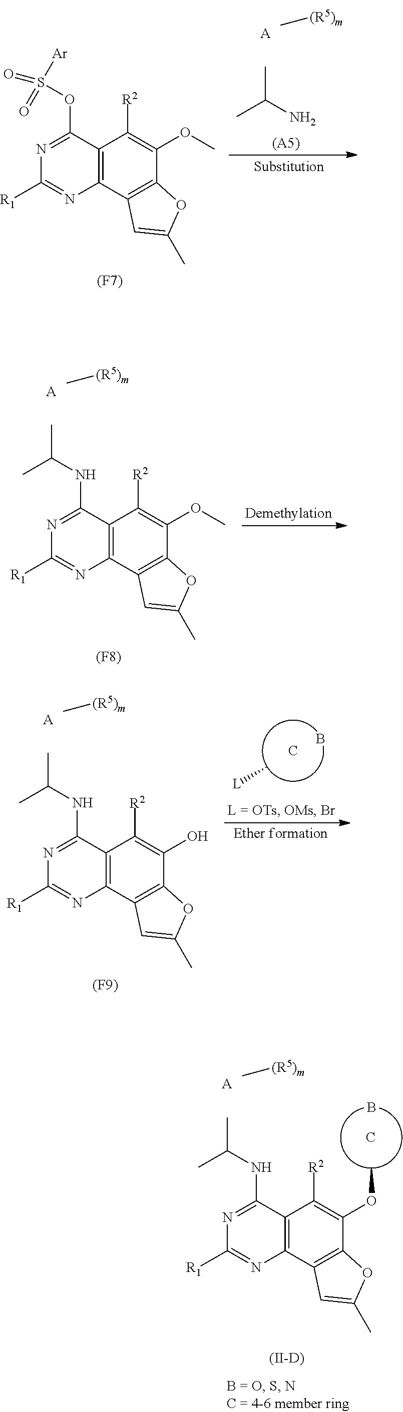

Compound of formula (F1) upon alkylation using propargyl bromide affords corresponding compound of formula (F2).

Nitration of compound of formula (F2) with nitrating reagents such as, although not limited to nitric acid, potassium nitrate, and the like, in acids such as, although not limited to tin (IV) chloride, sulphuric acid, trifluroacetic acid, acetic acid, and the like, anhydrides like acetic anhydride, trifluroacetic anhydride, and the like, or mixture(s) thereof to provides compound of formula (F3), which upon Claisen rearrangement and in situ cyclization at appropriate temperature, to affords compound of formula (F4). Such reactions can be carried out in either neat or in presence of high boiling solvents such as, although not limited to NMP, diphenyl ether, xylene, N,N-diethyl aniline, and the like or mixtures thereof and also in combination with bases such as, although not limited to cesium fluoride and high boiling solvents such as, although not limited to N,N-diethyl aniline, NMP, diphenyl ether, xylene, and the like or mixtures thereof.

Compound of formula (F4) was converted to corresponding aniline derivative of compound of formula (F5) through selective reduction of nitro group by using reducing agents, although not limited to, such reducing agents include hydrogenation with palladium on carbon, metal reductions like iron, tin or tin chloride and the like. Such reduction could be carried out in one or more solvents, e.g., ethers such as THF, 1,4-dioxane, and the like; alcohol such as methanol, ethanol and, the like; under acidic conditions involving ammonium chloride, acetic acid, hydrochloric acid, and the like or mixtures thereof.

Compound of formula (F5) could be further cyclized to give compound of formula (F6) as tricyclic building block. Such reaction can be carried out in polar solvent like acetonitrile using acids such as, but not limited to methane sulfonic acid or hydrochloric acid at appropriate temperature.

The compound of formula (F6) is treated with tri-isopropyl benzene sulfonyl chloride to afford corresponding sulfonate derivative of compound of formula (F7) in solvents such as ethers like THF or 1,4-Dioxane at appropriate temperature.

Compound of formula (F7) undergoes a nucleophilic substitution reaction with appropriate chiral benzylic amines leading to the compound of formula (F8) using organic basic reagents such as, but not limited to DIPEA or TEA in a polar aprotic solvent like dioxane or THF at appropriate temperature.

The compound of formula (F8) demethylated to corresponding hydroxy derivative of compound of formula (F9) by using reagents like Lewis acids such as, but not limited to BBr₃, AlCl₃, etc and basic reagents such as, but not limited to NaSEt, etc in polar solvents such as, although not limited to DMF, can, and the like or mixtures thereof, and halogenated solvents such as, although not limited chloroform, dichloromethane, and the like or mixtures thereof.

The compound of formula (F9) can be further alkylated by using inorganic bases such as, but not limited to K₂CO₃, Na₂CO₃, and Cs₂CO₃ etc in polar aprotic solvents like DMF, DMSO etc at appropriate temperature leading to final compound of formula (II-D).

Scheme-G Illustrates Formation of Compound of Formula (II-E) Starting from Compound of Formula (G1) (Reference: CN105884699)

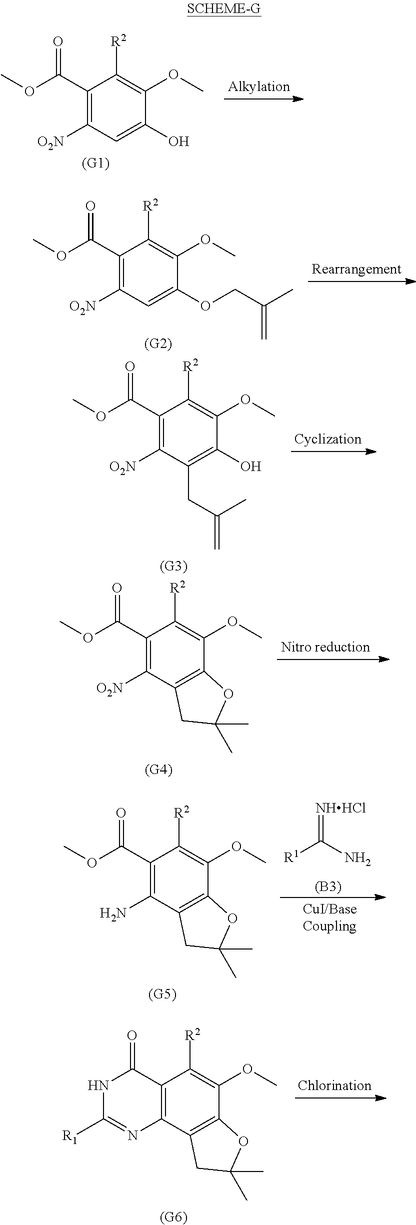

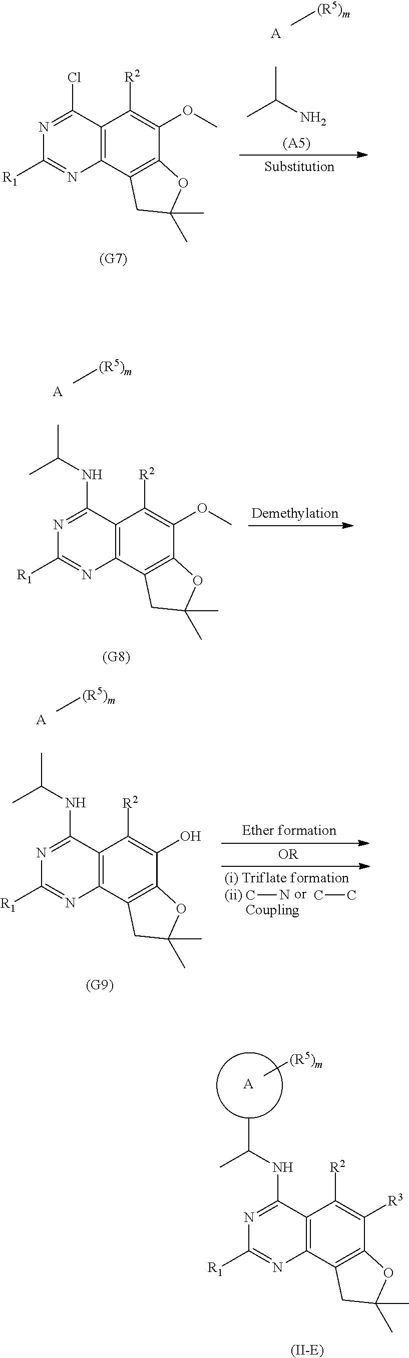

Compound of formula (G1) upon alkylation using 3-chloro-2-methylprop-1-ene afford compound of formula (G2). Such reaction could be carried out by using inorganic bases such as, although not limited to K₂CO₃, Cs₃CO₃, Na₂CO₃ and organic bases such as, although not limited to DIPEA, TEA, diisopropyl amine, and the like etc., and the polar aprotic solvents such as, although not limited to acetone, acetonitrile, and DMF or mixture(s) thereof.

The compound of formula (G2) upon Claisen rearrangement at appropriate temperature to affords hydroxyl derivative of compound of formula (G3). Such reactions can be carried out in either neat or in presence of high boiling solvents such as, but not limited to NMP, diphenyl Ether, xylene, N,N-diethyl aniline, and the like or mixtures thereof.

Compound of formula (G3) upon cyclization in solvents such as, although not limited to THF, Diethyl ether, dioxane, and ACN under acidic conditions such as, but not limited to formic acid, acetic acid, hydrochloric acid, and the like mixture(s) thereof at appropriate temperature to afford compound of formula (G4).

The compound of formula (G4) further converted to corresponding aniline derivatives of compound of formula (G5) through selective reduction of nitro group by using reducing agents such as, although not limited to, such reducing agents include hydrogenation with palladium on carbon, metal reductions like iron, tin or tin chloride, and the like. Such reduction reaction can be carried out in one or more solvents, e.g. ethers such as THF, 1,4-dioxane, and the like; alcohol such as methanol, ethanol, and the like; under acidic conditions involving ammonium chloride, acetic acid, hydrochloric acid, and the like or mixture(s) thereof.

The compound of formula (G5) could be further cyclized to give compound of formula (G6) as tricyclic building block. Such reaction can be carried out in polar solvent like acetonitrile using acids such as, but not limited to methane sulphonic acid, hydrochloric acid etc at appropriate temperature.

The compound of formula (G6) could be halogenated by using reagents such as, although not limited to, POCl₃ or POBr₃ in combination with organic bases such as, although not limited to DIPEA, TEA in halogenated solvents such as, although not limited to chlorobenzene, chloroform, DCM etc at appropriate temperature to give compound of formula (G7).

The compound of formula (G7) undergoes a nucleophilic substitution reaction with different chiral benzylic amines (A5) leading to the compound of formula (G8) using organic basic reagents such as, but not limited to DIPEA, TEA etc in a polar aprotic solvents like dioxane, THF etc at appropriate temperature.

The compound of formula (G8) demethylated to corresponding hydroxy derivative of compound of formula (G9) by using reagent such as, but not limited to BBr₃, NaSEt etc in polar solvents such as DMF, ACN, and the like; halogenated solvents such as chloroform, dichloromethane, etc.

The compound of formula (G9) can be further alkylated to form ether compound of general formula (I-E) by using organic bases such as, but not limited, DIPEA, TEA at appropriate temperature or the said alkylation can be carried out by using bases such as K₂CO₃, Na₂CO₃, Cs₂CO₃, etc in polar aprotic solvents like DMF, DMSO etc at appropriate temperature. The compound of formula (G9) could be converted to ether compound of general formula (II-E) via Mitsunobu reaction also.

However, the compound of formula (G9) could also be converted to corresponding triflate with triflic anhydride in halogenated solvents such as, but not limited to DCM, CHCl₃, etc and further reacting this triflate intermediate with appropriate aliphatic amines or boronic acid to afford compound of general formula (II-E). This reaction could be mediated by a suitable catalyst such as, e.g., Pd(PPh₃)₂Cl₂, Pd₂dba₃, Pd(PPh₃)₄, Pd(OAc)₂, or mixture(s) thereof; a suitable ligand such as, although not limited to Xantphos, BINAP, Ru-Phos, or mixture(s) thereof; in the presence of suitable base, preferably inorganic bases such as, although not limited to e.g., K₂CO₃, Na₂CO₃, Cs₂CO₃, NaO^{t}Bu, Potassium phosphate, or mixture(s) thereof. Such reactions can be carried out in solvents like, e.g., ethers such as THE, dioxane, and the like; hydrocarbons, e.g., toluene; amides such as DMF, DMA, or mixture(s) thereof.

Scheme-H Illustrates Formation of Compound of Formula (II-F) Starting from Compound of Formula (F4)

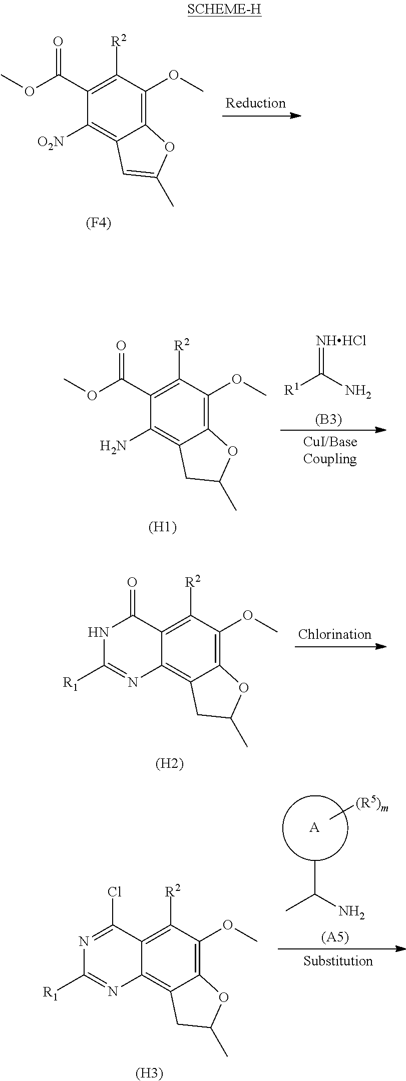

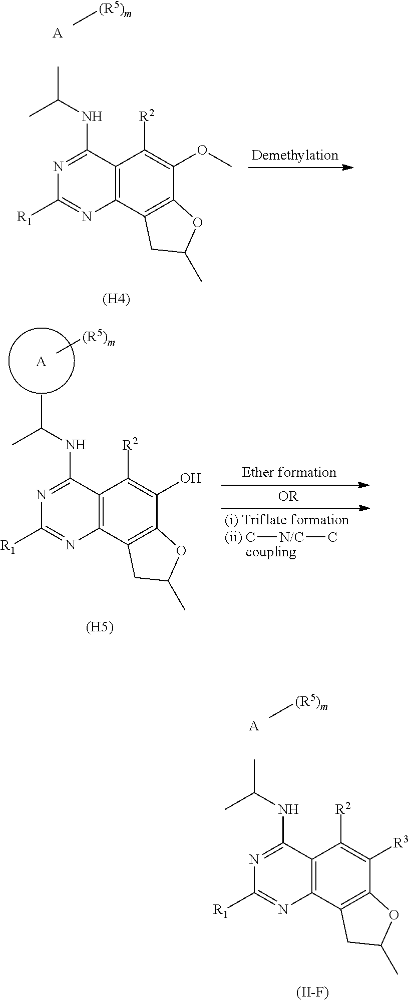

The compound of formula (F4) can be reduced to corresponding aniline derivative (H1) through selective reduction of nitro group and aromatic double bond by using reducing agents, such as, although not limited to, such reducing agents include hydrogenation with palladium on carbon, metal reductions like iron, tin or tin chloride, and the like. Such reduction reaction can be carried out in one or more solvents, although not limited to, e.g., ethers such as THF, 1,4-dioxane, and the like; alcohol such as methanol, ethanol, and the like; under either neutral or acidic conditions involving ammonium chloride, acetic acid, hydrochloric acid, and the like, or mixture(s) thereof.

The compound of formula (H1) can be further cyclized to give compound of formula (H2) as tricyclic building block. Such reaction can be carried out in polar solvent like acetonitrile using acids such as, but not limited to methane sulphonic acid, hydrochloric acid etc at appropriate temperature.

The compound of formula (H2) can be halogenated by using reagents such as, although not limited, POCl₃ or POBr₃ in combination with organic bases such as, although not limited to, DIPEA. TEA in halogenated solvents such as chlorobenzene, chloroform, DCM etc at appropriate temperature to give the compound of formula (H3).

The compound of formula (H3) undergoes a nucleophilic substitution reaction with different chiral benzylic amines of compound of formula (A5) leading to the compound of formula (H4) using organic basic reagents such as, but not limited to DIPEA, TEA etc in a polar aprotic solvents like dioxane, THE etc at appropriate temperature.

The compound of formula (H4) demethylated to corresponding hydroxy derivative of compound of formula (H5) by using Lewis Acids reagent such as, but not limited to BBr₃, AlCl₃ etc and basic reagents such as, but not limited to NaSEt, etc in polar solvents such as, although not limited to DMF, can, and the like; halogenated solvents such as, although not limited to chloroform. dichloromethane, etc.

The compound of formula (H5) could be further alkylated to form ether compound of general formula (I-F) by using organic bases such as, but not limited to DIPEA, TEA etc at appropriate temperature, the said alkylation can be carried out by using bases such as K₂CO₃, Na₂CO₃, Cs₂CO₃, etc in polar aprotic solvents like DMF, DMSO etc at appropriate temperature. The compound of formula (H5) could be converted to ether compound of general formula (II-F) via Mitsunobu reaction also.

However, the compound of formula (H5) could also be converted to corresponding triflate with triflic anhydride in halogenated solvents such as, but not limited to, DCM, CHCl₃, etc and further reacting this triflate intermediate with appropriate aliphatic amines or boronic acid to afford compound of general formula (II-F). This reaction could be mediated by a suitable catalyst such as, e.g., Pd(PPh₃)₂Cl₂, Pd₂dba₃, Pd(PPh₃)₄, Pd(OAc)₂, or mixture(s) thereof; a suitable ligand such as, although not limited to Xantphos, BINAP, Ru-Phos or mixture(s) thereof; in the presence of suitable base, preferably inorganic bases such as, although not limited to e.g. K₂CO₃, Na₂CO₃, Cs₂CO₃, NaO^{t}Bu, Potassium phosphate, or mixture(s) thereof. Such reactions can be carried out in solvents like ethers such as THF, dioxane, and the like; hydrocarbons, e.g., toluene; amides such as DMF, DMA, or mixture(s) thereof.

### Scheme-I Illustrates the Synthesis of Compounds of Formula (II-G) and (II-H)

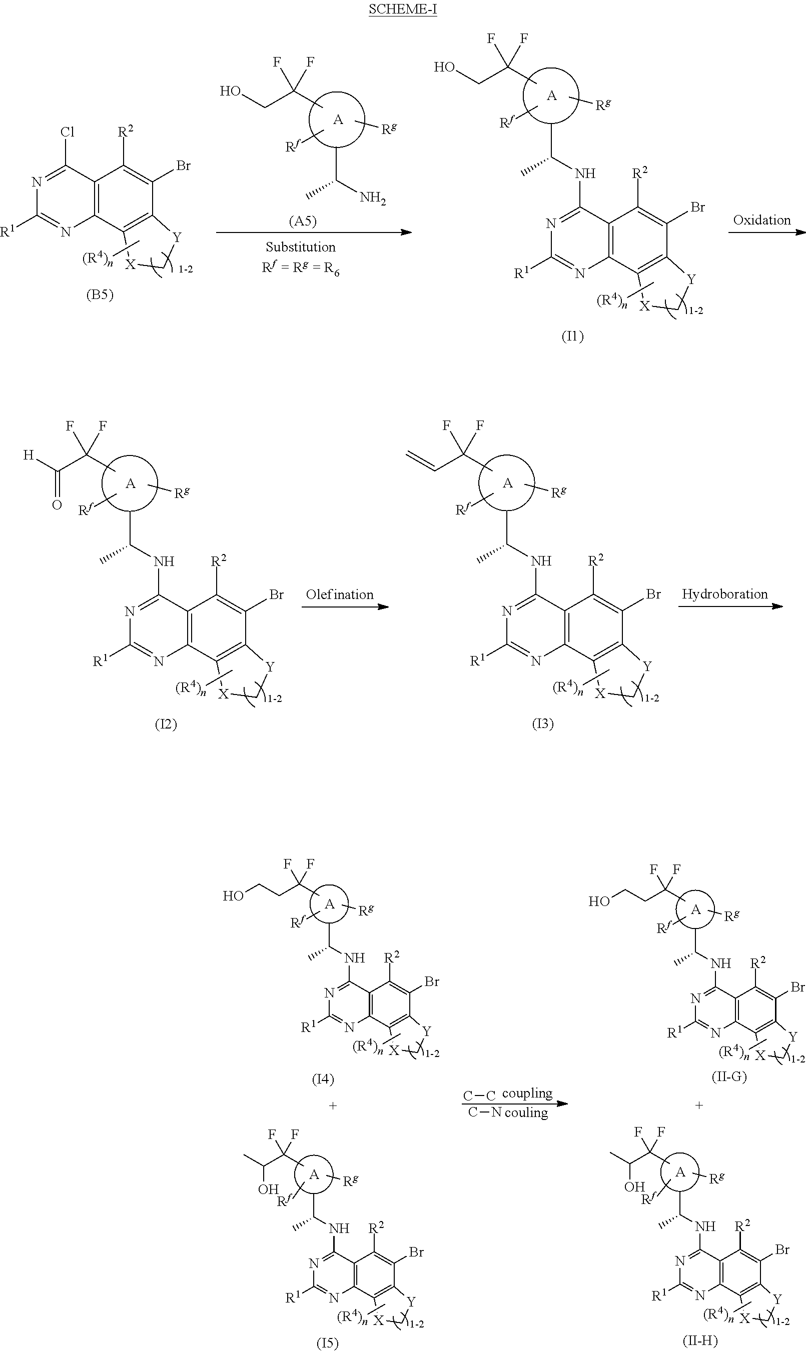

The compound of formula (B5) undergoes a nucleophilic substitution reaction with compound of formula (A5) in the presence of organic base such as, although not limited to TEA, pyridine, DIPEA, or DMAP leading to compound of formula (I1). Such reactions can be carried out in polar protic solvents such as MeOH, EtOH, IPA, and the like; amides such as DMF, DMA, and the like; ethers such as THF or 1,4-Dioxane and the like; halogenated solvents such as CHCl₃, DCE, chlorobenzene, and the like; polar aprotic solvents such as DMSO, can, and the like.

The compound of formula (I1) subjected to a controlled oxidation by using reagents such as, but not limited to, the said reagent is the combination oxalyl chloride and DMSO in organic solvents such as DCM, CHCl₃, DCE, and the like; in presence of organic base such as, but not limited to, triethylamine, N,N-diisopropylethylamine to give aldehyde compound of formula (I2).

The compound of formula (I2) was then subjected to the olefination reaction by using reagents such as, but not limited to, alkyltriphenyl phosphonium halide in presence of base such as, but not limited to, KHMDS, LDA in presence of ether solvent such as, but not limited to, THF, 1,4-dioxane, and like to obtain the compound of formula (I3).

The compound of formula (I3) undergoes hydroboration reaction by using a regents such as, but not limited to, Borane-THF complex, Borane-DMS complex or Per-acids like hydrogen peroxide in ether solvents such as, but not limited to, THF, 1,4-dioxane to gives the two regioisomers of compound of formula (I4) and racemic mixture (I5).

The compound of formula (H-G) and racemic mixture (II-H) could be prepared by the Buchwald coupling of compound of formula (I4) and racemic mixture (15) respectively with appropriate aliphatic amines. This reaction could be mediated by a suitable catalyst such as, but not limited to, Pd(PPh₃)₂CO₂, Pd₂dba₃, Pd(PPh₃)₄, Pd(OAc)₂, or mixture(s) thereof; a suitable ligand such as, but not limited to, 2-di-t-butylphosphino-2′-(N,N-dimethylamino)biphenyl, xantphos, BINAP, Ru-Phos, or mixture(s) thereof; in the presence of suitable base, preferably inorganic bases such as, but not limited to, alkali metal carbonates, e.g., Na₂CO₃, K₂CO₃, Cs₂CO₃, sodium tert-butoxide, potassium phosphate, or mixture(s) thereof. Such reactions could be carried out in solvents like ethers such as THF, dioxane, and the like; hydrocarbons, e.g., toluene and the like; amides such as DMF, DMA, and the like or mixture(s) thereof. The final separation through chiral chromatography would provide pure diastereomers of compound of formula (II-G).

Scheme-J Illustrates Formation of Compound of Formula (II-1) Starting from Compound of Formula (L1) (Reference: CN105884699)

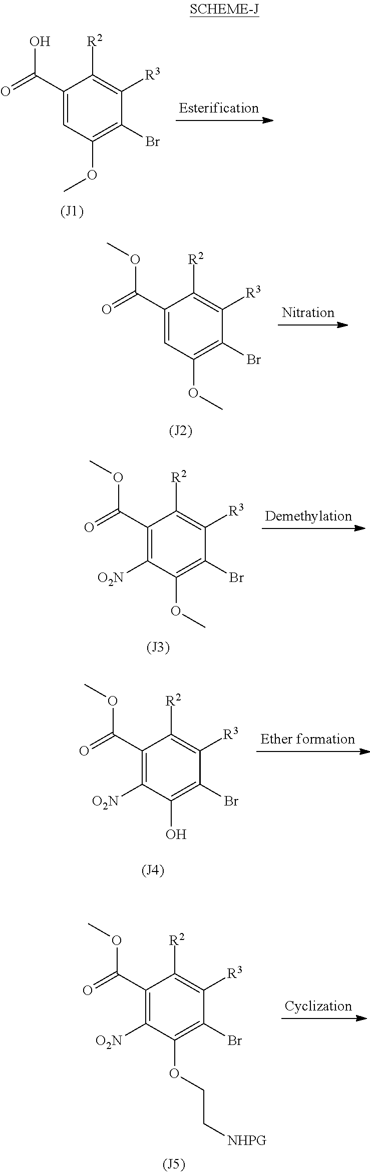

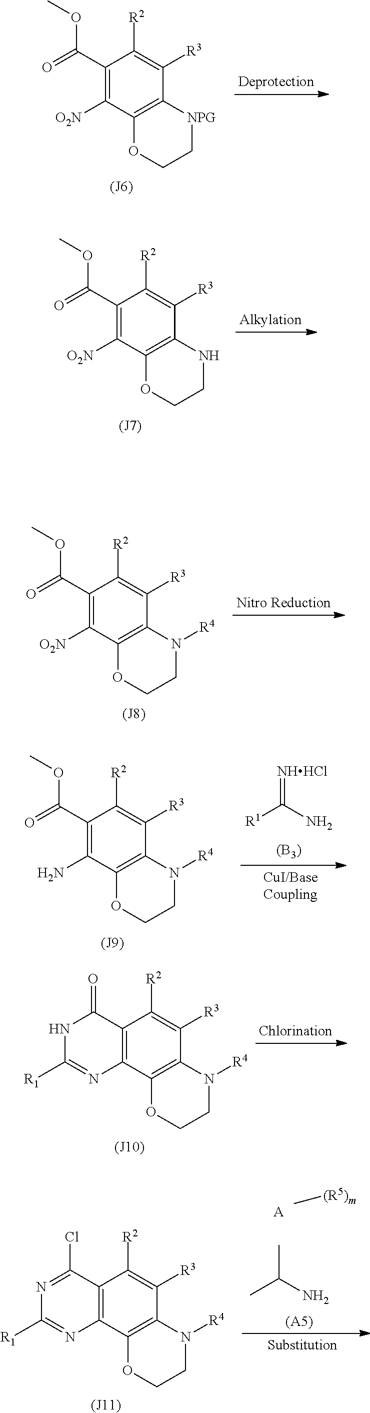

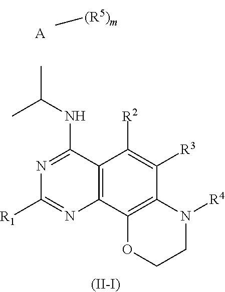

The compound of formula (J1) upon esterification using chlorinating reagents such as, but not limited to, thionyl chloride, oxalyl chloride in methanol affords the compound of formula (J2).

Nitration of compound of formula (J2) with nitrating reagents such as, although not limited to nitric acid, potassium nitrate, and the like in acids such as, although not limited to tin (IV) chloride, sulphuric acid, trifluroacetic acid, acetic acid, and the like, anhydrides like acetic anhydride, trifluroacetic anhydride, and the like, or mixture(s) thereof to provides the compound of formula (3).

The compound of formula (J3) selectively demethylated to corresponding hydroxy derivative of compound of formula (J4) by using reagent such as, but not limited to AlCl₃, BBr₃, NaSEt, etc in polar solvents such as DMF, can, and the like; halogenated solvents such as chloroform, dichloromethane, etc.

Compound of formula (J4) upon ether formation using protected amino alcohols like tert-butyl(2-hydroxyethyl)carbamate affords the compound of formula (J5). Such reaction could be carried out by using regents such as, although not limited to DIAD, DEAD, Triphenyl phosphine etc and solvents such as, but not limited to, ethers such as THF, dioxane, and the like; hydrocarbons, e.g., toluene or mixtures(s) thereof.

The compound of formula (J5) upon cyclization afford compound of formula (J6). This reaction could be mediated by a suitable catalyst such as but not limited to Pd(PPh₃)₂Cl₂, Pd₂dba₃, Pd(PPh₃)₄, Pd(OAc)₂, or mixture(s) thereof; a suitable ligand such as, although not limited to Xantphos, BINAP, Ru-Phos, or mixture(s) thereof; in the presence of suitable base, preferably inorganic bases such as, although not limited to e.g., K₂CO₃, Na₂CO₃. Cs₂CO₃, NaO^{t}Bu, Potassium phosphate, or mixture(s) thereof. Such reactions can be carried out in solvents like, e.g., ethers such as THF, Dioxane, and the like; hydrocarbons, e.g., toluene; amides such as DMF, DMA, or mixture(s) thereof.

The compound of formula (J6) under acidic condition undergoes deprotection to generate compound of formula (J7). The acids employed for the transformation may involve mineral acids such as hydrochloric acid or organic acids like trifluoroacetic acid.

The compound of formula (J7) upon alkylation or reductive amination using alkyl halides or aldehydes respectively afford compound of formula (J8). Such reaction could be carried out by using inorganic bases such as, although not limited to K₂CO₃, Cs₂CO₃, and Na₂CO₃, and the polar aprotic solvents such as, although not limited to acetone, acetonitrile, and DMF, or mixture(s) thereof, for alkylation and reducing agents like NaCNBH₄, Na(CH₃COO)₃BH etc in solvents like polar protic solvents such as but not limited to methanol, ethanol, acetic acid, and DME.

The compound of formula (J8) further converted to corresponding aniline derivatives of compound of formula (J9) through selective reduction of nitro group by using reducing agents, although not limited to, such reducing agents include hydrogenation with palladium on carbon, metal reductions like iron, tin or tin chloride, and the like. Such reduction reaction can be carried out in one or more solvents, e.g. ethers such as THF, 1,4-dioxane, and the like; alcohol such as methanol, ethanol, and the like; under acidic conditions involving ammonium chloride, acetic acid, hydrochloric acid, and the like, or mixture(s) thereof.

The compound of formula (J9) which on coupling with different amidines of compound of formula (B3) gives the compound of formula (J10) as tricyclic building block.

The compound of formula (J10) could be halogenated by using reagents such as, although not limited to, POCl₃ and POBr₃ or combination with organic bases such as, although not limited to DIPEA and TEA in halogenated solvents such as, although not limited to chlorobenzene, chloroform, and DCM at appropriate temperature to give the compound of formula (J11).

The compound of formula (J11) undergoes a nucleophilic substitution reaction with different chiral benzylic amines of compound of formula (A5) leading to the compound of formula (II-I) using organic basic reagents such as but not limited to DIPEA and TEA in a polar aprotic solvents like dioxane and THF at appropriate temperature.

Scheme-K Illustrates Formation of Compound of Formula (II-J) Starting from Compound of Formula (M1) (Reference: CN105884699)

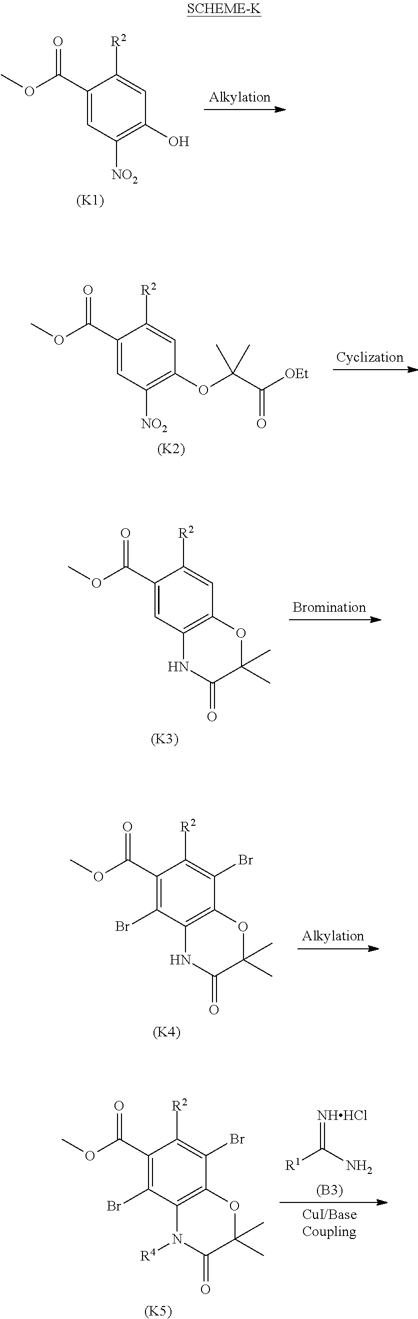

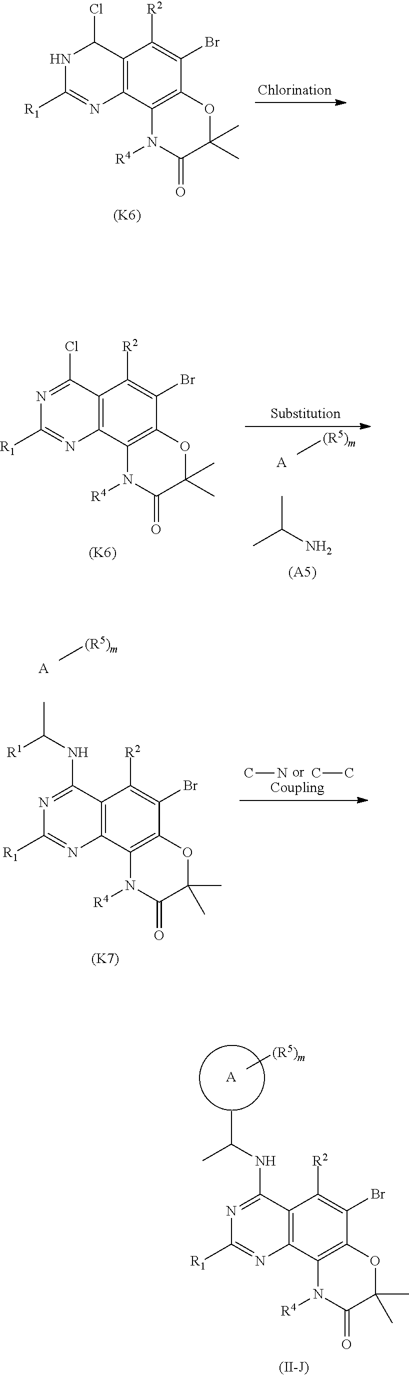

The compound of formula (K1) upon alkylation using ethyl 2-bromo-2-methylpropanoate afford the compound of formula (K2). Such reaction could be carried out by using inorganic bases such as, although not limited to K₂CO₃, Cs₃CO₃, and Na₂CO₃ and organic bases such as, although not limited to DIPEA, TEA, diisopropyl amine, and the like, and the polar aprotic solvents such as, although not limited to acetone, acetonitrile, and DMF, or mixture(s) thereof.

The compound of formula (K2) further converted to corresponding cyclized derivatives of compound of formula (K3) through selective reduction of nitro group by using reducing agents, although not limited to, such reducing agents include hydrogenation with palladium on carbon, metal reductions like iron, tin or tin chloride, and the like. Such reduction reaction can be carried out in one or more solvents, e.g. ethers such as THF, 1,4-dioxane, and the like; alcohol such as methanol, ethanol, and the like; under acidic conditions involving ammonium chloride, acetic acid, hydrochloric acid, and the like, or mixture(s) thereof.

The compound of formula (K3) undergoes halogenation using N-halosuccinimide reagent such as, but not limited to NBS, NIS, and NCS gives corresponding dihalo compound of formula (K4), which on alkylation using alkyl halides afford compound of formula (K5). Such reaction could be carried out by using inorganic bases such as, although not limited to K₂CO₃, Cs₂CO₃, and Na₂CO₃, and the polar aprotic solvents such as, although not limited to acetone, acetonitrile, and DMF, or mixture(s) thereof.

The compound of formula (K5), which on coupling with different amidines of compound of formula (B3) gives compound of formula (K6) (where R¹=alkyl) which could be halogenated by using reagents such as, although not limited to POCl₃ and POBr₃ in combination with organic bases such as, although not limited to DIPEA and TEA in halogenated solvents such as, although not limited to chlorobenzene, chloroform, and DCM at appropriate temperature to give compound of formula (K7).

The compound of formula (K7) undergoes a nucleophilic substitution reaction with different chiral benzylic amines (A5) leading to the compound of formula (K8) using organic basic reagents such as but not limited to DIPEA and TEA in a polar aprotic solvents like dioxane and THF at appropriate temperature.

The compound of formula (K8) could be further functionalized e.g. transition metal catalyzed C—C or C—N coupling reactions like Suzuki or Buchwald reaction utilizing corresponding counterpart, i.e. substituted amine or substituted boronate to gives the compound of formula (II-J).

All intermediates used for the preparation of the compounds of the present invention, were prepared by approaches reported in the literature or by methods known to people skilled in the art of organic synthesis. Detailed experimental procedures for the synthesis of intermediates are given below.

The intermediates and the compounds of the present invention can be obtained in a pure form by any suitable method, for example, by distilling off the solvent in vacuum and/or re-crystallizing the residue obtained from a suitable solvent, such as pentane, diethyl ether, isopropyl ether, chloroform, dichloromethane, ethyl acetate, acetone or their combinations or subjecting it to one of the purification methods, such as column chromatography (e.g., flash chromatography) on a suitable support material such as alumina or silica gel using an eluent such as dichloromethane, ethyl acetate, hexane, methanol, acetone and/or their combinations. Preparative LC-MS method can also be used for the purification of the molecules described herein.

Unless otherwise stated, work-up includes distribution of the reaction mixture between the organic and aqueous phase indicated within parentheses, separation of the layers and drying of the organic layer over sodium sulphate, filtration, and evaporation of the solvent. Purification, unless otherwise mentioned, includes purification by silica gel chromatographic techniques, generally by using a mobile phase with suitable polarity, and purification using selective crystallization.

Salts of SOS1 inhibitor of formula (I) and SOS1 inhibitor of formula (II) can be obtained by dissolving the compound in a suitable solvent, for example in a chlorinated hydrocarbon, such as methyl chloride or chloroform or a low molecular weight aliphatic alcohol, for example, ethanol or isopropanol, which is then treated with the desired acid or base as described in Berge S. M. et al., “Pharmaceutical Salts, a review article in Journal of Pharmaceutical sciences volume 66, page 1-19 (1977)” and in “Handbook of Pharmaceutical Salts—Properties, Selection, and Use,” by P. Heinrich Stahland Camille G. Wermuth, Wiley-VCH (2002). Lists of suitable salts can also be found in Remington's Pharmaceutical Sciences, 18th ed., Mack Publishing Company, Easton, P A, 1990, p. 1445, and Journal of Pharmaceutical Science, 66, 2-19 (1977). For example, the salt can be of an alkali metal (e.g., sodium or potassium), alkaline earth metal (e.g., calcium), or ammonium.

The stereoisomers of the SOS1 inhibitor of formula I and II can be prepared by stereospecific synthesis or resolution of racemic compound mixture by using an optically active amine, acid or complex forming agent, and separating the diastereomeric salt/complex by fractional crystallization or by column chromatography.

The SOS1 inhibitor of formula I and H can exist in tautomeric forms, such as keto-enol tautomer. Such tautomeric forms are contemplated as an aspect of the present invention and such tautomer's may be in equilibrium or predominant in one of the forms.

The present invention also embraces isotopically-labelled compounds of the present invention which are identical to those recited herein, but for the fact that one or more atoms are replaced by an atom having an atomic mass or mass number different from the atomic mass or mass number usually found in abundance in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²³I respectively.

In some embodiments, a method of treating and/or preventing cancer, wherein the method comprising administering to the subject in need with pharmaceutical combination of SOS1 inhibitor of formula (I) or formula (II) and an additional active ingredient selected from a KRAS inhibitor such as a KRAS G12C inhibitor and a KRASG12D inhibitor, KRAS G13C inhibitor, and panKRAS inhibitor; an EGFR inhibitor; an ERK1/2 inhibitor; a BRAF inhibitor; a pan-RAF inhibitor; a MEK inhibitor; a AKT inhibitor; a SHP2 inhibitor; protein arginine methyltransferases (PRMTs) inhibitor such as a PRMT5 inhibitor and Type 1 PRMT inhibitor; a PI3K inhibitor; a cyclin-dependent kinase (CDK) inhibitor such as CDK4/6 inhibitor; a FGFR inhibitor; a c-Met inhibitor; a RTK inhibitor; a non-receptor tyrosine kinase inhibitor; a histone methyltransferases (HMTs) inhibitor; a DNA methyltransferases (DNMTs) inhibitor; a Focal Adhesion Kinase (FAK) inhibitor; a Bcr-Abl tyrosine kinase inhibitor; a mTOR inhibitor; a PD1 inhibitor; a PD-L1 inhibitor; CTLA4 inhibitor; and chemotherapeutic agents such as gemcitabine, doxorubicin, cisplatin, carboplatin, paclitaxel, docetaxel, topotecan, irinotecan and temozolomide.

In some embodiments, this disclosure includes a pharmaceutical combination comprising SOS1 inhibitor of formula (I) or formula (II) and additional active ingredient can be used to treat and/or prevent various cancers which include or exclude: glioblastoma multiforme, prostate cancer, pancreatic cancer, mantle cell lymphoma, non-Hodgkin's lymphomas and diffuse large B-cell lymphoma, acute myeloid leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, head and neck cancer and cervical cancer.

The pharmaceutical compositions can be administered parenterally, e.g., intravenously, intraarterially, subcutaneously, intradermally, intrathecally, or intramuscularly. Thus, the invention provides compositions for parenteral administration that comprise a solution of the compound of the invention dissolved or suspended in an acceptable carrier suitable for parenteral administration, including aqueous and non-aqueous, isotonic sterile injection solutions.

Suitable doses and dosage regimens can be determined by conventional range-finding techniques known to those of ordinary skill in the art. Generally, treatment is initiated with smaller dosages that are less than the optimum dose of the compound of the present invention. Thereafter, the dosage is increased by small increments until the optimum effect under the circumstances is reached. The present method can involve the administration of about 0.1 μg to about 50 mg of at least one compound of the invention per kg body weight of the individual. For a 70 kg patient, dosages of from about 10 μg to about 200 mg of the compound of the invention would be more commonly used, depending on a patient's physiological response.

By way of example and not intending to limit the invention, the dose of the pharmaceutically active agent(s) described herein for methods of treating a disease or condition as described above can be about 0.001 to about 1 mg/kg body weight of the subject per day, for example, about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight per day. The dose of the pharmaceutically active agent(s) described herein for the described methods can be about 1 to about 1000 mg/kg body weight of the subject being treated per day, for example, about 1 mg, 2 mg, 5 mg, 10 mg, 15 mg, 0.020 mg, 25 mg, 50 mg, 75 mg, 100 mg, 150 mg, 200 mg, 250 mg, 500 mg, 750 mg, or 1000 mg/kg body weight per day.

In another embodiment the present invention also provides a method of treatment of cancer with aberrant activation of RTK, RAS RAF, and PI3K using a pharmaceutical combination described herein.

In yet another embodiment the present invention provides method of treatment using the combination as described herein by administering the active ingredients using a single unit dosage form or multiple dosage forms, and in case of multiple dosage forms they all can be administered simultaneously or subsequently.

The terms “treat,” “ameliorate,” and “inhibit,” as well as words stemming therefrom, as used herein, do not necessarily imply 100% or complete treatment, amelioration, or inhibition. Rather, there are varying degrees of treatment, amelioration, and inhibition of which one of ordinary skill in the art recognizes as having a potential benefit or therapeutic effect. In this respect, the disclosed methods can provide any amount of any level of treatment, amelioration, or inhibition of the disorder in a mammal. For example, a disorder, including symptoms or conditions thereof, may be reduced by, for example, 100%, 90%, 80%, 70%, 60%, 50%, 40%, 30%, 20%, or 10%. Furthermore, the treatment, amelioration, or inhibition provided by the inventive method can include treatment, amelioration, or inhibition of one or more conditions or symptoms of the disorder, e.g., cancer. Also, for purposes herein, “treatment,” “amelioration,” or “inhibition” can encompass delaying the onset of the disorder, or a symptom or condition thereof.

The notation “or1” and “or2” in structural formulae denote that chiral center is ascertained to be either R or S, herein absolute configuration is not determined.

According to a feature of the present invention, the compounds disclosed herein can be prepared by methods illustrated in the schemes and examples provided herein below.

### EXAMPLES

### Intermediate 1: Preparation of (R)-3-(1-aminoethyl)-5-(trifluoromethyl)aniline

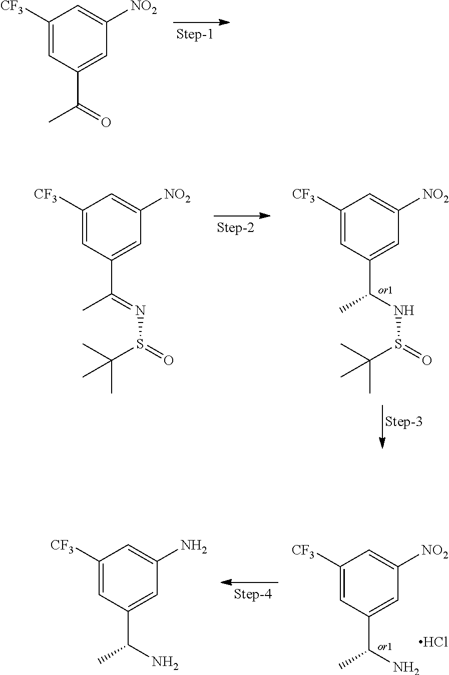 [SMILES: CC(=O)c1cc([N+](=O)[O-])cc(C(F)(F)F)c1 ; C/C(=N\[S@](=O)C(C)(C)C)c1cc([N+](=O)[O-])cc(C(F)(F)F)c1 ; C[C@@H](N[S@](=O)C(C)(C)C)c1cc([N+](=O)[O-])cc(C(F)(F)F)c1 ; C[C@@H](N)c1cc(N)cc(C(F)(F)F)c1 ; C[C@@H](N)c1cc([N+](=O)[O-])cc(C(F)(F)F)c1 ; Cl]

### Step 1: (R)-2-methyl-N-(1-(3-nitro-5-(trifluoromethyl)phenyl)ethylidene)propane-2-sulfinamide

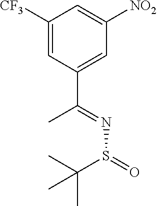 [SMILES: C/C(=N\[S@](=O)C(C)(C)C)c1cc([N+](=O)[O-])cc(C(F)(F)F)c1]

To a stirred solution of 1-(3-nitro-5-(trifluoromethyl)phenyl)ethan-1-one (60 g, 257 mmol) in THF (600 mL), (R)-2-methylpropane-2-sulfinamide (46.8 g, 386 mmol) and tetraethoxytitanium (135 mL, 643 mmol) were added at room temperature and the resulting reaction mixture was heated to 80° C. for 5 h. The reaction mixture was cooled to room temperature, quenched with cold water (100 mL) and diluted with ethyl acetate (600 mL). Resulting mixture was passed through celite bed and layers were separated. Organic layer was washed with brine (200 mL), dried over anhydrous Na₂SO₄ and evaporated. The crude product was purified by flash chromatography to provide the titled compound (61 g, 70.5% yield).

MS(ES+) m/z=337.2 (M+1).

### Step 2: (R)-2-methyl-N—((R/S)-1-(3-nitro-5-(trifluoromethyl)phenyl)ethyl)propane-2-sulfinamide

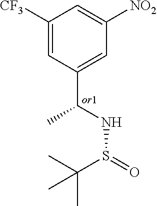 [SMILES: C[C@@H](N[S@](=O)C(C)(C)C)c1cc([N+](=O)[O-])cc(C(F)(F)F)c1]

To a stirred solution of (R, E)-2-methyl-N-(1-(3-nitro-5-(trifluoromethyl)phenyl)ethylidene) propane-2-sulfinamide (60 g, 178 mmol) in THF (300 mL) and water (6 mL), NaBH₄ (13.50 g, 357 mmol) was added at −78° C. The reaction was stirred at same temperature for 25 min, quenched with cold water and extracted with ethyl acetate (3×200 mL). Combined organic layer was washed with brine (100 mL), dried over anhydrous Na₂SO₄ and concentrated. The crude material (diastereomeric mixture) was purified using flash chromatography to yield titled compound as major product (40 g, 66.3% yield).

MS(ES+) m/z=339.1 (M+1).

### Step 3: (R/S)-1-(3-nitro-5-(trifluoromethyl)phenyl)ethan-1-amine hydrochloride

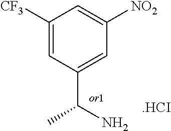

To a stirred solution of (R/S)-2-methyl-N—((R)-1-(3-nitro-5-(trifluoromethyl)phenyl)ethyl)propane-2-sulfinamide (30 g, 89 mmol) in DCM (100 mL) was added 4M HCl in dioxane (222 mL, 887 mmol) and stirred at room temperature for 30 min. Solvent was removed under reduced pressure to get solid compound. Diethyl ether (200 mL) was added and stirred for 15 min, precipitated solid was filtered, dried under vacuum to afford titled compound (21.2 g, 88% yield).

¹H NMR (400 MHz, DMSO-d₆) δ 8.92 (s, 2H), 8.80 (t, J=1.9 Hz, 1H), 8.53-8.47 (m, 2H), 4.83-4.69 (m, 1H), 1.60 (d, J=6.7 Hz, 3H).

### Step 4: (R)-3-(1-aminoethyl)-5-(trifluoromethyl)aniline

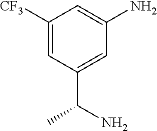

The (R/S)-1-(3-nitro-5-(trifluoromethyl)phenyl)ethan-1-amine hydrochloride (12 g, 44.3 mmol) was charged to Parr shaker containing MeOH (300 mL) and Pd—C(0.944 g, 8.87 mmol) was added carefully. The reaction was stirred for 3h under hydrogen pressure (40 psi). Reaction mixture was filtered through a celite bed. Filtrate was concentrated under vacuum and residue was basified with a sat. sodium bicarbonate solution. The bicarbonate layer was extracted with DCM (150 mL×3). The organic layer was dried over anhydrous Na₂SO₄ and concentrated under reduced pressure to afford titled compound (8.5 g, 95% yield) Chirality of the compound was confirmed as ‘R’ by VCD experiment.

¹H NMR (400 MHz, DMSO-d₆) δ 6.85-6.77 (m, 2H), 6.70-6.65 (m, 1H), 5.46 (s, 2H), 3.92-3.83 (m, 1H), 1.20 (d, J=6.6 Hz, 3H).

### Intermediate 2: Preparation of (R)-1-(3-(1-aminoethyl)-2-fluorophenyl)-1,1-difluoro-2-methylpropan-2-ol hydrochloride & (S)-1-(3-(1-aminoethyl)-2-fluorophenyl)-1,1-difluoro-2-methylpropan-2-ol hydrochloride

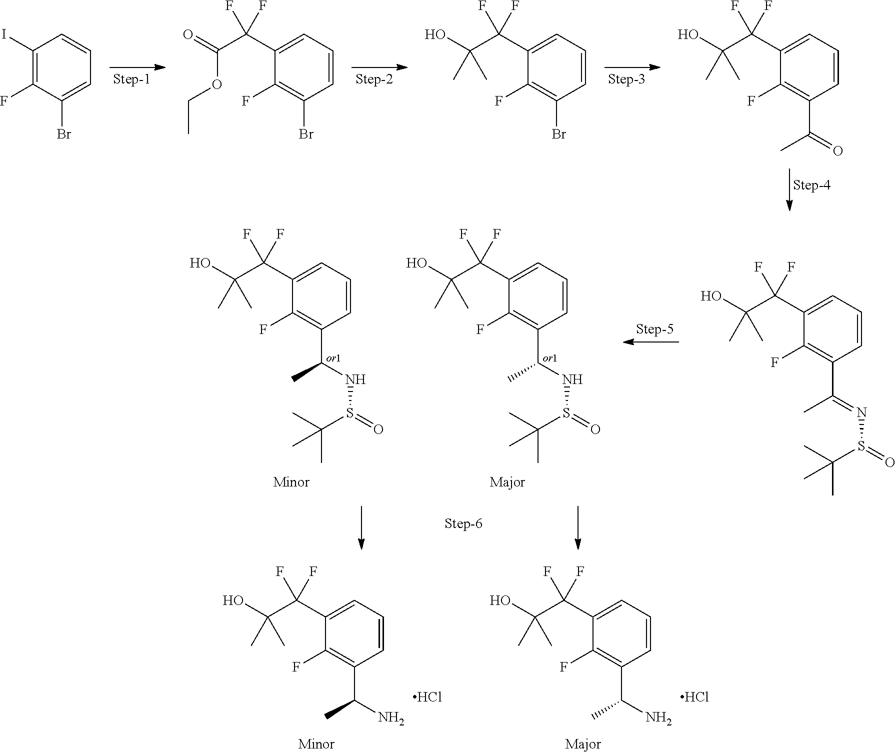

### Step 1: Ethyl 2-(3-bromo-2-fluorophenyl)-2,2-difluoroacetate

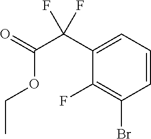 [SMILES: CCOC(=O)C(F)(F)c1cccc(Br)c1F]

To a stirred solution of ethyl 2-bromo-2,2-difluoroacetate (69.1 g, 341 mmol) in DMSO (200 mL) was added copper powder (21.65 g, 341 mmol) and reaction was stirred for 30 min followed by addition of 1-bromo-2-fluoro-3-iodobenzene (41 g, 136 mmol). The reaction was stirred at 70° C. for 2 h. The reaction was cooled to room temperature, quenched with water (400 mL) and filtered through a celite bed. Celite bed was washed with diethyl ether (400 mL). The Organic layer was separated, dried over anhydrous Na₂SO₄ and concentrated under reduced pressure. The crude residue was purified by flash chromatography hexane-Ethyl acetate gradient to afford the titled compound (24.1 g, 59.5% yield) as a colorless liquid.

MS(ES+) m/z=297.90 (M+1).

¹H NMR (400 MHz, Chloroform-d) δ 7.77-7.70 (m, 1H), 7.65-7.59 (m, 1H), 7.21-7.15 (m, 1H), 4.39 (q, J=7.1 Hz, 2H), 1.36 (t, J=7.1 Hz, 3H).

### Step 2: 1-(3-bromo-2-fluorophenyl)-1,1-difluoro-2-methylpropan-2-ol

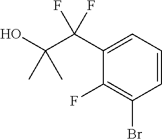

To a stirred solution of ethyl 2-(3-bromo-2-fluorophenyl)-2,2-difluoroacetate (10 g, 33.7 mmol) in THE (100 mL) was added methyl magnesium bromide in diethyl ether (3M, 33.7 mL, 101 mmol) in dropwise at 0° C. and the reaction was stirred at same temperature for 30 min. The reaction was quenched with saturated aqueous NH₄Cl solution and extracted with diethyl ether (100 mL). The organic layer was separated, dried over anhydrous Na₂SO₄ and concentrated under reduced pressure. The crude product was purified by flash chromatography in hexane-Ethyl acetate gradient to afford the titled compound (9.2 g, 97% yield) as a colorless liquid.

¹H NMR (400 MHz, DMSO-d₆) δ 7.89-7.84 (m, 1H), 7.50-7.44 (m, 1H), 7.30-7.23 (m, 1H), 5.43 (s, 1H), 1.21 (s, 3H), 1.20 (s, 3H).

### Step 3: 1-(3-(1,1-difluoro-2-hydroxy-2-methylpropyl)-2-fluorophenyl)ethan-1-one

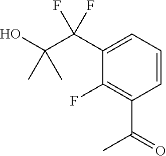 [SMILES: CC(=O)c1cccc(C(F)(F)C(C)(C)O)c1F]

To a stirred solution of 1-(3-bromo-2-fluorophenyl)-1,1-difluoro-2-methylpropan-2-ol (12.5 g, 44.2 mmol) in toluene (150 mL), tributyl(I-ethoxyvinyl)stannane (19.14 g, 53 mmol), TEA (15.39 mL, 110 mmol) was added and reaction was purged with N2 for 10 min. PdCl₂(PPh₃)₂ (1.24 g, 1.766 mmol) was added and reaction was stirred at 100° C. for 16 h. The reaction was cooled to room temperature and filtered through celite bed. The filtrate was evaporated under reduced pressure to afford 11.5 g crude product. The crude product as such was dissolved in THF (50 mL) and HCl:water (1:1) (3 mL) was added to it at 0° C. The reaction mixture was warmed to room temperature and stirred for 15 min. The reaction mixture was neutralized with saturated NaHCO₃(5 mL) and extracted with Ethyl acetate (100 mL×3). The organic layer was separated, dried over anhydrous Na₂SO₄, and concentrated under reduced pressure. The crude product obtained was purified by flash chromatography in hexane-Ethyl acetate gradient to afford the titled compound (8.8 g, 81% yield) as an oily compound.

¹H NMR (400 MHz, CDCl₃) δ 7.99-7.94 (m, 1H), 7.69-7.63 (m, 1H), 7.34-7.29 (m, 1H), 2.68 (d, J=5.3 Hz, 3H), 1.39 (s, 3H), 1.38 (s, 3H).

### Step 4: (R)—N-(1-(3-(1,1-difluoro-2-hydroxy-2-methylpropyl)-2-fluorophenyl)ethylidene)-2-methylpropane-2-sulfinamide

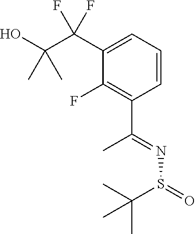 [SMILES: C/C(=N\[S@](=O)C(C)(C)C)c1cccc(C(F)(F)C(C)(C)O)c1F]

To a stirred solution of I-(3-(1,1-difluoro-2-hydroxy-2-methylpropyl)-2-fluorophenyl)ethan-1-one (8.7 g, 35.3 mmol) in THF (100 mL), (R)-2-methylpropane-2-sulfinamide (6.42 g, 53 mmol) and Titanium (IV)isopropoxide (25.9 mL, 88 mmol) were added at room temperature. The resulting reaction mixture was heated at 100° C. for 16 h. Reaction was quenched with ice-cold water (100 mL) and diluted with Ethyl acetate (100 mL). The mixture was filtered through celite bed. Organic layer of the filtrate was separated, dried over anhydrous Na₂SO₄, and concentrated under reduced pressure. The crude product was purified by flash chromatography using hexane-Ethyl acetate gradient to afford the titled compound (8.9 g, 72.1% yield).

MS(ES+) m/z=350.28 (M+1).

¹H NMR (400 MHz, DMSO-d₆) δ 7.78-7.70 (m, 1H), 7.62-7.54 (m, 1H), 7.41-7.34 (m, 1H), 5.40 (s, 1H), 2.82-2.75 (m, 3H), 1.22 (s, 15H).

### Step 5: (R&S)—(R)—N-(1-(3-(1,1-difluoro-2-hydroxy-2-methylpropyl)-2-fluorophenyl)ethyl)-2-methylpropane-2-sulfinamide

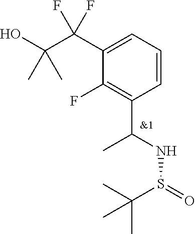 [SMILES: CC(N[S@](=O)C(C)(C)C)c1cccc(C(F)(F)C(C)(C)O)c1F]

To a stirred solution of (R)—N-(1-(3-(1,1-difluoro-2-hydroxy-2-methylpropyl)-2-fluorophenyl)ethylidene)-2-methylpropane-2-sulfinamide (8.7 g, 24.90 mmol) in THF (90 mL) was added NaBH₄ (1.13 g, 29.9 mmol) at 0° C. The reaction mixture was stirred at room temperature for 1 h. Reaction was diluted with water (100 mL) and extracted with Ethyl acetate (100 mL×3). The organic layer was separated, dried over anhydrous Na₂SO₄ and concentrated under reduced pressure. The crude product was purified by flash chromatography in hexane-Ethyl acetate gradient to afford titled compound as mixture of diastereomers. The two diastereomers were separated by preparative HPLC.

### (a) (R)—N—((R/S)-1-(3-(1,1-difluoro-2-hydroxy-2-methylpropyl)-2-fluorophenyl)ethyl)-2-methylpropane-2-sulfinamide (42.3% yield, Major isomer)

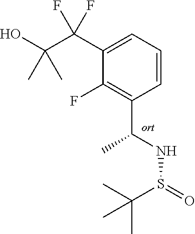 [SMILES: C[C@@H](N[S@](=O)C(C)(C)C)c1cccc(C(F)(F)C(C)(C)O)c1F]

¹H NMR (400 MHz, DMSO-d₆) δ 7.70-7.64 (m, 1H), 7.37-7.31 (m, 1H), 7.30-7.24 (m, 1H), 5.84 (d, J=7.7 Hz, 1H), 5.33 (s, 1H), 4.74-4.62 (m, 1H), 1.40 (d, J=6.8 Hz, 3H), 1.20 (bs, 6H), 1.10 (s, 9H).

### (b) (R)—N—((S/R)-1-(3-(1,1-difluoro-2-hydroxy-2-methylpropyl)-2-fluorophenyl)ethyl)-2-methylpropane-2-sulfinamide (15% yield, Minor isomer)

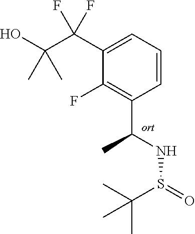 [SMILES: C[C@H](N[S@](=O)C(C)(C)C)c1cccc(C(F)(F)C(C)(C)O)c1F]

¹H NMR (400 MHz, DMSO-d₆) δ 7.63-7.57 (m, 1H), 7.38-7.31 (m, 1H), 7.30-7.23 (m, 1H), 5.50 (d, J=6.0 Hz, 1H), 5.34 (s, 1H), 4.78-4.64 (m, 1H), 1.49 (d, J=6.8 Hz, 3H), 1.20 (bs, 6H), 1.10 (s, 9H).

### Step 6a: (R)-1-(3-(1-Aminoethyl)-2-fluorophenyl)-1,1-difluoro-2-methylpropan-2-ol hydrochloride

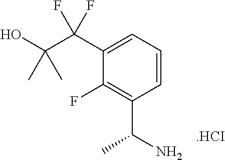

To a stirred solution of (R)—N—((R)-1-(3-(1,1-difluoro-2-hydroxy-2-methylpropyl)-2-fluorophenyl)ethyl)-2-methylpropane-2-sulfinamide (Step-5a, 3.65 g, 10.39 mmol) in DCM (30 mL) was added 4M HCl in dioxane (12.98 mL, 51.9 mmol) at 0° C. The reaction mixture was stirred at room temperature for 30 min. The solvent was evaporated, and the residue was crystallized from diethyl ether to give titled compound. (2.7 g, 92.0% yield) as a white solid. The chirality of the compound was confirmed as ‘R’ by X-ray crystallography.

¹H NMR (400 MHz, DMSO-d₆) δ 8.73-8.67 (m, 2H), 7.87-7.80 (m, 1H), 7.51-7.44 (m, 1H), 7.42-7.35 (m, 1H), 5.48-5.36 (m, 1H), 4.70-4.58 (m, 1H), 1.54 (d, J=6.8 Hz, 3H), 1.22 (bs, 6H).

### Step 6b: (S)-1-(3-(1-aminoethyl)-2-fluorophenyl)-1,1-difluoro-2-methylpropan-2-ol hydrochloride

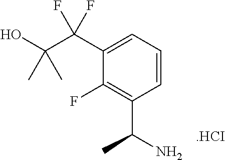

Titled compound was prepared using analogous protocol mentioned in Step-6a (90% yield). The chirality of the compound was confirmed as ‘S’ by VCD experiment.

¹H NMR (400 MHz, DMSO-d₆) δ 8.78-8.71 (m, 2H), 7.88-7.81 (m, 1H), 7.51-7.44 (m, 1H), 7.41-7.35 (m, 1H), 4.72-4.57 (m, 1H), 1.54 (d, J=6.8 Hz, 3H), 1.22 (bs, 6H).

### Intermediate 3: (R/S)-1-(3-(1-aminoethyl)phenyl)-1,1-difluoro-2-methylpropan-2-ol hydrochloride

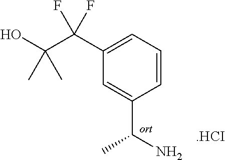

Intermediate 3 was prepared by using procedure described for intermediate 2 using corresponding raw materials.

### Example 1: Preparation of (R)-4-((1-(3-(1,1-Difluoro-2-hydroxy-2-methylpropyl)-2-fluorophenyl)ethyl)amino)-2,6,8,8-tetramethyl-6,8-dihydro-7H-pyrrolo[2,3-g]quinazolin-7-one (Compound 1)

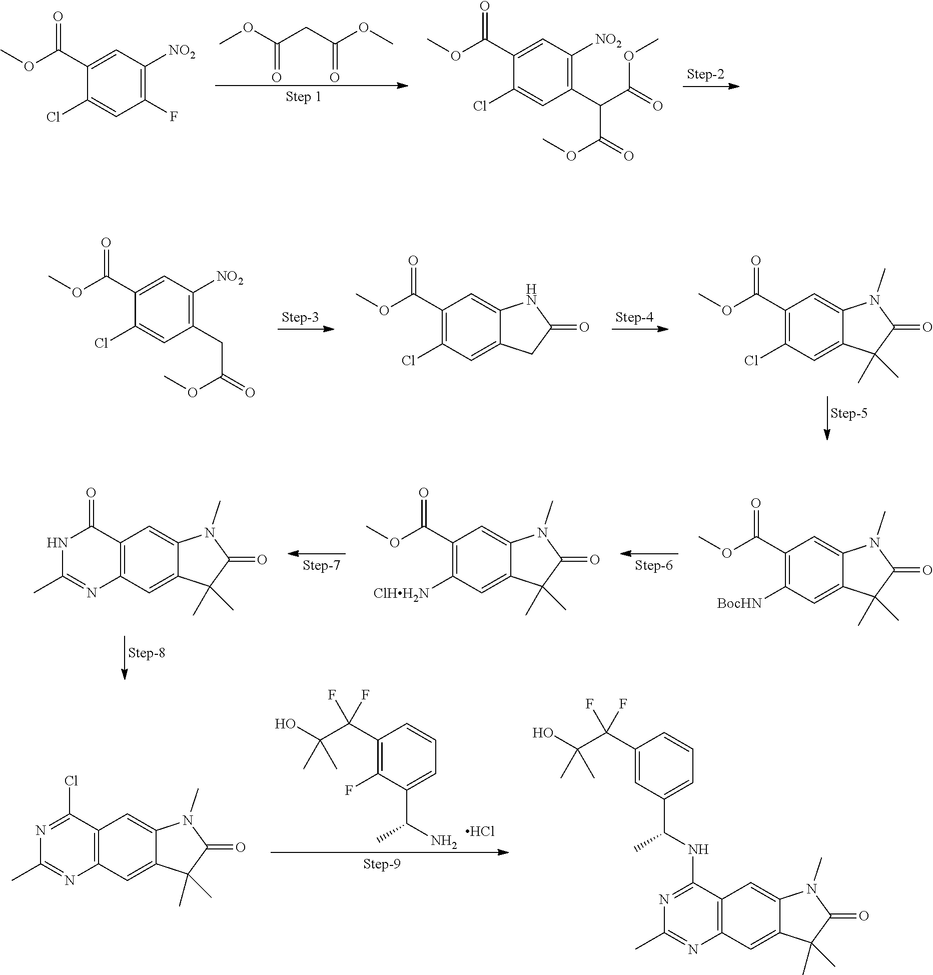

### Step 1: Dimethyl 2-(5-chloro-4-(methoxycarbonyl)-2-nitrophenyl)malonate

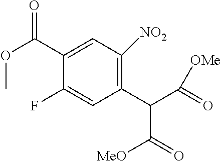 [SMILES: COC(=O)c1cc([N+](=O)[O-])c(C(C(=O)OC)C(=O)OC)cc1F]

To a cooled (0° C.) solution of dimethyl malonate (12.17 mL, 106.0 mmol) in DMF (165 mL) were added methyl 2-chloro-4-fluoro-5-nitrobenzoate (16.5 g, 70.6 mmol) and K₂CO₃ (29.3 g, 212 mmol). The reaction mixture was stirred overnight at room temperature. The reaction was poured in ice cold 2 M aqueous HCl and extracted with ethyl acetate (2×200 mL), combined organic layer was washed with water (200 mL), brine (150 mL), and dried over anhydrous Na₂SO₄. Removal of solvent under reduced pressure and the crude material obtained was purified by flash chromatography in 10% ethyl acetate-n-hexane to afford titled compound (18.2 g, 74.5% yield).

MS(ES+) m/z=346.14 (M+1).

### Step 2: Methyl 2-chloro-4-(2-methoxy-2-oxoethyl)-5-nitrobenzoate

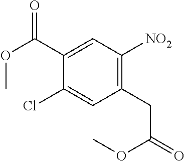 [SMILES: COC(=O)Cc1cc(Cl)c(C(=O)OC)cc1[N+](=O)[O-]]

A solution of dimethyl 2-(5-chloro-4-(methoxycarbonyl)-2-nitrophenyl)malonate (10.0 g, 28.9 mmol), LiCl (2.453 g, 57.9 mmol) in DMSO (100 mL) and water (1.042 mL, 57.9 mmol) was heated at 90′C for 5 h. The reaction mixture was cooled to room temperature and poured on ice water (200 mL). The aqueous layer was extracted with ethyl acetate (3×100 mL). The combined organic layers were washed with brine (100 mL), dried over anhydrous Na₂SO₄, and concentrated in vacuum. The crude product was purified by flash chromatography in ethyl acetate-n-hexane gradient to afford the titled compound (5.6 g, 67.3%).

¹H NMR (400 MHz, CDCl₃) δ 8.68 (s, 1H), 7.51 (s, 1H), 4.07 (s, 2H), 4.01 (s, 3H), 3.75 (s, 3H).

### Step 3: Methyl 5-chloro-2-oxoindoline-6-carboxylate

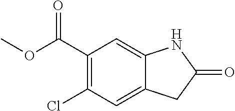

To a stirred solution of methyl 2-chloro-4-(2-methoxy-2-oxoethyl)-5-nitrobenzoate (5.6 g, 19.47 mmol) in ethanol-acetic acid (60 mL, ratio 1:1), iron (2.19 g, 38.9 mmol) was added at 25° C. and the reaction was stirred at 100° C. for 2 h. The reaction was cooled to room temperature, solvent was removed under vacuum and the residue was neutralized with aq. NaHCO₃(30 mL). Ethyl acetate (60 mL) was added and resulting mixture was filtered through celite bed. Separated aqueous layer from filtrate was extracted with ethyl acetate (50 mL). The combined organic layers were washed with brine (100 mL), dried over anhydrous Na₂SO₄ and concentrated in vacuum to give crude product. The crude product was purified by flash chromatography using 0-50% ethyl acetate-n-hexane as eluent to yield the titled compound (1.2 g, 27.3% yield) as a white solid.

MS(ES+) m/z=225.19 (M+), 227.14(M+2).

### Step 4: Methyl 5-chloro-1,3,3-trimethyl-2-oxoindoline-6-carboxylate

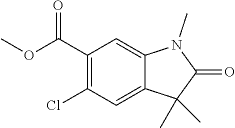 [SMILES: COC(=O)c1cc2c(cc1Cl)C(C)(C)C(=O)N2C]

To a stirred solution of methyl 5-chloro-2-oxoindoline-6-carboxylate (1.2 g, 5.32 mmol) in DMF (20 mL), methyl iodide (0.998 mL, 15.96 mmol) was added. The reaction was cooled to −10° C. and portion wise NaH (0.64 g, 15.96 mmol) was added. The reaction was stirred at −10° C. for 1 h. The reaction was quenched with aq. ammonium chloride solution (20 mL) extracted with ethyl acetate (3×30 mL). The combined organic layers were washed with water (30 mL), brine (30 mL), dried over anhydrous Na₂SO₄, and concentrated in vacuum to give crude product. The crude product was purified by flash chromatography using 0-20% ethyl acetate-n-hexane as eluent to yield titled compound (1.2 g, 84% yield).

MS(ES+) m/z=268.40 (M+1).

### Step 5: Methyl 5-((tert-butoxycarbonyl)amino)-1,3,3-trimethyl-2-oxoindoline-6-carboxylate

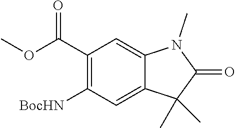 [SMILES: COC(=O)c1cc2c(cc1NC(=O)OC(C)(C)C)C(C)(C)C(=O)N2C]

To a solution of methyl 5-chloro-1,3,3-trimethyl-2-oxoindoline-6-carboxylate (1.2 g, 4.48 mmol) in dry 1,4-dioxane (15 mL), were added tert-butyl carbamate (0.683 g, 5.83 mmol) and Cs₂CO₃ (2.63 g, 8.07 mmol). The suspension was degassed with nitrogen for 10 min. Xantphos (0.311 g, 0.538 mmol) and Pd₂(dba)₃ (0.205 g, 0.224 mmol) were added and resulting reaction mixture was heated at 120° C. for 16 h. The reaction was cooled to room temperature and solvent was removed under reduced pressure. The crude product was purified by flash chromatography using ethyl acetate-hexane gradient to afford titled compound (1.1 g, 70.4% yield).

MS(ES+) m/z=349.2 (M+1).

### Step 6: Methyl 5-amino-1,3,3-trimethyl-2-oxoindoline-6-carboxylate hydrochloride

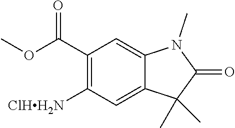

To a solution of methyl 5-((tert-butoxycarbonyl)amino)-1,3,3-trimethyl-2-oxoindoline-6-carboxylate (1.1 g, 3.16 mmol) in 1,4-dioxane (10.0 mL), was added HCl (4M in 1,4-dioxane, 8.0 mL) at 0° C. and the reaction was warmed to 70° C. for 2 h. The reaction mixture was concentrated in vacuum to get sticky residue. The residue was triturated with diethyl ether to afford the titled compound (0.85 g, 95% yield). The crude material was used as such for the next reaction.

MS(ES+) m/z=249.27 (free amine).

### Step 7: 2,6,8,8-Tetramethyl-6,8-dihydro-3H-pyrrolo[2,3-g]quinazoline-4,7-dione

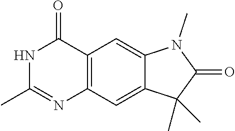 [SMILES: Cc1nc2cc3c(cc2c(=O)[nH]1)N(C)C(=O)C3(C)C]

To a solution of methyl 5-amino-1,3,3-trimethyl-2-oxoindoline-6-carboxylate hydrochloride (0.45 g, 1.580 mmol) in acetonitrile (10 mL), methanesulfonic acid (1.026 ml, 15.80 mmol) was added and the resulting reaction mixture was stirred at 100° C. for 16 h. Solvent was evaporated under vacuo and obtained residue was dissolved in ethyl acetate (25 mL). Organic layer was washed with aq. Sodium bicarbonate (2×10 mL) and water (10 mL). The separated organic layer was dried over anhydrous Na₂SO₄ and concentrated under reduced pressure to afford titled compound (0.4 g, 198% yield). Crude material was used as such for the next step.

MS(ES+) m/z=258.1 (M+1).

### Step 8: 4-chloro-2,6,8,8-tetramethyl-6,8-dihydro-7H-pyrrolo[2,3-g]quinazolin-7-one

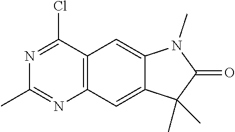 [SMILES: Cc1nc(Cl)c2cc3c(cc2n1)C(C)(C)C(=O)N3C]

To a suspension of 2,6,8,8-tetramethyl-6,8-dihydro-3H-pyrrolo[2,3-g]quinazoline-4,7-dione (0.380 g, 1.477 mmol) in chlorobenzene (6 mL) was added DIPEA (0.696 ml, 3.99 mmol) followed by addition of POCl₃ (0.344 ml, 3.69 mmol) drop wise at room temperature. Resulting reaction mixture was heated at 90° C. for the 2.5 h. Reaction mixture was poured in cold water and extracted with ethyl acetate (2×20 mL). Combined organic layer was washed with brine (25 mL), dried over anhydrous Na₂SO₄, concentrated under high vacuo to afford titled compound (0.4 g, 98% yield).

MS(ES+) m/z=276.2 (M+1).

### Step 9: (R)-4-((1-(3-(1,1-difluoro-2-hydroxy-2-methylpropyl)-2-fluorophenyl)ethyl)amino)-2,6,8,8-tetramethyl-6,8-dihydro-7H-pyrrolo[2,3-g]quinazolin-7-one (Compound 1)

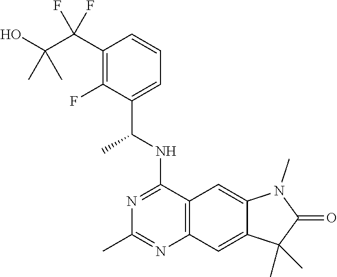 [SMILES: Cc1nc(N[C@H](C)c2cccc(C(F)(F)C(C)(C)O)c2F)c2cc3c(cc2n1)C(C)(C)C(=O)N3C]

To the stirred solution of 4-chloro-2,6,8,8-tetramethyl-6,8-dihydro-7H-pyrrolo[2,3-g]quinazolin-7-one (100 mg, 0.363 mmol) in 1,4-Dioxane (3 mL), (R)-1-(3-(1-aminoethyl)-2-fluorophenyl)-1,1-difluoro-2-methylpropan-2-ol hydrochloride (86 mg, 0.302 mmol) and DIPEA (0.264 ml, 1.511 mmol) were added at room temperature. The resulting mixture was stirred at 120° C. for 30 h. Reaction mixture was concentrated under vacuum to get crude product. The crude product was purified by RP HPLC to afford titled compound (25 mg, 17.00% yield).

MS(ES+) m/z=487.2 (M+1).

¹H NMR (400 MHz, DMSO-d₆) δ 8.21 (d, J=7.4 Hz, 1H), 7.89 (s, 1H), 7.62-7.58 (m, 2H), 7.34-7.28 (m, 1H), 7.25-7.17 (m, 1H), 5.85-5.79 (m, 1H), 5.34 (s, 1H), 3.28 (s, 3H), 2.32 (s, 3H), 1.60 (d, J=7.0 Hz, 3H), 1.35 (s, 3H), 1.34 (s, 3H), 1.24 (s, 3H), 1.22 (s, 3H).

Table 1: Compound-2 was synthesized by following the analogous procedure as described in Example 1 using corresponding intermediate and appropriate chiral amine.

TABLE 1

| Example | Chemical structure | LCMS and ¹H NMR data |
|---|---|---|
| 2 | 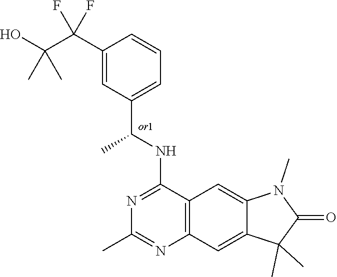 [SMILES: Cc1nc(N[C@H](C)c2cccc(C(F)(F)C(C)(C)O)c2)c2cc3c(cc2n1)C(C)(C)C(=O)N3C] (R/S)-4-((1-(3-(1,1-difluoro-2-hydroxy- 2-methylpropyl)phenyl)ethyl)amino)- 2,6,8,8-tetramethyl-6,8-dihydro-7H- pyrrolo[2,3-g]quinazolin-7-one (Compound 2) | MS(ES+) m/z = 469.42 (M + 1) ¹H NMR (400 MHZ, DMSO-d₆) δ 8.18 (d, J = 7.8 Hz, 1H), 7.85 (s, 1H), 7.61- 7.53 (m, 3H), 7.43-7.37 (m, 1H), 7.35- 7.31 (m, 1H), 5.68-5.59 (m, 1H), 5.25 (s, 1H), 3.26 (s, 3H), 2.36 (s, 3H), 1.63 (d, J = 7.0 Hz, 3H), 1.34 (s, 3H), 1.33 (s, 3H), 1.18-1.09 (m, 6H). |

### Example 3: 4-(((R)-1-(3-((R&S)-1,1-Difluoro-2,3-dihydroxy-2-methylpropyl)-2-fluorophenyl) ethyl)amino)-2,6,8,8-tetramethyl-6,8-dihydro-7H-pyrrolo[2,3-g]quinazolin-7-one (Compound 3)

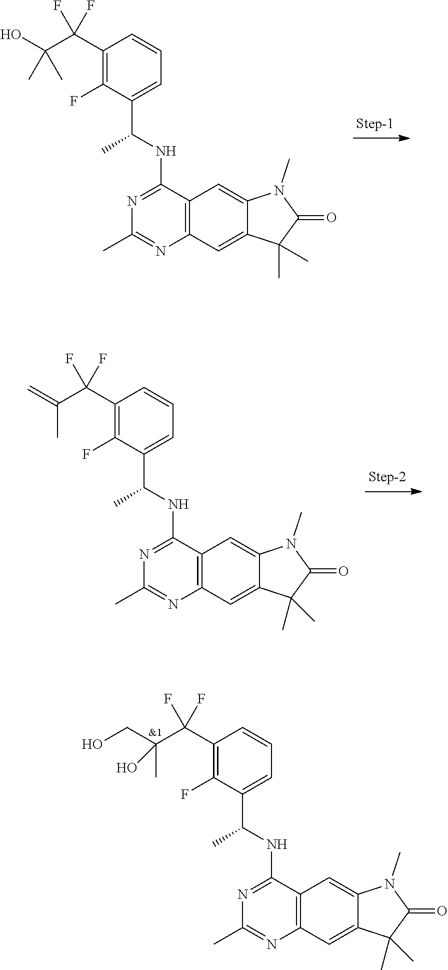 [SMILES: Cc1nc(N[C@H](C)c2cccc(C(F)(F)C(C)(C)O)c2F)c2cc3c(cc2n1)C(C)(C)C(=O)N3C ; C=C(C)C(F)(F)c1cccc([C@@H](C)Nc2nc(C)nc3cc4c(cc23)N(C)C(=O)C4(C)C)c1F ; Cc1nc(N[C@H](C)c2cccc(C(F)(F)C(C)(O)CO)c2F)c2cc3c(cc2n1)C(C)(C)C(=O)N3C]

### Step 1: (R)-4-((1-(3-(1,1-Difluoro-2-methylallyl)-2-fluorophenyl)ethyl)amino)-2,6,8,8-tetramethyl-6,8-dihydro-7H-pyrrolo[2,3-g]quinazolin-7-one

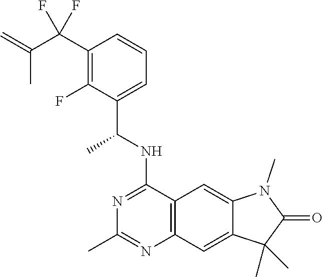 [SMILES: C=C(C)C(F)(F)c1cccc([C@@H](C)Nc2nc(C)nc3cc4c(cc23)N(C)C(=O)C4(C)C)c1F]

To a stirred solution of (R)-4-((1-(3-(1,1-difluoro-2-hydroxy-2-methylpropyl)-2-fluorophenyl)ethyl)amino)-2,6,8,8-tetramethyl-6,8-dihydro-7H-pyrrolo[2,3-g]quinazolin-7-one (Compound 1) (0.25 g, 0.514 mmol) in DCM (5 mL) was added DAST (1.82 g, 11.3 mmol, 1.49 mL) at −70° C. The reaction was stirred at −70° C. for 0.5 h, and gradually warmed to 0° C. for another 0.5 h under N2 atmosphere. The reaction mixture was quenched with saturated NaHCO₃(30 mL), extracted with DCM (50 mL×2). The organic phase was dried over anhydrous Na₂SO₄ and concentrated to give a residue. The residue was purified by flash chromatography in 5% MeOH in DCM to afford the titled compound (0.2 g, 83% yield).

MS(ES+) m/z=469.53 (M+1).

### Step 2: 4-(((R)-1-(3-((R&S)-1,1-Difluoro-2,3-dihydroxy-2-methylpropyl)-2-fluorophenyl) ethyl)amino)-2,6,8,8-tetramethyl-6,8-dihydro-7H-pyrrolo[2,3-g]quinazolin-7-one (Compound 3)

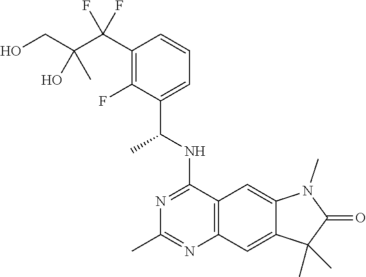 [SMILES: Cc1nc(N[C@H](C)c2cccc(C(F)(F)C(C)(O)CO)c2F)c2cc3c(cc2n1)C(C)(C)C(=O)N3C]

To a stirred solution of (R)-4-((1-(3-(1,1-difluoro-2-methylallyl)-2-fluorophenyl)ethyl)amino)-2,6,8,8-tetramethyl-6,8-dihydro-7H-pyrrolo[2,3-g]quinazolin-7-one (0.20 g, 0.427 mmol) in acetone (2 mL), tert-butanol (0.800 mL) and water (0.800 mL), NMO (0.125 g, 1.067 mmol) and osmium tetroxide (6.70 A1, 0.021 mmol) was added at 0° C. The reaction was stirred at room temperature for 18 h. The reaction was quenched with sodium thiosulfate solution extracted with DCM (2×25 mL) and concentrated under reduced pressure to get crude compound. Crude product was purified by flash chromatography to get titled compound (0.17 g). The diastereomers were separated by chiral chromatography.

Chiral separation method: CHIRALPAK IE CRL-005 HEX_IPA_50_50_A_B_1.0 ML_8MIN_241NM: 1.0 mL/min.

### Peak 1: 4-(((R)-1-(3-((R/S)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3a)

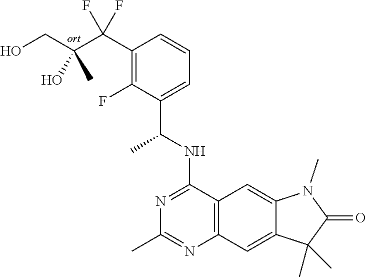 [SMILES: Cc1nc(N[C@H](C)c2cccc(C(F)(F)[C@](C)(O)CO)c2F)c2cc3c(cc2n1)C(C)(C)C(=O)N3C]

tₘ(min)=4.45

MS(ES+) m/z=503.42 (M+1).

¹H NMR (400 MHz, DMSO-d₆) δ 8.23 (d, J=7.4 Hz, 1H), 7.90 (s, 1H), 7.64-7.56 (m, 2H), 7.32 (m, 1H), 7.24-7.18 (m, 1H), 5.84-5.80 (m, 1H), 5.24 (s, 1H), 4.70 (t, J=6.1 Hz, 1H), 3.54-3.39 (m, 2H), 3.28 (s, 3H), 2.33 (s, 3H), 1.60 (d, J=7.0 Hz, 3H), 1.35 (s, 3H), 1.34 (s, 3H), 1.21 (s, 3H).

### Peak 2: 4-(((R)-1-(3-((S/R)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3b)

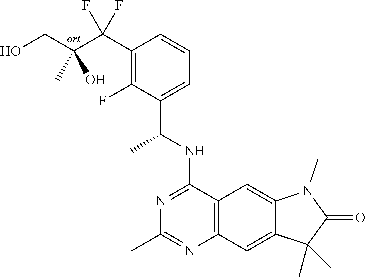 [SMILES: Cc1nc(N[C@H](C)c2cccc(C(F)(F)[C@@](C)(O)CO)c2F)c2cc3c(cc2n1)C(C)(C)C(=O)N3C]

tᵣₑₜ(min)=5.18

MS(ES+) m/z=503.42 (M+1).

¹H NMR (400 MHz, DMSO-d₆) δ 8.21 (d, J=7.5 Hz, 1H), 7.89 (s, 1H), 7.64-7.56 (m, 2H), 7.33-7.30 (m, 1H), 7.22-7.20 (m, 1H), 5.84-5.81 (m, 1H), 5.27 (s, 1H), 4.71-4.69 (m, 1H), 3.50-3.36 (m, 2H), 3.28 (s, 3H), 2.34 (s, 3H), 1.60 (d, J=7.0 Hz, 3H), 1.35 (s, 3H), 1.34 (s, 3H), 1.23 (s, 3H).

### Example 4: Preparation of (R&S)-4-(((R)-1-(3-(1,1-difluoro-2-hydroxy-2-methylpropyl)-2-fluorophenyl) ethyl) amino)-8-methoxy-2,6,8-trimethyl-6,8-dihydro-7H-pyrrolo[2,3-g]quinazolin-7-one (Compound 4)

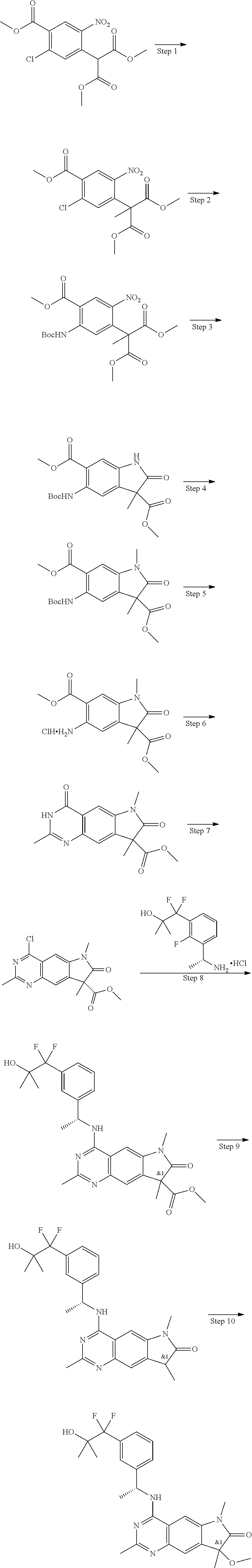

### Step 1: Dimethyl 2-(5-chloro-4-(methoxycarbonyl)-2-nitrophenyl)-2-methylmalonate

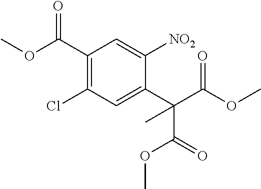

To a solution of dimethyl 2-(5-chloro-4-(methoxycarbonyl)-2-nitrophenyl)malonate (65 g, 188 mmol) in DMF (250 mL), K₂CO₃ (36.4 g, 263 mmol) and methyl iodide (16.46 mL, 263 mmol) were added at 0° C. subsequently. The reaction mixture was stirred overnight at room temperature. Reaction mixture was poured into ice water and extracted with ethyl acetate (2×500 mL). Combined organic layer was washed with water (2×500 mL), brine (500 mL) and dried on anhydrous Na₂SO₄. Organic layer was evaporated on rotavapor to afford the titled compound. (60 g, 89% yield). 10

MS(ES+) m/z=360.22 (M+1).

### Step 2: Dimethyl 2-(5-((tert-butoxycarbonyl)amino)-4-(methoxycarbonyl)-2-nitrophenyl)-2-methylmalonate

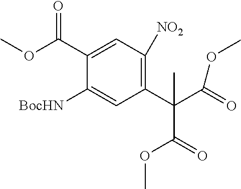

To a solution of dimethyl 2-(5-chloro-4-(methoxycarbonyl)-2-nitrophenyl)-2-methylmalonate (10 g, 27.8 mmol) in dry 1,4-Dioxane (150 ml), were added tert-butyl carbamate (4.89 g, 41.7 mmol), Cs₂C03 (11.78 g, 36.1 mmol). The resulting suspension was degassed with nitrogen for 10 min. Xantphos (1.930 g, 3.34 mmol) and Pd₂(dba)₃ (2.55 g, 2.78 mmol) were added and the reaction mixture was heated at 120° C. for 2 h. The reaction was cooled to room temperature and solvent was removed under reduced pressure. The crude product was purified by flash chromatography in ethyl acetate-n-hexane gradient to afford titled compound (10 g, 82% yield).

MS(ES+) m/z=441.23 (M+1).

### Step 3: Dimethyl 5-((tert-butoxy carbonyl)amino)-3-methyl-2-oxoindoline-3,6-dicarboxylate

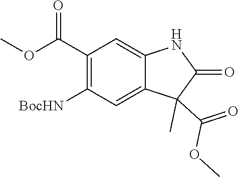 [SMILES: COC(=O)c1cc2c(cc1NC(=O)OC(C)(C)C)C(C)(C(=O)OC)C(=O)N2]

To a stirred solution of dimethyl 2-(5-((tert-butoxycarbonyl)amino)-4-(methoxycarbonyl)-2-nitrophenyl)-2-methylmalonate (10 g, 22.71 mmol)) in Ethanol (120 mL) and acetic acid (20 mL), iron (2.54 g, 45.4 mmol) was added. The resulting reaction mixture was stirred at 100° C. for 2 h. The reaction mixture was concentrated, and the residue was partitioned between ethyl acetate (200 mL) and water (100 mL). Organic layer separated, dried over anhydrous Na₂SO₄, and concentrated under vacuum to afford titled compound (8.51 g, 99% yield).

MS(ES+) m/z=379.35 (M+1).

### Step 4: Dimethyl 5-((tert-butoxy carbonyl)amino)-1,3-dimethyl-2-oxoindoline-3,6-dicarboxylate

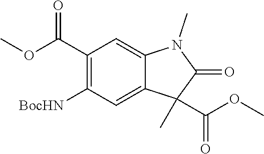

To a stirred solution of dimethyl 5-((tert-butoxycarbonyl)amino)-3-methyl-2-oxoindoline-3,6-dicarboxylate (8.5 g, 22.46 mmol) in DMF (100 mL), K₂CO₃ (4.04 g, 29.2 mmol) and methyl iodide (1.826 ml, 29.2 mmol) were added subsequently. The resulting reaction mixture was stirred at room temperature for 12 h. reaction was diluted with ethyl acetate (200 mL), washed it with water (2×300 mL) and brine (100 mL). Organic layer was dried over anhydrous Na₂SO₄, filtered and concentrated to get crude product. The crude product was purified by column chromatography (Silica gel, Eluent used: 0 to 30% EtOAc in Hexane) to afford dimethyl 5-((tert-butoxycarbonyl)amino)-1,3-dimethyl-2-oxoindoline-3,6-dicarboxylate (7 g, 17.84 mmol, 79% yield).

MS(ES+) m/z=393.2 (M+1).

### Step 5: Dimethyl 5-amino-1,3-dimethyl-2-oxoindoline-3,6-dicarboxylate hydrochloride

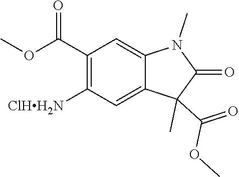

To a solution of dimethyl 5-((tert-butoxycarbonyl)amino)-1,3-dimethyl-2-oxoindoline-3,6-dicarboxylate (7.0 g, 17.84 mmol) in 1,4-dioxane, was added HCl (4M in 1,4-dioxane, 12 mL) at 0° C. and the reaction was stirred at 70° C. for 2 h. Reaction was cooled to room temperature and solvent was evaporated under vacuum to get crude material. Crude product was triturated with diethyl ether to afford titled compound (5.6 g, 95% yield). Crude product was used as such for next step.

MS(ES+) m/z=293.34 (M+1, salt free amine).

### Step 6: Methyl 2,6,8-trimethyl-4,7-dioxo-4,6,7,8-tetrahydro-3H-pyrrolo[2,3-g]quinazoline-8-carboxylate

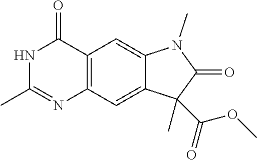

To a solution of dimethyl 5-amino-1,3-dimethyl-2-oxoindoline-3,6-dicarboxylate hydrochloride (5.5 g, 16.73 mmol) in acetonitrile (20 mL), methanesulfonic acid (10.86 ml, 167 mmol) was added and the reaction was stirred at 100° C. for 16 h. Solvent was evaporated and to the residue was dissolved ethyl acetate (50 mL) was added washed it with aq. sodium bicarbonate (2×15 mL) and water (15 mL). The separated organic layer was dried over anhydrous Na₂SO₄, filtered and concentrated to get crude methyl 2,6,8-trimethyl-4,7-dioxo-4,6,7,8-tetrahydro-3H-pyrrolo[2,3-g]quinazoline-8-carboxylate (3.2 g, 10.62 mmol, 63.5% yield).

MS(ES+) m/z=302.2 (M+1).

### Step 7: Methyl 4-chloro-2,6,8-trimethyl-7-oxo-7,8-dihydro-6H-pyrrolo[2,3-g]quinazoline-8-carboxylate

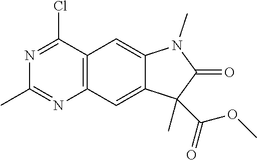

To a suspension of methyl 2,6,8-trimethyl-4,7-dioxo-4,6,7,8-tetrahydro-3H-pyrrolo[2,3-g]quinazoline-8-carboxylate (1 g, 3.32 mmol) in chlorobenzene (15 mL) was added DIPEA (1.739 ml, 9.96 mmol) followed by addition of POCl₃ (0.773 ml, 8.30 mmol) in drop wise manner at room temperature. Resulting reaction mixture was heated at 90° C. for the 2.5 h. Reaction mixture was poured in cold water and extracted with ethyl acetate (2×30 mL). Combined organic layer was washed with brine (25 mL), dried over Na₂SO₄, concentrated to dryness under vacuum to afford titled compound (1 g, 94% yield).

MS(ES+) m/z=319.96 (M+).

### Step 8: Methyl 4-(((R)-1-(3-(1,1-difluoro-2-hydroxy-2-methylpropyl)-2-fluorophenyl) ethyl) amino)-2,6,8-trimethyl-7-oxo-7,8-dihydro-6H-pyrrolo[2,3-g]quinazoline-8-carboxylate

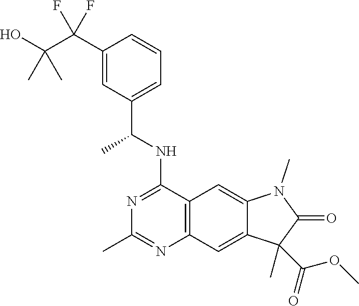 [SMILES: COC(=O)C1(C)C(=O)N(C)c2cc3c(N[C@H](C)c4cccc(C(F)(F)C(C)(C)O)c4)nc(C)nc3cc21]

To a suspension of methyl 4-chloro-2,6,8-trimethyl-7-oxo-7,8-dihydro-6H-pyrrolo[2,3-g]quinazoline-8-carboxylate (1 g, 3.13 mmol) in dioxane (15 mL), were added (R)-1-(3-(1-aminoethyl)-2-fluorophenyl)-1,1-difluoro-2-methylpropan-2-ol hydrochloride (0.887 g, 3.13 mmol) and DIPEA (2.73 ml, 15.64 mmol) at room temperature. Resulting reaction mixture was heated at 120° C. for 16 h. Solvent was evaporated and crude material was purified by flash chromatography in MeOH-DCM gradient to afford titled compound (1.2 g, 72.3% yield).

MS(ES+) m/z=531.44 (M+1).

### Step 9: (R&S)-4-(((R)-1-(3-(1,1-difluoro-2-hydroxy-2-methylpropyl)-2-fluorophenyl)ethyl)amino)-2,6,8-trimethyl-6,8-dihydro-7H-pyrrolo[2,3-g]quinazolin-7-one

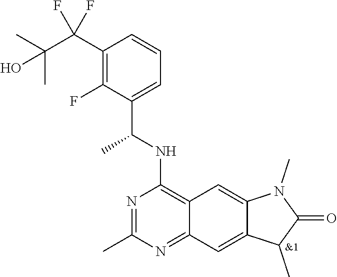 [SMILES: Cc1nc(N[C@H](C)c2cccc(C(F)(F)C(C)(C)O)c2F)c2cc3c(cc2n1)C(C)C(=O)N3C]

To a solution of methyl 4-(((R)-1-(3-(1,1-difluoro-2-hydroxy-2-methylpropyl)-2-fluorophenyl)ethyl)amino)-2,6,8-trimethyl-7-oxo-7,8-dihydro-6H-pyrrolo[2,3-g]quinazoline-8-carboxylate (1 g, 1.885 mmol) in TFA (1.452 ml, 18.85 mmol), H2S04 (3.35 ml, 18.85 mmol) was added and reaction was stirred at 80° C. for 6 h. Reaction was cooled to room temperature, poured in an ice and precipitated solid was filtered. Solid was dissolved in DCM, dried over anhydrous Na₂SO₄ and solvent was evaporated to afford titled compound (0.8 g, 90% yield).

MS(ES+) m/z=473.36 (M+1).

¹H NMR (400 MHz, DMSO-d₆) δ 8.24-8.16 (m, 1H), 7.88-7.82 (m, 1H), 7.60 (s, 1H), 7.57-7.52 (m, 1H), 7.35-7.27 (m, 1H), 7.26-7.16 (m, 1H), 5.84-5.79 (m, 1H), 5.37-5.31 (m, 1H), 3.70-3.61 (m, 1H), 3.27 (s, 3H), 2.32 (d, 1=1.6 Hz, 3H), 1.60 (d, J=7.0 Hz, 3H), 1.45-1.37 (m, 3H), 1.23 (s, 3H), 1.22 (s, 3H).

### Step 10: (R&S)-4-(((R)-1-(3-(1,1-difluoro-2-hydroxy-2-methylpropyl)-2-fluorophenyl) ethyl) amino)-8-methoxy-2,6,8-trimethyl-6,8-dihydro-7H-pyrrolo[2,3-g]quinazolin-7-one (Compound 4)

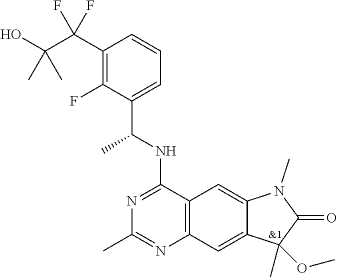 [SMILES: COC1(C)C(=O)N(C)c2cc3c(N[C@H](C)c4cccc(C(F)(F)C(C)(C)O)c4F)nc(C)nc3cc21]

To a stirred solution of 4-(((R)-1-(3-(1,1-difluoro-2-hydroxy-2-methylpropyl)-2-fluorophenyl)ethyl)amino)-2,6,8-trimethyl-6,8-dihydro-7H-pyrrolo[2,3-g]quinazolin-7-one (0.8 g, 1.693 mmol) in methanol (15 mL) was added ceric ammonium nitrate (2.042 g, 3.72 mmol) at 25° C. under inert atmosphere and the resulting reaction mixture was stirred at same temperature for 20 h. Reaction mixture was concentrated under reduced pressure to get sticky compound which was dissolved in DCM (20 ml) and washed with water (3×10 mL). Organic layer was dried over anhydrous Na₂SO₄ and concentrated to get crude product. Crude product was purified by RP HPLC to afford titled compound as mixture of diastereomers (0.17 g, 20% yield).

¹H NMR (400 MHz, DMSO-d₆) δ 8.30 (d, J=7.3 Hz. 1H), 7.95 (s, 1H), 7.65-7.58 (m, 1H), 7.56 (d, J=1.8 Hz, 1H), 7.35-7.28 (m, 1H), 7.26-7.18 (m, 1H), 5.87-5.78 (m, 1H), 5.35 (s, 1H), 3.30 (s, 3H), 2.90 (S, 3H), 2.33 (s, 3H), 1.61 (d, J=7.0 Hz, 3H), 1.49 (s, 3H), 1.24 (s, 3H), 1.22 (s, 3H).

(NMR spectra of Diastereomeric mixture)

MS(ES+) m/z=503.43 (M+1).

The diastereomers of compound 4 were separated by preparative chiral HPLC

HPLC method: CHIRALPAK IC CRL-087 HEX0.1% DEA_IPA-DCM_70_30_A_B_1.2 ML_10MIN_290 nm 1.2 mL/min.

### Peak 1: (S/R)-4-(((R)-1-(3-(1,1-difluoro-2-hydroxy-2-methylpropyl)-2-fluorophenyl)ethyl)amino)-8-methoxy-2,6,8-trimethyl-6H-pyrrolo[2,3-g]quinazolin-7(8H)-one (0.015 g, 1.763% yield) (Compound 4a)

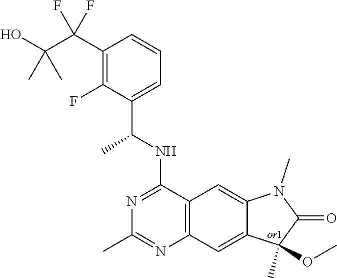 [SMILES: CO[C@]1(C)C(=O)N(C)c2cc3c(N[C@H](C)c4cccc(C(F)(F)C(C)(C)O)c4F)nc(C)nc3cc21]

tᵣₑₜ(min)=4.58

MS(ES+) m/z=503.43 (M+1)

¹H NMR (400 MHz, DMSO-d₆) δ 8.35 (d, J=7.3 Hz, 1H), 7.96 (s, 1H), 7.64-7.58 (m, 1H), 7.57 (s, 1H), 7.35-7.29 (m, 1H), 7.25-7.19 (m, 1H), 5.87-5.78 (m, 1H), 5.35 (s, 1H), 3.30 (s, 3H), 2.91 (s, 3H), 2.34 (s, 3H), 1.61 (d, J=7.0 Hz, 3H), 1.48 (s, 3H), 1.24 (s, 3H), 1.22 (s, 3H).

### Peak 2: (R/S)-4-(((R)-1-(3-(1,1-difluoro-2-hydroxy-2-methylpropyl)-2-fluorophenyl)ethyl) amino)-8-methoxy-2,6,8-trimethyl-6H-pyrrolo[2,3-g]quinazolin-7(8H)-one (0.020 g, 2.3% yield) (Compound 4b)

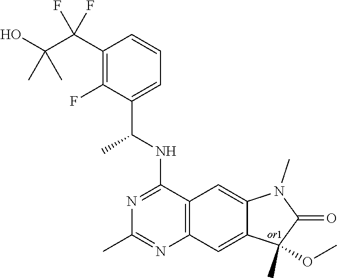 [SMILES: CO[C@@]1(C)C(=O)N(C)c2cc3c(N[C@H](C)c4cccc(C(F)(F)C(C)(C)O)c4F)nc(C)nc3cc21]

tᵣₑₜ(min)=5.39

MS(ES+) m/z=503.44 (M+1)

¹H NMR (400 MHz, DMSO-d₆) δ 8.31 (d, J=7.1 Hz, 1H), 7.95 (s, 1H), 7.66-7.58 (m, 1H), 7.56 (s, 1H), 7.35-7.28 (m, 1H), 7.26-7.19 (m, 1H), 5.87-5.78 (m, 1H), 5.35 (s, 1H), 3.30 (s, 3H), 2.89 (s, 3H), 2.34 (s, 3H), 1.61 (d, J=7.0 Hz, 3H), 1.49 (s, 3H), 1.24 (s, 3H), 1.22 (s, 3H).

### Example 5: (R)-5-(4-((1-(3-amino-5-(trifluoromethyl) phenyl) ethyl) amino)-2-methyl-8,9-dihydro-7H-cyclopenta[h]quinazolin-6-yl)-1-methylpyridin-2(1H)-one (Compound 5)

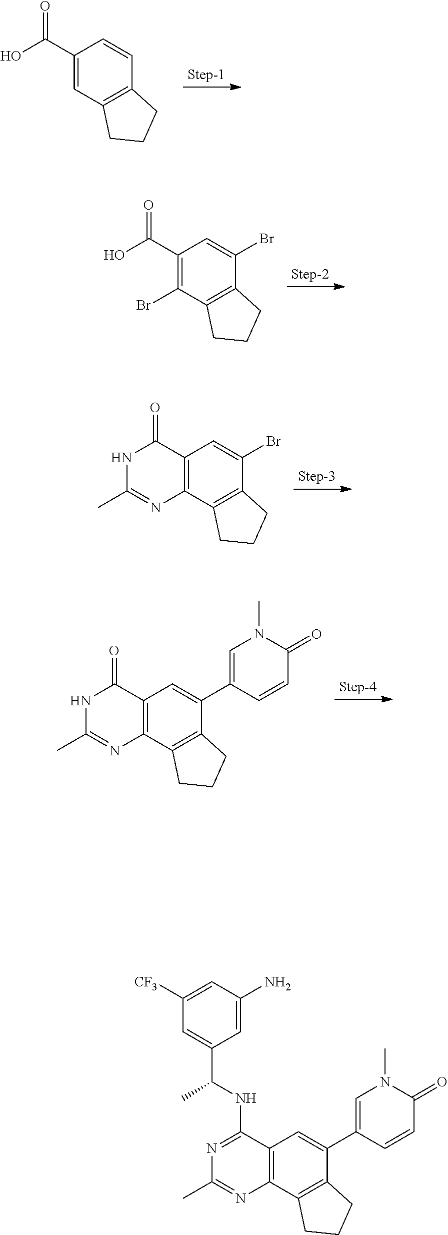 [SMILES: O=C(O)c1ccc2c(c1)CCC2 ; O=C(O)c1cc(Br)c2c(c1Br)CCC2 ; Cc1nc2c3c(c(Br)cc2c(=O)[nH]1)CCC3 ; Cc1nc2c3c(c(-c4ccc(=O)n(C)c4)cc2c(=O)[nH]1)CCC3 ; Cc1nc(N[C@H](C)c2cc(N)cc(C(F)(F)F)c2)c2cc(-c3ccc(=O)n(C)c3)c3c(c2n1)CCC3]

### Step 1: 4,7-dibromo-2,3-dihydro-1H-indene-5-carboxylic acid

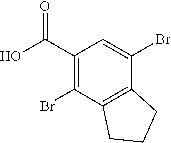 [SMILES: O=C(O)c1cc(Br)c2c(c1Br)CCC2]

NBS (5.49 g, 30.8 mmol) was added portion wise to a solution of 2,3-dihydro-1H-indene-5-carboxylic acid (Commercially available) (2 g, 12.33 mmol) in Conc. H₂SO₄ (20 ml) at room temperature and the mixture was stirred overnight at room temperature, then poured the reaction mass onto crushed ice cold water solution. The solution was stirred for 30 min, the solid was filtered, air dried and precipitated with EtOAc and hexane to get 4,7-dibromo-2,3-dihydro-1H-indene-5-carboxylic acid (3.81 g, 97% yield (crude) as a brown solid.

MS (ES+) m/z=319.94 (M+).

¹H NMR (400 MHz, DMSO-d₆) δ 13.49 (s, 1H, D20 exchangeable), 7.71 (s, 1H), 3.11-3.96 (m, 4H), 2.15-2.04 (m, 2H).

### Step 2: 6-bromo-2-methyl-3,7,8,9-tetrahydro-4H-cyclopenta [h]quinazolin-4-one

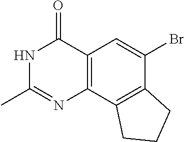 [SMILES: Cc1nc2c3c(c(Br)cc2c(=O)[nH]1)CCC3]

A mixture of 4,7-dibromo-2,3-dihydro-1H-indene-5-carboxylic acid (70 g, 219 mmol), acetamidamide hydrochloride (31 g, 328 mmol), copper(I)iodide (8.33 g, 43.8 mmol) and cesium carbonate (143 g, 438 mmol) in DMF (500 ml) were heated at 70° C. for 16 hours. After completion of reaction, poured the reaction mass into water and extracted with EtOAc, washed the organic layer with water (100 ml), brine (50 ml) and dried over anhydrous sodium sulphate and concentrated under reduced pressure to get a crude compound (45.2 g). The crude compound was purified by column chromatography using 20-30% ethyl acetate in hexane to afford the titled compound 6-bromo-2-methyl-3,7,8,9-tetrahydro-4H-cyclopenta[h]quinazolin-4-one (27 g, 44.2% yield) as a white solid.

MS (ES+) m/z=279.15 (M+).

¹H NMR (400 MHz, DMSO-d₆) δ 12.27 (s, 1H), 7.99 (s, 1H), 3.20 (t, J=7.6 Hz, 2H), 3.01 (t, J=7.5 Hz, 2H), 2.34 (s, 3H), 2.20-2.09 (m, 2H).

### Step 3: 2-methyl-6-(1-methyl-6-oxo-1,6-dihydropyridin-3-yl)-3,7,8,9-tetrahydro-4Hcyclopenta[h]quinazolin-4-one

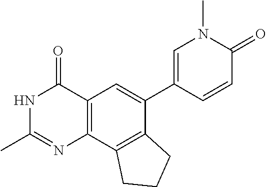 [SMILES: Cc1nc2c3c(c(-c4ccc(=O)n(C)c4)cc2c(=O)[nH]1)CCC3]

To a stirred solution of 6-bromo-2-methyl-3,7,8,9-tetrahydro-4H-cyclopenta[h]quinazolin-4-one (1 g, 3.58 mmol) in 1,4-Dioxane (10 ml) and Water (2 ml) were added 1-methyl-5-(4,4,5,5-tetramethyl-1,3,2-dioxaborolan-2-yl)pyridin-2(1H)-one (1.263 g, 5.37 mmol) (commercially available), cesium carbonate (3.50 g, 10.75 mmol) and PdCl₂(dppf). DCM adduct (0.146 g, 0.179 mmol) at room temperature. The resulting reaction mixture was purged with nitrogen for 15 min and heated at 80° C. for 3 h in a sealed vial. After completion of reaction, reaction mixture was evaporated to get crude (1.9 g) which was purified by flash column chromatography by using gradient elution of 0-1% MeOH in DCM to afford 2-methyl-6-(1-methyl-6-oxo-1,6-dihydropyridin-3-yl)-3,7,8,9-tetrahydro-4Hcyclopenta[h]quinazolin-4-one (0.780 g, 70.8% yield) as light yellow solid.

MS (ES+) m/z=308.09 (M+1).

¹H NMR (400 MHz, DMSO-4₆) δ 12.12 (s, 1H), 7.94 (d, J=2.7 Hz, 1H), 7.84 (s, 1H), 7.70-7.63 (m, 1H), 6.50-6.44 (m, 1H), 3.51 (s, 3H), 3.14 (t, J=7.5 Hz, 2H), 3.06 (t, J=7.4 Hz, 2H), 2.37 (s, 3H), 2.16-2.02 (m, 2H).

### Step 4: (R)-5-(4-((1-(3-amino-5-(trifluoromethyl)phenyl)ethyl)amino)-2-methyl-8,9-dihydro-7H-cyclopenta[h]quinazolin-6-yl)-1-methylpyridin-2(1H)-one (Compound 5)

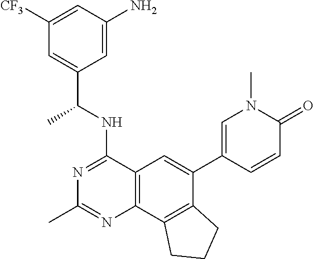 [SMILES: Cc1nc(N[C@H](C)c2cc(N)cc(C(F)(F)F)c2)c2cc(-c3ccc(=O)n(C)c3)c3c(c2n1)CCC3]

To a solution of 2-methyl-6-(1-methyl-6-oxo-1,6-dihydropyridin-3-yl)-3,7,8,9-tetrahydro-4H-cyclopenta[h]quinazolin-4-one (150 mg, 0.488 mmol) and (R)-3-(1-aminoethyl)-5-(trifluoromethyl)aniline (149 mg, 0.732 mmol) in ACN (15 ml) was added benzotriazol-1-yloxytris(dimethylamino)-phosphonium hexafluorophosphate (324 mg, 0.732 mmol) and DBU (0.368 ml, 2.440 mmol) at 0° C. and allowed to stirred 100° C. for 16 h. After completion of reaction, reaction mixture was concentrated and purified by flash column chromatography by using gradient elution of 0-5% MeOH in DCM to afford (R)-5-(4-((1-(3-amino-5-(trifluoromethyl)phenyl)ethyl)amino)-2-methyl-8,9-dihydro-7H-cyclopenta[h]quinazolin-6-yl)-1-methylpyridin-2(1H)-one (10 mg, 4.15% yield).

MS (ES+) m/z=494.17 (M+1).

¹H NMR (400 MHz, DMSO-d₆) δ 8.26 (d, J=8.0 Hz, 1H), 8.16 (s, 1H), 7.94-7.90 (m, 1H), 7.74-7.69 (m, 1H), 6.91-6.88 (m, 1H), 6.87-6.84 (m, 1H), 6.71-6.68 (m, 1H), 6.55-6.50 (m, 1H), 5.64-5.48 (m, 3H), 3.54 (s, 3H), 3.20-3.13 (m, 2H), 3.12-3.01 (m, 2H), 2.42 (s, 3H), 2.18-2.03 (m, 2H), 1.55 (d, J=7.0 Hz, 3H).

### Example 6: (R&S)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6)

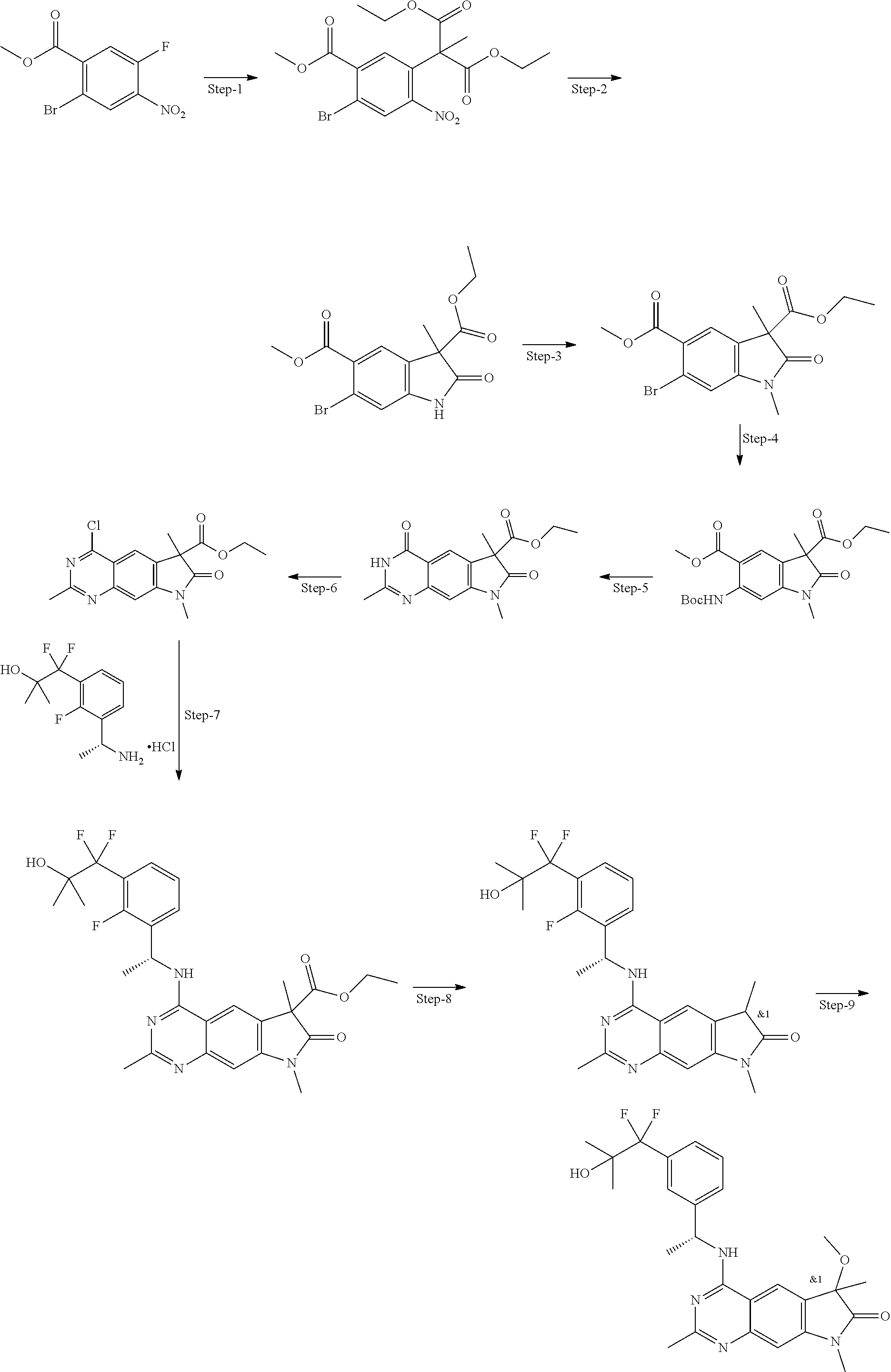

### Step 1: Diethyl 2-(4-bromo-5-(methoxycarbonyl)-2-nitrophenyl)-2-methylmalonate

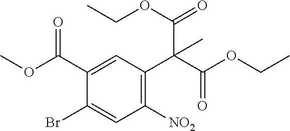 [SMILES: CCOC(=O)C(C)(C(=O)OCC)c1cc(C(=O)OC)c(Br)cc1[N+](=O)[O-]]

To a stirred solution of methyl 2-bromo-5-fluoro-4-nitrobenzoate (5 g, 17.98 mmol) in DMF (50 mL) was added K₂CO₃ (7.46 g, 54.0 mmol) followed by addition of diethyl 2-methylmalonate (4.70 g, 27 mmol). Resulting reaction mixture was heated at 70° C. for the 20 h. Reaction mixture was filtered and washed with DMF (20 mL). Filtrate was poured in 2N HCl and extracted with MTBE (2×100 mL). Organic layer was washed with brine, dried over anhydrous Na₂SO₄ and concentrated in vacuum. Crude product was purified by column chromatography in ethyl acetate-hexane gradient to afford titled compound (3.7 g, 47.6% yield).

¹H NMR (400 MHz, CDCl₃) δ 8.31 (s, 1H), 7.82 (s, 1H), 4.31-4.21 (m, 4H), 4.00 (s, 3H), 2.03 (s, 3H), 1.26 (t, J=7.1 Hz, 6H).

### Step 2: 3-Ethyl 5-methyl 6-bromo-3-methyl-2-oxoindoline-3,5-dicarboxylate

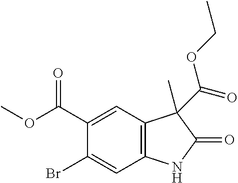 [SMILES: CCOC(=O)C1(C)C(=O)Nc2cc(Br)c(C(=O)OC)cc21]

To a stirred solution of diethyl 2-(4-bromo-5-(methoxycarbonyl)-2-nitrophenyl)-2-methylmalonate (3.700 g, 8.56 mmol) in Ethanol (37 mL) and acetic acid (37 mL), iron (0.956 g, 17.12 mmol) was added and reaction was stirred at 100° C. in an oil bath for 2 h. Reaction mixture was cooled to room temperature and concentrated under vacuum. The residue was stirred in ethyl acetate and solid was filtered off. The Filtrate was washed with water (2×150 mL), brine (50 mL), dried over anhydrous Na₂SO₄ and concentrated under reduced pressure to afford the titled compound (2.600 g, 85% yield).

¹H NMR (400 MHz, DMSO-d₆) δ 11.13 (s, 1H), 7.70 (s, 1H), 7.20 (s, 1H), 4.20-4.02 (m, 2H), 3.82 (s, 3H), 1.54 (s, 3H), 1.07 (t, J=7.1 Hz, 3H).

### Step 3: 3-Ethyl 5-methyl 6-bromo-1,3-dimethyl-2-oxoindoline-3,5-dicarboxylate

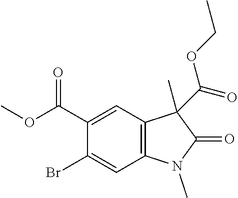 [SMILES: CCOC(=O)C1(C)C(=O)N(C)c2cc(Br)c(C(=O)OC)cc21]

To a stirred solution of 3-ethyl 5-methyl 6-bromo-3-methyl-2-oxoindoline-3,5-dicarboxylate (2.600 g, 7.30 mmol) in DMF (25 ml), K₂CO₃(1.513 g, 10.95 mmol) and iodomethane (0.502 ml, 8.03 mmol) were added and reaction was stirred at room temperature for the 3h. Reaction mixture was poured in ice water and extracted with MTBE (2×150 mL). Combined organic layer was washed with brine (60 mL), dried over anhydrous Na₂SO₄ and concentrate in vacuum. Crude product was purified by column chromatography in ethyl acetate-hexane gradient to afford the titled compound (2.400 g, 89% yield).

¹H NMR (400 MHz, DMSO-d₆) δ 7.73 (s, 1H), 7.54 (s, 1H), 4.18-3.98 (m, 2H), 3.83 (s, 3H), 3.22 (s, 3H), 1.56 (s, 3H), 1.07 (t, J=7.1 Hz, 3H).

### Step 4: 3-Ethyl 5-methyl 6-((tert-butoxycarbonyl)amino)-1,3-dimethyl-2-oxoindoline-3,5-dicarboxylate

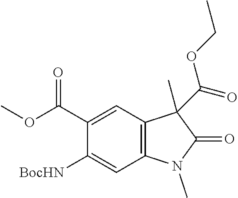 [SMILES: CCOC(=O)C1(C)C(=O)N(C)c2cc(NC(=O)OC(C)(C)C)c(C(=O)OC)cc21]

To a stirred solution of 3-ethyl 5-methyl 6-bromo-1,3-dimethyl-2-oxoindoline-3,5-dicarboxylate (2.250 g, 6.08 mmol) in dry 1,4-Dioxane (50 ml), tert-butyl carbamate (0.854 g, 7.29 mmol), Pd₂(dba)₃ (0.278 g, 0.304 mmol), xantphos (0.422 g, 0.729 mmol) and Cs₂CO₃ (3.56 g, 10.94 mmol) were added subsequently under inert atmosphere. Resulting reaction mixture was stirred at 110° C. for 16h. Reaction mixture was cooled to room temperature, diluted with DCM (50 mL) and filtered through celite. Celite bed was washed with DCM (3×50 mL). Filtrate was concentrated under vacuum and the crude product was purified by column chromatography in ethyl acetate-hexane gradient to afford titled compound.

¹H NMR (400 MHz, DMSO-d₆) δ 10.66 (s, 1H), 7.98 (s, 1H), 7.80 (s, 1H), 4.17-4.00 (m, 2H), 3.85 (s, 3H), 3.20 (s, 3H), 1.51 (s, 9H), 1.37 (s, 3H), 1.06 (t, J=7.1 Hz, 3H).

### Step 5: Ethyl 2,6,8-trimethyl-4,7-dioxo-4,6,7,8-tetrahydro-3H-pyrrolo[3,2-g]quinazoline-6-carboxylate

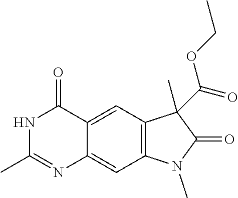 [SMILES: CCOC(=O)C1(C)C(=O)N(C)c2cc3nc(C)[nH]c(=O)c3cc21]

To a solution of 3-ethyl 5-methyl 6-((tert-butoxycarbonyl)amino)-1,3-dimethyl-2-oxoindoline-3,5-dicarboxylate (0.900 g, 2.214 mmol) in acetonitrile (10 mL) was added MSA (0.863 ml, 13.29 mmol) and resulting reaction mixture was heated at 110° C. for the 40h in seal tube. Reaction mixture was evaporated and basify with aq. NaHCO₃ slowly. Aqueous layer was extracted with ethyl acetate (3×150 mL). Combined organic layer was dried over anhydrous Na₂SO₄ and concentrated under vacuum. Crude material was purified by column chromatography in MeOH-Ethyl acetate to afford titled compound (0.402 g, 57.6% yield).

MS(ES+) m/z=316.04 (M+1).

### Step 6: Ethyl 4-chloro-2,6,8-trimethyl-7-oxo-7,8-dihydro-6H-pyrrolo[3,2-g]quinazoline-6-carboxylate

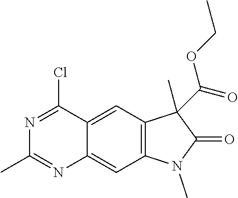 [SMILES: CCOC(=O)C1(C)C(=O)N(C)c2cc3nc(C)nc(Cl)c3cc21]

To a suspension of ethyl 2,6,8-trimethyl-4,7-dioxo-4,6,7,8-tetrahydro-3H-pyrrolo[3,2-g]quinazoline-6-carboxylate (0.3 gm, 0.951 mmol) in Chlorobenzene (8 ml), DIPEA (0.548 ml, 3.14 mmol) was added at room temperature followed by addition of POCl₃ (0.284 ml, 3.04 mmol) in dropwise manner. Resulting reaction mixture was stirred at room temperature for 10 min and then at 90° C. for the 3 h. Reaction mixture was concentrated and vacuum and diluted with DCM (20 ml). Organic layer was washed with brine (10 mL), dried over anhydrous Na₂SO₄ and concentrated under vacuum to furnish titled compound (0.3 gm, 94% yield). It was used as such for the next reaction.

MS(ES+) m/z=334.34 (M+1).

### Step 7: Ethyl 4-(((R)-1-(3-(1,1-difluoro-2-hydroxy-2-methylpropyl)-2-fluorophenyl)ethyl)amino)-2,6,8-trimethyl-7-oxo-7,8-dihydro-6H-pyrrolo[3,2-g]quinazoline-6-carboxylate

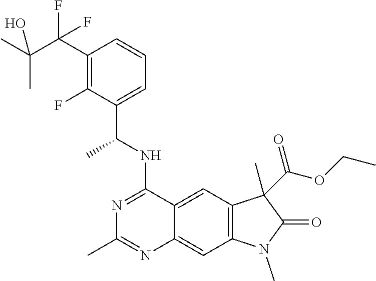 [SMILES: CCOC(=O)C1(C)C(=O)N(C)c2cc3nc(C)nc(N[C@H](C)c4cccc(C(F)(F)C(C)(C)O)c4F)c3cc21]

To a suspension of (R)-1-(3-(1-aminoethyl)-2-fluorophenyl)-1,1-difluoro-2-methylpropan-2-ol hydrochloride (0.367 g, 1.294 mmol) in 1, 4-Dioxane (10 mL) was added DIPEA (0.942 ml, 5.39 mmol) at room temperature followed by addition of ethyl 4-chloro-2,6,8-trimethyl-7-oxo-7,8-dihydro-6H-pyrrolo[3,2-g]quinazoline-6-carboxylate (0.360 g, 1.079 mmol). Resulting reaction mixture was stirred at 120° C. for the 48 h. Reaction mixture was concentrated in vacuum to dryness and residue was purified by column chromatography in MeOH-DCM gradient to afford titled compound (0.380 g, 64.7% yield).

MS(ES+) m/z=545.20 (M+1).

### Step 8: (R&S)-4-(((R)-1-(3-(1,1-difluoro-2-hydroxy-2-methylpropyl)-2-fluorophenyl)ethyl) amino)-2,6,8-trimethyl-6,8-dihydro-7H-pyrrolo[3,2-g]quinazolin-7-one

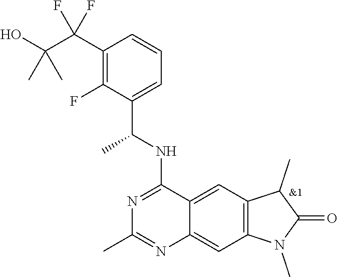 [SMILES: Cc1nc(N[C@H](C)c2cccc(C(F)(F)C(C)(C)O)c2F)c2cc3c(cc2n1)N(C)C(=O)C3C]

To a stirred ethyl 4-(((R)-1-(3-(1,1-difluoro-2-hydroxy-2-methylpropyl)-2-fluorophenyl)ethyl)amino)-2,6,8-trimethyl-7-oxo-7,8-dihydro-6H-pyrrolo[3,2-g]quinazoline-6-carboxylate (0.3 g, 0.551 mmol) in TFA (0.424 ml, 5.51 mmol) was added H₂SO₄ (0.979 ml, 5.51 mmol) and the reaction was stirred at 80° C. for 6 h. Reaction was poured in to ice water and solid product was filtered. It was further dried under vacuum to afford titled compound (0.2 g, 77%). It was used as such for next step.

MS(ES+) m/z=473.42 (M+1).

### Step 9: (R&S)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6)

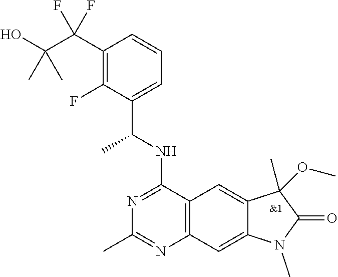 [SMILES: COC1(C)C(=O)N(C)c2cc3nc(C)nc(N[C@H](C)c4cccc(C(F)(F)C(C)(C)O)c4F)c3cc21]

To a stirred solution of 4-(((R)-1-(3-(1,1-difluoro-2-hydroxy-2-methylpropyl)-2-fluorophenyl)ethyl) amino)-2,6,8-trimethyl-6,8-dihydro-7H-pyrrolo[3,2-g]quinazolin-7-one (7.5 g, 15.87 mmol) in MeOH (150 mL) was added CAN (9.14 g, 34.9 mmol) at 25° C. under inert atmosphere. The reaction mixture was stirred at same temperature for 12 h. The reaction mixture was concentrated under reduced pressure to get sticky compound which was dissolved in DCM (200 mL) and washed with water (3×100 mL). The organic layer was separated, dried over anhydrous Na₂SO₄ and concentrated to get a crude product. The crude product was purified by preparative HPLC to afford the titled compound as mixture of diastereomers. Two diastereomers were separated by chiral preparative HPLC—

Chiral separation method: CHIRALPAK IG CRL-086 HEX_0.1% DEA_IPA_80_20_A_B_0.7ML_15MIN_265NM

### Peak 1: (S/R)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (0.60 g, 7.52% yield) (Compound 6a)

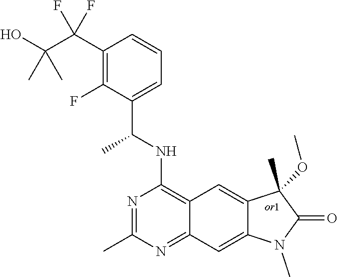 [SMILES: CO[C@]1(C)C(=O)N(C)c2cc3nc(C)nc(N[C@H](C)c4cccc(C(F)(F)C(C)(C)O)c4F)c3cc21]

MS(ES+) m/z=503.43 (M+1).

RT: tᵣₑₜ(min)=9.96.

¹H NMR (400 MHz, DMSO-d₆) δ 8.46 (s, 1H), 8.38 (d, J=6.9 Hz, 1H), 7.65-7.56 (m, 1H), 7.34-7.28 (m, 1H), 7.27-7.19 (m, 1H), 7.13 (s, 1H), 5.82-5.77 (m, 1H), 5.33 (s, 1H), 3.22 (s, 3H), 2.92 (s, 3H), 2.32 (s, 3H), 1.58 (d, J=7.0 Hz, 3H), 1.54 (s, 3H), 1.24 (s, 3H), 1.21 (s, 3H).

### Peak-2: (R/S)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (0.45 g, 5.64% yield) (Compound 6b)

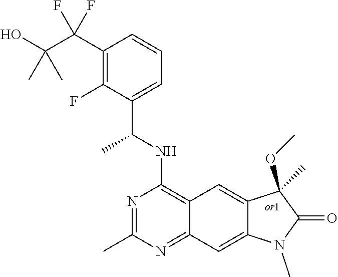 [SMILES: CO[C@@]1(C)C(=O)N(C)c2cc3nc(C)nc(N[C@H](C)c4cccc(C(F)(F)C(C)(C)O)c4F)c3cc21]

MS(ES+) m/z=503.43 (M+1).

RT: tᵣₑₜ(min)=10.61

¹H NMR (400 MHz, DMSO-d₆) 8.44 (s, 1H), 8.37 (s, 1H), 7.65-7.60 (m, 1H), 7.35-7.29 (m, 1H), 7.27-7.23 (m, 1H), 7.13 (s, 1H), 5.81-5.76 (m, 1H), 5.33 (s, 1H), 3.22 (s, 3H), 2.93 (s, 3H), 2.34 (s, 3H), 1.58 (d, J=7.0 Hz, 3H), 1.53 (s, 3H) 1.24 (s, 3H), 1.23 (s, 3H).

### Example 7: Preparation of (S)-4-(((R)-1-(3-amino-5-(trifluoromethyl) phenyl) ethyl)amino)-8-methoxy-2,6,8-trimethyl-6,8-dihydro-7H-pyrrolo[2,3-g]quinazolin-7-one (Compound 7)

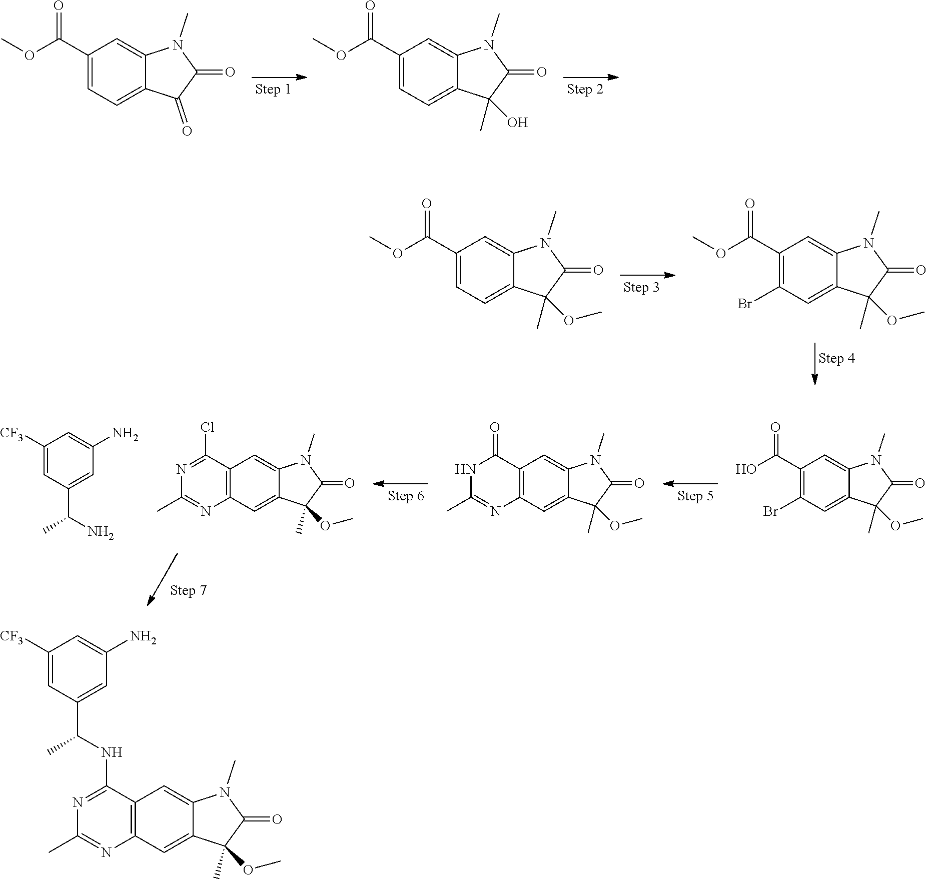

### Step 1: Preparation of Methyl 3-hydroxy-1,3-dimethyl-2-oxoindoline-6-carboxylate

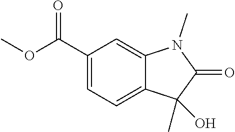 [SMILES: COC(=O)c1ccc2c(c1)N(C)C(=O)C2(C)O]

To a solution of the (3R,5R)-1-benzyl-5-(hydroxydiphenylmethyl)pyrrolidin-3-ol (10.04 g, 27.9 mmol, available, CAS no: 648424-71-9) in toluene (350.0 mL), dimethyl zinc (47.9 mL, 47.9 mmol) was added and reaction was stirred for 30 min at room temperature. 2-methylbutan-2-ol (5.25 mL, 47.9 mmol) was added and stirring was continued for further 30 min. The mixture was cooled to −40° C. and methyl 1-methyl-2,3-dioxoindoline-6-carboxylate (35.0 g, 160 mmol) was added, followed by the drop wise addition of dimethyl zinc (351.0 mL, 351 mmol) over 8 h at −40° C. The reaction was warmed to room temperature and stirred for 15h at same temperature. The reaction mixture was quenched by 10% citric acid solution and extracted with Ethyl acetate (3×500.0 mL). Combined organic layer was dried over Na₂SO₄ and solvent was removed under vacuum. The crude solid was purified by flash column chromatography in ethyl acetate-hexane gradient to titled compound (32.0 g, 85% yield)

¹H NMR (400 MHz, DMSO-d₆) δ 7.74-7.70 (m, 1H), 7.51-7.47 (m, 2H), 6.12 (s, 1H), 3.88 (s, 3H), 3.16 (s, 3H), 1.41 (s, 3H).

Chiral HPLC method: HEX_IPA_DCM_70_30_B_C_1.0 ML_12MIN_225 nm, 1.0 ml/min CHIRALPAK OX-H CRL-081

tᵣₑₜ(min): 6.91 min (11.70%)

tᵣₑₜ(min): 7.74 min (88.30%)

### Step 2: Preparation of Methyl 3-methoxy-1,3-dimethyl-2-oxoindoline-6-carboxylate

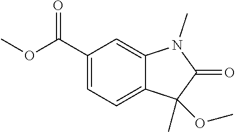

To a solution of methyl 3-hydroxy-1,3-dimethyl-2-oxoindoline-6-carboxylate (50.0 g, 213 mmol) and methyl iodide (19.94 mL, 319 mmol) in DMF (100.0 mL) was added sodium hydride (12.75 g, 319 mmol) at −5° C. the resulting mixture was stirred at −5° C. to 0° C. for 30 min. The reaction mass was quenched with sat. ammonium chloride solution (100.0 mL). The resulting mixture was extracted with ethyl acetate (3×250 mL). The combined organic layer was washed with brine (200.0 mL), dried over anh. Na₂SO₄ and evaporated under reduced pressure. The crude oil was purified by flash column chromatography to provide the titled compound (45.0 g, 85% yield)

¹H NMR (400 MHz, DMSO-d₆) δ 7.79-7.74 (m, 1H), 7.57-7.48 (m, 2H), 3.89 (s, 3H), 3.21 (s, 3H), 2.87 (s, 3H), 1.44 (s, 3H).

Chiral HPLC method: HEX_0.1% TFA_IPA_90_10_A_B_1.2ML_20MIN 1.2 ml/min CHIRALPAK ID CRL-065

tᵣₑₜ(min): 10.22 min (88.85%)

tᵣₑₜ(min): 11.87 min (11.25%)

### Step 3: Preparation of Methyl 5-bromo-3-methoxy-1,3-dimethyl-2-oxoindoline-6-carboxylate

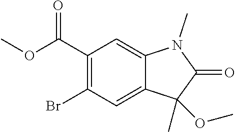

To a solution of methyl 3-methoxy-1,3-dimethyl-2-oxoindoline-6-carboxylate (40.0 g, 160 mmol) in acetonitrile (480.0 mL), TFA (12.36 mL, 160 mmol) and NBS (31.4 g, 177 mmol) were added. The reaction was stirred at 25° C. for 1 h. Reaction was quenched with aq. sodium thiosulphate solution and aq. sodium bicarbonate solution. Acetonitrile was evaporated under reduced pressure and the resulting mixture was stirred for 10 min. Solid was filtered and dried under vacuum to provide the titled compound (50.0 g, 95% yield) as off-white solid

¹H NMR (400 MHz, DMSO-d₆) δ 7.73 (s, 1H), 7.41 (s, 1H), 3.89 (s, 3H), 3.16 (s, 3H), 2.89 (s, 3H), 1.45 (s, 3H)

### Step 4: Preparation of 5-bromo-3-methoxy-1,3-dimethyl-2-oxoindoline-6-carboxylic acid

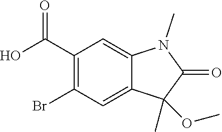 [SMILES: COC1(C)C(=O)N(C)c2cc(C(=O)O)c(Br)cc21]

To a solution of methyl 5-bromo-3-methoxy-1,3-dimethyl-2-oxoindoline-6-carboxylate (50.0 g, 152 mmol) in Methanol (200.0 mL), Tetrahydrofuran (200.0 mL), and Water (100.0 mL), lithium hydroxide (9.12 g, 381 mmol) was added at room temperature. The reaction was heated to 60° C. for 2 h. The reaction was cooled to room temperature and solvent was removed under reduced pressure. The crude oil was acidified with 1N HCl. The solid was filtered, washed with water and dried under vacuum to provide the titled compound (40 g, 84% yield).

¹H NMR (400 MHz, DMSO-d₆) δ 13.61 (s, 11H), 7.68 (s, 1H), 7.38 (s, 1H), 3.17 (s, 3H), 2.89 (s, 3H), 1.44 (s, 3H).

### Step 5: Preparation of 8-methoxy-2,6,8-trimethyl-6,8-dihydro-3H-pyrrolo[2,3-g]quinazoline-4,7-dione

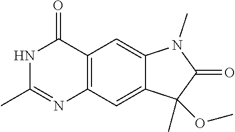

To a suspension of 5-bromo-3-methoxy-1,3-dimethyl-2-oxoindoline-6-carboxylic acid (47.0 g, 150 mmol) in DMF (500.0 mL), acetimidamide hydrochloride (21.22 g, 224 mmol), cesium carbonate (146 g, 449 mmol) and Copper(I) iodide (5.70 g, 29.9 mmol) were added subsequently. The reaction was purged with nitrogen for 15 min and stirred at 85° C. for 3h. Reaction was cooled to room temperature and poured into ice water. The solid was filtered and dried under reduced pressure to afford the titled compound (30 g, 73.4% yield).

¹H NMR (400 MHz, DMSO-d₆) δ 12.29 (s, 1H), 7.56 (s, 1H), 7.55 (s, 1H), 3.24 (s, 3H), 2.89 (s, 3H), 2.35 (s, 3H), 1.48 (s, 3H).

### Step 6: Preparation of (S)-4-chloro-8-methoxy-2,6,8-trimethyl-6,8-dihydro-7H-pyrrolo[2,3-g]quinazolin-7-one

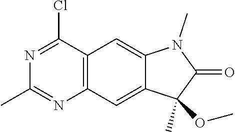

To a suspension of 8-methoxy-2,6,8-trimethyl-6,8-dihydro-3H-pyrrolo[2,3-g]quinazoline-4,7-dione (15 g, 54.9 mmol) in chlorobenzene (160.0 mL), DIPEA (25.9 mL, 148 mmol) was added. POCl₃ (12.79 mL, 137 mmol) was added in drop wise manner at room temperature and reaction mixture was heated at 90° C. for 2.5 h. The reaction was cooled to room temperature and poured in ice cooled water. The resulting mixture was extracted with ethyl acetate (2×500 mL). Combined organic layer was washed with brine (˜250.0 mL), dried over anhydrous Na₂SO₄ and concentrated under reduced pressure to give the titled compound (11.5 g, 71.8% yield).

¹H NMR (400 MHz, DMSO-d₆) δ 8.00 (s, 1H), 7.56 (s, 1H), 3.33 (s, 3H), 2.94 (s, 3H), 2.74 (s, 3H), 1.55 (s, 3H).

MS(ES+) m/z=292.02(M+1)

The enantiomerically enriched chloro intermediate was converted to the methoxy intermediate by SnAr displacement by methoxide anion. The major isomer of this methoxy product (Peak 2 in chiral HPLC) was compared with the retention time of the isomer confirmed to have the S-configuration determined by X ray crystallography.

### Step 7: Preparation of (S)-4-(((R)-1-(3-amino-5-(trifluoromethyl) phenyl) ethyl)amino)-8-methoxy-2,6,8-trimethyl-6,8-dihydro-7H-pyrrolo[2,3-g]quinazolin-7-one (Compound 7)

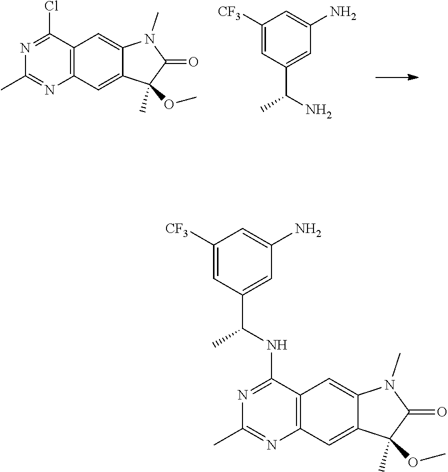

To a solution of (S)-4-chloro-8-methoxy-2,6,8-trimethyl-6,8-dihydro-7H-pyrrolo[2,3-g]quinazolin-7-one (5.0 g, 17.14 mmol) in dioxane (15.0 mL), DIPEA (29.9 mL, 171 mmol) and (R)-3-(1-aminoethyl)-5-(trifluoromethyl)aniline (3.67 g, 18.0 mmol) were added and the reaction mixture was stirred at 120° C. for 48h. The reaction was cooled and solvent was removed under reduced pressure. The crude product was purified by preparative HPLC to provide the 3.2 g compound. It was further purified by chiral preparative HPLC to provide the titled compound (1.95 g)

¹H NMR (400 MHz, DMSO-d₆) δ 8.19 (d, J=7.9 Hz, 1H), 7.91 (s, 1H), 7.56 (s, 1H), 6.91 (s, 1H), 6.89-6.85 (m. 1H), 6.84-6.62 (m, 1H), 5.72-5.47 (m, 3H), 3.27 (s, 3H), 2.90 (s, 3H). 2.40 (s, 3H), 1.57 (d, J=7.1 Hz, 3H), 1.49 (s, 3H).

MS (ES+) m/z=460.43 (M+1)

Chiral HPLC: Hexane_0.1% Diethylamine_isopropyl alcohol-Dichloromethane_60_40_A_B_1.2ML_10MIN_290NM CHIRALPAK IC CRL-087

tᵣₑₜ(min): 4.71 min (100%)

### Example 8: In Vitro Experiments

Compound 1, 2, 3, 3a, 3b, 4, 4a, 4b, 5, 6, 6a, 6b and 7 were tested for inhibition of colony formation potential in combination with one or more of the following agents in MIA PaCa-2 or SW1990 pancreatic cancer cells:
EGFR inhibitor: Afatinib, KRAS-G12C inhibitor: AMG 510, KRAS-G12C inhibitor: MRTX849, KRAS-G12D inhibitor: MRTX1133, ERK1/2 inhibitor: LY3214996 and BVD-523, BRAF inhibitor: Encorafenib, pan-RAF inhibitor: LXH254, PRMT5 inhibitor: compound 24 of WO 2019116302, Type I PRMT inhibitor: GSK3368715, PI3K inhibitor: BYL719, FGFR inhibitor: Nintedanib, CDK4/6 inhibitor: Abemaciclib, and other chemotherapeutic agent: Gemcitabine.

Colony formation assay: MIA PaCa-2 cells or SW1990 cells were seeded at 500 cells per well or 1500 cells/well, respectively in 48 well tissue culture plate and cells were allowed to settle overnight (16 to 20 h). On the following day, cells were treated with various concentrations of targeted agents to generate IC₅₀ with or without increasing concentrations of SOS1 inhibitor (as depicted in the figures), and the assay plates were incubated under normal cell culture conditions. After 7 days of drug treatment, media was removed from each well and plates were washed with PBS. Cell colonies were stained with crystal violet solution for 2-5 min. Plate was then washed carefully under tap water and air dried. For quantitation, 200 μL destaining solution containing 10% Glacial acetic acid was added to each well and stained colonies were allowed to solubilize for 20-30 min on plate shaker. After solubilization, absorbance of the extracted stain was recorded in BioTek Synergy Neo U plate reader at 590 nm. Absorbance values were directly proportional to colony growth.

Compound 1, 2, 3, 3a, 3b, 4, 4a, 4b, 5, 6, 6a, 6b and 7 demonstrated significant potentiation of activity of these agents leading to inhibition of colony forming activity in MIA PaCa-2 or SW1990 pancreatic cancer cells.

### Example 9: In Vivo Efficacy Experiments

Compound 1 or Compound 4b were combined with AMG 510 in an in vivo efficacy study in MIA PaCa-2 human pancreatic cancer xenograft model in nude mice.

MIA PaCa-2 tumor fragments were implanted subcutaneously in the right flank region of nude mice. Tumor-bearing mice were randomized once the tumors reached an average volume of ˜141-142 mm³ (tumor volume range 72-242 mm³). The mice were divided into the following groups (n=10/group): Vehicle control, AMG 510 (10 mg/kg; q.d.), Compound 1 (30 mg/kg; b.i.d.), Compound 4b (30 mg/kg; b.i.d.). The tumor regression obtained for the combination of Compound I and AMG 510 was found to be 93.55±3.65% whereas the combination of AMG 510 and Compound 4b showed a tumor regression of 93.13±3.50%. As a single agent Compound 1 and Compound 4b showed a tumor growth inhibition of 63.82±8.32% and 65.71±7.41% respectively, whereas AMG 510, as a single agent showed a tumor regression of 39.43±15.22%.

Compound 4b was combined with Afatinib or Compound 24 of WO 2019116302 in an in vivo efficacy study in MIA PaCa-2 human pancreatic cancer xenograft model in nude mice.

MIA PaCa-2 tumor fragments were implanted subcutaneously in the right flank region of nude mice. Tumor-bearing mice were randomized once the tumors reached an average volume of ˜150-152 mm³ (tumor volume range 61-262 mm³). The mice were divided into the following groups (n=10/group): Vehicle control, Compound 4b (15 mg/kg; b.i.d.), Afatinib (12.5 mg/kg; q.d.) or Compound 24 of WO 2019116302 (1.0 mg/kg; b.i.d.).

The combination of Compound 4b with Afatinib or with Compound 24 of WO 2019116302 led to tumor growth inhibition of 85% and 75%, respectively. As a single agent, Compound 4b, Afatinib and Compound 24 of WO 2019116302 showed a tumor growth inhibition of 47%, 38% and 52%, respectively.

Compound 5 was tested in combination with Compound 24 of WO 2019116302 in vivo in MIA PaCa-2 xenograft model in nude mice.

20×10⁶ MIA PaCa-2 cells were injected subcutaneously in the presence of PBS and Matrigel in 1:1 ratio in nude mice. The tumor-bearing mice were randomized once the tumors reached an average volume of approximately 154-159 mm³ (Tumor volume range 107-248 mm³). The mice were divided into the following groups (n=7-8/group): Vehicle control and Compound 5 (50 mg/kg; b.i.d.).

The combination of Compound 5 with Compound 24 of WO 2019116302 led to tumor growth inhibition of 89%. As a single agent, Compound 5 and Compound 24 of WO 2019116302 showed a tumor growth inhibition of 59/o and 73%, respectively.

Compound 7 was combined with Afatinib (EGFR inhibitor), Compound 24 of WO 2019116302 (PRMT5 inhibitor) or Ulixertinib (ERK1/2 inhibitor) in an in vivo efficacy study in MIA PaCa-2 human pancreatic cancer xenograft model in nude mice.

MIA PaCa-2 tumor fragments were implanted subcutaneously in the right flank region of nude mice. Tumor-bearing mice were randomized once the tumors reached an average volume of ˜137-144 mm³ (tumor volume range 60-331 mm³). The mice were divided into the following groups (n=09/group): Vehicle control, Compound 7 (15 mg/kg; b.i.d.), Compound 7 (15 mg/kg; b.i.d.)+Afatinib (12.5 mg/kg; q.d.), Compound 7 (15 mg/kg; b.i.d.)+Compound 24 of WO 2019116302 (1 mg/kg; b.i.d.), and Compound 7 (15 mg/kg; b.i.d.)+Ulixertinib (25 mg/kg; b.i.d.).

The combination of Compound 7 with Afatinib or Compound 24 of WO 2019116302 or Ulixertinib led to 60.70%, 86.14% and 59.77% inhibition in tumor growth, respectively. As a single agent, Compound 7 showed 32.86% inhibition in tumor growth.

Compound 7 was combined with LXH254 (pan-RAF inhibitor) in an in vivo efficacy study in MIA PaCa-2 human pancreatic cancer xenograft model in nude mice.

MIA PaCa-2 tumor fragments were implanted subcutaneously in the right flank region of nude mice. Tumor-bearing mice were randomized once the tumors reached an average volume of ˜209-214 mm³ (tumor volume range 54-376 mm³). The mice were divided into the following groups (n=08/group): Vehicle control, Compound 7 (5 mg/kg; q.d.), LXH254 (50 mg/kg; b.i.d.), Compound 7 (5 mg/kg; q.d.)+LXH254 (50 mg/kg; b.i.d.).

The combination of Compound 7 with LXH254 led to 63.82%, inhibition in tumor growth. As a single agent, Compound 7 and LXH254 showed 39.82% and 34.96% inhibition in tumor growth respectively.

Compound 7 was combined with AMG 510 (KRAS G12C inhibitor) in an in vivo efficacy study in MIA PaCa-2 human pancreatic cancer xenograft model in nude mice.

MIA PaCa-2 tumor fragments were implanted subcutaneously in the right flank region of nude mice. Tumor-bearing mice were randomized once the tumors reached an average volume of ˜155-164 mm³ (tumor volume range 66-298 mm³). The mice were divided into the following groups (n=09/group): Vehicle control, AMG 510 (3 mg/kg; q.d.), Compound 7 (5 mg/kg; q.d.), Compound 7 (10 mg/kg; q.d.), Compound 7 (20 mg/kg; q.d.), Compound 7 (5 mg/kg; q.d.)+AMG 510 (3 mg/kg; q.d.), Compound 7 (10 mg/kg; q.d.)+AMG 510 (3 mg/kg; q.d.) and Compound 7 (20 mg/kg; q.d.)+AMG 510 (3 mg/kg; q.d.).

The combination of Compound 7 at dose 5, 10 and 20 mg/kg with AMG 510 led to tumor regression of 67.02% (Complete regression (CR)—3/9 mice), 79.69% (CR—5/9 mice) and 96.39% (CR—8/9 mice), respectively. As a single agent, AMG-510 showed 77.69% whereas Compound 7 at dose of 5, 10 and 20 mg/kg led to 30.40%, 43.42% and 52.71% inhibition in tumor growth respectively.

Compound 7 was combined with Adagrasib (KRAS G12C inhibitor) in an in vivo efficacy study in MIA PaCa-2 human pancreatic cancer xenograft model in nude mice. MIA PaCa-2 tumor fragments were implanted subcutaneously in the right flank region of nude mice. Tumor-bearing mice were randomized once the tumors reached an average volume of ˜163-165 mm3. The mice were divided into the following groups (n=9/group): Vehicle control, Adagrasib (8 mg/kg; q.d.) alone, Compound 7 (5 mg/kg; q.d.)+Adagrasib (8 mg/kg; q.d.), Compound 7 (10 mg/kg; q.d.)+Adagrasib (8 mg/kg; q.d.) and Compound 7 (20 mg/kg; q.d.)+Adagrasib (8 mg/kg; q.d.).

The combination of Compound 7 at dose level of 5, 10 & 20 mg/kg, q.d. in combination with Adagrasib (8 mg/kg; q.d.) led to 71.93%, 95.15% and 97.95% tumor growth inhibition, respectively. As a single agent, Adagrasib (8 mg/kg; q.d.) showed 58.12% inhibition in tumor growth.

1. A pharmaceutical combination for treating and/or preventing cancer comprising a SOS1 inhibitor of formula (I) or formula (II), its stereoisomer, or its pharmaceutical acceptable salt, and at least one additional active ingredient selected from a KRAS inhibitor; an EGFR inhibitor; an ERK1/2 inhibitor; a BRAF inhibitor; a pan-RAF inhibitor; a MEK inhibitor; a AKT inhibitor; a SHP2 inhibitor; protein arginine methyltransferases (PRMTs) inhibitor; a cyclin-dependent kinase (CDK) inhibitor; a FGFR inhibitor; a c-Met inhibitor; a RTK inhibitor; a non-receptor tyrosine kinase inhibitor; a histone methyltransferases (HMTs) inhibitor; a DNA methyltransferases (DNMTs) inhibitor; a Focal Adhesion Kinase (FAK) inhibitor; a Bcr-Abl tyrosine kinase inhibitor; a mTOR inhibitor; a PD1 inhibitor; a PD-L1 inhibitor; CTLA4 inhibitor; and chemotherapeutic agents; wherein the SOS1 inhibitor of formula (I) is, 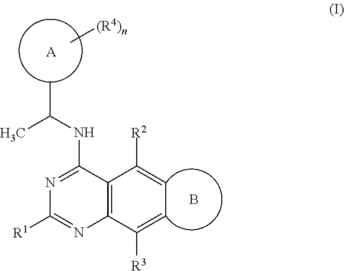
wherein,
Ring A is selected from aryl, heteroaryl, and heterocyclyl;
Ring B is selected from a substituted or unsubstituted 5 or 6 membered carbocyclic ring and a substituted or unsubstituted 5 or 6 membered heterocyclic ring containing 1 to 3 heteroatoms independently selected from S, O, and N;
when ring B is carbocyclic ring, it is substituted with 1 to 8 substituents independently selected from R^{c} and R^{d};
when ring B is heterocyclic ring, it is substituted with 1 to 7 substituents; when the heterocyclic ring B is substituted on a ring nitrogen atom, the heterocyclic ring B is substituted with substituents selected from R^{a} and R^{b}; and when the heterocyclic ring B is substituted on a ring carbon atom, the heterocyclic ring B is substituted with substituents selected from R^{c} and R^{d};
R^{a} and R^{b} are independently selected from hydrogen, —C(═O)R^{g}, —C(═O)NR^{h}(Rⁱ), substituted or unsubstituted alkyl, substituted or unsubstituted cycloalkyl, substituted or unsubstituted aryl, substituted or unsubstituted heteroaryl, and substituted or unsubstituted heterocyclyl;
R^{c} and R^{d} are independently selected from hydrogen, halogen, oxo, —C(═O)R^{g}, —NR^{h}(Rⁱ), —C(═O)NR^{h}(Rⁱ), —OR^{j}, substituted or unsubstituted alkyl, substituted or unsubstituted cycloalkyl, substituted or unsubstituted aryl, substituted or unsubstituted heteroaryl, and substituted or unsubstituted heterocyclyl; optionally R^{c} and R^{d} groups together with the carbon atom which they are attached forming a substituted or unsubstituted carbocyclic ring and substituted or unsubstituted heterocycle;
R¹ is selected from hydrogen, substituted or unsubstituted alkyl, and substituted or unsubstituted cycloalkyl.
R² and R³ are independently selected from hydrogen, halogen, cyano, substituted or unsubstituted alkyl, and substituted or unsubstituted cycloalkyl;
R⁴ is selected from halogen, cyano, —NR^{e}R^{f}, —OR^{j}, —C(═O)R^{g}, —C(═O)NR^{h}(Rⁱ), substituted or unsubstituted alkyl, substituted or unsubstituted cycloalkyl, cycloalkyl substituted with substituted or unsubstituted alkyl, substituted or unsubstituted aryl, substituted or unsubstituted heteroaryl, substituted or unsubstituted heterocyclyl, and heterocyclyl substituted with substituted alkyl;
R^{e} and R^{f} are independently selected from hydrogen, —C(═O)R^{g}, —C(═O)NR^{h}(Rⁱ), substituted or unsubstituted alkyl, alkyl substituted with substituted or unsubstituted heterocyclyl, substituted or unsubstituted cycloalkyl, substituted or unsubstituted aryl, substituted or unsubstituted heteroaryl, and substituted or unsubstituted heterocyclyl;
R^{g} is selected from substituted or unsubstituted alkyl, substituted or unsubstituted cycloalkyl, substituted or unsubstituted aryl, substituted or unsubstituted heteroaryl, and substituted or unsubstituted heterocyclyl;
R^{h} and Rⁱ are independently selected from hydrogen, substituted or unsubstituted alkyl, substituted or unsubstituted cycloalkyl, and substituted or unsubstituted heterocyclyl;
optionally R^{h} and Rⁱ groups together with the nitrogen atom to which they are attached forming a substituted or unsubstituted heterocycle;
R^{j} is selected from hydrogen, substituted or unsubstituted alkyl, alkyl substituted with substituted or unsubstituted cycloalkyl, and substituted or unsubstituted cycloalkyl;
‘n’ is an integer selected from 0, 1, 2, and 3;
when an alkyl group is substituted, it is substituted with 1 to 5 substituents independently selected from oxo (═O), halogen, cyano, cycloalkyl, aryl, heteroaryl, heterocyclyl, —OR⁵, —C(═O)OH, —C(═O)O(alkyl), —NR⁶R^{6a}, —NR⁶C(═O)R⁷, and —C(═O)NR⁶R^{6a};
when an cycloalkyl group is substituted, it is substituted with 1 to 4 substituents independently selected from oxo (═O), halogen, alkyl, hydroxyalkyl, cyano, aryl, heteroaryl, heterocyclyl, —OR⁵, —C(═O)OH, —C(═O)O(alkyl), —NR⁶R^{6a}, —NR⁶C(═O)R⁷, and —C(═O)NR⁶R^{6a};
when the aryl group is substituted, it is substituted with 1 to 4 substituents independently selected from halogen, nitro, cyano, alkyl, perhaloalkyl, cycloalkyl, heterocyclyl, heteroaryl, —OR⁵, —NR⁶R^{6a}, —NR⁶C(═O)R⁷, —C(═O)R⁷, —C(═O)NR⁶R^{6a}, —SO₂-alkyl, —C(═O)OH, —C(═O)O-alkyl, and haloalkyl;
when the heteroaryl group is substituted, it is substituted with 1 to 4 substituents independently selected from halogen, nitro, cyano, alkyl, haloalkyl, perhaloalkyl, cycloalkyl, heterocyclyl, aryl, heteroaryl, —OR⁵, —NR⁶R^{6a}, —NR⁵C(═O)R⁷, —C(═O)R⁷, —C(═O)NR⁶R^{6a}, —SO₂-alkyl, —C(═O)OH, and —C(═O)O-alkyl;
when the heterocycle group is substituted, it is substituted either on a ring carbon atom or on a ring hetero atom, and when it is substituted on a ring carbon atom, it is substituted with 1 to 4 substituents independently selected from oxo (═O), halogen, cyano, alkyl, alkoxyalkyl, hydroxyalkyl, cycloalkyl, perhaloalkyl, —OR⁵, —C(═O)NR⁶R^{6a}, —C(═O)OH, —C(═O)O-alkyl, —N(H)C(═O)(alkyl), —N(H)R⁶, and —N(alkyl)₂; and when the heterocycle group is substituted on a ring nitrogen, it is substituted with substituents independently selected from alkyl, cycloalkyl, aryl, heteroaryl, —SO₂(alkyl), —C(═O)R⁷, and —C(═O)O(alkyl); when the heterocycle group is substituted on a ring sulfur, it is substituted with 1 or 2 oxo (═O) group(s);
R⁵ is selected from hydrogen, alkyl, perhaloalkyl, and cycloalkyl;
R⁶ and R^{6a} are each independently selected from hydrogen, alkyl, and cycloalkyl;
or R⁶ and R^{6a} together with nitrogen to which they are attached form a heterocyclyl ring; and
R⁷ is selected from alkyl and cycloalkyl;
and wherein the SOS1 inhibitor of formula (II), its tautomeric form, its stereoisomer, its pharmaceutical acceptable salt, its polymorph, or solvate thereof, 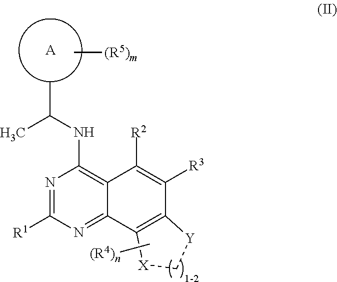
wherein
Ring A is selected from aryl, heteroaryl, and heterocyclyl;
‘ [symbol] ’ is either a single bond or double bond;
X and Y are independently selected from C, O, and NR^{c}, provided that both X and Y cannot be O at the same time;
R¹ is selected from hydrogen and substituted or unsubstituted alkyl;
R² is selected from hydrogen, halogen, alkyl, and cycloalkyl;
R³ is selected from —OR⁶, —NR^{a}R^{b}, substituted or unsubstituted alkyl, substituted or unsubstituted cycloalkyl, alkyl substituted with substituted or unsubstituted heterocyclyl, substituted or unsubstituted heteroaryl, and substituted or unsubstituted heterocyclyl;
R⁴ is selected from oxo and substituted or unsubstituted alkyl;
R⁵ is selected from halogen, cyano, —NR^{c}R^{d}, substituted or unsubstituted alkyl, —C(═O) substituted or unsubstituted alkyl, substituted or unsubstituted cycloalkyl, substituted or unsubstituted aryl, and substituted or unsubstituted heteroaryl; optionally two R⁵ groups attached to the adjacent carbon atoms forming substituted or unsubstituted heterocycle;
R⁶ is selected from substituted or unsubstituted alkyl, substituted or unsubstituted heterocyclyl, and alkyl substituted with substituted heterocyclyl;
R^{a} and R^{b} are independently selected from hydrogen, substituted or unsubstituted alkyl, and substituted or unsubstituted heterocyclyl;
R^{c} and R^{d} are independently selected from hydrogen and alkyl;
m is an integer selected from 0, 1, 2, and 3;
n is an integer selected from 0, 1, 2, 3, and 4;
when an alkyl group is substituted, it is substituted with 1 to 5 substituents independently selected from oxo (═O), halogen, cyano, cycloalkyl, aryl, heteroaryl, heterocyclyl, —OR⁷, —C(═O)OH, —C(═O)O(alkyl), —NR⁸R^{8a}, —NR^{g}C(═O)R⁹, and —C(═O)NR⁸R^{8a};
when an cycloalkyl group is substituted, it is substituted with 1 to 4 substituents independently selected from oxo (═O), halogen, alkyl, hydroxyalkyl, cyano, aryl, heteroaryl, heterocyclyl, —OR⁷, —C(═O)OH, —C(═O)O(alkyl), —NR⁸R^{8a}, —NR⁸C(═O)R⁹, and —C(═O)NR⁸R^{8a};
when the aryl group is substituted, it is substituted with 1 to 4 substituents independently selected from halogen, nitro, cyano, alkyl, haloalkyl, perhaloalkyl, cycloalkyl, heterocyclyl, heteroaryl, —OR⁷, —NR⁸R^{8a}, —NR⁸C(═O)R⁹, —C(═O)R⁹, —C(═O)NR⁸R^{8a}, —SO₂-alkyl, —C(═O)OH, and —C(═O)O-alkyl;
when the heteroaryl group is substituted, it is substituted with 1 to 4 substituents independently selected from halogen, nitro, cyano, alkyl, haloalkyl, perhaloalkyl, cycloalkyl, heterocyclyl, aryl, heteroaryl, —OR⁷, —NR⁸R^{8a}, —NR⁷C(═O)R⁹, —C(═O)R⁹, —C(═O)NR⁸R^{8a}, —SO₂-alkyl, —C(═O)OH, and —C(═O)O-alkyl;
when the heterocycle group is substituted, it is substituted either on a ring carbon atom or on a ring hetero atom, and when it is substituted on a ring carbon atom, it is substituted with 1 to 4 substituents independently selected from oxo (═O), halogen, cyano, alkyl, haloalkyl, alkoxyalkyl, hydroxyalkyl, cycloalkyl, perhaloalkyl, —OR⁷, —C(═O)NR⁸R^{8a}, —C(═O)OH, —C(═O)O-alkyl, —N(H)C(═O)(alkyl), —N(H)R⁸, and —N(alkyl)₂; and when the heterocycle group is substituted on a ring nitrogen, it is substituted with substituents independently selected from alkyl, haloalkyl, cycloalkyl, aryl, heteroaryl, —SO₂(alkyl), —C(═O)R⁹, and —C(═O)O(alkyl); when the heterocycle group is substituted on a ring sulfur, it is substituted with 1 or 2 oxo (═O) group(s);
R⁷ is selected from hydrogen, alkyl, perhaloalkyl, and cycloalkyl;
R⁸ and R^{8a} are each independently selected from hydrogen, alkyl, and cycloalkyl; and
R⁹ is selected from alkyl and cycloalkyl.

2. The pharmaceutical combination of claim 1,
wherein the SOS1 inhibitor of is selected from the group consisting of:
(R)-4-((1-(3-(1,1-Difluoro-2-hydroxy-2-methylpropyl)-2-fluorophenyl)ethyl)amino)-2,6,8,8-tetramethyl-6,8-dihydro-7H-pyrrolo[2,3-g]quinazolin-7-one (Compound 1);
R/S)-4-((1-(3-(1,1-difluoro-2-hydroxy-2-methylpropyl)phenyl)ethyl)amino)-2,6,8,8-tetramethyl-6,8-dihydro-7H-pyrrolo[2,3-g]quinazolin-7-one (Compound 2);
-(((R)-1-(3-((R&S)-1,1-Difluoro-2,3-dihydroxy-2-methylpropyl)-2-fluorophenyl) ethyl)amino)-2,6,8,8-tetramethyl-6,8-dihydro-7H-pyrrolo[2,3-g]quinazolin-7-one (Compound 3);
4-(((R)-1-(3-((R/S)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3a);
4-(((R)-1-(3-((S/R)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3b);
(R&S)-4-(((R)-1-(3-(1,1-difluoro-2-hydroxy-2-methylpropyl)-2-fluorophenyl) ethyl) amino)-8-methoxy-2,6,8-trimethyl-6,8-dihydro-7H-pyrrolo[2,3-g]quinazolin-7-one (Compound 4);
(S/R)-4-(((R)-1-(3-(1,1-difluoro-2-hydroxy-2-methylpropyl)-2-fluorophenyl)ethyl)amino)-8-methoxy-2,6,8-trimethyl-6H-pyrrolo[2,3-g]quinazolin-7(8H)-one (Compound 4a);
(R/S)-4-(((R)-1-(3-(1,1-difluoro-2-hydroxy-2-methylpropyl)-2-fluorophenyl)ethyl) amino)-8-methoxy-2,6,8-trimethyl-6H-pyrrolo[2,3-g]quinazolin-7(8H)-one (Compound 4b);
(R)-5-(4-((1-(3-amino-5-(trifluoromethyl) phenyl) ethyl) amino)-2-methyl-8,9-dihydro-7H-cyclopenta[h]quinazolin-6-yl)-1-methylpyridin-2(1H)-one (Compound 5);
(R&S)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6);
(S/R)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6a);
(R/S)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6b); and
(S)-4-(((R)-1-(3-amino-5-(trifluoromethyl) phenyl) ethyl)amino)-8-methoxy-2,6,8-trimethyl-6,8-dihydro-7H-pyrrolo[2,3-g]quinazolin-7-one (Compound 7);
or a pharmaceutically acceptable salt, a hydrate, or a stereoisomer thereof.

3. The pharmaceutical combination claim 1, wherein the additional active ingredient is one or more selected from a KRAS inhibitor, KRASG12C inhibitor, and KRAS-G12D inhibitors.

4. The pharmaceutical combination of claim 3, wherein the additional active ingredient is selected from Sotorasib (AMG510), MRTX849, JDQ443, LY-3537982, JNJ-74699157, JAB-21822, GDC-6036, D-1553, YL-15293, BI-1823911, BEBT-607, MRTX1133 and BI-2852.

5. The pharmaceutical combination of claim 1, wherein the additional active ingredient is an EGFR inhibitor.

6. The pharmaceutical combination of claim 5, wherein the EGFR inhibitor is selected from Afatinib, Osimertinib, Erlotinib and Gefitinib

7. The pharmaceutical combination of claim 1, wherein the additional active ingredient is an ERK1/2 inhibitor.

8. The pharmaceutical combination of claim 7, wherein the ERK1/2 inhibitor is selected from LY-3214996, BVD-523 (Ulixertinib), MK-8353 and ravoxertinib.

9. The pharmaceutical combination of claim 1, wherein the additional active ingredient is a pan-RAF inhibitor.

10. The pharmaceutical combination of claim 9, wherein the pan-RAF inhibitor is selected from Dabrafenib, Regorafenib Encorafenib, and LXH254.

11. The pharmaceutical combination of claim 1, wherein the additional active ingredient is selected from an AKT inhibitor.

12. The pharmaceutical combination of claim 11, wherein the AKT inhibitor is selected from GSK690693, AZD5363 and Ipatasertib.

13. The pharmaceutical combination of claim 1, wherein the additional active ingredient is a SHP2 inhibitor.

14. The pharmaceutical combination of claim 13, wherein the SHP2 inhibitor is TNO155, JAB-3068, RMC-4630 or RLY-1971 or any other agent that inhibits activity of the SHP2 phosphatase.

15. The pharmaceutical combination of claim 1, wherein the additional active ingredient is a PRMT inhibitor.

16. The pharmaceutical combination of claim 15, wherein the PRMT inhibitor is JNJ-64619178, PF-06939999, GSK-3326595, PRT543, PRT811, MS023, GSK3368715, Type I PRMT inhibitors or (1S,2R,5R)-3-(2-(2-amino-3-chloro-5-fluoroquinolin-7-yl)ethyl)-5-(4-amino-7H-pyrrolo[2,3-d]pyrimidin-7-yl)cyclopent-3-ene-1,2-diol (Compound 24 WO 2019116302).

17. The pharmaceutical combination of claim 1, wherein the additional active ingredient is a PI3K inhibitor.

18. The pharmaceutical combination of claim 17, wherein PI3K inhibitor is selected from Alpelisib, Copanlisib, Duvelisib, BEZ-235, Gedatolisib, Buparlisib.

19. The pharmaceutical combination of claim 1, wherein the additional active ingredient is a CDK4/6 inhibitor.

20. The pharmaceutical combination of claim 19, wherein CDK4/6 inhibitor is Abemaciclib.

21. The pharmaceutical combination of claim 1, wherein the additional active ingredient is a FGFR inhibitor.

22. The pharmaceutical combination of claim 21, wherein the FGFR inhibitor is selected from Dovitinib, AZD4547, BGJ398 and JNJ 42756493.

23. The pharmaceutical combination of claim 1, wherein the additional active ingredient is a c-Met inhibitor.

24. The pharmaceutical combination gf claim 23, wherein the c-Met inhibitor is selected from Tivantinib, Cabozantinib, Crizotinib and Capmatinib.

25. The pharmaceutical combination of claim 1, wherein the additional active ingredient is a Bcr-Abl kinase inhibitor.

26. The pharmaceutical combination of claim 25, wherein the Bcr-Abl kinase inhibitor is selected from imatinib, Dasatinib, nilotinib and ponatinib.

27. The pharmaceutical combination of claim 1, wherein the additional active ingredient is a PD1 inhibitor.

28. The pharmaceutical combination of claim 27, wherein the PD1 inhibitor is selected from Pembrolizumab and Nivolumab.

29. The pharmaceutical combination of claim 1, wherein the additional active ingredient is selected from a PD-L1 inhibitor.

30. The pharmaceutical combination of claim 29, wherein the PD-L1 inhibitor is selected from Atezolizumab and Avelumab.

31. The pharmaceutical combination of claim 1, wherein the additional active ingredient is a CTLA-4 inhibitor.

32. The pharmaceutical combination of claim 31, wherein the CTLA-4 inhibitor is Ipilimumab.

33. The pharmaceutical combination of claim 1, wherein the additional active ingredient is a gemcitabine, topotecan, irinotecan, paclitaxel, cisplatin, carboplatin, doxorubicin or any other agent that is classified as chemotherapeutic.

34. The pharmaceutical combination of claim 1, wherein, an additional therapeutic agent is selected from EGFR inhibitor, KRAS G12C inhibitor, ERK1/2 inhibitor, RAF inhibitor, PRMT5 inhibitor, pan-RAF inhibitor, SHP2 inhibitor, PI3K inhibitor, Type I PRMT inhibitor, FGFR inhibitor, CDK4/6 inhibitor, and Chemotherapeutic agent.

35. The pharmaceutical combination of claim 1, wherein, an additional therapeutic agent is selected from Afatinib, AMG510, LY3214996, BVD-523, Encorafenib, Compound 24 of WO 2019116302, LXH254, TNO155, MRTX849, MRTX1133, BYL-719, GSK3368715, Nintedanib, Abemaciclib, and Gemcitabine.

36. The pharmaceutical combination of claim 1, wherein the SOS1 inhibitor is selected from (R)-4-((1-(3-(1,1-Difluoro-2-hydroxy-2-methylpropyl)-2-fluorophenyl)ethyl)amino)-2,6,8,8-tetramethyl-6,8-dihydro-7H-pyrrolo[2,3-g]quinazolin-7-one (Compound 1), (R/S)-4-((1-(3-(1,1-difluoro-2-hydroxy-2-methylpropyl)phenyl)ethyl)amino)-2,6,8,8-tetramethyl-6,8-dihydro-7H-pyrrolo[2,3-g]quinazolin-7-one (Compound 2), 4-(((R)-1-(3-((R/S)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3a), (R/S)-4-(((R)-1-(3-(1,1-difluoro-2-hydroxy-2-methylpropyl)-2-fluorophenyl)ethyl) amino)-8-methoxy-2,6,8-trimethyl-6H-pyrrolo[2,3-g]quinazolin-7(8H)-one (Compound 4b), (R)-5-(4-((1-(3-amino-5-(trifluoromethyl) phenyl) ethyl) amino)-2-methyl-8,9-dihydro-7H-cyclopenta[h]quinazolin-6-yl)-1-methylpyridin-2(1H)-one (Compound 5), (S/R)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6a), and (S)-4-(((R)-1-(3-amino-5-(trifluoromethyl) phenyl) ethyl)amino)-8-methoxy-2,6,8-trimethyl-6,8-dihydro-7H-pyrrolo[2,3-g]quinazolin-7-one (Compound 7); and an additional therapeutic agent is selected from EGFR inhibitor, KRAS G12C inhibitor, ERK1/2 inhibitor, RAF inhibitor, PRMT5 inhibitor, pan-RAF inhibitor, SHP2 inhibitor, PI3K inhibitor, Type I PRMT inhibitor, FGFR inhibitor, CDK4/6 inhibitor, and Chemotherapeutic agent.

37. The pharmaceutical combination of claim 1, wherein the SOS1 inhibitor is selected from (R)-4-((1-(3-(1,1-Difluoro-2-hydroxy-2-methylpropyl)-2-fluorophenyl)ethyl)amino)-2,6,8,8-tetramethyl-6,8-dihydro-7H-pyrrolo[2,3-g]quinazolin-7-one (Compound 1), (R/S)-4-((1-(3-(1,1-difluoro-2-hydroxy-2-methylpropyl)phenyl)ethyl)amino)-2,6,8,8-tetramethyl-6,8-dihydro-7H-pyrrolo[2,3-g]quinazolin-7-one (Compound 2), 4-(((R)-1-(3-((R/S)-1,1-difluoro-2,3-dihydroxy-2-methylpropyl)-2-fluorophenyl)ethyl)amino)-2,6,8,8-tetramethyl-6,8-dihydro-7H-pyrrolo[2,3-g]quinazolin-7-one (Compound 3a), (R/S)-4-(((R)-1-(3-(1,1-difluoro-2-hydroxy-2-methylpropyl)-2-fluorophenyl)ethyl) amino)-8-methoxy-2,6,8-trimethyl-6H-pyrrolo[2,3-g]quinazolin-7(8H)-one (Compound 4b), (R)-5-(4-((1-(3-amino-5-(trifluoromethyl) phenyl) ethyl) amino)-2-methyl-8,9-dihydro-7H-cyclopenta[h]quinazolin-6-yl)-1-methylpyridin-2(1H)-one (Compound 5), (S/R)-4-(((R)-1-(3-(1,1-difluoro-2-hydroxy-2-methylpropyl)-2-fluorophenyl)ethyl)amino)-6-methoxy-2,6,8-trimethyl-6,8-dihydro-7H-pyrrolo[3,2-g]quinazolin-7-one (Compound 6a), and (S)-4-(((R)-1-(3-amino-5-(trifluoromethyl) phenyl) ethyl)amino)-8-methoxy-2,6,8-trimethyl-6,8-dihydro-7H-pyrrolo[2,3-g]quinazolin-7-one (Compound 7); and an additional therapeutic agent is selected from Afatinib, AMG510, LY3214996, BVD-523, Encorafenib, Compound 24 of WO 2019116302, LXH254, TNO155, MRTX849, MRTX1133, BYL-719, GSK3368715, Nintedanib, Abemaciclib, and Gemcitabine.

38. A pharmaceutical combination comprising a SOS1 inhibitor (S)-4-(((R)-1-(3-amino-5-(trifluoromethyl) phenyl) ethyl)amino)-8-methoxy-2,6,8-trimethyl-6,8-dihydro-7H-pyrrolo[2,3-g]quinazolin-7-one (Compound 7), and an additional therapeutic agent selected from Afatinib, AMG510, LY3214996, BVD-523, Encorafenib, LXH254, TNO155, MRTX849, MRTX1133, BYL-719, GSK3368715, Nintedanib, Abemaciclib, and Gemcitabine.

39. The pharmaceutical combination of claim 1, wherein the SOS1 inhibitor is administered simultaneously, concurrently, sequentially, successively, alternately or separately with the additional active ingredient.

40. A method of treating or preventing cancer, wherein the method comprising administering to the subject in need with pharmaceutical combination of claim 1.

41. The method of claim 40, wherein the cancer is glioblastoma multiforme, prostate cancer, pancreatic cancer, mantle cell lymphoma, non-Hodgkin's lymphomas and diffuse large B-cell lymphoma, acute myeloid leukemia, acute lymphoblastic leukemia, multiple myeloma, non-small cell lung cancer, small cell lung cancer, breast cancer, triple negative breast cancer, gastric cancer, colorectal cancer, ovarian cancer, bladder cancer, hepatocellular cancer, melanoma, sarcoma, oropharyngeal squamous cell carcinoma, chronic myelogenous leukemia, epidermal squamous cell carcinoma, nasopharyngeal carcinoma, neuroblastoma, endometrial carcinoma, head and neck cancer, cervical cancer, or cancers harboring overexpression, amplification of wild type KRAS, NRAS or HRAS, cancers having amplification, overexpression or mutation of KRAS, NRAS, or HRAS, cancers harboring KRAS mutations, cancers harboring NRAS mutations.
